(19) United States
(12) Patent Application Publication
(10) Pub. No.: US 2011/0129825 A1
(43) Pub. Date: Jun. 2, 2011

(54) COMPOSITIONS, METHODS AND SYSTEMS FOR THE SIMULTANEOUS DETERMINATION OF PARENTAGE, IDENTITY, SEX, GENOTYPE AND/OR PHENOTYPE AND BREED DETERMINATION IN ANIMALS

(76) Inventor: Melba Stinnett Ketchum, Timpson, TX (US)

(21) Appl. No.: 12/733,032

(22) PCT Filed: Aug. 3, 2008
(86) PCT No.: PCT/US08/72044

§ 371 Date: Feb. 3, 2011

Related U.S. Application Data
(60) Provisional application No. 60/935,298, filed on Aug. 3, 2007.

(51) Int. Cl. C12Q 1/68; C12M 1/34

(52) U.S. Cl. 435/6.11; 435/287.2

(57) ABSTRACT

The invention provides for a universal genetic evaluation system capable of simultaneously determining multiple genetic characteristics in domestic and wild animals. In particular, the invention provides for the use of polymorphisms, such as single nucleotide polymorphisms (SNPs), insertions, deletions, inversions, and/or other mutations within gene sequences, as determinants of genetic characteristics, such as parentage, identity, sex, genotype and/or phenotype. The universal genetic evaluation system is utilized to simultaneously determine multiple genetic characteristics in horses and wild horses, dogs and wild canids, cats, goats and wild goats, sheep and wild sheep, cattle, bison, deer (cervidae), donkeys, mules, swine and wild swine, camelids and wild camelids, other domestic and certain species of wild animals (deer, elk, red deer, antelope, caribou and reindeer, moose and other exotic deer and antelope species), birds (including pet birds and commercial bird species), reptiles, amphibians, fish and rodents, concurrently for each species.

### CROSS REFERENCE TO RELATED APPLICATIONS

The present invention claims benefit of priority to U.S. patent application Ser. No. 60/935,298 filed on Aug. 3, 2007, the contents of which are herein incorporated by reference in their entirety.

### FIELD OF THE INVENTION

The invention relates to a universal genetic evaluation system capable of simultaneously determining multiple genetic characteristics in domestic and wild animals. In particular, the invention provides for the concurrent detection of polymorphisms, such as single nucleotide polymorphisms (SNPs), insertions and/or deletions and other mutations within gene sequences, as determinants of genetic characteristics, such as parentage, identity, sex, genotype and/or phenotype and breed determination, and providing corresponding profiles.

### BACKGROUND OF THE INVENTION

The present invention provides for a universal genetic evaluation system capable of simultaneously determining multiple genetic characteristics in domestic animal. This universal system for identification and determination of key characteristics of individual animals maximizes their individual potential performance and traits as well as health and facilitates management and care of individual animals. The invention methods allow predictive (predisposition) diagnostics, character and trait determination such that nutritional therapies and pharmaceutical therapeutics can be administered to domestic animals when and if appropriate. Traits determined by the invention can be utilized to promote selective breeding to increase the value of the animals tested. The methods of the invention provide systems to collect, record, analyze and store data associated with multiple genetic characteristics in individual animals so that the data is usable to improve future performance, desirable traits and health of animals. The methods and systems of the present invention utilize information regarding genetic diversity among domestic and wild animals, particularly single nucleotide polymorphisms (SNPs), insertions, deletions, inversions and other mutations, and then correlate the presence of SNPs, insertions, deletions and other mutations of selected nucleotide marker sequences with important characteristics such as parentage, identity, sex, genotype and phenotype of domestic and wild animals.

The present invention is based, in part, on the discovery of domestic and wild animal markers containing mutations, including but not limited to, single nucleotide polymorphisms (SNP), insertions, deletions or inversions that can be utilized to identify individual animals, determine or verify parentage of a single animal from any breed, and predict or determine phenotype and/or genotype. Specifically, the present invention provides compositions, methods and systems for the identification of at least two characteristics, where the characteristics are parentage, breed, identity as well as forensic identity, sex, genotype and/or phenotype. These compositions, methods and systems aid in management of individual animals or groups of animals to maximize their individual potential performance and health, and are important with respect to livestock evaluation. Compositions, methods and systems of the present invention utilized to determine parentage and identity can be used to:
1) assign or verify parentage in disputed cases or as a quality control check for breed registries or for breed certification. These panels are currently utilized by domestic animal breed registries for verifying parentage of a defined set of parents and progeny;
2) match and verify the identity of a lost or stolen animal or to verify the identity of unknown evidentiary samples when compared to a known animal sample. When combined with a database of genotypes and animals, the panel can be used to match unknown animals to itself, if a genotype has been previously recorded, or to parents and siblings;
3) verify the identity of a cloned animal or frozen or split and/or cloned embryo;
4) verify the identity of banked and/or frozen semen, or verify cultured cell lines; and
5) link an known animal, animal hair or animal biological samples to a crime scene evidentiary sample for forensic applications.

DNA analysis provides a powerful tool for determining the parentage, breed, identity and/or phenotype of individual animals. Microsatellite marker panels have been developed for cattle (Sherman et al., Anim Genet. 35(3):220-6; Heyen et al., Arnim Genet. 28(1):21-27) and canine (See e.g., U.S. Pat. No. 5,874,217; Ostrander et al., Mammalian Genome, 6: 192-195; Franscisco et al., Mammalian Genome 7:359-362) that are highly polymorphic and amenable to standardization among laboratories performing these tests. However, microsatellite scoring requires considerable human oversight and microsatellite markers have high mutation rates. Single nucleotide polymorphisms (SNP) have also been utilized because of the ease of scoring, low cost assay development and high-throughput capability. There have been limited studies to evaluate the usefulness of SNP markers in small populations of animals (Heaton et al., Mamm Genome. 13(5):272-81; Werner et al., Anim. Genet. 35(1):44-9). In addition, the utilization of SNPs alone does not provide coverage for certain important nucleotide marker polymorphisms of interest.

Parentage and identity panels are the first applied technology of using genomic analysis to begin managing domestic animals. For example, panels have been developed utilizing microsatellite marker panels (DeNise et al., 2004. Anim. Genetics. 35(1): 14-17; Halverson et al., 1995. U.S. Pat. No. 5,874,217; Ostrander et al., 1993. Genomics 16: 207-213, Ostrander et al., 1995. Mammalian Genome, 6: 192-195; Franscisco et al., 1996. Mammalian Genome 7:359-362.

Compared with other types of DNA markers, single nucleotide polymorphisms (SNPs) are attractive because they are abundant, genetically stable, and amenable to high-throughput automated analysis. In animal husbandry and the management of health and performance, one challenge has been the development of a cost-efficient system to simultaneously identify parentage, breed, identity and phenotype. Another challenge has been the development of a system that can be applied to more than genera or species of animal, e.g., a universal system that can be utilized to identify parentage, breed, identity and phenotype in horse, cattle, dogs, cats, sheep, goat, bison, deer, elk, antelope, caribou, reindeer, moose, donkeys, mules, swine, camelids and other domestic and wild animals. A further challenge has been the identification of a minimal set of SNPs with sufficient power to identify parentage, identity, sex, genotype and phenotype simultaneously in one species of animal, and a minimal set of SNPs with sufficient power to identify parentage, identity, sex, genotype and phenotype in more than one species of animal.

Accordingly, there remains a need in the art for compositions, methods and systems that provide for cost-efficient analysis where at least two characteristics selected from the group consisting of parentage, identity, sex, genotype and phenotype can be simultaneously identified in an animal, or more than one species of animal. In addition, there remains a need in the art for compositions, methods and systems that are capable of providing this type of analysis by utilizing various polymorphic nucleotide marker sequences, including nucleotide marker sequences have single nucleotide polymorphisms (SNPs), insertions and/or deletions or other mutations at their polymorphic sites.

### BRIEF SUMMARY OF THE INVENTION

The present invention provides a method for simultaneously identifying a plurality of polymorphisms in a nucleic acid sample isolated from an animal comprising the steps of: (a) placing said nucleic acid sample in at least two recesses of an assay plate; (b) hybridizing said nucleic acid sample to a pair of forward and reverse primers; (c) contacting said nucleic acid sample with a first oligonucleotide probe and with a second oligonucleotide probe; (c) performing PCR amplification; and (d) detecting the presence of said plurality of polymorphisms in said nucleic acid sample.

In specific embodiments of the invention, the first oligonucleotide probe is capable of detecting a first allele of a nucleotide marker sequence and the second oligonucleotide probe is capable of detecting a second allele of a nucleotide marker sequence; wherein the nucleotide marker sequence is any one of the nucleotide marker sequences as set forth in Tables 1-11; and wherein said nucleotide marker sequence correlates with at least one of the characteristics of an animal selected from the group consisting of: (i) parentage; (ii) identity; (iii) sex (iv) genotype and (v) phenotype; and wherein said method is capable of simultaneously identifying at least two characteristics of said animal selected from the group consisting of: (i) parentage; (ii) identity; (iii) sex (iv) genotype and (v) phenotype.

In certain embodiments of the invention, the plurality of polymorphisms correlates with all five characteristics. In other embodiments of the invention, the plurality of polymorphisms is simultaneously identified in more than one nucleic acid sample, where each of the nucleic acid samples can be isolated from more than one individual animal of the same species, or different species.

In other embodiments of the invention the nucleic acid sample is isolated from an animal, where the animal is of a family selected from the group consisting of Equidae, Bovidae, Canidae, and Felidae. In further embodiments, animals of the family Bovidae are of a species selected from the group consisting of Bos, Ovis, and Capra. In further embodiments, animals of the family Equidae are of a species selected from the group consisting of Equus. In further embodiments, animals of the family Canidae are of a species selected from the group consisting of Canis. In further embodiments, animals of the family Felidae are of a species selected from the group consisting of Felis.

In other embodiments of the invention, the plurality of polymorphisms comprises between about 20 and about 10,000 polymorphisms and extending up to whole genome analysis, between about 20 and about 3000 polymorphisms, between about 20 and 200 polymorphisms. In further embodiments, the plurality of polymorphisms comprises about 60, 100, 3000, 6000 or 9000 polymorphisms, about 64, 128, 3072, 6344 or 9216 polymorphisms, or about 20, 30, 40, 50, 60, 70, 80, 90, 100, 110, 120, 130, 140, 150, 160, 170, 180, 190, or 200 polymorphisms.

In preferred embodiments, the plurality of polymorphisms comprises the polymorphisms associated with each of the nucleotide marker sequence according to Tables 2, 4, 6, 8 and/or 11.

In certain other embodiments, each of the primers of the invention is about 8 to about 30 nucleotides in length.

In certain embodiments of the invention, the phenotype is a trait. In further embodiments, the trait is selected from the group consisting of coat color, hair color, hair length, eye color, marbling, tenderness, quality grade, muscle content, fat thickness, feed efficiency, red meat yield, average daily weight gain, disease resistance, disease susceptibility, feed intake, protein content, bone content, maintenance energy requirement, mature size, amino acid profile, fatty acid profile, milk production, a milk quality susceptibility to the buller syndrome, stress susceptibility and response, temperament, digestive capacity, production of calpain, caplastatin and myostatin, pattern of fat deposition, ribeye area, fertility, ovulation rate, conception rate, fertility, and susceptibility to infection with and shedding of pathogens. In certain other embodiments, the trait is a coat color is selected from the group consisting of cream, silver, tobiano, sabino, agouti, chestnut, brown, dilution, melanistic mask, albinism, recessive black, points, Burmese shading, cinnamon, red, and merle.

In certain embodiments of the invention, the phenotype correlates with a disease. In further embodiments, the disease is selected from the group consisting of Lethal White Overo syndrome (LWO), Glycogen Branching Enzyme deficiency (GBE1), junctional epidermolysis bullosa (JEB), Severe Combined Immune Deficiency Syndrome (SCID), and Hyperkalemic Periodic Paralysis (HYPP). In additional embodiments, the disease is selected from the group consisting of congenital myotonia, muscular dystrophy, globoid cell leukodystrophy, GM-gangliosidosis, Hemophilia B, hereditary cataracts, phosphofructokinase deficiency, thrombasthenic thrombopathia, retinal dystrophy, type-2 von Willebrand's disease, and Type III von Willebrand. In certain other embodiments, the disease is selected from the group consisting of hypertrophic cardiomyopathy, polycystic kidney disease and mucopolysaccharidosis.

In certain embodiments of the invention, each of the oligonucleotide probes is detectably labeled, for example, with a fluorescent label, where the fluorescent label can be selected from the group consisting of ROX, VIC®, HEX, NED and FAMT™.

In further embodiments, the assay plate comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 arrays. In certain other embodiments, the characteristics are identified using a single array, and/or the plurality of polymorphisms is simultaneously identified using one, two or three assay plates.

In certain other embodiments, the method of the invention provides for a forward primer that is capable of hybridizing to a region within a nucleotide marker sequence that is about 30 to about 60 nucleotides upstream of the polymorphic site present within said nucleotide marker sequence. In further embodiments, the method of the invention provides for a reverse primer that is capable of hybridizing to a region within a nucleotide marker sequence that is about 30 to about 60 nucleotides downstream of the polymorphic site present within said nucleotide marker sequence.

In certain embodiments, the simultaneous identification of said plurality of polymorphisms and determination of said characteristics is performed using a processor-based system.

The invention further provides for a computer readable device having computer readable code embodied therein, said code embodying instructions for causing a processor-based system to identify a plurality of polymorphisms in a nucleic acid sample, comprising: instructions that cause a processor-based system to identifying a plurality of polymorphisms in a nucleic acid sample according to any one of claims 1-37 as originally presented; instructions that cause the processor-based system to hybridize said nucleic sample to said primer sequences and to said oligonucleotide probes; and instructions that cause the processor-based system to detect the presence of said plurality of polymorphisms in said nucleic acid sample.

The invention also provides for an assay plate to be used in the method of the invention. Thus, the invention provides for an assay plate comprising a plurality of recesses, wherein each of said recesses contains a composition, wherein each of said compositions comprises: (a) a pair of forward and reverse primers; (b) a first oligonucleotide probe; (c) a second oligonucleotide probe; and (d) a nucleic acid sample isolated from an animal; wherein said first oligonucleotide probe is capable of detecting a first allele of a sequence said nucleotide marker sequence; wherein said second oligonucleotide probe is capable of detecting a second allele of said nucleotide marker sequence; wherein said nucleotide marker sequence is any one of the nucleotide marker sequences as set forth in Tables 1-11; wherein said nucleotide marker sequence correlates with at least one of the characteristics of an animal selected from the group consisting of: (i) parentage; (ii) identity; (iii) sex, (iv) genotype and (v) phenotype; wherein said assay plate is capable of simultaneously identifying a plurality of polymorphisms; and wherein said plurality of polymorphisms correlates with least two characteristics of said animal selected from the group consisting of: (i) parentage; (ii) identity; (iii) sex, (iv) genotype and (v) phenotype.

The invention further provides for a composition comprising a plurality of nucleotide marker sequences, wherein each of said nucleotide marker sequences comprises a polymorphism, and wherein said plurality of nucleotide marker sequences correlates with at least two characteristics selected from the group consisting of: (i) parentage; (ii) identity; (iii) sex, (iv) genotype and (v) phenotype; wherein each of said nucleotide marker sequences is any one of the nucleotide marker sequences as set forth in Tables 1-11.

The invention also provides for a method of identifying a plurality of nucleotide marker polymorphisms comprising (a) contacting a nucleic acid sample with the composition comprising a plurality of nucleotide marker sequences; (b) hybridizing said nucleic acid sample to a pair of forward and reverse primer sequences; (c) performing PCR amplification of said nucleic acid sample; (d) hybridizing said amplified nucleic acid sample obtained from step (c) to said plurality of nucleotide marker sequences in said composition; and (e) identifying said plurality of nucleotide marker sequences; wherein said plurality of nucleotide marker polymorphisms correlates with at least two characteristics selected from the group consisting of parentage, identity, genotype and phenotype.

With regard to the methods above, the invention provides for a computer readable device having computer readable code embodied therein, said code embodying instructions for causing a processor-based system to identify at least two characteristics selected from the group consisting of parentage, identity and phenotype, comprising: instructions that cause a processor-based system to contact a nucleic acid sample with the composition comprising a plurality of nucleotide marker sequences; instructions that cause the processor-based system to hybridize said nucleic acid sample to said plurality of nucleotide marker sequences in said composition; and instructions that cause the processor-based system to detect oligonucleotide sequences within said nucleic sample that have hybridized to said plurality of nucleotide marker sequences; wherein said plurality of nucleotide marker sequences correlates with at least two characteristics selected from the group consisting of parentage, identity and phenotype.

The invention also provides for a method of determining at least two characteristics of an animal selected from the group consisting of: parentage, identity and phenotype, comprising (a) contacting a nucleic acid sample with the composition comprising a plurality of nucleotide marker sequences; (b) hybridizing said nucleic acid sample to a pair of forward and reverse primer sequences; (c) performing PCR amplification of said nucleic acid sample; (d) hybridizing said amplified nucleic acid obtained from step (c) to said plurality of nucleotide marker sequences in said composition; and (e) identifying a plurality of nucleotide marker polymorphisms within said nucleic acid sample that have hybridized to said plurality of nucleotide marker sequences; wherein said plurality of nucleotide marker polymorphisms correlates with at least two characteristics selected from the group consisting of parentage, identity and phenotype.

The invention further provides a computer database comprising the nucleotide marker sequences as set forth in Tables 1-11.

### BRIEF DESCRIPTION OF THE DRAWINGS

FIG. 1 provides an exemplary assay plate or panel upon which a plurality of samples or assays may be stored for processing in accordance with any of the methods of the present invention. The assay plate includes an array of recesses, which may be implemented as wells or through-holes.

FIG. 2 provides an exemplary processor-based system which may be used to process nucleic acid samples.

FIGS. 3A-J provides a series of scatter plots depicting identity data generated by the present invention. In each plot, homozygous populations are provided in the upper left and lower right and heterozygous populations are provided in the upper right. Specifically FIGS. 3A-J provide examples of identity, forensic and parentage markers for various species. FIGS. 3A-C provide examples of identity, forensic and parentage markers for cats. FIGS. 3 D-F provide examples of identity, forensic and parentage markers for dogs. FIGS. 3G-I provide examples of identity, forensic and parentage markers for horses. FIGS. 3 J provides examples of identity, forensic and parentage markers for cattle. The chart below is an example of the assay name correlating with the genomic location in cats.

FIGS. 3 A-C

| Cat Assay Na [symbol] | Cat Genomic Location |
|---|---|
| FC07 | B1: 156,143,186 |
| FC22 | C1: 123,746,252 |
| FC24 | A3: 14,410,638 |
| FC25 | F1: 33,007,663 |
| FC27 | E2: 35,480,527 |
| FC44 | A3: 48,181,817 |
| FC48 | B3: 149,673,110 |
| FC52 | B2: 159,389,942 |
| FC01 | Un: 51,831,052 |
| FC09 | A2: 17,611,273 |
| FC10 | B3: 107,303,663 |
| FC17 | A1: 15,263,737 |

[symbol]

indicates data missing or illegible when filed

FIGS. 4A-D provide a series of scatter plots depicting non-disease trait data generated by the present invention. This can include but is not limited to color, color patterns, hair length, or other physical characteristics. Data points positioned in the upper left include those homozygous for the first allele the lower right provides those homozygous for the second allele and data points in the upper right provide the heterozygous population. FIG. 4A includes scatter plots demonstrating the presence of polymorphisms associated with color or other physical characteristics in cats. Examples included are DILUT which is dilute coat color in cats, CHOC2 (brown) which is chocolate coat coloration in cats, BLK (black) which causes recessive black located in the agouti gene in cats and CINNAM which is cinnamon coat color in cats. Sequences are provided in Table 8 under the name of the marker for example; Cinnam is the assay name and is the CINNAMON sequence in Table 8 DILUT is MLPH DILUTION in Table 8 FIG. 4B includes scatter plots demonstrating the presence of polymorphisms associated with color or other physical characteristics in dogs, Examples are TYRP1-MC1R-S41C which denotes one SNP responsible for brown coat color in dogs, DOG-MASK-MASK causes a dark coloration or facial mask on dogs, MC1R-Yello-Yell is responsible for red to yellow coloration in some breeds of dog, and AGOUTI_DOG-R96c is associated with black coloration and it located in the agouti gene in dogs. Sequences for these markers are in Table 6 under trait names. FIG. 4C includes scatter plots demonstrating the presence of polymorphisms associated with color or other physical characteristics in horses, Examples are HORSE-MC1R-RED which denotes one SNP responsible for red coat color in horses, TOBIANO-TOB causes a white pattern or painted appearance in horses, SILVERH-SILH is silver coloration in horses. E AGOUTI-10 is bay pattern in horses. Sequences are in Table 2 under a similar trait name. FIG. 4D includes scatter plots demonstrating the presence of polymorphisms associated with color or other physical characteristics in cattle, Examples are BLCK which is responsible for red or the lack of red (black) coat color in cattle. The sequence can be found in Table 11, as RED. ALBIN causes a lack of pigment or white animals with pink or blue eyes and pink skin. The sequence can be found in Table 11, as Albino. In FIG. 4 scatter plots depict animals negative for the trait or disease in Red (VIC).

FIG. 5 provides a series of scatter plots depicting of sex determination data generated by the present invention. Data is shown from 3 species cat, dog, and cattle. ZFXY2 is cats, ZFXY1 is cattle and zfxy1_CF-xy2 is dog. Vic (Red) color denotes females and Green color (heterozygotes) denotes male animals. In FIG. 5 scatter plots depict animals negative for the trait or disease in Red (VIC).

FIGS. 6A-C provide a series of scatter plots depicting disease trait data generated by the present invention. FIG. 6A includes scatter plots demonstrating the presence of polymorphisms associated with diseases in cats, Examples include MPS 1 which is Mucopolysaccharidosis Type VI and MPSM which is Mucopolysaccharidosis Type VI Mild Form. BLDAB is B blood type in cats responsible for neonatal isoerythrolysis. Sequences are available by name in Tables 7-11, FIG. 6A also includes 1 scatter plot demonstrating the presence of polymorphisms associated with diseases in dogs as does FIG. 6B. In FIG. 6A MDR1-MDR is Multi-drug resistance in cancer in dogs. In FIG. 6B, SCID is severe combined immunodeficiency in dogs, VW GERM-VW1 is von Willibrand's Disease Type 2 in dogs and CYST_DOG-CYST is Cystinurea in dogs. Sequences can be found in Table 6 under disease names. FIG. 6B also includes 1 scatter plot demonstrating the presence of polymorphisms associated with diseases in horses as does FIG. 6C. In FIG. 6B, HORSE_JEB-JEB is Junctional Epidermolysis Bullosa (JEB) and is Sequence ID 62 in Table 2. FIG. 6C, Examples include HYPP_NEW-HYP which is Hyperkalemic Periodic Paralysis in horses and is Sequence ID 64 in Table 2 and HORSE_{—} LWO-LWO which is Lethal White Overo in horses and is Sequence ID 60 in Table 2. In FIG. 6 scatter plots depict animals negative for the trait or disease in Red (VIC).

### DETAILED DESCRIPTION OF THE INVENTION

### Definitions

It is to be noted that the term “a” or “an” entity refers to one or more of that entity; for example. “a nucleotide marker,” is understood to represent one or more nucleotide markers. As such, the terms “a” (or “an”), “one or more,” and “at least one” can be used interchangeably herein.

As used herein, “about” means within ten percent of a value. For example, “about 100” would mean a value between 90 and 110.

The term “plurality” or “multiple” refers to two or more, between about 20 and about 10,000, between about 20 and about 5000, between about 20 and 200; 3000 or more, 200 or more and extending up to whole genome analysis, 100 or more preferably about 20, 30, 40, 50, 60, 70, 80, 90, 100, 110, 120, 130, 140, 150, 160, 170, 180, 190, 200, 1000, 3000, or 9000; more preferably about 64, 128, 3072, 6344 or 9216.

The term “nucleotide” or “polynucleotide” or “nucleic acid” is intended to encompass a singular nucleic acid as well as plural nucleic acids, and refers to an isolated nucleic acid molecule or construct, e.g., messenger RNA (mRNA) or plasmid DNA (pDNA). A poly nucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. In other embodiments, a polynucleotide of the present invention is cDNA, genomic DNA, mitochondrial DNA (mtDNA), or RNA, for example, in the form of messenger RNA (mRNA).

By “isolated” nucleic acid or nucleotide is intended a nucleic acid molecule, DNA or RNA, which has been removed from its native environment. For example, a recombinant nucleic acid corresponding to a nucleotide marker contained in a vector is considered isolated for the purposes of the present invention. Further examples of an isolated nucleic acid include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

By “derived from” is intended an isolated nucleotide, a synthesized nucleotide (e.g. an automated synthesizer), or a nucleotide whose sequence has been obtained from a genomic database and subsequently isolated or synthesized.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. In addition, a vector, polynucleotide, or nucleic acid of the invention may encode heterologous coding regions, either fused or unfused to a nucleic acid. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

The “target oligonucleotide sequence” or “target nucleic acid” may be a portion of a gene, a regulatory sequence, genomic DNA, cDNA, and RNA (including mRNA and rRNA). Genomic DNA samples are usually amplified before being brought into contact with a nucleotide marker sequence. Genomic DNA can be obtained from any tissue source or circulating cells (other than pure red blood cells). For example, convenient sources of genomic DNA include whole blood, semen, saliva, tears, urine, fecal material, sweat, buccal cells, skin and hair. Amplification of genomic DNA containing a polymorphic site generates a single species of target oligonucleotide sequence if the individual animal from which the sample was obtained is homozygous at the polymorphic site, or two species of target molecules if the individual is heterozygous. RNA samples also are often subject to amplification. In this case, amplification is typically preceded by reverse transcription. Amplification of all expressed mRNA can be performed as described in, for example, WO 96/14839 and WO 97/01603 which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the PCR methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction.

As used herein, the term “polymorphism” refers to an allelic variant that occurs in a population that can be a single nucleotide difference present at a locus, or can be an insertion or deletion of one, a few or many consecutive nucleotides, or can be an inversion. A single nucleotide polymorphism (SNP) is characterized by the predominance in a population of certain nucleotides at a particular locus in a genome, such as the horse, dog, cat, cattle, or human genome. Typically, less than all four nucleotides (i.e., adenosine, cytosine, guanosine or thymidine) will predominate at a particular locus. For example, a particular locus in a genome of a specific population may contain either an adenosine or guanosine at the polymorphic site and thus two of the four nucleotides predominate at this particular locus. However, polymorph one or two, three or four nucleotides. It will be recognized that, while the methods of the invention are exemplified primarily by the detection of SNPs, the disclosed methods or others known in the art similarly can be used to identify other types of poly morphisms, such as an insertion or a deletion, which typically involve more than one nucleotide.

A “single nucleotide polymorphism” or “SNP” occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the population). A single nucleotide polymorphism usually arises due to a substitution of one nucleotide for another at the polymorphic site. Single nucleotide polymorphisms can also arise from a deletion of a nucleotide or an insertion of a nucleotide relative to a reference allele.

The terms “nucleotide marker” and “marker” are used herein interchangeably to refer to a nucleotide sequence having a single nucleotide polymorphism (SNP), insertion or deletion, where the SNP, insertion or deletion renders the marker suitable as a molecular identifier of particular animal(s), and where the molecular identifier correlates with parentage, identity and/or phenotype of particular animal(s). A polymorphic site within the nucleotide marker (e.g. the site of an SNP, insertion or deletion) is the locus at which divergence occurs. Preferred markers have at least two alleles (allele 1 and allele 2), each occurring at a frequency of greater than 1%, and more preferably greater than 10% or 20% of a selected population.

An “oligonucleotide probe” is defined herein as a nucleic acid sequence about 10, 12, 15, 18, 20, 21, 22, 23, 24, 25, 26, 27, 28, 29, 30, 31, 32, 33, 34 or 35 nucleotides in length that spans a region of a nucleotide marker containing a polymorphic site (e.g., an SNP, and insertion or deletion). The polymorphic site may be positioned about the center of the oligonucleotide probe, within about 5 nucleotides of the center of the oligonucleotide probe, within about 10 nucleotides of the center of the oligonucleotide probe and the like. Such an oligonucleotide probe can be used in polymerase chain reaction (PCR) for allele discrimination or identification of an allelic variation. An oligonucleotide probe can also be used for hybridization to a target oligonucleotide sequence. Hybridization may occur through the use of arrays of nucleotide probes.

The term “allele discrimination” refers to the determination of whether a DNA fragment contains two of the same alleles (either two allele 1's or two allele 2's) or two different alleles (one allele 1 and one allele 2) within a given nucleotide marker sequence. To achieve allele discrimination, two oligonucleotide probes can be labeled with two spectrally distinct dyes each identifying either allele 1 or allele 2. Results can be analyzed by measuring the level of fluorescence of each dye. Results can be plotted for comparison, such as on a scatter plot. In particular, if the fluorescent value of the DNA sample is high for allele 1 and low for allele 2, then the sample is homozygote for allele 1. Similarly, if the fluorescent value of the DNA sample is high for allele 2 and low for allele, then the DNA sample is homozygote for allele 2. If the DNA sample generates intermediate values for both dyes, it is heterozygote for both alleles.

A “first oligonucleotide probe” refers to an oligonucleotide probe that hybridizes to either allele 1 or allele 2. A “second oligonucleotide probe” refers to an oligonucleotide probe that hybridizes to allele 2 when the first oligonucleotide probe hybridizes to allele 1, or that hybridizes to allele 1 when the first oligonucleotide probe hybridizes to allele 2.

The term “quencher” is a compound used in PCR experiments that absorbs the energy of the reporter dye in its excited state. The quencher can emit its own fluorescent signal or emit no fluorescent signal.

The term “reference dye” is used in PCR experiments for normalization of the fluorescence signal of the reporter fluorophore. The reference dye fluoresces at a constant level during the reaction. Reference dyes include ROX, VIC®, HEX, NED and FAMT™.

The term “reporter dye” or “reporter fluorophore” refers to the fluorescent dye used to monitor PCR product accumulation of an oligonucleotide target sequence. This can be attached to a probe (such as with TaqMan or Molecular Beacons) or free in solution. This is also known as a fluorophore. Examples of reporter dyes are ROX, VIC®, HEX, NED and FAM™.

As used herein, the term “mutation” refers to a sequence variation in a gene, such as a single nucleotide difference, an insertion, a deletion, or an inversion, that is associated or believed to be associated with a phenotype. The term “gene” refers to a segment of the genome that codes for a functional product protein control region. Polymorphic nucleotide markers used in accordance with the present invention for determination of parentage, identity and/or phenotype in an animal may be located in coding or non-coding regions of the genome.

As used herein, the term “correlates with” refers to having a causal, complementary, parallel, or reciprocal relationship, especially a structural, functional, or qualitative correspondence between two comparable entities. In the present invention, for example, the identification of particular polymorphic sites (e.g., those within nucleotide marker sequences of the invention) in a nucleic acid sample derived from an animal, may correspond to the substantial likelihood of a particular animal having a certain identity, phenotypic trait, parentage, or combination thereof. The correlation between the presence of particular SNPs and the substantial likelihood of a particular animal having a certain parentage, identity, and/or phenotype has been established or demonstrated. The term “correlates with” can also be used in reference to drawing a conclusion about the parentage, identity and/or phenotype of an animal using a process of analyzing individually or in combination, nucleotide occurrence(s) of one or more SNP(s), which can be part of one or more haplotypes, in a nucleic acid sample of the subject, and comparing the individual or combination of nucleotide occurrence(s) of the SNP(s) to known relationships of nucleotide occurrence(s) of the SNP(s) in other animals. As disclosed herein, the nucleotide occurrence(s) can be identified directly by examining nucleic acid molecules, or indirectly by examining a polypeptide encoded by a particular gene where the polymorphism is associated with an amino acid change in the encoded polypeptide.

The term “animal,” as used herein refers to an individual animal providing a nucleic acid sample from which target oligonucleotides are obtained for the purpose of identifying parentage, identity and/or phenotype of that animal. Animals are identified according to known classes of scientific taxonomy, such as family, genus and/or species. Animals of the present invention are of families including but not limited to Equidae, Bovidae, Canidae, Felidae, Camelidae, Cervidae, and Suidae. In particular, animals of the present invention include but are not limited to the family and genera Bovidae Bos (cattle), Bovidae Ovis (sheep), Bovidae Capra (goat), Bovidae Bison (bison) Equidae Equus (horse, donkey, mule), Canidae Canis (dog), Felidae Felis (cat), Camelidae Vicugna (alpaca), Camelidae Lama (llama), Camelidae Camelus (camel), Cervidae Cervus (deer), Cervidae Alces (moose, elk), Cervidae Axis (deer), Cervidae Muntiacus (deer), Cervidae Dama (deer), Cervidae rangifer (reindeer, caribou) and Suidae Sus (pig).

As used herein, “hybridization” refers to the binding, annealing, duplexing, or hybridizing of a first nucleic acid molecule preferentially to a particular second nucleotide molecule. The stability of a hybridization complex varies with sequence composition, length and external conditions. Hybridization methods include those that rely on the control of stringency in reaction conditions to destabilize some but not all hybridization complexes formed in a mixture. Using these methods, it is possible to distinguish complete complementarity from partial complementarity between probe and target sequences that form a hybridization complex.

The term “specific hybridization” refers to the binding, duplexing, or hybridizing of a molecule only to a particular nucleotide sequence under stringent conditions when that sequence is present in a complex mixture (e.g., total cellular) DNA or RNA. Stringent conditions are conditions under which a target oligonucleotide sequence will hybridize to a nucleotide marker sequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the nucleotide marker sequences complementary to target oligonucleotide sequences hybridize to the target sequence at equilibrium. (As the target oligonucleotide sequences are generally present in excess, at Tₘ, 50% of the nucleotide markers are occupied at equilibrium). Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide or tetraalkyl ammonium salts. For example, conditions of 5×SSPE (750 mM NaCl, 50 mM Na Phosphate, 5 mM EDTA, pH 7.4) and a temperature of 25-30° C. are suitable for allele-specific nucleotide marker hybridizations.

A perfectly matched nucleotide marker has a sequence perfectly complementary to a particular target oligonucleotide sequence. Such a nucleotide marker sequence is typically perfectly complementary to a portion (subsequence) of the target sequence.

The term “hapolotype” refers to the genetic constitution of an individual chromosome. Haplotype may refer to only one locus or to an entire genome. In the case of diploid organisms, a genome-wide haplotype comprises one member of the pair of alleles for each locus (that is, half of a diploid genome). The term “haplotype” also refers to a set of single nucleotide polymorphisms (SNPs) on a single chromatid that are statistically associated. It is thought that these associations, and the identification of a few alleles of a haplotype block, can unambiguously identify all other polymorphic sites in its region.

The term “assay plate” refers to panel upon which a plurality of samples or assays may be stored for processing in accordance with any of the techniques described below. The assay plate includes an array of recesses, which may be implemented as wells or through-holes.

As used herein, universal polymorphism identification system is synonymous with universal genetic evaluation.

### Polymorphic Nucleotide Markers

The present invention is based on the utilization of known nucleotide marker sequences containing single nucleotide polymorphisms (SNPs), insertions and/or deletions and other mutations that can be used to determine parentage, breed, identity, sex, genotype and/or phenotype in an animal. Accordingly, provided herein is an assay plate comprising a plurality of compositions, wherein each composition is capable of identifying a polymorphism contained within a nucleotide marker sequence of the invention. The polymorphic nucleotide marker sequences of the invention each have an occurrence of a polymorphism, wherein the occurrence of the polymorphism correlates with parentage, identity, sex, genotype and/or phenotype, or breed determination associated with that animal.

Single nucleotide polymorphisms (SNPs) are positions at which two alternative bases occur at appreciable frequency (>1%) in a given population, and are the most common type of genetic variation. The site is usually preceded by and followed by highly conserved sequences of the allele (e.g., sequences that vary in less than 1/100) or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Though in most embodiments a single nucleotide polymorphism is detected, the present invention also encompasses the dection of the presence, absence or substitution of a short series of nucletides in sequential alignment. In some embodiments two nucleotides in direct sequenctial alignment are present, deleted or substituted. In other embodiments, three nucleotides in direct sequential alignment are present, deleted or substituted. In other embodiments four nucleotides in direct sequential alignment are present, deleted or substituted. In other embodiments, five nucleotides in direct sequential alignment are present, deleted or substituted. In other embodiments, six nucleotides in direct sequence alignment are present, deleted or substituted.

Single nucleotide polymorphisms may be functional or non-functional. Functional polymorphisms affect gene regulation or protein sequence whereas non-functional polymorphisms do not. Depending on the site of the polymorphism and importance of the change, functional polymorphisms can also cause, or contribute to diseases.

SNPs can occur at different locations of the gene and may affect its function. For instance, polymorphisms in promoter and enhancer regions can affect gene function by modulating transcription, particularly if they are situated at recognition sites for DNA binding proteins. Polymorphisms in the 5′ untranslated region of genes can affect the efficiency with which proteins are translated. Polymorphisms in the protein-coding region of genes can alter the amino acid sequence and thereby alter gene function. Polymorphisms in the 3′ untranslated region of gene can affect gene function by altering the secondary structure of RNA and efficiency of translation or by affecting motifs in the RNA that bind proteins which regulate RNA degradation. Polymorphisms within introns can affect gene function by affecting RNA splicing.

A polymorphic site can also contain an insertion, or additional base pairs within a region of DNA on one allele. In addition, a polymorphic site can contain a deletion, generated by the removal of base pairs within a region of DNA on one allele. The present invention can simulataneously detect deletions, substitutions and additions.

The term genotyping or genotype refers to the determination of the genetic information an individual animal carries at one or more positions in the genome. For example, genotyping may comprise the determination of which allele or alleles an individual carries for a single SNP or the determination of which allele or alleles an individual carries for a plurality of SNPs. In making this determination, the alleles can be discriminated (allele discrimination). For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Each allele may be present at a different frequency in a given population, for example 30% of the chromosomes in a population may carry the A allele and 70% the C allele. The frequency of the A allele would be 30% and the frequency of the C allele would be 70% in that population. Those individuals who have two copies of the C allele are homozygous for the C allele and the genotype is CC, those individuals who have two copies of the A allele are homozygous for the A allele and the genotype is AA, and those individuals who have one copy of each allele are heterozygous and the genotype is AC.

Using the teachings herein, genotyping can be accomplished by determination of polymorphic sites within a nucleic acid sample. The genotypic determination can then be correlated with the parentage, identity and/or phenotype of an individual animal. Therefore, the compositions of the present invention can be used to determine the parentage, identity and/or phenotype of an animal regardless of breed. For example, the compositions can be used to determine the parentage, sex, identity, genotype and/or phenotype of an individual animal of a particular breed of cattle including, but not limited to, Angus, Limousin, Brahman, Jersey, Chianina, Brown Swiss, Santa Gertrudis, Shorthorn, Guernsey, Maine-Anjou, Simmental, Hereford, Holstein. Gelbvieh, Charolais or Beefmaster cattle, or a particular breed of horse including, but not limited to American Saddlebred, Andalusian. Appaloosa, Arabian, Miniature Horse, Quarter Horse, Paint, Paso Fino, Thoroughbred. AkalTeke, Standardbred, Tennessee Walking Horse and Icelandic, or a particular breed of dog including, but not limited to Afghan Hound, Australian Cattle Dog, Australian Shepherd, Basenji, Basset Hound, Beagle, Belgian Tervuren, Bernese Mountain Dog, Borzoi, Chihuahua, Chinese Shar-Pei, Chinese Crested, Corgi, Labradoodle, Cocker Spaniel. Collies, Dachshund, Doberman Pinscher, German Shepherd Dog, German Shorthaired Pointer, Golden Retriever. Greyhound, Labrador Retriever, Maltese, Mastiff Miniature Schnauzer, Poodle, Pug, Rottweiler, Saluki, Samoyed, Shetland Sheepdog. Siberian Husky, St. Bernard, Whippet and Yorkshire Terrier.

Since genomic DNA is double-stranded, each SNP can be defined in terms of either the plus strand or the minus strand. Thus, for every SNP, one strand will contain an immediately 5′-proximal invariant sequence and the other strand will contain an immediately 3′-distal invariant sequence. In the present invention, the invariant sequence spanning the SNP is between about 20 and about 35 nucleotides in length, and more preferably 30 nucleotides in length.

For the identification of multiple genetic characteristics, the present invention provides for a panel comprising a plurality of assay compositions, wherein each assay composition is capable of identifying at least one of the nucleotide markers as set forth in Table 1 below. Table 1 lists the name of the marker (SNP ID), the chromosome from which the marker is derived (Chr), the position of the polymorphic site within the chromosome (Position), a nucleotide that occurs at the polymorphic site (genomic allele (G)), the alternate nucleotide that can occur at the same polymorphic site (alternate allele (A)), other SNPs that occur within 30 by of the genomic/alternate allele (O), percent repeat (P) (percent of sequence that is repeated bases), the discovery breed (the breed(s) in which the SNP was identified) and the discovery read (the sequencing read where the SNP was identified):

TABLE 1
HORSE SNP PANEL SEQUENCES (SET #1)

| SNP ID | Chr | Position | G | A | O | P | Discovery Breed | Discovery Read |
|---|---|---|---|---|---|---|---|---|
| BIEC323 | chr1 | 1585996 | C | T | 0 | 0 | Andalusian, Arabian | S257P6129FJ20.T0, |
| | | | | | | | | S255P69RP21.T0 |
| BIEC35895 | chr1 | 86195760 | A | G | 0 | 0 | QuarterHorse, | S256P6119RI2.T0, |
| | | | | | | | Thoroughbred, | S261P6121RN11.T0, |
| | | | | | | | AkalTeke | S259P6122RG18.T0 |
| BIEC67750 | chr1 | 156029252 | A | G | 0 | 0 | QuarterHorse, | Twilight, S256P6104FI11.T0, |
| | | | | | | | Standardbred | S260P630FE3.T0 |
| BIEC372460 | chr2 | 10491958 | G | C | 0 | 3 | QuarterHorse, | S256P656FM18.T0, |
| | | | | | | | Arabian | S255P6118RD21.T0 |
| BIEC382016 | chr2 | 27765519 | G | A | 0 | 11 | Andalusian, Arabian, | Twilight, S257P633RL14.T0, |
| | | | | | | | QuarterHorse | S255P6124FH24.T0, |
| | | | | | | | | S256P6101FL20.T0 |
| BIEC404000 | chr2 | 68717792 | T | G | 0 | 0 | Icelandic, Arabian | Twilight, S258P678FH6.T0, |
| | | | | | | | | S255P6124FO9.T0 |
| BIEC645002 | chr3 | 1175654 | T | C | 0 | 0 | Thoroughbred, | Twilight, S261P630RM15.T0, |
| | | | | | | | QuarterHorse | S256P673FD9.T0 |
| BIEC661467 | chr3 | 47244981 | A | G | 0 | 0 | QuarterHorse, | S256P633FA11.T0, |
| | | | | | | | Arabian | S255P6123FI16.T0 |
| BIEC717039 | chr4 | 17776766 | A | G | 0 | 0 | Thoroughbred, | S261P623FO1.T0, S260P6114RM21.T0 |
| | | | | | | | Standardbred | |
| BIEC733312 | chr4 | 63503371 | G | A | 0 | 0 | QuarterHorse, | S256P673FA10.T0, |
| | | | | | | | Arabian, Andalusian | S255P653FC24.T0, |
| | | | | | | | | S257P61RP9.T0 |
| BIEC748249 | chr5 | 2999858 | A | G | 0 | 0 | Icelandic, | Twilight, S258P676RM14.T0, |
| | | | | | | | QuarterHorse, | S256P622RC15.T0, |
| | | | | | | | Thoroughbred | S261P667FD1.T0 |
| BIEC754184 | chr5 | 15457472 | A | G | 0 | 0 | QuarterHorse, | S256P69FE6.T0, S255P6110RL6.T0, |
| | | | | | | | Arabian, AkalTeke | S259P623RA18.T0 |
| BIEC778319 | chr5 | 69493593 | T | C | 0 | 0 | AkalTeke, | Twilight, S259P611RI24.T0, |
| | | | | | | | Thoroughbred | S261P643FO17.T0 |
| BIEC797384 | chr6 | 43616437 | T | G | 0 | 0 | Standardbred, | Twilight, S260P692RE18.T0, |
| | | | | | | | Andalusian, Quarter | S257P6104FE12.T0, |
| | | | | | | | Horse | S256P624FO14.T0 |
| BIEC810015 | chr6 | 69737444 | G | A | 0 | 0 | QuarterHorse, | Twilight, S256P682RD24.T0, |
| | | | | | | | Arabian | S255P652FM2.T0 |
| BIEC823988 | chr7 | 10927001 | C | T | 0 | 0 | Thoroughbred, | Twilight, S261P6144FH15.T0, |
| | | | | | | | AkalTeke, | S259P6116RA17.T0, |
| | | | | | | | Standardbred | S260P610FI11.T0 |
| BIEC846563 | chr7 | 65694972 | C | G | 0 | 21 | Thoroughbred, | S261P635RN2.T0, S256P670RA7.T0, |
| | | | | | | | QuarterHorse, | S255P648FJ16.T0 |
| | | | | | | | Arabian | |
| BIEC866619 | chr8 | 10338538 | T | C | 0 | 0 | Icelandic, Arabian, | S258P650FJ8.T0, S255P665FK13.T0, |
| | | | | | | | Standardbred | S260P666FN18.T0 |
| BIEC880212 | chr8 | 35993310 | C | T | 0 | 0 | Arabian, Icelandic | Twilight, S255P6115FI12.T0, |
| | | | | | | | | S255P678FP17.T0, |
| | | | | | | | | S258P6124FM7.T0 |
| BIEC903524 | chr9 | 4109222 | C | T | 0 | 0 | QuarterHorse, | Twilight, S256P625RM11.T0, |
| | | | | | | | Andalusian | S257P640FF1.T0 |
| BIEC933800 | chr9 | 62529880 | T | A | 0 | 0 | QuarterHorse, | Twilight, S256P662RK10.T0, |
| | | | | | | | Arabian, Andalusian | S255P621FP4.T0, |
| | | | | | | | | S257P6114FB2.T0 |
| BIEC100227 | chr10 | 18562230 | A | C | 0 | 0 | AkalTeke, | Twilight, S259P616RP5.T0, |
| | | | | | | | QuarterHorse | S256P655FH20.T0 |
| BIEC119261 | chr10 | 59078213 | C | T | 0 | 0 | QuarterHorse, | Twilight, S256P669RC20.T0, |
| | | | | | | | Standardbred | S260P629RG15.T0 |
| BIEC123028 | chr11 | 48708 | T | C | 0 | 0 | Thoroughbred, | Twilight, S261P633RF4.T0, |
| | | | | | | | AkalTeke | S259P6129FA13.T0 |
| BIEC141078 | chr11 | 37812203 | T | C | 0 | 0 | Arabian, | Twilight, S255P6108FD2.T0, |
| | | | | | | | Thoroughbred | S261P631RN14.T0 |
| BIEC159353 | chr12 | 7954220 | T | C | 0 | 0 | Arabian, Andalusian, | Twilight, S255P63RA17.T0, |
| | | | | | | | QuarterHorse | S257P6130RL4.T0, |
| | | | | | | | | S256P646FD3.T0 |
| BIEC167336 | chr12 | 18561559 | T | C | 0 | 0 | Thoroughbred, | S261P6122RB7.T0, |
| | | | | | | | Standardbred, | S260P6111RH8.T0, |
| | | | | | | | Andalusian | S257P69FB1.T0 |
| BIEC170689 | chr13 | 4859954 | A | G | 0 | 3 | QuarterHorse, | Twilight, S256P630RP16.T0, |
| | | | | | | | Arabian | S255P64RA9.T0 |
| BIEC177534 | chr13 | 13499460 | G | A | 0 | 0 | Icelandic, | Twilight, S258P613FO9.T0, |
| | | | | | | | QuarterHorse | S256P663RG14.T0 |
| BIEC187185 | chr14 | 10519408 | G | A | 0 | 19 | QuarterHorse, | Twilight, S256P620RE10.T0, |
| | | | | | | | Standardbred | S260P671FF16.T1 |
| BIEC214463 | chr14 | 84065438 | C | G | 0 | 0 | AkalTeke, | Twilight, S259P652FE18.T0, |
| | | | | | | | QuarterHorse, | S256P651FD8.T0, |
| | | | | | | | Icelandic | S258P665FI14.T0 |
| BIEC220494 | chr15 | 54151 | A | G | 0 | 0 | Arabian, Andalusian, | Twilight, S255P694FO14.T0, |
| | | | | | | | QuarterHorse | S257P655RG14.T0, |
| | | | | | | | | S256P68FJ9.T0 |
| BIEC252403 | chr15 | 57437448 | A | G | 0 | 0 | Andalusian, Quarter | Twilight, S257P638FE3.T0, |
| | | | | | | | Horse | S256P67RA6.T0 |
| BIEC270317 | chr16 | 18502832 | A | G | 0 | 0 | Standardbred, | Twilight, S260P671RN2.T1, |
| | | | | | | | Arabian | S255P673FD16.T0 |
| BIEC304838 | chr16 | 87373220 | T | C | 0 | 0 | Thoroughbred, | Twilight, S261P6144FF22.T0, |
| | | | | | | | Arabian | S255P641RD20.T0 |
| BIEC306934 | chr17 | 5112116 | A | G | 0 | 0 | Arabian, | S255P680FH18.T0, |
| | | | | | | | QuarterHorse | S256P6135RN8.T0 |
| BIEC323723 | chr17 | 78516552 | T | C | 0 | 0 | Andalusian, | Twilight, S257P651RH19.T0, |
| | | | | | | | Standardbred | S260P683RP22.T0 |
| BIEC338343 | chr18 | 45058553 | A | G | 0 | 0 | AkalTeke, Arabian, | Twilight, S259P619RP3.T0, |
| | | | | | | | Andalusian | S255P682FI21.T0, |
| | | | | | | | | S257P694RG13.T0 |
| BIEC347016 | chr19 | 8846168 | T | C | 0 | 0 | Arabian, | Twilight, S255P654RL1.T0, |
| | | | | | | | Thoroughbred | S261P641RH13.T0 |
| BIEC450770 | chr20 | 56244068 | T | G | 0 | 0 | QuarterHorse, | Twilight, S256P6139FI18.T0 |
| | | | | | | | Thoroughbred, | S261P625RO17.T0 |
| | | | | | | | Andalusian | S257P682FK1.T0 |
| BIEC465101 | chr21 | 27637059 | A | T | 0 | 0 | Thoroughbred, | S261P649RK22.T0, |
| | | | | | | | Arabian, | S255P648FB16.T0, |
| | | | | | | | Standardbred | S260P629FO7.T0 |
| BIEC486760 | chr22 | 16193666 | T | C | 0 | 0 | Arabian, | Twilight, S255P665FA16.T0, |
| | | | | | | | Thoroughbred, | S261P649RK17.T0, |
| | | | | | | | Standardbred | S260P630RD15.T0 |
| BIEC507792 | chr23 | 6819375 | C | T | 0 | 0 | Arabian, | S255P652FC10.T0, |
| | | | | | | | Thoroughbred, | S261P612RL13.T0, |
| | | | | | | | Standardbred | S260P696FL5.T0 |
| BIEC521111 | chr24 | 7335306 | T | C | 0 | 0 | Thoroughbred, | Twilight, S261P697FO18.T0, |
| | | | | | | | QuarterHorse | S256P694FL8.T0 |
| BIEC547263 | chr25 | 6279709 | T | C | 0 | 15 | QuarterHorse, | Twilight, S256P678RH13.T0, |
| | | | | | | | Thoroughbred | S261P612RK8.T0 |
| BIEC574261 | chr26 | 27538107 | C | T | 0 | 0 | Arabian, | Twilight, S255P64RN2.T0, |
| | | | | | | | Standardbred | S260P615FP22.T0 |
| BIEC585067 | chr27 | 7673552 | C | T | 0 | 0 | Arabian, AkalTeke, | Twilight, S255P6102RK14.T0, |
| | | | | | | | Thoroughbred | S259P633RF19.T0, |
| | | | | | | | | S261P63RB23.T0 |
| BIEC609174 | chr28 | 7989217 | C | T | 0 | 0 | Icelandic, | Twilight, S258P622FB13.T0, |
| | | | | | | | Andalusian, | S257P6115RF14.T0, |
| | | | | | | | Standardbred | S260P631RE16.T0 |
| BIEC628735 | chr29 | 1807945 | G | C | 0 | 0 | Icelandic, | Twilight, S258P6108RC18.T0, |
| | | | | | | | Standardbred, | S260P6127RA4.T0, |
| | | | | | | | QuarterHorse | S256P631RE3.T0 |
| BIEC688595 | chr30 | 4306752 | A | G | 0 | 0 | Arabian, AkalTeke | Twilight, S255P6127FC14.T0, |
| | | | | | | | | S259P654FN1.T0 |
| BIEC697335 | chr31 | 2733738 | C | T | 0 | 0 | QuarterHorse, | Twilight, S256P640RP19.T0, |
| | | | | | | | Andalusian | S257P67FB15.T0 |
| BIEC938831 | chrX | 4928692 | A | G | 0 | 0 | Thoroughbred, | S261P664RM16.T0, |
| | | | | | | | Arabian | S255P653FJ4.T0 |
| CREAM | Chr21 | | G | A | | | | |
| SILVER | Chr6 | | C | T | | | Icelandic, Rocky Mtn | |
| TOBIANO | Chr3 | | C | G | | | | |
| SABINO | Chr3 | | T | A | | | Tenessee Walker | |
| AGOUTI | Chr22 | | + | GAAAAGAAGCA | | | | |
| MC1R | Chr3 | | C | T | | | | |
| LWO | Chr17 | | TC | AG | | | | |
| GBE1 | Chr26 | | C | A | | | Quarter Horse | |
| JEB | Chr5 | | + | C | | | Belgian | |
| SCID | Chr9 | | + | TCTCA | | | Arabian | |
| HYPP | Chr11 | | C | G | | | Quarter Horse | |

The nucleic acid sequences of the markers as set forth above in Table 1 are provided in Table 2 below, where the position of the polymorphic site (e.g., the single nucleotide polymorphism (SNP), insertion and/or deletion) is bracketed and indicated in bold (e.g., [T/C] indicates that this position is polymorphic and that the nucleotide at this position is either a “T” or a “C”). Thus, allele 1 of this marker would contain a “T” at the position indicated and allele 2 of the marker would contain a “C” at the position indicated. The determination of a T or a C at this position is correlative of at least one characteristic, such as parentage, identity, sex or phenotype):

TABLE 2
HORSE SNP PANEL (SET #1) NUCLEOTIDE MARKER SEQUENCES

| | |
|---|---|
| BIEC323 | GCCTTGTGACATCACAGCTGGATGTGTGTGGCCATGTTCAGAACTTGGTC |
| (SEQ ID | CCAGGAACTGGTGGGCACTCGCTCACATGTGGGTCTCTGGCTCTACCTCC |
| NO: 1) | TGCCTGCTGGCCCAAACTTTGGGCCAGAGCCACACAAACTCCTTCTCTTT |
| | AAACACCACTGCTTCCCTCCTCCTCGCTGATCTGTAGCTTTCCCCCGATT |
| | [T/C]GGGATGTTCTTACTGCACATCCTGGGCATTTCTCGTCTACATCACCT |
| | GGTTTAGCGCCGTGGCATGCTGGCTCACATGTGCCACCACAGCTGCATG |
| | AGGGTTTCTCCAGGAGCAGGAGGGTTGGGCGAAGAGGCCAAGATTCCTC |
| | GCTGAGCACTTGTCACATGGAGATTGCTGAGAAAATTCTGTAGATTTCA |
| | AAGGAT |
| BIEC35895 | GGGTAGATTTAGAGATAAAGAGAGAGATGAGAGTCTAGGGTTNGATTTT |
| (SEQ ID | ATGGCCCTCGTAATATTATCCGCACTAGGAGTTGATATGAGCTCTGCTGA |
| NO: 2) | ATATGGCCTGGTCGTAAAGAGTGTGCTGGGAGGATGCTGGCACGTGTGC |
| | TCAATGTCTACAGCCTTGAGGAAGCCTTGCACATCAGGCACCCCGAGTC |
| | AAA[G/A]GGAGAGTGGTTCGTGGCGGGATCAACTTACGGATTGGAATCT |
| | GGTGTCTTTGTAGATCGAGGCTATGAACTCTAGCTGGGCACCNCGACCA |
| | CCTTCCCTCCTTGTCACAGGCAAAGGAGCCATGCCGCATCTCTGAAGAA |
| | GTGCAGGGAAGATGCGACAGAAGGCGAAGGGACCCAAACACCACCAAG |
| | AAGGCGCATTGA |
| BIEC67750 | GCTGCACATGGTTGAGTTATGAATCAAGCTTGTTCTCCCGATGCGGGAA |
| (SEQ ID | AATGGGCCGATGAATCCATTTCCATCACGACACTACCCATAAGTCATGG |
| NO: 3) | ATTGAAGGGCACTTTTCTTCCTTCTGGAATTTCCAATGACAAAGATTTCA |
| | TTATCCAAAGCAATAATTTGTAACCAGAGCAATGCATGATTTCACTCTAC |
| | CT[G/A]AATTAGGTCCATTGTGAGAGGGAGGCTGGGACAACCTCAGTGT |
| | CTGGAGGGGCAGAAGAGGGTGAAATTGGACTCCTTCTTTGCTCTCGCCC |
| | CTCCCCCATTCTTTCCCTTTTTCTCTCTGTGTGGGTGTTCACTTTCTTTGTT |
| | CTCAGTCTCCTTTCTCTTGAGCCAACCCATCTCCTGGTGCTCTGCACTGG |
| | CCATGA |
| BIEC372460 | CCAAAGTCCTAAACTCTAGCCTCCCGTTGGTTCTCACCCTCGCGTTTTAG |
| (SEQ ID | CGTTCTAATAGTGATCTTGAGANTCTTTGGCACGGAGCAAAGCTCCTCTT |
| NO: 4) | TGACCCAGTGCAGCGAGGGCCTCCACAACCTGCCCTACCCCTTTCGATTC |
| | CCTTCCTCCCCTTAGCCCCACCTGGCTCAGGAAGAGTACAGAACGCATC |
| | A[C/G]GGCGAGGAGGGGCTCCGAAAGTTACAAACCTGCGCTGCCCGCCA |
| | CCTGCCCCTCCGAGCGCGCCGCGCGGCTTAAAGTGCCGCTGGGGATGCC |
| | CCCCTTCCCCCCGTACCCCAACCCCGACCGTCACCTGGAGCCGGAAGCG |
| | CTGCGCAGCCTTGTCCATGTTCTCCAGGGCCGCTTGCTCGCCGCTGTCGC |
| | GCCCGGGC |
| BIEC382016 | GTAAAATAAGAGATGCACTATCTCTCTGATATTCTCTGCTCTGGACCTGA |
| (SEQ ID | GCTGCACCTCAGAAAGGGGCTCTTTCTAGCAGAGAGGGGAGTGAGGTTG |
| NO: 5) | CCATTTTGCTGCCACTCCCAAGAGGGCAGGCCCTGAGACTATTTCTGTCT |
| | CTCCTCTCCTGATTCCCCACCCCTCCTGTCTAGACTGAGCTGAGTAACTG |
| | T[A/G]TCTCCATGCTGGTGGCGGGGAACGACCGTGTGCAGACCATCATCA |
| | CTCAGCTGGAGGACTCCTGTCGAGTGACAAAGGTGAGAGGAAGAATAG |
| | CTCTGCCTGGGGCTTCGAAGCCTCCCAGTGTGGCCCGGTCTCTGGGGTGA |
| | AGGCTCACCCATGGGATGTAACTAAAGGTTGGGGCCTGGAGCCTGGAGT |
| | GTTCAGGC |
| BIEC404000 | ATATTAAAATTGTTGTCTACCCTCAGACCACACCAGGCACAGTATCCCA |
| (SEQ ID | AGGGCATGTGAATAGGAAGTGCTCAAGCCATAGGCATTGCCTTGATCCT |
| NO: 6) | TCCACAGAGCTGCTAATTAGCCTTGGGTCAGTCAGAAAACTGGTTACTC |
| | AGCTTTGAAACTTCTAGGCAAATTTTGCTCTTAAGTATATAGCACGACAA |
| | AAA[G/T]AAGTGATAAAGACCTTTTAACAACGTGTCTTCATTTTACTTAT |
| | ACTCTTCCTATTGTCATCACCTCCTGAGGAAGCCTTTATAACCAAAACAC |
| | ACACTTGATGAAGAGAACGTGAGCAGTCAAAAAAACACATATGTACAC |
| | CGAAGACACAACTTAATTTTGGTTGGGTCCTCATGCAGCCAGGACAGAG |
| | AAAACTTTTG |
| BIEC645002 | ATGTCTAGATTCCAATTTTATGAAACCTCAGATATGCAATATTATTTAAG |
| (SEQ ID | AGGTTAGAAGTGACTTTAATTTAAATTCAGATAAATAAGTTAATTTAGA |
| NO: 7) | GTTTTTGATATGATGAGAGTTTTTGTAACGCTGGATAAAGTTATGTTAGA |
| | ACTTGACAAAACTCCTGAAAGGTTGATAACCAAAAGAAAGGTAGACTTG |
| | TT[C/T]GTTGTCTTCTTGATATAGCTTCCATTTCTCTTTAGCTCCTTGACAG |
| | TAAAGCAACTNTTTCTATTGTAATATCAGTACATGTTCTTCTTATTTCAAT |
| | TTAGAAAAAAGCTGGTCTGTATCTGCCTGTAACATCTCTGAAGGGAGAA |
| | ATCAGCTCACTTAACTTCCCTTCTCTTCCCCAAATGAATATTGTACCTTCC |
| | ACT |
| BIEC661467 | TTGAAGTTTCGGTTGACTTATAGCTCTTTTTTGACCTTTGCTACTAATAGC |
| (SEQ ID | TGGCAGAGAATAAAACCAGACATGTCAATCACTGTCTAGATTTTATAAA |
| NO: 8) | CTATTGACTGTTTCTTGAAGGATGGTAATGCTTATTTTATTGGCCTCTTGT |
| | ACCATAGAAGGGGATTTATAGAGTGCCAAAAAAATGAAAACCATGCCT |
| | A[G/A]CTAGCATCATCCAGATCTGCTGGTAATAAAGTCACTGAAATTAAT |
| | AACTATCAATTAAATATAAATGAGCTGAAACCACACCAAGGAGAGAATC |
| | AACAGCATTTTAATTCCTAGATCTCTTTTGGCATTATCATATTTAATGCTC |
| | TGTAAAGAGTTGAATTAACTAGTTTTCCTG |
| BIEC717039 | AAAACAGCTTCTTTGCAAATGAGAGCACAGCCGTGCTTCCCTGACTCCA |
| (SEQ ID | AATAGCCAAGTGAGAAGATGTCACCACAGGCTGCCCCTTCGTGACCTGG |
| NO: 9) | GCTAAGCCGAGTCCCCCAAATTTCCTCTGAACCTCTGGCTACAAAGAAA |
| | TGTGCTTAGCTTGCTCAGGAGAGTTTGTCCAAACTCATGGAATTCATGGA |
| | GGT[G/A]TTAAGTGTCAAGCTATTTTCTTCAGTTTCCCCTTTTACCCTCAC |
| | ATGTCACCCCCTCTTAAAAGTTTTTTTAAAGTGAAATACAAAATTTATCC |
| | AAAGAGAAATGAGATTTCCTGGATAAAGCATTTGTGTGATACTATTTCT |
| | GAGTCTGTGTCTCTGAAAAAATGGCTGGGAAATCCCCTCGTCTCTACATT |
| | TAAGCAT |
| BIEC733312 | GCTTTCCAAAGTGTTGGACAAGCTAAGTCCTATGCAATTATGCAATACTT |
| (SEQ ID | TTAAAAAAAACTTGAATTTTAAAATATGCTTGAAAAATAGATGCTGATC |
| NO: 10) | CAACAAGAACAGAGACTATCGATGAAAAGAATGTGTTCTCTGTGCACCT |
| | AAGGGAAGCCAACACACAGGCAGCCTCATGTGGCCTGAAGCTTCAGGA |
| | CTTT[A/G]GGAAGTTGCTTGCAGATGAATTTCTTTGAAATGAATAGCTCA |
| | GGGCGGCATATGCCCTCCCTCTAGATTTGACTTCTGTGGTTTATGTATAA |
| | GCTGGGGAAGACCTCAGAGTCTGACCTAGACTCACGTTATGTGCCTCTG |
| | AAGTCTGGTGAAAGGCCAGACTTTAGGATTCCGCAGAGTTGAGAGTTGA |
| | GTGAGGAACC |
| BIEC748249 | ATCAGTCCTTCAGGTTCTGGAAGCCCTCCATGCTGAGGCAATTTATGTTT |
| (SEQ ID | CTCTGCCTAGGGCACGGGGAACGCCTTCACCTCCACTCAGACCCTTCTGG |
| NO: 11) | ATTTCTCCCAGCCAAGGAAGTATGGAGCTCCAGAAGAATCTTACGAAAA |
| | GTTTCTGAGCATAGGAGAGAGTACGTTTACCTTTAGGGCAGCGTTTCCTG |
| | C[G/A]CGTCTGCTGCATCACCCCAGCCTAGCTTAACTACCTGAACACACG |
| | GCTGGACTTGAGACCCTCTTAAGAATTCAAGTTTGTGGGGAATGGAAGC |
| | TGGGAGGGAGTTGAGCAAGAAGGAAGGTTCCTATAGCTCTAGCAGCAC |
| | GCCTAGTTCAGGGAGGAAGGACAGACGAGAGGGCATCATACTCACAAA |
| | GAAAGTCTTC |
| BIEC754184 | CCCGCCATTGGCGGGGAGACCCGGCCTGGTGCTCGGGGCNCCCGGAGGG |
| (SEQ ID | TCCCAGAGAGAGACACGGAGGGCACGGAGGTCTNCCAGCTGCCGTTGCC |
| NO: 12) | CGCCCCNTGGGACTAGGGATTGCCGGAGATCTCGGAGAGGACCGGGGC |
| | GGGGGAACTTTCAAAGGCGGGTCCGGCGACCCGGTGGGGAAGCGCCGG |
| | AGCTCC[G/A]CCAGGCAGCAGACAAAACTCTCTGTCTGCCGGTAGCAGA |
| | GGGGCCACGCTGAGNACTCAGGGCTCCCGGCAGAGGCCCGGANAGAAG |
| | CCCAGAGGGCGGGGCGACCCCCAGCTGCCGTTGCCCGCCCCGNGGGACT |
| | NGGGATTGCCGGAGATCTCGGAGAGGACCGGGGCGGGGGAACTTTCAA |
| | AGGCCGGATCGGCGAC |
| BIEC778319 | TTCATAAATCCTGGAGTAATGGTGAAGCCTTTTTGGCTTAAATTTTACTT |
| (SEQ ID | GGGTTGTAAAATTAACCCCACTTTTATTGGGTAACTAATACCATAAACCT |
| NO: 13) | ATGGGGAGAAAGAAGTTTCTCGATGACCCAGTCTTGGATCTCGGAAACC |
| | GAAGCCCTGAGACCAGAATGCCCCCCACGCCACCCCCTGCATTACAGAT |
| | GA[C/T]GCTCTTCCACCCCCAGGCATTAGCAGAGCCCTGGATTTTTGAGG |
| | TGTCATTATGAGAACACGTCTTCCTCCCCCACATTAAGCTCTCAAGGCCT |
| | CAATTTATATCTCTTGGGAGGACAAAACCCTGGAATTACCCCAGATATA |
| | AATCCCGACCTACTGGGAGTTCCCATATTTTATGTGAGGCTTAGGCTAAA |
| | CTTCTTA |
| BIEC797384 | GTCATTTGCATCTCTAGCATTATTACAATTCTGAAAGTCATTTCAAATAA |
| (SEQ ID | GGTAAGTTTTAGAAGTGAAAGGAAACTTCTGGCATATTAGACATAAGTC |
| NO: 14) | AAGGACTCTTGTTTATGTCAAGCAATTCTACCACATATCTTTGTATGATT |
| | AGGATAATTGTTTAGAATATTCTCCCAGGCACATTACTGTCAATTACTAA |
| | A[G/T]ATTATATAGTCAGAGTCCCCACCTTGTATTCCGTTTGAATCACACT |
| | GTTTTGCATTATTTTAAATGGCCACATTTTTATTTTTATCGAGGGGAACG |
| | TAACTGCAAGGAATGTGGTTATCATGAGCTAATCTTACCCTTGGGGTATG |
| | TGAGATATTTTCTAACTCTGAGATTGTGATTGCTTTCTGATTGCCATTCTG |
| | CTC |
| BIEC810015 | CAGAGTGTTTTACTCCAAAGCGTAACTCNCATCACCCAGAGGGCTCCCT |
| (SEQ ID | GAATTCCACTTCTTCCTCTTGGAAGTCCTCCCACACGCGTCAGAAAAGAG |
| NO: 15) | CTCGTGGCTTCCTCTTCTTCACTCCCTGCCCCACCTGGGTCACCCACAGC |
| | TACTCTCTTCCATGTTTATGACTCTCCATCGGCCCCAATCCCTGGAAATA |
| | C[A/G]TTTGTTTTATAGCAAGAACACCTTGCTGCTTTCCTCCATCAGACGA |
| | CTGCCCATCCCTCAGTGCTGGATATGTCACCCATACCAGTTTTTTGATTT |
| | ATCTTTGAGAACAGTCTCTGCCAAGAATTCTTGAGTAGAATGTCATTCAA |
| | CCATTGGCCATAACCATTCTCTCAGACAGCACATCTACAAAGGTCTCTCT |
| | CGCA |
| BIEC823988 | AATAGGAATGCATTGCTTTCTGCAATCTGTCTTTGCTTGGAATCAGTAAC |
| (SEQ ID | AATATGTTCTGCAACTGTTATAAATTGAATGCATTTTCTTTATTGAGATA |
| NO: 16) | CATTNCTTTTTTTCATATAAATATTTAATTGGCCCTGAGAGAAAAGCTGT |
| | TGGCATATTTCCTTCATTTGCTGTGGGGTTGGTGAATGATTCAGTCTTCA |
| | [T/C]TGAATAGGGCAATTTCCTGGGGTTGACTACTGCGAGCTAAAAAGCT |
| | CATCCATTTTCAGTCCTCCTTTAGAAAATGAAATAACTACAAATTTGTCC |
| | TCTGTAAGCCATCAGAAAAAATGAAACAATTGACAAATCAGGTTCTAAG |
| | AAGGAGAAACAGTTTATATTTTGTTTGTCTACTACTGTCATTTAAGTGTT |
| | TACCT |
| BIEC846563 | CACTTCTCCTATGAATACTTCTCCAAACGGGATTCATGTCATCTCACTCC |
| (SEQ ID | ATTCTCATCCTGTTTTGGCCACAGTACAGTCACTGCCCAGTCCTTGAGCG |
| NO: 17) | AGCAAGACACAGCTGCTGTTTCATCAAGATGGTGTCTTGATACCTGAGTT |
| | TCTCTGAGACCCTGGGTTCTGTGAGCTCTGCCCAGAGTCCAGAAGCCCTT |
| | [G/C]AGTACAATCTCGCTATTAACCCTGGATCTCTCTTCACATCCTTTCCC |
| | CCTTCTAGGCCACTTTTCCCTTCTCTCCATCCACTTGGATCCACAGCCTTT |
| | AAGTCCGTCNCCAGCAGTcatcttcacttcaaggtctgacagcacctcaacttagtatgaccaggtggag |
| | ctcttcattccactgtcaacccccaacttggt |
| BIEC866619 | GCGGGGAGAGAGCTAGCACACTGAGTCGGCTGCAGGCTCTGGCTGACG |
| (SEQ ID | GGCGAGGCTTACCTCTTGCCTAAGAAAGTGGCTTCCCCACATTTGAGACT |
| NO: 18) | TAAACTCATGCCTCAGAAACATCACCAGCAGCCCTTTTGCATGAATCTCA |
| | GAACCTCCTTGGCAGCCGTAAACACACTTTACAAGTGGTCAACACTGGC |
| | ATG[C/T]CAGAGGTCTGTGGGTTTCACACAGATTCCTTGGCGGGGCAAGC |
| | TGGCTGGGGGACGGAAGCCCTCTGTGGCCTGACGCGCTGTCGTAGCCTT |
| | GACCATGGCCTTTTTGTTTAAACAGACATTTCCAGGGAAGCCCTCAAAA |
| | CATATTCGATTGGGAATGTCTCGTTCAGCAAAGCACATCTGATAGAGAG |
| | AGATCTTGGT |
| BIEC880212 | TCAGAAGGAACCTCCCAGCCTCACCAACCACATATCTCCTTCAGTCACTG |
| (SEQ ID | AGCCTTCCAATTCATTTCCTTGCTGTTACTGTCTACTTTCTTCTATATATA |
| NO: 19) | GCACACATCCAAGGATGAGCATTTCTGAGATAGCCTTCTTTAGAATTGA |
| | AAGACAGATCTCATTTAAAAGAGATGTAGTCTCTACTCCAAATTAGAAG |
| | C[T/C]ATACAACTTCAACATGCTAGAAGTGTCTTAAAGAACTGATGCAAT |
| | TTACATCAATGGCAACAACTTAGTGAACAACTTAAATCATATACAGTTT |
| | ATAATTCAGATGTCAAAAAAACACTTAATATAACGTAACATCATAAAGG |
| | NGATTGTGAAATATATGGACCTAAATTTTTGCTCTCTTTAAGAAACATTA |
| | CAAAGTG |
| BIEC903524 | GTGACTGTGGCAGAACCTCATTACCAAAACTAAGGGTCCACCCTTACCT |
| (SEQ ID | TCCCCAAATGACATGTTATGCCTGAAGGTATCCAGACAATGTCANGTTG |
| NO: 20) | GTAGTAAACTTTTCTTTTNNTATGACCCCACTTCTGAAGGTAATCACTTC |
| | TGATTTTTATTTTGCTCGTGACTTCACTAACCTGACTAAGACTGATTTTAT |
| | G[T/C]CATCTGTTCCTGACTCTCAATATATTTTAACAAGTCAGAAACTAG |
| | NGGGCTTAAGTCNGCATTTTCTGGACACAGATAAACNTTTTNCTTTTGTT |
| | TTGTTTTTAANAATTCATTGAAAAGANNCAAGAAGGAAAACTTCCTCAA |
| | GACAAGNGAGTTTGATTTGTTTTGTTTTTTACTAAGTTCCTCCAATATCA |
| | AAGCTG |
| BIEC933800 | CCATAATCCTGTCCTTATTTCTCCACTTGGATGTGGCAGCACTGTAGCTG |
| (SEQ ID | CAGCATGGCCTTTTTTCTTCCAGAATCAGCTCTTCCTCCGGCAATCCCCG |
| NO: 21) | CCTCAGTGGATCATCCACATGAGCAAAAACCTGGAGTCATAATTGACTG |
| | ACTCTCCTCCTTCACTCCCAATAACCACATTTCAGTTATTTTGCCTTTTAG |
| | [A/T]TATTTCTCAAATCTGTTTTCTCTTTGTGCCCTGGTCCTTTTTCCCAGC |
| | ACTTGTCTTTACTTATGTCCTTCCTTGTGGCTGAAATGCTTCTCTCACTTT |
| | TGTTGTTATTGTTGTTAACACAGCTAGTGTGTTCTTGCTCATTCTTAAAGA |
| | CTCNCTTCAGGCTGTATTGCCCTTTACTTCTATGCATGTGTCTTTCTTAA |
| BIEC100227 | TCACTTTTTCCATTTTGGGCTCATTGCTTGCCTAACTCAACTGCATCACTT |
| (SEQ ID | AGTATTCTTTTGGCTCAGTGGGAAAATAGTAAAATATCTAAGAACTTGA |
| NO: 22) | AGATCCAGAAATGCCTTCTTTCACTCTCGTTCTCTTTATCAAGTTACTTGG |
| | AAACATTTTCTTATTTCAAATAAAGTGTAGGGTTCAAAGTGCTAGGAGA |
| | [C/A]AAAAATCCATCAAGGATCCATCCCCCTAAAGCTCTTCTTGTCTCTCA |
| | TGAAAACATGGCCCCACGTGGTGGGTTTAACCTGTGAGATTCAGGTCGG |
| | AGTCTCCTGCTTGGGGACTTGCCCCTGCTGACNGTTTCTCCTTTGTCCCTT |
| | AAAAATAATTTGGCTCCATATACAATTCTCCACAGACTCCTAATTCCTGG |
| | AAA |
| BIEC119261 | CACAGAAAGGAAAGATACCCCCAAACATTTTCATGATGCGGCAGACTCT |
| (SEQ ID | ACAGAAAACTCGTGAATTAAGGCATTTCAGTAACAATAACTAATTCTAC |
| NO: 23) | CAACACCATTACTATAAAACCATTAACTAACTGACCAAAAAAATTAAGA |
| | AAAAATGAGGAAATAGCAAAAGCCATAATTATGCTCCTGTAAGCAGACT |
| | GAAA[T/C]TTTTGAAAAGTACACCATGTACGAACTACCAACATATAGAA |
| | GTTTGAGCAATGGGCTGAGCACAAAGGAAAAGCTTACACTCACTCTTTG |
| | AGGGTGTAGGGGTGTAGTGGGAAGGGAAGATGGTGATAAAAAAAACAG |
| | TAGTCCCAATTCTGTATTGTGTTACCTACGCAATGTACCTACACAATGTC |
| | ATCAATGACAA |
| BIEC123028 | AACATTCTAACTTGCTCCAATCAGACACAACGCCAAGGTTTCANGCAGG |
| (SEQ ID | TTAATGGAGAACCAAGAGATGGCACACAGCTCTGTGAGACGATGCCAG |
| NO: 24) | GGGACAGCCCAGCACAGAGCACAGGCCCTGGGATTCTCACTGGTCACGT |
| | GGGAGTGGAGGACGCGGCATGAAGCAAGGGCATCTCCGCTCAGAAGGT |
| | TCCCTG[C/T]GGAGCCCCACAGACAAGGCAGCGCGAGCAAGGCCCAAAG |
| | AACAGGCTCCCCCGCCATGGGCTCCTCTCTGGCCCCAACGTGAGGACAG |
| | CCATACTATGAAGACACAGCACTAAGGCAAAAAGCTCCTCATGTGGGAC |
| | AGAAACCCACACCCCACCAAGATGGGTTCTGACTCCTCTATCGTTTTGGA |
| | CTCCCTGAGAACC |
| BIEC141078 | GGGAACTGACCTACTCAGATCTGCCTCCAAGANGTCAGGAAGGAANTGC |
| (SEQ ID | AGACAGAGAAACNCTCACGCACAAACTGGAGAGTGGGGTTGGGCATGG |
| NO: 25) | CCGATCCCCGGAGCTGGTTGGCAGGTGGACACCAGCATCTAGCAGTAGC |
| | CAGGTGGTCTGACTCCAGGACCAGAGCTGGGCTCTTCCGACAACATGAT |
| | GCTCC[C/T]TGCATGGAAAGCCACGAGAGCTCTCCTCTCTTCTCTTCAGG |
| | AAGCCAGTGGAAGAGGAGAACGGAGGATCGGAAGAGTTGTGCATGCAT |
| | CTCCGGCAGTCTGGGGGTGGATGTGAGTCCAGGGGGGTAGGGCCGACTG |
| | GGAAAATAGGAGCGAGGAGCCTGGTGGGGTGGCCCCCAGAATGGAGGT |
| | GTCTGTGCCTGTGG |
| BIEC159353 | TCCAAACAGCCTGGCGGGCTTTCCCTGATATATCATCCTCACCCAGAGCC |
| (SEQ ID | GGTCTCGTGTCACTCCAGGACACCGAGGCAGGAAAAAGACTGACAGCCT |
| NO: 26) | GATGCGATATAATGTGAGTCCCCCCACCATGGGACACCCCCTGAGGTTC |
| | TGTGGCCAGCCTGGCCCATGCCCAGGAGCTGTCACCCACCCAGCCTGAC |
| | CTC[C/T]GGGCTCCCTCTCCCAACTGTGCCGAGGATCAAATGATAAGGAG |
| | ACAAAAAGAAAACAGGGAGCTGGGGCCACACGTGAGATCGGCACCACT |
| | TAGTCATCATCGCGCCCCCACCCCATGCTTACTCGTGACCAGGCCGATGC |
| | CGGGGACGTGGTCTGCCAGCAGCTGGAGCAGGAAGAGGATCCTGGCCCT |
| | GCAGGCGGGA |
| BIEC167336 | GCTCCCAGCCCGATCCCCAGCCAGCCTGGAGGACACTCTTCCCAGTGAT |
| (SEQ ID | CTCCCCCTGCTGCAGAGCTCACTATGGGCACAGTTCTGCACATGAGGAG |
| NO: 27) | GGGTCTCCACCAACATCTGCTGCCTGGATGGTGGCCNGCGACGTCCCCA |
| | TCCCTGACATTTGCCCAGCACCCTGTTGGGCCAGAGCCTTTTCCACCCAT |
| | GAC[C/T]TCCTTTGCTCCTTTTGTGAACTACCTTCCTGGTCTGACCCCCTC |
| | ACATGCCCCCGGGCACCCTGCACGGCTCAGGACGGAGACCCGGGGTGG |
| | GAAAGTCCAGGGTGCCTCGTGCTGGGCTGGGACCTGGGGTGGACTTGCC |
| | CTCCCGAGGCTNGGGGCTCCATGCACANTTGCCCCACAGGCCTGTGTGC |
| | CCCCAAGCTC |
| BIEC170689 | tagtccgtccctcACACACCCAAGATGGCATCCCTGTCTGGTCCAAAGCTGCAC |
| (SEQ ID | AATGTCCCACCCCTCTNGCTGCCCATTCCTGAGCATGAGGAGGTATTTCT |
| NO: 28) | GCTTCTCTGCCCCATTCCTGGGTTCTCTCTCTCATCTTCTGCTTGGGTTGG |
| | CTGCACANACTCAGAGCTGCCTCAGGCCACTCCACAGTTTGGTCA[G/A]A |
| | CATCCCATTGGAGTGAATCAGGATGCCTGGCGTGGTGCCTTCTACCTCCT |
| | GCTGCCTGGACCACCATGTCCCCTTCCACCACTAGCATTTCATGAAGACA |
| | CGTGTTTTCCAAGGCCTGTTCTGCTCCTTTAAAATGCAGTGTACATTTGA |
| | AAGAGCGAGGGGCATTCTGGAGGCTAAGCTTGGGCATGTCTTTAGGGTA |
| BIEC177534 | TCAGATCTTTAACCAAGAATCGATTGATGGAGGCAGCATGGCCAGTGGG |
| (SEQ ID | ACAAAGAGGTAGTCAGGGCCACTGGTGTCACATGTGGCACTGAAATGGG |
| NO: 29) | TTCTTGGAGGTGCCAATAGCCAACCTTCCATCTGCCCAGTCTTTAAAGGG |
| | AAATGAATGGGAGAATGGACTGCGTGGGGCATCAAGATCACTATTATTC |
| | CCC[A/G]TTCCCTTTGCAATATTTTCAAACAGAGGATAGCATATCAAAAT |
| | AAACACCAAGAACAAAGACATCTCTGATGTGCTTTTGTGCTGGCAGGAG |
| | AGTGTTGTCTGCTCTCACAGATGGACTTAGCTTTGTCCAATGAAGAATTC |
| | TGCAAGGGGTGTTATCACCTGAGCTTACCATAGACACCAGAATCTTGAA |
| | TGAGATGGG |
| BIEC187185 | aagcaagaaagggaggaaggaaggaagcaaggaagggagggaggaaggaaggaagcaagcaaggaaggaagg |
| (SEQ ID | aaggaGAAACAGTAAAATAAAAAAACCAAAGGAAAAACTCAGGCAGAAG |
| NO: 30) | ATATGACAAATGGAAAAGATATGTTCTTCTTGTAGATCTGAAGTTCTCAC |
| | TTGCAAGATGAGATAATACATCTTTGCTT[A/G]TTAATCTGGAATTATAA |
| | TGTTTGAACCCTTGAAGTCCTCCAAGAAACTCAACCCTTAGAAAAACCC |
| | ACAGCTGTCTCCTATAGGTTTTCAAATAATTGACAAGTATCTCTCAAACT |
| | TGGAAGAATACCTTTAAGACTTCAGTACACACTCTCTGTCTTGACTAACT |
| | GACAAAGCAGAGGAATTGAAACAGATACTTCACT |
| BIEC214463 | TGCATAGATTCAGAAGCCAGCTGGTGAGACAGCGTTATAAAGGAGGTAT |
| (SEQ ID | TTTAGAAAGANAAAAGTCTTGAGCAGAGGGTTTTGTTCACAAAAAGGGC |
| NO: 31) | AACAAACTACCTGTGCTAATAAGCTTATTCACNATAAAGTGACTGCTGT |
| | GAGACTNTGTGAGGTCAGCTCATCACAGAAAGCTGTCACTCTACTGTAT |
| | TACT[G/C]TAATAGACGTTTAAATACATGTTTCATGCCTACATCTCACTGT |
| | TGTACGACTCGAACACATATAAACCTCAAATGTCAGGGCTACATTCAAT |
| | TCAAGACGGTATGCTGTTAGCTCTCACAATCATAACTTGTATTCCCTGGG |
| | AGGAATGTTCAGAAATGTTCCCCTTCCGATGTGAAGGCCTCTCTACCTCC |
| | AGTCCAGT |
| BIEC220494 | GCGGGCCCTTGGCAGGAAGGACCTAGGGACTGTGCCGGGGCTTAGAGTG |
| (SEQ ID | CAGCCTTCAGTCCTGAGAGCTGATCAAGGAGAAGGGGCAGTTCCATGGC |
| NO: 32) | TCTGGAGAGGGTCTCCCCCTGCATACCCTGGCCACCTCGATCCACCGCTC |
| | CAGGACCTTGGCCCTGCCCTGGGCCGTCATGTTTGGGTCCCCAAGGCAG |
| | GAC[G/A]TCATGACACAATTTGCTACTCTGTTGAACTGCACCACTGTGGC |
| | CCGGACGGTGGGTGCCACGTTCTCATGTCCAGGCTGGGTTCTTTTGCCCC |
| | AGGTGGAGCTCAGATACTCAGAGGGCACGACGTTCTTGAACAATNCCTG |
| | GTGAAGAGGGGGAGAGTCACTCAGCCCTGTCACAGCCCANCTCAGTGTC |
| | CAGGCAGGA |
| BIEC252403 | AGAATGCCCCCTCTCTTTANGTAGAAACGGGCATGTGGGTGTTTCAGGC |
| (SEQ ID | CTCGCATTTAGATCATAGGAGATGGAAAGATCTCCCAGAGCCTGTTCCA |
| NO: 33) | CACTGCAGTTGTCCCCCAGTTCAATGACACTATTTTCTTAGGAGAAAACC |
| | AAGTTATACCGCCACTTTGCCCTTTTGAAAACTGCGTCTGCTCATTTATTT |
| | T[G/A]CTTGGTGCATATCTTAGACAGGCATTACCACAGTGGTTTGTAACC |
| | TTCGATTTGTCCGTGTTCCCTCTTTGGCTTCAAAATGCCACACGACCCCC |
| | TTTTGAGGTTGGAAAGAACCTCCTTTTCNCTATAATTTCAAGGGGAACTT |
| | GCAAAGTATCACGATAATTGAAAAAAATCTGTATCATAATATCAGGATC |
| | CAGGTT |
| BIEC270317 | TCGGCTCGAAACGTTTTCAAAGTAAACAAATGAGTTAGCAATTTACCAC |
| (SEQ ID | TTAGGATTCTCAAAGTGAGAGTTTATCCCACCAAAAGTAATTTTCCANCT |
| NO: 34) | CCTCCCCCTCAAGCCTATGCTGTCCTTTTGGCTACAGCATGGGCCAAAGG |
| | TTGATAATACTTCTGTATACATTTAGCAAACCCAACCTCTACCAAACTAG |
| | G[G/A]GAACGGCAAATGATACCGGTGGATAGAGACCCAGGGTGCTTTAA |
| | CGTCAAATGCACAACTTGATGGCCGTCTCTCACCGTAGGACAGTGGAAC |
| | AAGCAACTGCAGTGACTCAACATGAAGGGCAGGAATCTCCATAAAGTA |
| | ATCTCCTGTTATCAGGAAATGTATTTATAACTATTTTGTAGATGGGTACC |
| | CATGTCTCA |
| BIEC304838 | GAAGCAAACCGTGGGATAAGGGACCTGTCACTTTATAAGCAGCTCAGAC |
| (SEQ ID | TAACTGAAAGCGTGAAATACCTGTGGTTAGAGCTAATAACAAAATAACA |
| NO: 35) | GTTATACTCGTCACAAAAACATCTTACACAGGTGAAAACATGAGTAGTG |
| | GAAAATGCAAGCTGCCATATGCAGGAGCAGAGCTGGCAACCCTGGAAG |
| | ACTGT[C/T]GCTTGCCTCCCGAGGGTTCAGCAGAGGGCCTTGACGCCCCC |
| | TCTCCGTAAGGAAAAGTCCAGGACACGGAGAGGGGAGGCAGTTTCTACC |
| | AGAGAACCCATCTTACTCAACACCCTCCCCCCAAAGAGGATGGCAGCCC |
| | CTGCGGCCTTGAAAACCCCAAAGCCTCAAAGCTCGGTGCCTCCCGCCTG |
| | GCCCGAGAAAGG |
| BIEC306934 | TCAGGTCCTCCACATCCAGTTAAATTTATCCTGGAAGCAATAAAAATGTT |
| (SEQ ID | AAATATTACTTGGTTAGAGTTTCTCCTCCTTTATCTAGACGTAACTGTGT |
| NO: 36) | AGTGGGGGATAAATGGTTGTAATGCAGATATTCGAGAAGGTTCACTGAT |
| | TCCTTCAGGCTACCTGGGGCCACTCATGACATGTTAACGAGTATTTACTG |
| | T[G/A]TGTCTACTCTATGTCCCTATGCAATTTGACCCGATATTTTTAGTAT |
| | TTCAGCTTGAGTTACCAAGTGATTCGGTAGGTATGTGGGAAAGTTTAAT |
| | ATGTCTCCAATAACCAGTAACTTATTAAAAATGGATCTTCTCACATAGAA |
| | CAGAGAGTTACTCTACCCAATCACCGAATAATTTCCAAAAATTACCCCA |
| | GTTTAC |
| BIEC323723 | GACTTTTAATATTTGGATATGTGAAGATGTTTATAAATTGGTAGTATGGA |
| (SEQ ID | GAGTTCTGAATTNTATGCCCACGTTCTTGCCCAGGCAGAGTGACATTTCC |
| NO: 37) | CCTTCCTCCCGTCCAAACTAGGAGCAAAGGACTTTTTTAAGTAAATTATT |
| | TTTAGATTTCCCAGAGATGCTTTTTAAAGAGCCGTTTGTTTTCGTAAACA |
| | [C/T]GTTCGACGTGAGTTATTGTGAATTTTTGCTAATAGAGGCTGACAGTA |
| | CACATAACACGAGCAGACAGCAAGGTACAGCCCCGGGCACCCGTCTTTG |
| | GTGGCTGACAGGCTGAGGGATGAATGTTGAGAGCCCCGGACAGCCCCG |
| | GGACCGACGTGTCAGGTCGGGCATGTGCCAGGCTCTCCCCTCTTTCTCGT |
| | CTCCAG |
| BIEC338343 | ACAGTTGATGCTTAAATTGTCCTTGGACCCTGTAGCTGCTAGATTAGGAA |
| (SEQ ID | TTCTTCCTAATCTGTTCCCAACTTTATGTTTAGCACCAAAATATCTGTTTT |
| NO: 38) | ATTTCTTCTACTTCTCACAAATTGGAGAAATAAGAGTAATTCATAAATCT |
| | TTCACCTCAATTATTCTCTTTTCTATAAATGACTAAAAAAATACATTCA[G/ |
| | A]GTAGGATTGTAAAAATCTATGACCAGACCCTTTGTTTTCAAGGTTATA |
| | GAAACAACAAAAGCTTGAAATTTTACAAGGAAATTGTAATAATTTAGCT |
| | CAGGTAAAGTGTAATTTTCTTCATGAAAAAGCAAATATTAATCTAAATA |
| | GTTTTCAAGTGAGATATCAAAAAGAGACCTCATTAAGTAAATAAAATAC |
| | CAATT |
| BIEC347016 | AACCCCCACAGCTGACTCGTCAGTCTGTCTTCAGCCACTAGTAACAAGC |
| (SEQ ID | CCAGTTCTATAGATGAGCCTCTCGAGGCCCAGAGCAGCTTGTTTATAGTC |
| NO: 39) | CTCTAGTTCGTAAGTGATGGAACCAGTCTTTACTTCTCCTACCTCCCTCA |
| | AGCAGTGCACTGAGAGATAGAAACTAAGGATCAAAGAAACCACAATAC |
| | TTC[C/T]TAGTGCTTCTTCTACAGATCTGAGAGTTTTTGAAAATGAGACC |
| | AATTTGAGATAAAAGCCTTCAGCACTTGTTTCCAAAACTTAATTCATTCA |
| | ACAAATATACCTGGCCCCCACTAGGAAATAACGGAGTCGGGGCAAGGC |
| | AGTAAGGTCTCCACTCTTTTTGATGTACATGCTTGGTGGTGATGGTGGTG |
| | GTGGGGTGA |
| BIEC450770 | ACGTATTGTGTTTTCTTTCTAAGTCTCCTACCAATTGTAAGTTCTCTCCCT |
| (SEQ ID | TGACCAAAATTGTGCAAATTTTCTTCTATATAGTCTTCTGTTTTTATTTTT |
| NO: 40) | ATATTTAGTGTTTACATTTTGTTCATATTAAAATTTCATTTACAGCATCTG |
| | GATGTATCTTGCGCACAACAGCACCTAGGAGTGGATTAAACGTTTTT[G/ |
| | T]ATAATTTGGAAGCATTCTTTTGGTTTTCTATCACCTTCGGGCTATGATT |
| | ATACAAACAGATTTTCACTGATCTGTGATTATACTTGTTAATAGAGCGCC |
| | AGGCCACNGTCCTTCGAAGTGGCTGCACCCACCTGAAATCTATTCTTACA |
| | CCTGCTCTAACACTGTCCTGAGTCCCCATTCTTGGGAATTCTGACCCATTA |
| BIEC465101 | CTGCCCCGGGAAAGCGGCTGGGGAACCAGTGTTCAGGGCCTATCCTCCC |
| (SEQ ID | ACAGATCCCACTCAGCATTCAGGCTATGCTGGTCAGTGGACTGGGGATT |
| NO: 41) | TTCCCTGGAGCCTTCTACAACACCAGGACTCCTGCTCCGTGATCTTCGGA |
| | TGGAAATCCTTGTGCCCACTCTCACCCCTGCCCGCCCGGCCTGGCTTGTC |
| | CC[T/A]AGAAGGATGATGTTCTATTCTTTCCCATCCCTGGAGTCCCTCTAG |
| | TTGATTCAAAGAAGTGGGAATCATAGTAAAAAAGAAGAGAGACTCATCT |
| | TCTGGTGGGTCTCGGTTCAGGATTTCTTACCTTCCTGATGTGTCTCCGTTT |
| | GCAAGGTGGTCGTAAGGATGGATTCCTCTGGAGGGAGGGAAGGAACAG |
| | GAGGAAG |
| BIEC486760 | CAAAGCTCCTTTTCTATGTCCAATGAATGGACAAATCCCTCAAACGATTA |
| (SEQ ID | AATAATTGCCCCAGGATCTAGGTGTAGCTTAATTCAGTTAGATTTAGAAT |
| NO: 42) | GTGAGGATTATAAGGAAGTCAAATCAAAATATGGAAAAAAACAAACAA |
| | GACTTCTTTCTGAGCTAAGCTTCTCCAAAGGCTTTGGGAAAATCAACAG |
| | AAC[C/T]AGTGATTTGATTTGAAAGTCCCTTCCTGTCTTAGGGTTGCACTT |
| | TGAATGCCCCATACTTGTTCTTATGGACTGCTGACCAGAGTACCTCCACT |
| | CCTTGATTTTCTCCTTAAGAACAAATTTCAGCATCCTAGAGAGAATGCTT |
| | TCTAAATGGCAATTATCCTTAGGTTCCTGTTACCTAGGAATGTGTTTACC |
| | AGTTGT |
| BIEC507792 | TTATACTTTGTATTATTTAAAGTACCTGATGCCATGCTACACAACTAGTG |
| (SEQ ID | TGTGTGCATCCGTTCATCTGACAGATATTTANTGACTCACCTNCCAGCTG |
| NO: 43) | ACTCCCTGGGAAGCCAGAGCAGTTTGCCAACTAATATGGTTAATTGGGA |
| | TTTGATAAGTGCTATAAAGACACCAGAGATTCAGCAGTTTCTCCCAGCT |
| | AT[T/C]GAACTTATTTTGGCTAATGGATATCACTCTCACTCCCATTTCAAT |
| | CTTACAAAGGGATGCTAGAAGAGGATTGACCAGTCAGAAGGTGGAAAC |
| | TTAATAAAAATTGNAAGTCAGAGACGGGAGGGAATGAAAGCAGCAGAG |
| | GAAGAAGGAGGCAGAGACTGGACAGCCAGCAAGGCCTCAGTGCCCTGC |
| | ACAGTTTAAGC |
| BIEC521111 | CAGTGTTTGCCACTCTTGCCAGCATTGAGAAGTCTCCCAGGCTGGTTAGG |
| (SEQ ID | GAAGCGGAGTGTGGCTGCTTGTCATGCTTTTCACCGGAGGGCAGTGTCT |
| NO: 44) | GATCCTCCTCGTGTCCCCACTCCTTCCCAGGTGACCCACATCACCACTCC |
| | ACTTTATTCCTAAATCTTTGACTCGCTAAATCCTCCCTAAATCTCAGAAC |
| | A[C/T]GGAACATGTTGAGGAGCTGGCATGCCCACAAACTCCTAAAAGGC |
| | AACATGGGCCCCAGAAGGGCGCCNGGCAGGCAGGGAATCCTGATCTCT |
| | AAGATAGGATTGTTAAACCCTGCAGACTCGGCTCCTTAGGAAATGCACT |
| | GGTCTCAGAGAGAACNGAGACCCTGTCGGGAGCTCTTGGGATTTGTCTC |
| | TCACTGTCCC |
| BIEC547263 | TTAGTTGTGGTGTATGTTAAGGCTAATTGCCTCCCCCACTAATTATTGAA |
| (SEQ ID | CAGCTGTCGATGCCATTTAATAATAGTCCACATTTTCCCAATTGATTTTA |
| NO: 45) | AATGATACCTTCATAATATATTCAATTTGACTTCTTGCTCCGATAACGTA |
| | GCAGACTGAGGAATGTGGCCTCCTCCTGCTGAGCAACGTCAACCAAACT |
| | T[C/T]GCAACAACAACAAAAGTAATTAATCTTGAAAGAAAGAAGGAATG |
| | GGAGATGCCCAGGTGCTGGAAGATGGGAGGGAAAAACCAGAACCGGAA |
| | GCTATTCCATGNCTGAGGACCCCAAAAAGGCAAGATCCTCAGTGAAGAg |
| | agctcacagctgaaaatagagaccacaggctggaacgatctaccacgagggagagtcag |
| BIEC574261 | CAGATGAAGGAAATATACAAGCATTTGTAAAGCCCTTTTTAAAATAAAA |
| (SEQ ID | GAGAATTCAAAGACTTAAAAATATATCAACTTATTGTGACAAAACAATA |
| NO: 46) | TTCCTCTTGTCTTACCAGACTTCAAGAGAAAACTTCGAAGATGTCAGGA |
| | NAACAGGTGTAGTGTTCTCTTTAAGATCGGCTCAGCCTCTGAATGTTGTA |
| | AAC[T/C]TGCCAGCCTGACTTGCAAGAGTCAAGACGAGCACACAGGCGT |
| | TTCTTACAGGCGGCACCAGCTCTCTGCCCAGAGGGAGCCAGCAAAATCC |
| | CGGAAGCCTGTACACAGTTTTTCTCAGACCATGTATATGTTTAGAAGATA |
| | GTACCNGGATGGCTCTAGGGAAAATTATTGGCTTCCATGTAAAACCCAA |
| | AAGAAAGAAA |
| BIEC585067 | TTGCCACCAAAGGCAGACTCTGAATTTATGTTCATAATCCTGAGTCTGGG |
| (SEQ ID | TCACCAACGAATGTCTTTCTGGTGAGGCCTGAAATCTAAATGTTGGGAA |
| NO: 47) | TCCAACGGGTCTTGGCAGTGCATGGAAGGCTGTTTGCCAAATATCTGGA |
| | TCTTATTTATTCCCTCCAGTCTCCCCAGAAAATGCTCTTGTTCATTTAAAT |
| | A[T/C]GGACGTGACTACATTTGTTGGGGACCGTGTACTTTTTTCTTTAAAT |
| | AGAAACGCCATGTGTGTGATGTTTTCTTTGAAAAGGAAAGCCCAGGAAT |
| | TGTCTGCATCAGATTATAAAATGATCCCAGGGTCCATTCCTGGCTCTAAG |
| | CAAGTTGAGTATACATCACCGCGTTTAATTCAGCAATATCATCAATGTCA |
| | GTGCG |
| BIEC609174 | CTATCCTTGGGGGATTAGATGTTGAATATGTTTGGTATATAAATGTCATA |
| (SEQ ID | CTCAGATAAAATTTTTTGTTGGCAACACAAAAGAGCACAACATGCGACT |
| NO: 48) | AAAGCTAGAGAAATGTTCTGATTTCTATTAACTTTTATGTTCCTGTATAA |
| | GCTAAAGCAGTTGAGACAATAGTAATTAGTCTTCCAATCTCCCATTCCTG |
| | A[T/C]GTAATGGCTTGGCAGCTTTGAGGGTAGGGGAAGACTAATNAAGA |
| | GTAGGTAGGTCAGTGTTCTGATGGACAGCAGCAATCGGCAGGACCATTA |
| | ATACAGGACTGGAGTCCAGTTGGAAGTCTTATTATTATGGTGCTCATCTT |
| | GCTTGTTCAGTTGCTGACCCACAACCCAATTTTACCCTTTACCTTTTCTTC |
| | CGTGGA |
| BIEC628735 | TCTTCACTTCAAGAGCAGTTATCAGAACTTAGGGGTCTGAACTACAGAC |
| (SEQ ID | TTTTAGAGAACTAGATGGCAGATGATTCCCGTCACTGGGAATGNGGCAA |
| NO: 49) | TATCCCAATNACACAGCTGTTGCCACCAGCTGTGAGAACACAGACACTA |
| | CACTGGATCAACTGGAAACACAAATGACCCCGGGTACAGACCACACGCT |
| | AGGG[C/G]GCCACGACCATAACAAAAATACCTGCTGAGAGGAGGAGAA |
| | CTGGCCAACAACGCTACCTCATGCAAAAGACACACTTCTGAGGAACACG |
| | CCTGTGTCATCAAGATGATGTCCACTTGGGGCCATGCTACCTTCTGAGCC |
| | ACTTCTGTTTCTCTGGCAGAATGTATCCAGCCTTCTGATTCAGGGCGTCA |
| | GGATCACATCT |
| BIEC688595 | TTTGGACTAATTTAATGGCTTAATTTCAACATGATCACGATTAAATTGGA |
| (SEQ ID | TCTGCTGTTGAAAAAATAAAGAAAGAAAANACACTCACCCAGAACACTT |
| NO: 50) | CCAAAAGAGTGGATTTGAAAAGGAAAAACCAACCAACTCACCCCAGGG |
| | CCCCAGCCCCCGCTAAGGTTCAGCCTCAGCGTTAAACACATTAACATGG |
| | AGGA[G/A]AGGTGTGTGTTTCTTGCTCTGTAACAGGCACACGTTTCAAAC |
| | ATAGGATTCAACATATGAGATTTGAAACATCTTTGGCATTTTGAAAAAA |
| | TTTACAAACTAGAATCCCAGCTCGTTGGTTTTCTGAGTTGTTTAGGAAGT |
| | CACTTAAAGGAAAATAAACATTCTCCNTGTGTTCGATGAATGCTACAGA |
| | ACTTCCGTGC |
| BIEC697335 | CTTTACATTGCTTCTTGCTGATGATGAGTACTTTCATGATACCCTGTGCCT |
| (SEQ ID | GATCTACACATAATCAGAACCCTTCATTTCGTTCGGATGTGGTTCCATTT |
| NO: 51) | ATAAAATGTCAGGGGTGCAGACCAGCCCAGAACATCCTAAAACTTCCAG |
| | AAGACAATAAAGTTTAGGCAACGGAGAAGTTGGTCGGGTGTTCCTCCCT |
| | C[T/C]GTCCCTCGAGGTACAAGCTTCGGTTCTGCACGACTCAGCTTTCCA |
| | AGGTGAGCTGCGTTGTCAGGTTGGAACACAGTTAGGTCTCAGACTGACT |
| | TAGCGCTCACACCCACCCCTGTAGGCCTCTTACTTCTGTCCTCATGCCAC |
| | CCTCCCATCCTCTTTCTTGGCAACAACGCCATGAAAAACGTGCAGATGG |
| | GCTCCAT |
| BIEC938831 | TAGAGTGGTAGCATATTAAACAACACACAATATTCTATGGCTTGAATAA |
| (SEQ ID | TGGTGTTTATTTTCTTTAAATACGCTTAAATATGAAACAACAGACAGTAG |
| NO: 52) | TTCTGTAAACTCAAAACTAAAAGCTGGACGGTAATCAGAAAATCAGTGC |
| | TGATCATACAAATCAAAATCTGCATAAGAGAATCAGAATAATTCACAAG |
| | ACA[G/A]TTGCAATTAGCAAAGGACTCGGAGACTATGCGAATTGCCTCGT |
| | CTGCTCTGAGTAATCAGTCACAGGGCACCTGTCATTCCATGATGATCAA |
| | ACCTTTTTCCTTCACCAAAAAAAAAAAGGGTTATAGAGGTTTCCTCCTCT |
| | CCATTCTTTGTTGTGCGGAGGCATCTTCAGTTAGAAGACATCTGTCAGTG |
| | AACCAGGG |
| CREAM | CTTTGATTGCTGACCGAAGGAAGAAGCTGACCTGGGCCATAACCATCAC |
| (SEQ ID | CATGATAGGTGTGGTTCTCTTT[G/A]ATTTTGCTGCTGACTTCATTGATGG |
| NO: 53) | GCCCATCAAAGCCTACTTATTTGATGTCTGCTCCCATCAGGACAAGGAG |
| | AGGGGCCTCCACCACCACGCTCTCTTCACAGGTAGGGAATATTCCGGAA |
| | AGTCTCTCCTTTAGCTCCCCAGACAGGGAGGTTCTTACACTGAAGCCATC |
| | CAGTGTCTCTGCATGTCAAAGTTTTTGAATGAATGGATCAGCTGATGGA |
| | ATGCTCTCATCACGCGGGCTCAGTCTCCCAGTGCATTTCTCTAAATAAAG |
| | TCAACTTGTGACCAGGCTGAAGGGTTTTGCAAAGGAAGTTTACGTAAGA |
| | GCTTTCTGAAGAACTATTTGTGGAGACATTTGCAAGTGAAATGAAAAGA |
| | GGCATGGTGTACTTTTGGGGTGATTTTTTCTTTTTAAATCAGCAATGTGTT |
| | TTTTTTAAGACAGCAATGTAGCATCGGTATTATTTAGGAGCTTGTTAAAA |
| | ATGCAGTTCCATTGGGCCGGCCCAGTGGCGCAGTGGTTAAGTGCATACC |
| | GTGTTGCTTCGGTGGCCCCAGGGTTACCAGCCTGGATC |
| SILVER | CAGGTGAGGGCCCCACCATCCAGCGTACACCCCCTTATCCCTTATTACC |
| (SEQ ID | ACCACTCACTCTTCCTCAAGGGGAGAAGGAACCACCACTCCCTGTGAGG |
| NO: 54) | AAGCATGGTGTACAGGAAGGAGCCCAGACTTGGAAGTTAAACAGGCCT |
| | GGCTTGCAGTCTTGCTGGTGAGACCTTGGAGGAAGTAGCCTAACCTTTCT |
| | GAGCCTCTGAAAAGTAGGAAAATTAATACCTGCCCTGTGGGGGATGTTG |
| | TCAGGATTAGAGACAATGTGAGTAAAGCTGGTTCTGAGGCAAGAGTGTA |
| | ATAAAGGATCATATTGATGATTGTTATTAATAAGATAAAAAGTGGAGGA |
| | GGTTGGCTGAACTGAGTTCTTCACCTGTAAGAGGGGCAGATCCCCAGGC |
| | CTGGAATGCCAACGTCCTCAAAGCAGGGAAGCTTGTAGAGTGAGAGGG |
| | GAATGGACAGAGGTTACCATATAAACAAGAGAAATGAACCCTGTTTGTG |
| | AGGAGAAGAGGAGGCAGCTAGGATCAAGGCCAAGTAAACCTGGGATGT |
| | GGGTGTGTCCTCTTCTTTGGAGAAGCACAGACAGGCTGCCCTTGTCCATT |
| | GCTTACCAGTTTCCTTCTTCTTCTCCCAAATCAGG[C/T]GCAGACTTATGA |
| | AGCAGGGCTCAGCTCTCCCCCTTCCCCAGCTGCCACACGGTAGAACCCA |
| | CTGGCTGCGTCTGCCCTGGGTCTTCCGCTCTTCCCCCTTTGGTGAGAGCA |
| | GCCCCCTCCTCAGTGGGCAGCAGGTCTGA |
| TOBIANO | CAAGCGCTCATTTAACGGAACGAGAAGCCCTAATGTCTGAACTCAAAGT |
| (SEQ ID | CTTGAGTTACCTTGGTAATCACATGAATATTGTGAATCTTCTCGGAGCAT |
| NO: 55) | GCACCGTTGGAGGTAAAGCCGTGACCCGCTTGCATTTTATCACCTGTCGA |
| | ATTATCAGAAGGGGGAGATTTTGATATGATTTTGACAATGCTTGATTATA |
| | AGCTCCTTGCAAGATTTTTACCCAAGTTGTTGTTACCTCTTGCTAGAGTC |
| | CCCCCTGCAAGAGTTATTGTTGTTAAGAGTGTATATTTTAGTTTTCTCATC |
| | GTGGGGTGGGATAGTCCATGACATACCCTAGTTACTATCACGTATGTTGC |
| | ATCCAAAGCATTGACCTCTTGAATATCTCGAGAATGTCTCATCTTGTGCC |
| | CAAAGCTTTTCTTTTTATCTTGCAAGCTTTGTTTTGCTGGATCATATTTAG |
| | TCACTAAAGGGTTAATATTCATTTGCATATCATAATTAAAAATAGGCCTT |
| | TAAGCCTATCAGCTCACAAATATACACCAAATGAAGCAAACACCTCACT |
| | CCTGTAAAAAATAAATTTCCAATTCTAAAGCATTAGTCAAGCCTCCCATC |
| | AAGGATTTCTGTGGTGTTTCCAGAAAGCATTTTGGTCTTAAAGAAACAA |
| | AGATTAGTAAAACGAGCTGTTCCTTGAGAACTGGAGAGATCCATGGTCA |
| | CGTTGACAGCTTTATATAATTTCTTAAAGCAGACCCCATACCTTTTTGCC |
| | TCACCACGTCATGACTCATTCGTGAGAAATTTCCGCC[C/G]GAGTTAATT |
| | AGTTGCTTGTTACCTTCAGAGCTGCAGTTTTAGGCATTCACAACACCAAA |
| | AAACATTTTCGCCTAGTAGTGCTCAGAACGCAGGTGACGCCACCTCATTT |
| | CAGGTTGTCACCCGCTTTCCAGTCTCGTCTCAACGAGCTGGGCTGTTCCG |
| | TGTGGTTGGTTTTCCTCTTGTGCCGCAGTTTGCGCTCTCATCGTTTCCGTA |
| | ATAACGTTAACCGAGAGGCGTGATGCCCCACTGTAAACTGAAGGAAATC |
| | AAAGGATTTATGTAGGACGGTTGATAGTAGCGTAAGTAACTCCTGTCTG |
| | TAAAACCAGCAGTCACACCTGGCCCCTAATCTCTCAGAAATTCAGGTAA |
| | AAAGGGGCTTGCTCATTTAGTTTTACTCCAAATAATGCCGGGTTTTGATA |
| | AGCAATGTTAATAGAGAGGATTGTATTGGAACTAAGTAGGCTCATCCAT |
| | TGAACCTGAATATTAATGACTCTGATCACCCTTGGGTATTTTAATGGGAG |
| | GCAAGAATTGTCTATATGTCTCACCTCTTTCTACCCTTTTCCTATATGCTC |
| | GTAGGGCCCACCCTGGTCATTACGGAATATTGTTGCTATGGTGATCTTCT |
| | GAATTTTTTGAGAAGAAAACGTGATTCATTTATTTGCTCAAAGCAGGAA |
| | GATCACGCAGAAGCAGCA |
| SABINO | ACGAGCTGGCCCTAGACCTGGAAGACCTGCTCAGCTTTTCTTACCAGGT |
| (SEQ ID | GGCAAAGGGCATGGCGTTCCTTGCCTCAAAGAACGTAAGTGGGAAGAGT |
| NO: 56) | CCTTTTTTTTTTCCTTAATCGTGGAGCATTTTAGAGCCCTAGTTAGAATGC |
| | AGAGTGTCATTTTGAAGTGTGGTAACCAAAAGCACAGGAAATTTAGTTT |
| | CTTCATGTTCCAACTGCTGTCTCTTTGGAATTCCTGTTCTCATTTATAAGC |
| | TTTAATGTGTAAGCCTGTCTAAATGAGCTTTCTATGAATATATTTTTGTAT |
| | GCAATGAATTCATGTAAAACTTTTGGCTTTTAGGATATAGGAGCTGCTCT |
| | GAGAAAATAGAGAAATAATTATTTTATCAGCAAAAGGAGCAGGTACCTC |
| | ATGTAGTTGCAGTGCTTGGTGAAGCATATACTTGAGTCTTATTAAAGTTA |
| | GACCCCAAATATTGCGTGTGGGTTTGTGTAGTGTAGGGGAAGAACCAAT |
| | CAGGATAATAAACATTTGGGAAAAAGACGGGGGGGAGAGAGAATGATG |
| | GAACCATAACATGGAACATGGTCCCTGGATAGGAGAGAGGAGTTCCCTA |
| | GGACATGGGACTAGCAGAATAGAATAAGATTACAGATTCTGCCCTTAAG |
| | TGTCGTTGGTGACATTTCCAAACAATTACCAAACTAAAAGAGGATATAG |
| | GATGGCTGAAATAGCCTCTTCCCTGTGTCCTTGGGAGATGTCAAATTGAA |
| | GTTGCAAAGACATTTTAGAAACTCTGTAAAAAGACAGTGAAAGAGAAG |
| | CATGCAAAATGAGTCTCAGTTTAAAAAATATGATACAACTATTTATAAT |
| | GTATTTTCCTGTGAATGAAAGCCGTCCTAAGAAGAAGAAAAGCATTTAT |
| | TGGAGTTGGTTTTGAAAGTGATTATAGTAATTAAGGTCCTAACGATGAG |
| | AAACACAAGTTTTGAACATCATTCAGAGCATAATTTAGATATTTATTTTT |
| | GGTGCACTGAATAGTTTAAATGTAAAGCAAAAAGTATTGGATTGGTAGA |
| | ACATAAGCAGCATTCTAGCATTAAACATAGTTTCACTCTTTAAAAGTTTA |
| | AAATAAATTTAAATGGCTTTCTTTTCTCCCCC[T/A]CTCTCCTAATAGTGT |
| | ATTCATAGGGACTTGGCAGCCAGAAATATCCTCCTTACTCATGGTCGAAT |
| | CACAAAGATTTGTGATTTTGGTCTAGCCAGAGACATCAAGAATGATTCT |
| | AATTATGTGGTCAAAGGAAATGTGAGTACTCACTCTCTGCTTGACAGTCC |
| | AGTGAAGGATTTTAGTTTCACATTTTTATAATAAGTGTTTTTTATGATTTT |
| | CGTAATGCAAATGCTCCCTTTGAGATAGCATGCATTTTAGCAGTCAAATT |
| | AAGTGTACTTCAGCAAAATTTGTGTGGTATTGCTGAACCTTACTACAACT |
| | AACAT |
| AGOUTI | CCTCCCAATTCTCTGCAGTTCATGGGGTAAGGGGCGGTGGGGGAAGAGC |
| (SEQ ID | AAGGGGGAAAAGACCAGAAACATCTGGCTTTGCTCCTTTTGTCTCTCTTT |
| NO: 57) | GAAGCATTGAACAAGAAATCCAAAAAGATCAGCA[GAAAAGAAGCA/*] |
| | GAAAAGAAGAAGAGATCTTCCAAGGTAGGCCTTGGACTTCTCATTGTAG |
| | GGGTGGGACCAGACTTAAAAGGGGAGGACCCTGACCCTCAAGCTCTGGC |
| | TAGGAACTAAATGAAGGATTTTTCAGGCCTACATGAACAAAAGAAGCTG |
| | AAAGCTACCAAAAGGCTTCCTGGCCTGGAGCCCTGAACCAGACCCCACA |
| | GAAGCTCAGGGAGCTGATGT |
| MC1R | CACCCTCCCAGCCACCCCCTACCTCGGGCTGACCACCAACCAGACGGAG |
| (SEQ ID | CCCCCGTGCCTGGAAGTGTCCATTCCTGATGGGCTCTTCCTCAGCCTGGG |
| NO: 58) | GCTGGTGAGCCTAGTGGAAAATGTACTGGTGGTGACTGCCATCGCCAAG |
| | AACCGCAACCTGCACTCACCCATGTACTACTTCATCTGCTGCCTGGCCGT |
| | GT[C/T]CGACCTGCTGGTGAGCATGAGCAACGTGCTGGAGATGGCAATCT |
| | TGCTGCTGCTGGAGGCCGGAGTCCTGGCCACCCAGGCCTCGGTGTTGCA |
| | GCAGCTGGACAACATCATTGATGTGCTCATCTGCGGCTCCATGGTGTCCA |
| | GCCTCTGCTTCCTGGGCAGCATTGCCGTAGACCGCTACATCTCCATCTTC |
| | TATGCGCTGCGGTACCACAGCATCATGATGCTGCCCCGTGTGTGGCGTG |
| | CCATCGTGGCCATCTGGGTGGTTAGTGTCCTCTCTAGCACCCTCTTCATC |
| | GCTTACTACAACCACACGGCTGTCCTGCTCTGTCTCGTCACCTTCTTTGT |
| | GGCCATGCTGGTGCTCATGGCAGTGCTGTACGTGCACATGCTCGCCAGG |
| | GCGTGCCAGCACGCCCGGGGCATCGCCCGGCTCCACAAGAGGCAGCACC |
| | CCATCCACCAGGGCTTTGGCCTCAAGGGTGCCGCCACCCTCACCATCCTG |
| | CTGGGCGTTTTCTTCCTCTGCTGGGGCCCCTTTTTCCTGCACCTCTCACTC |
| | CTTATCCTCTGCCCTCAACACCCCACCTGCGGCTGTGTCTTCAAGAACTT |
| | CAAGCTCTTCCTCACCCTCATCCTGTGCAGCGCCATCGTCGACCCCCTCA |
| | TCTATGCCTTCCGCAGCCAGGAACTTCGAAAGACG |
| LWO | GCGACGCACCCTCCCTCCTCCCCCGTGCGAAAGAACCATCGAGATCAAG |
| (SEQ ID | GAGACTTTCAAGTACATCAACACAGTAGTGTCCTGCCTAGTGTTCGTGCT |
| NO: 60) | GGGCATCA[TC/AG]GGAAACTCCACACTGCTGAGAATCATTTACAAGAA |
| | CAAGTGCATGCGGAACGGCCCTAATATCTTGATCGCCAGCCTGGCTCTG |
| | GGAGACTTGCTGCACATCATCATTGACATCCCCATCAATGTCTACAA |
| GBE1 | ACGTGCCCGACCTGGGCCGCCTTCTGGAGGTCGACCCGTA[C/A]CTGAA |
| (SEQ ID | GCCCTACGCCCCGGACTTCCAGCGCA |
| NO: 61) | |
| JEB | TGTTACTCAGGGGATGAGAACCCTGACATCCCTGAGTGTGCTGACTGCC |
| (SEQ ID | CCATTGGTTTCTACAACGATCCACAAGA[*/C]CCCCGCAGCTGCAAGCCG |
| NO: 62) | TGCCCCTGTCGCAATGGGTTCAGCTGCTCCGTGATGCCTGAGACAGAGG |
| | AGGTGGTGTGCAATAACTGCCCCCAG |
| SCID | TAGGAGCTCACTTTATAAGTTGGTCTTGTCATTGAGCTGTGGATATAGTC |
| (SEQ ID | ATTCTCTAATATTATTTTTAGGTAATTTATCA[TCTCA/*]AATTCCCCTTA |
| NO: 63) | AGAGACTTCTAAAAACCTGGACAAACAGATATCCGGATGCTAAAATGGA |
| | CCCAATGAACATCTGGGATGACATCATCACAAATCGATGTTTCTTTCTCA |
| | GCAAAATAGAAGAAAAACTGACTATTCCTCCAGATGATCATAGTATGAA |
| | CACAGATGGAGATGAAGATTCCAGTGACAGAATGAAAGTGCA |
| HYPP | GGGGAGTGTGTGCTCAAGATGTTCGCCCTGCGCCAAAACTACTTCACCG |
| (SEQ ID | TTGGCTGGAACATCTT[C/G]GACTTCGTGGTTGTCATCCTGTCCATTGTG |
| NO: 64) | |

In further embodiments, the present invention provides a panel comprising a plurality of assay compositions, wherein each assay composition is capable of identifying at least one of the nucleotide markers as set forth in Table 1 above, and in the alternative, or in addition, is capable of identifying at least one of the nucleotide markers as set forth in Table 3 below. Table 3 also lists the name of the marker (SNP ID), the chromosome from which the marker is derived (Chr), the position of the polymorphic site within the chromosome (Position), a nucleotide that occurs at the polymorphic site (genomic allele (G)), the alternate nucleotide that can occur at the same polymorphic site (alternate allele (A)), other SNPs that occur within 30 by of the genomic/alternate allele (O), percent repeat (P), the discovery breed and the discovery read.

TABLE 3
HORSE SNP PANEL SEQUENCES (SET #2)

| SNP ID | Chr | Position | G | A | O | P | Discovery Breed | Discovery Read |
|---|---|---|---|---|---|---|---|---|
| BIEC20186 | chr1 | 43890382 | T | C | 0 | 0 | Andalusian, Thorough | Twilight, S257P677RC21.T0, |
| | | | | | | | bred | S261P69RD10.T0 |
| BIEC41954 | chr1 | 97804750 | T | G | 0 | 0 | Thoroughbred, Quarter | Twilight, S261P666RO24.T0, |
| | | | | | | | Horse | S256P6135FG2.T0 |
| BIEC51268 | chr1 | 120359811 | G | C | 0 | 0 | Thoroughbred, Standard | Twilight, S261P61RG20.T0, |
| | | | | | | | bred | S260P642FI21.T0 |
| BIEC367927 | chr2 | 1285181 | C | T | 0 | 10 | AkalTeke, Thorough | Twilight, S259P6108RA4.T0, |
| | | | | | | | bred | S261P6125FN13.T0 |
| BIEC392749 | chr2 | 46413743 | A | G | 0 | 0 | Arabian, Thoroughbred | Twilight, S255P698FP19.T0, |
| | | | | | | | | S261P614FG10.T0 |
| BIEC654812 | chr3 | 30798767 | C | G | 0 | 0 | Standardbred, Quarter | S260P65FD9.T0, S256P618FP14.T0, |
| | | | | | | | Horse, Icelandic | S258P675RK1.T0 |
| BIEC683021 | chr3 | 100303903 | G | A | 0 | 0 | Arabian, Andalusian | S255P6100RJ24.T0, |
| | | | | | | | | S257P662FE7.T0 |
| BIEC674509 | chr3 | 82645698 | T | C | 0 | 0 | QuarterHorse, Thorough | Twilight, S256P688RE3.T0, |
| | | | | | | | bred | S261P669RD15.T0 |
| BIEC724885 | chr4 | 39520660 | T | C | 0 | 0 | AkalTeke, QuarterHorse | Twilight, S259P623FE3.T0, |
| | | | | | | | | S256P627RF19.T0 |
| BIEC744445 | chr4 | 95126218 | G | T | 0 | 0 | AkalTeke, Standardbred | Twilight, S259P661RO24.T0, |
| | | | | | | | | S260P653RJ11.T0 |
| BIEC745623 | chr4 | 96486556 | G | C | 0 | 0 | Arabian, QuarterHorse | Twilight, S255P6102RL20.T0, |
| | | | | | | | | S256P674FB16.T0 |
| BIEC751023 | chr5 | 7672131 | G | A | 0 | 0 | Standardbred, Arabian | Twilight, S260P628RO8.T0, |
| | | | | | | | | S255P6130FM9.T0 |
| BIEC758316 | chr5 | 29037866 | C | G | 0 | 0 | Arabian, Standardbred | Twilight, S255P6113RE17.T0, |
| | | | | | | | | S260P648FF20.T0 |
| BIEC784849 | chr6 | 7074858 | T | A | 0 | 14 | Andalusian, Icelandic | Twilight, S257P615FD15.T0, |
| | | | | | | | | S258P699RK7.T0 |
| BIEC787485 | chr6 | 12786580 | A | G | 0 | 0 | Arabian, Standardbred | Twilight, S255P698RC13.T0, |
| | | | | | | | | S260P686FF24.T0 |
| BIEC818096 | chr6 | 81075556 | A | C | 0 | 0 | Arabian, Thoroughbred | Twilight, S255P6125FC8.T0, |
| | | | | | | | | S261P696FA19.T0 |
| BIEC818829 | chr7 | 626858 | G | C | 0 | 0 | Standardbred, Andalusian | Twilight, S260P6127FP10.T0, |
| | | | | | | | | S257P6107FN7.T0 |
| BIEC837622 | chr7 | 32398895 | A | G | 0 | 0 | Thoroughbred, Icelandic | Twilight, S261P63FC21.T0, |
| | | | | | | | | S258P6142FA24.T0 |
| BIEC855571 | chr7 | 81268410 | A | C | 0 | 0 | Icelandic, AkalTeke | Twilight, S258P612RC12.T0, |
| | | | | | | | | S259P635FJ23.T0 |
| BIEC859610 | chr8 | 868737 | T | C | 0 | 0 | QuarterHorse, Andalusian | Twilight, S256P6118RH14.T0, |
| | | | | | | | | S257P621RJ15.T0 |
| BIEC870924 | chr8 | 16053999 | A | G | 0 | 11 | Andalusian, Quarter | Twilight, S257P633FI11.T0, |
| | | | | | | | Horse | S256P628FA18.T0 |
| BIEC888973 | chr8 | 53031026 | G | A | 0 | 0 | Arabian, Standardbred | Twilight, S255P659FM8.T0, |
| | | | | | | | | S260P625RF12.T0 |
| BIEC921711 | chr9 | 38627022 | G | A | 0 | 18 | QuarterHorse, Thorough | Twilight, S256P631RJ5.T0, |
| | | | | | | | bred | S261P644RM12.T0 |
| BIEC930198 | chr9 | 56464461 | C | T | 0 | 0 | Arabian, Standardbred | Twilight, S255P645RO24.T0, |
| | | | | | | | | S260P631RM5.T0 |
| BIEC111585 | chr10 | 44275930 | G | A | 0 | 16 | Standardbred, Thorough | Twilight, S260P634FN16.T0, |
| | | | | | | | bred | S261P65FO14.T0 |
| BIEC122736 | chr10 | 77229308 | A | G | 0 | 0 | Standardbred, Thorough | S260P669FC24.T0, |
| | | | | | | | bred | S261P6113RJ4.T0 |
| BIEC134739 | chr11 | 25480666 | T | C | 0 | 0 | QuarterHorse, Standard | Twilight, S256P625RN3.T0, |
| | | | | | | | bred | S260P6135RK10.T0 |
| BIEC150644 | chr11 | 54946970 | T | C | 0 | 0 | AkalTeke, Arabian | S259P682RB14.T0, |
| | | | | | | | | S255P6131RF14.T0, |
| | | | | | | | | S259P681RC6.T0 |
| BIEC157008 | chr12 | 5419708 | T | C | 0 | 0 | Andalusian, Standard | Twilight, S257P633FK3.T0, |
| | | | | | | | bred | S260P625RJ22.T0 |
| BIEC161407 | chr12 | 10322290 | A | G | 0 | 0 | Andalusian, Quarter | Twilight, S257P639RP9.T0, |
| | | | | | | | Horse | S256P68RP19.T0 |
| BIEC169520 | chr13 | 2271955 | A | G | 0 | 0 | Standardbred, Andalusian | Twilight, S260P618FF19.T0, |
| | | | | | | | | S257P668RB15.T0 |
| BIEC171790 | chr13 | 6672587 | G | T | 0 | 0 | Thoroughbred, Akal | Twilight, S261P637F116.T0, |
| | | | | | | | Teke, Icelandic | S259P692FD1.T0, |
| | | | | | | | | S258P63FG24.T0 |
| BIEC198778 | chr14 | 37386709 | G | C | 0 | 0 | Arabian, Andalusian | Twilight, S255P659FC13.T0, |
| | | | | | | | | S257P614RK2.T0 |
| BIEC219170 | chr14 | 91058897 | T | G | 0 | 10 | Arabian, Standardbred | Twilight, S255P674FO17.T0, |
| | | | | | | | | S260P622RE1.T0 |
| BIEC238830 | chr15 | 25486024 | G | A | 0 | 0 | Icelandic, AkalTeke, | Twilight, S258P6117FA8.T0, |
| | | | | | | | Thoroughbred | S259P640FM2.T0, |
| | | | | | | | | S261P625RL10.T0 |
| BIEC263072 | chr15 | 78561852 | C | T | 0 | 0 | Thoroughbred, Quarter | S261P6134RL4.T0, |
| | | | | | | | Horse | S256P651FB4.T0 |
| BIEC281414 | chr16 | 39841120 | G | A | 0 | 0 | Arabian, Thoroughbred | Twilight, S255P657FI17.T0, |
| | | | | | | | | S261P612RK6.T0 |
| BIEC193770 | chr16 | 61569545 | A | C | 0 | 0 | Arabian, QuarterHorse | S255P638RI15.T0, S256P6141FM5.T0 |
| BIEC317564 | chr17 | 59719397 | T | C | 0 | 0 | Thoroughbred, Arabian | S261P641RI19.T0, S255P649FG19.T0, |
| | | | | | | | | S255P664RN14.T0 |
| BIEC324581 | chr18 | 140840 | T | C | 0 | 19 | QuarterHorse, Icelandic | Twilight, S256P620RN20.T0, |
| | | | | | | | | S258P618FJ22.T0 |
| BIEC328312 | chr18 | 3797273 | G | C | 0 | 0 | QuarterHorse, Thorough | Twilight, S256P663FF5.T0, |
| | | | | | | | bred | S261P67F11.T0 |
| BIEC343880 | chr19 | 2139046 | G | A | 0 | 23 | Thoroughbred, Quarter | Twilight, S261P6104RE8.T0, |
| | | | | | | | Horse | S256P6142FG11.T0 |
| BIEC358651 | chr19 | 39747577 | C | G | 0 | 0 | Arabian, Thoroughbred | Twilight, S255P6104FA7.T0, |
| | | | | | | | | S261P672RF7.T0, |
| | | | | | | | | S255P6104FK20.T0 |
| BIEC425746 | chr20 | 7131135 | G | T | 0 | 16 | Arabian, Thoroughbred | Twilight, S255P628RJ1.T0, |
| | | | | | | | | S261P619FL19.T0 |
| BIEC441654 | chr20 | 40579748 | G | A | 0 | 0 | AkalTeke, Thorough | Twilight, S259P6109FH22.T0, |
| | | | | | | | bred | S261P626RB20.T0 |
| BIEC455646 | chr21 | 6675166 | A | G | 0 | 0 | Thoroughbred, Akal | Twilight, S261P643FK9.T0, |
| | | | | | | | Teke | S259P612RO15.T0 |
| BIEC476263 | chr21 | 48458497 | A | C | 0 | 0 | AkalTeke, Thorough | Twilight, S259P634RE12.T0, |
| | | | | | | | bred | S261P629RG8.T0 |
| BIEC480445 | chr22 | 4678114 | A | G | 0 | 0 | Thoroughbred, Quarter | Twilight, S261P695FM4.T0, |
| | | | | | | | Horse | S256P671RM7.T0 |
| BIEC500415 | chr22 | 40767079 | A | G | 0 | 0 | AkalTeke, Andalusian | Twilight, S259P672FK10.T0, |
| | | | | | | | | S257P685RC14.T0, |
| | | | | | | | | S257P699RA5.T0 |
| BIEC514026 | chr23 | 18527196 | A | G | 0 | 0 | QuarterHorse, Arabian | Twilight, S256P635RJ23.T0, |
| | | | | | | | | S255P6123FA8.T0 |
| BIEC526317 | chr24 | 16363399 | C | A | 0 | 0 | QuarterHorse, Thorough | S256P63FO14.T0, S261P635RL3.T0, |
| | | | | | | | bred, Standardbred | S260P646RA8.T1 |
| BIEC542390 | chr24 | 45955554 | T | C | 0 | 0 | Thoroughbred, Standard | Twilight, S261P631RC21.T0, |
| | | | | | | | bred | S260P6112FC19.T0 |
| BIEC544278 | chr25 | 1928873 | A | C | 0 | 0 | Thoroughbred, Andalusian | Twilight, S261P619RD12.T0, |
| | | | | | | | | S257P6104FE8.T0 |
| BIEC555903 | chr25 | 23640999 | A | G | 0 | 8 | Arabian, QuarterHorse | S255P636FN18.T0, |
| | | | | | | | | S256P630RC4.T0 |
| BIEC562394 | chr26 | 866401 | A | G | 0 | 0 | Icelandic, Thorough | Twilight, S258P69RH5.T0, |
| | | | | | | | bred | S261P660FD13.T0 |
| BIEC580675 | chr26 | 38547619 | T | G | 0 | 0 | Arabian, Standardbred | Twilight, S255P614R16.T0, |
| | | | | | | | | S260P672FF19.T0 |
| BIEC590604 | chr27 | 18662204 | C | T | 0 | 0 | AkalTeke, QuarterHorse | Twilight, S259P6119RJ11.T0, |
| | | | | | | | | S256P662RG24.T0 |
| BIEC600682 | chr27 | 33575633 | G | T | 0 | 0 | Icelandic, Arabian | Twilight, S258P613FK19.T0, |
| | | | | | | | | S255P626FP3.T0 |
| BIEC622581 | chr28 | 36023181 | T | G | 0 | 0 | AkalTeke, Arabian | Twilight, S259P645RI2.T0, |
| | | | | | | | | S255P6107FP11.T0 |
| BIEC633730 | chr29 | 12652411 | G | A | 0 | 8 | Arabian, QuarterHorse | Twilight, S255P648RL24.T0, |
| | | | | | | | | S256P624RP9.T0, |
| | | | | | | | | S255P688FO12.T0 |
| BIEC637205 | chr29 | 20034612 | C | T | 0 | 0 | QuarterHorse, Thorough | Twilight, S256P618RB4.T0, |
| | | | | | | | bred | S261P625RJ24.T0 |
| BIEC687178 | chr30 | 2391118 | G | A | 0 | 0 | Arabian, AkalTeke | Twilight, S255P649F023.T0, |
| | | | | | | | | S259P6119RJ13.T0 |
| BIEC706272 | chr31 | 18788056 | G | T | 0 | 16 | Thoroughbred, Andalusian | Twilight, S261P642FF10.T0, |
| | | | | | | | | S257P647FD8.T0 |
| BIEC942271 | chrX | 14686957 | T | C | 0 | 0 | Thoroughbred, Quarter | S261P61FA21.T0, S256P612RL8.T0 |
| | | | | | | | Horse | |

The nucleic acid sequences of the nucleotide markers of Table 3 are provided in Table 4 as follows, where the position of the polymorphic site (e.g., the position of the single nucleotide polymorphism (SNP), insertion and/or deletion) is bracketed and indicated in bold:

TABLE 4
HORSE SNP PANEL (SET #2) NUCLEOTIDE MARKER SEQUENCES

| | |
|---|---|
| BIEC20186 | AGCTTGCTAAAGTACATTTTTCTTTTTTTTGCAATTTGGAAACTGCATGTG |
| (SEQ ID | ACTGATTGGCTTATCCAATCTGTAGGTAGTAAAAGATCCGTATTGTATTT |
| NO: 65) | GATGCCAAGCCCACAAATCCTCGAAAGAGGGAAGAATTCAGAAGGAAT |
| | TACTGGACAGCTCACAAGGAATTTCAGCCTCACAAAACTTCTAAAAGCT |
| | AG[C/T]TTCTAAGTAACACATTTTTGTCTCGCATTATATCTAAACCTTGGA |
| | TAGGTTTTTCTTTTATGCAATGTAATAAANATTTTCCTAAAATACATAGC |
| | CTGGCTATTCTATACTCGCTGGGAGGTTGCATTTTTACNACTTAACNAGT |
| | AAAGATNAGGAAACGATCTTCATAAAATGTAAGCTAGAGACATGGCAC |
| | ATGCATA |
| BIEC41954 | CAGCCCGGCATCTCTGCTTCGCTTGCTTTCTTCCCTTTTTTCCACCCGCTC |
| (SEQ ID | TTCTTCTTTCTTTTTTTTACACTTGGCTACGGAACATTAGATTTGACGAAG |
| NO: 66) | GGAAAGCGATTTAACATCTTTTAACTGAAGGAGGTTGATTTTCATTTGAG |
| | ACCAAATGGCTCATTTTTACCTTTTTAGCTCCTGGAAGAGTAAAAAGT[G/ |
| | T]AGAGGTCAGCCCTGCGTCACCATGGCAACCCACACAGCTCGGGCCAG |
| | GAGCAGATGAACGGAGGTGATAATCGGGGGGAAGTTTCTCCCTCTTCCT |
| | TACATGGCATTTTCTTCCCATTCAGGGATGTGCCTGGCTGTATGAAAACA |
| | ATTANGTCTTTTTGGAAATATAAGTTTTAAAATATTACTGCTAAATTGTC |
| | TGAC |
| BIEC51268 | CCATTCACAGGAGGAGATTAGCATCTGGTCCCGCTTGGACTGTCCCCAG |
| (SEQ ID | CACTTGATGCATTCATGGCTGGTCTTGANCCTGCCCTGCTCCTCCCCTCC |
| NO: 67) | CATCGCCACACCTTCGTGTGTGCCGTGGCCCGTGCCTGTTCTTCCCTCTTT |
| | TGTCCTGTCTGTCCCTCTGAATCCTTCTTCCCAAGGTGCTGCTCGTCCCT |
| | [C/G]ACCTGTTCCCAAAGTTCCTCCCACTCCTGCAGCTCCCCTGGGCGCTT |
| | CCTTCACCACTCTTAAGGGCTCTGAGGCATTGGGAAGTACCGTGAAGGA |
| | CCCTAGGAAATTCAACTCCCTTGTTTTTACAGCTCTAGAAACGCAGGAGC |
| | AGCGAGAAGTGACCTGAAGAAGAGCAGCAACGGTACCTCCTCGACTTA |
| | GCTGTT |
| BIEC367927 | gagccaaactatatctttctcgccccaaacccaggctctCTTTCCCCAAAGAAAGTATTTGGTCT |
| (SEQ ID | TCTCCAAACCTTGAATGTCAGCCTTTCCCTTGGAAGTGTTGCTCAGACAC |
| NO: 68) | AAAATTTTTTTCACCTCTTGCCCATGAAAGTTTATATATTACTCCTGATAT |
| | CAATTTTACAGTTTTTGCCCAAATCACCAGTAGC[T/C]CGATGATAGCAA |
| | TTTTCAGACCCTCTATGAATTACAGGTGCGTGAAAATGGTCCCTTAGTTA |
| | TTGGAGAAGAAATGAATTCAGGGTTAATCCATCTGACCTCTTTCTTCATA |
| | GAGAGCAGAGTTTATAATAGACTTTTATGTTGAACTGTTGGTACATGTTT |
| | TAAGAGGAGGAATGTATCCATACCTGGGATTTTAAATA |
| BIEC392749 | TGAGGGTGGAGGCAAAGTCATTTCAAAGTATTCNGGTATCTACTGGGTG |
| (SEQ ID | CCCTGGATCCAATATAGAACAGTACTGGTGGAGAGAGGAAGCATAAGA |
| NO: 69) | CAGGCAATTTTAGAAAAGTTCTCATGAGGGAAGTGACTGATGCATATGT |
| | GGTACATAAGGTTGCNAGCCCGAAGGTCTCACTTAGGGACCCAGTCCCC |
| | ATCAG[G/A]GACTTGGGTAAAGTCTGATACAGGATGCACCAGGGGGTCC |
| | CCATGACCCGGAGTGGACTGAGCTCAAGGAAGACAAATCCAGCAGTCC |
| | ACATCTTGCATTGCATTAGCAATGGTTTGTGCAATGCATACCAGGGAGTT |
| | GGGAGTTGTCCAGTCAGGCAATAACTGGAAANTGTAAGGAATACAGAT |
| | GAATTAGCAATGTG |
| BIEC654812 | CCACTCCATCCTCTGTGGTCAGACTTTTGTGTCTACACGTAGTTTTATGG |
| (SEQ ID | AGACAACATTTAGTTCTTCATTAATTCCGTCTGCAGGAGACTTCTACCTA |
| NO: 70) | CCCTGCCCACACACATCTAGGACATGCCCGATTATATGTTTCATTTTAAC |
| | TCTTCAAACCCAGAAAACTGTCCTTCATTCATTGATTTATTTGTTGGTAG |
| | [A/G]TTCCACAAGCATCCATGGAGTCTGTTCCATGCCTGGAATTGTACTAG |
| | GTACTGGATACTAATAAGATGTCTGGGTGCTATCCCTGCCCCCAAGGGA |
| | CCTTGAGGACAGTGGGAAATCCAGGTACATGGGTGATGGCAGCTCAACA |
| | GTCACATCCTATTGTGATAATAAAGATTTATTGAGACTTTATGCCATGTT |
| | CTAAA |
| BIEC683021 | CTGTGCTCTGGTGCAGATCCAAGGTCTTCGCAGCAAAGAAGGGAAGACC |
| (SEQ ID | GGAAGATGGACCAGGCTGTTGTGGATTTAAAAAGGGATTTTTGGAATAT |
| NO: 71) | GTTTCCCACATACATTGTTTATTCCTGAATTATCCTTTCCCAGGAAGCAG |
| | TATATCCATACCTAGATCTTCATGCAGAAGAAAGAAACTGAGGAAGTCT |
| | GTA[C/T]GTTTCCTTCTTTTAGAAGGGAGAGCCAGGGACGCTAATTGTTA |
| | CTTAGTGGAAACTAAACTATATTTTCCCTTCCCCTGACAAATCCTCCCAT |
| | AGTTTTCTTGCTTTGTCATTCAAAAAATACTGGTGTTTCTAAGAGCCTGA |
| | GTTCTTGAGACAGTCTTTCCAGATGAGTTAAGGAAGAATCTAGAGAGGA |
| | AGTCCCTG |
| BIEC674509 | AGCACACCAACAGCAAACGTTTCTGCAGACCAACTGGAGTTTAATGGTC |
| (SEQ ID | TTTCTCAGTGAAAAAATATTTAAATATCTATTGTTTATGCCTATTTTCATC |
| NO: 72) | AAATAAATTAGTGAAATTGGCGAGTTTTCAATCATTACTCATGTTAATAA |
| | CCTTTCACTTGAACTCCCACTAAGGCTTCTGGTCATTAAAAATTGAACAC |
| | [C/T]GGGGCTGGTTTGTGTTCCCTCCCCTGGCTTCCTCATTCCGCTTCTCT |
| | TTTTCCTTTACATGGTGTCCAGTTCTCAAGCACACGTTCCCCCACCCCCA |
| | CGCCTGACCTGTCACAACCAAACCTCAGGGAATGAGTCATTCATTCTCAT |
| | TAGACGGTTGGTGACCACTTAAGCATTGCTGGGTCCTTTCCTGAGGTATG |
| | TGG |
| BIEC724885 | ATCAAGATTTCCCCAACTTGAGAGTGAAAAGAAGGAGAGGTGAGAAGA |
| (SEQ ID | AGATGAGTTTGATTTCGAATGTGCTGAATTTGAAAAATCTCAAGATAGA |
| NO: 73) | GTGTCTAGAAGGGAGATATGGGAATGAGCAACACAGAGATAATAATTA |
| | GAATGGATGATACAAGGGACATATTATAAAATGAAAAAAGATTTCACCA |
| | TAAAGC[T/G]AAGATGTTGGATCATCTTGCTTTAGGCATACTAAGCTATT |
| | CCTCCCAGAGACTGGTCACATATTGGCAGGAGAATCTGAAGACCTCTAA |
| | TTCAACAGCAATAAAGAGAACTAAACAATGGGATCATGCTCCAGAGTTT |
| | GGCGTCTAGCCTTAGCAAACTCGTTTGTAATGAAGGCGCCAGCTTTGTA |
| | GTTAAACATACCT |
| BIEC744445 | ACCCACAGTCCTTTTGGCGAATTCTTATCTCGTGGGGCAGTGCAGACAAT |
| (SEQ ID | GGGGGTTTGTGCTGACGGCTGCTGGTACTCTGCTAATAGTCCTGGAAGCT |
| NO: 74) | TCCACGTTGCTTATTTGGGCATCTTTTCACACCATCACCTGCAACCAGCA |
| | GTCCCTGGTGATCCCGACTTTGTCAGCCTGGCACCCCCATCCAGATACGT |
| | [C/G]CGGAGGAAAGAGCTTGGTCTGAATGGGGGCGGAGGGGAGCTGAG |
| | GAGCACCGGGAGGAAGGAGTCAAATACTGTTATCTGGGTGTTTTCGCAT |
| | TTTGTTTCTCCTGCACACCCTTCCCTCCCCTTCCCACCCCCGCTCCCAGTA |
| | TCATTTCTCTTTGAAATGTCATGAACTTGGCGCTTTCAGACCAAATCGCC |
| | GGGCTC |
| BIEC745623 | GGAGAGGAGNGCAGGGAAGACAAATCGCCTGTTTGCTTGAAAGGGAAA |
| (SEQ ID | GGCTACAAATCAACGCTGGGGGAGCGAGAGGAAGAGGGGGGCAAAGGT |
| NO: 75) | CAGGGGTAGGAACGAGGGGGAGACAGGGCAGCTACAGCGCAGGAGTAG |
| | GACAGGAAGCATTTAAACGAAATCCCAGTTTTACACCTAAAAATATAAA |
| | GCTGTCA[A/G]TTTTCATTAAGGATGAAGCAAATGAAATCTAGAGGGTGC |
| | CAAATAATTAATGACTTTTAATAACCCTAATAAATTTGGATTTCATCAAA |
| | ACCATGTTGCCTATCATGGACGATCACCAGCAAGAATGGGGAGCTGTCA |
| | AGGGGGGCTAACACCTGTGTGAAAATGGACGCCNCTGACATCCTTCCCA |
| | GGTACATCAATTA |
| BIEC751023 | TCCACGCAACGGTTCTTCTCTGAACAGCAACAGAGCAAACAGATAGGAG |
| (SEQ ID | GCAAAGCTCAGAAAGTGGACAGTGATTCCAGTAAACCCGAAGCGCTGA |
| NO: 76) | CTGACCCTCCTGGTGTCTGTCAGGAAAAAGGAGAAGAAAAACCACCTCC |
| | TGCACCTGCCCTAGCCGCCAAACCTGTCAGAACTGGACCCATCAAGCCT |
| | CAGGC[G/C]ATCAAAACTGAAGAAACAAAATCTTAAAGGCTGTGGTTTA |
| | TTGCCAGGGATTGGGGGAGGGGAGAGGGGAACGAGGGAGAATGAAGTC |
| | AGATAATGCCAGCAGCCAAAGGGGTAAAACGGTCTGTGACATTATCCTG |
| | TCCAGAGCTTGGAGGTGCACAAGGGACATAGGAGCAATTTACACTGACA |
| | CACAGCTGCTACAC |
| BIEC758316 | taataaaccactttccTCATTTCAGTACTGATAGGCTTATGGGAATATGCCATCTTT |
| (SEQ ID | GGAATCTAATCTTTGCATTATCTTATCTATCCTTNGTATTATCTTTACAAC |
| NO: 77) | CAATGTTATAAGGCCATAGAAACAGAGTATACCCTCTCATGGGCAATGT |
| | GTGGTAAATTTTCCCCCTTTGTTTATTTCTCTTCAGGCCTTCA[A/T]TGTA |
| | GCACCCTCCCAAACCATCCCTTCCCGCTCAGCCTACACACCCTTCACTCT |
| | TACAGTTCAAATATAGTACTCCTAATTTTTACTGAAAAATAAGGCCAGG |
| | ATTTTTTCACTCTCACTTGCTTCTCTTGCCACTCTGGCCACAACAATTTAC |
| | ATGCCTctcacaaaagcctcatctcattatatcactggctcaaagt |
| BIEC784849 | TCNTAGGACAAGACTAGTGGGGAGGAGACCTGAACTCCAGTGAATTGTC |
| (SEQ ID | CCATCAAGTTCATTATCTCTGGAATTGTTTTTACTCATGGGAGTTCTTAC |
| NO: 78) | AGTAACTCCTATAAGGAGGTTAATGGGAGGGAGAGGTGGCCTATGAAG |
| | NCAGGGAGAGGGGAATCCTGACAGCGGAAATAAACCCTCTCCACAAAA |
| | GTCAG[G/A]GCTGTCTATTGACAGAAAGGCTGTGTGTGTGTGTGTTGCAC |
| | ACGCATGAGTATCGATGTGTATATGTGTGCGTTTCTCATCTACTTCTCCT |
| | AAACTTGCTCTCAGAAAGAACCACTTTTTCTTCTTTTTCTTTTTAACGTAT |
| | TTGGTTTCCACTAAATCAGGATTAGTGGCCATATTCAGCCTCGAAAGAC |
| | AGTTGGAAG |
| BIEC787485 | TTGTAAAAAGTTTCAACTTTAATTTTAATTGAAGTGCAAACTACAACCAT |
| (SEQ ID | GAGATATGCTTTTGTTTATCAGATTGGCAAACCTTAAACACATCCTTTAT |
| NO: 79) | CAGTGGTTCATCTACTGCTGGTAGGAAGGAAAAAATGGTACAGTATTTC |
| | TGTTACTTCTGGTGTGAATATTATTGCCAAAATAAGATTTAGAAGATAAA |
| | A[C/A]GGGTTTGTGGACTGTGTTTTGTGATGCACCATGTGATTGCAGACT |
| | GCTCTTGTTTTTTCCAGTGATAAAAAGTTGATTGCAGAAGGCCCTGGGGA |
| | GACAGTGCTGGTTGCGGAAGAAGAAGCTGCTCGCGTGGCGCTTCGGAAA |
| | CTCTATGGGTTCGCTGAGAATAGACGGCCCTGGGACTATTCCAAGCCCA |
| | AAGAGGG |
| BIEC818096 | CAGTAATCACATTCTGGCTTAGCCCTTCCGTAGAAGCCACAGGCAAAGG |
| (SEQ ID | CAATGGTCTCTGTTTTCAGTTTCTGCCATGAAGAATCACAAGTCTCTGGA |
| NO: 80) | GAGGACACATGTCTCTTAGGTTTACAGAACATTCAAGAAACAAGGCTGT |
| | GAGTTTGGGTCCTAAGTCACGGTGGTCACTTGAAGACGTGGTTCTGGAA |
| | GTC[C/G]CCATTTCGCATTAGCCACAGTCCAGCTTTGCCACAATCCAGAT |
| | TCAAAAGAGACGTATTCCGGCAATTCTCTGAGAAACATACTGCATATGT |
| | TGCTGCATTAGAAACTAAGCCAGGCCCCACAGGAGGCCCTAAAGAAATG |
| | GGGCTCAGGCGCGTGCAGACACAAGGGGGTGGTGAGAGCTGTTATTCGA |
| | CACGTGCACT |
| BIEC818829 | AGCCTGCCATGCAGGAGCAGATGCCCACTCCCGGGGACCCNCAGACACC |
| (SEQ ID | CCCACACCCCCAGCCAATCCTGCAGCTCCTCCTGTGCAGCCCCCCGGCTC |
| NO: 81) | CCCATGCCTGCCCCCACCTGCTCATAGGCCATGAACTTGATAGCCGACTC |
| | AGGGGCAATCTTGAGCACGTTGATCCCGTTGCCACGCCACAGGGAACGC |
| | AC[G/A]CCCCCTTCTCGGATCATGCTCCGTAGGCCCCCCAAGATATTCAG |
| | CCGGTTGGTCTTGGAGGCGTGGACCTGCGCGGGGAGACGAGGTGGCCTT |
| | GGGGTCCCCTCCGGGGGGCCCAGGCCCAGGAGGAGGTGGGGGTTCCTCC |
| | AGGCCCCCTCACCTGCATGAAGACCTTGAGGCGGTCCAGAGGGGCGGTG |
| | CCTGTCCGA |
| BIEC837622 | GTTCTGGACATCACTGTACTTCAGCAATAAGTGGTGTGTGTGTGCGGAGT |
| (SEQ ID | GGGGTGGCGGATGGATGGAGGCTTGGAGAGGGGATGATAGGGCTTCTA |
| NO: 82) | GACCGCCAGGAAAAATCCCCCCATGACATGTGGGGAGAGGCCTCCATTG |
| | GCCAGCTCTTTGCCTCCACTTCCAGGAAGGAGATGGATGGTGTTTACCTC |
| | CCG[C/A]TTTCCACGCCCTGGCCGGGACTGTACCAGAACAACTCTACAAG |
| | GAACAGGATTCACCCATGCTGGCCACTATTTCCATGGCTTTAACTTGTCA |
| | CCAGTGTACCAGGGAAAGCTGACACTTATTTAATCCTCACAGTGGTCTTG |
| | ACTGTGTACTCACGAAAACATTGTATGCTTTTGGAAGACGTTTGTTTCCA |
| | AGCAGCT |
| BIEC855571 | CTTGGTACTCTCATCTTAGAGCCTATCTTACATGACTGTGGATCTTAACT |
| (SEQ ID | TACTGTTAAGTAGTTACTGAATGCTGACTATGCNATGGACCCTGTAATGG |
| NO: 83) | ACACAAAGAGGAATAAGGTGCTGTCCTGCCTGAGGGACCGAGAGGCTA |
| | TTCAGAGACCCGTGTGTTCAAATGTATCTATTTACATCCCACAGTCAGCT |
| | GT[C/T]TCCAAAGTCAGGGGGGCTAGGGCAGCTCACAGAAGGAGGATGC |
| | TATATGTGCTAGATTGAGTTAGGCCAGTCCAGAGAGGACATGGCACTTG |
| | GCAGTTGAATTGCTGTCTAGGGAGAAGCCTAAGAATTCATTCACTCCAC |
| | AAGTATTTATTTTTAATTTATTGTGTCTCAGAAACAAAAAGGGAAAATTA |
| | ATTTAATTA |
| BIEC859610 | tgaggcaggacttccctgagctccgagatgcttgtgtgcactgCACTGTCCAGGGCGCTTGGCTGT |
| (SEQ ID | TTCCCAGGCTCGCGAGAGCATGGCNTCTGATTTTTACATGGCCTCCTGTT |
| NO: 84) | TAGCCCCCACACCCCCTCACACTCCCTCACACTGTTCTGGCGCTGCTGGG |
| | GCAGACGTATGGAGGAATAATCATCGTGAGGGGC[G/A]TTGAACTCAGG |
| | GAAGGTACCAAAGTGCATTGTGGGGTTTGGCCCCAGTGAGCTTAAAACA |
| | GTCTTTTTCAGTGAGCTCTGAACCCCTTCCCGTCCCAGTGCTGTTCTTGTT |
| | CAGAAGCCTGGGATGAACCCCAGCTTCTTTCCAGAGACGATTTCAGGCA |
| | CACAGGGATCTTTTATCCTTGTTTCTCCTGGTACCTTGGA |
| BIEC870924 | TTCTGTTTGTTTAGAACNGCCTGATGAGAGAATTGGATCCTGAGCTCTCA |
| (SEQ ID | TAGGGACATCGCCATAAAATCATCTGCCCGTATCGTTGGAGAGTGGGAA |
| NO: 85) | AACCTTCCTCNAGAGAGTAAATAGTCTAAACAACATTGTTTAGATTTAG |
| | NTAGTCTCGTTTATCCACAGCTCAGAGACCTAAAATAGTTCTGCAAAAG |
| | GAC[A/G]CATGAGTAGGAAAACCCCTAGGCTCCTAGGATGCCGTCTGTG |
| | GCCCAGGTGGCAGGTGTCCTCCCGGAACACCCTGTGACCGGGAGGANTC |
| | ATGGGAAAANGAGGCTCTGCNGAAGCCACCACCNCCTCCCAGAACCTGC |
| | TGTCCAGGAGCCCACTGTTTATTTCTATTTTTTCACTTCATTTGTTTTTAA |
| | TACTAGGAT |
| BIEC888973 | gatatgtttgaggtggtggtttcagccattttggtgagttactcagcttgcccgagcaactcccatgtatacatTGACA |
| (SEQ ID | AATAGAAATGGCAAGTAATAGGATATATGGGAGTACATGAGGAAGCCA |
| NO: 86) | TTTGGTGTAAACCTAATTCGGCCTGACTTTGCTATTTTTTCCAAAAGGGC |
| | CTGACCGAGGCTGTTGAGCATGC[A/G]TTGTATATCTGCTTTAGATATTC |
| | CCTATGGCAAGAACAAAGGCCCTTGAGATAAAGGTGCAACTTCCCTCNC |
| | CCTCCCAANGTAGACATTTCCTTAAGGATTAAGCATCTTTCCTTAGGCTA |
| | GGAACTGATTGCTTTGCTTACCTGTGACCACCCAGCTGGAGACAATAGA |
| | CTTGCCTCCTGCTACGCCCACAGAGATAG |
| BIEC921711 | CCAAACTGAACTAAGAAAACATGTGGTGGCGAGGGGGCATCTGGTGCTA |
| (SEQ ID | TAAGACANGGGTGGAGGAAACCACAGCCCCAATGCTGTAGAGTGACTC |
| NO: 87) | GATAAGCTTGTCCCTGTGGGAGCGAAAGGAGAGACACAGGGAGAAGGG |
| | CATCTGAAAGGGGTCTTCTCCTGAAAGAGCTTCATTCCCCTCCCCTCTGG |
| | CGGGC[T/C]GGATTCTTTGCACAGATAATTCTAAACCTGTCTTTCCCAGC |
| | ACTTTCGGCTGGAGAGGTAGTCTGCAAGGTTTTCTGCCTTATCCTATTTT |
| | ATTACCAACCTACCTCCACACATTCCAGTTTTGAAGAGAAAACTAATAA |
| | GGAAATGGCTGAAATAGTGTCTTGATGGCAGAAGGAAGAAATAGCTCCT |
| | TAAAATTTGTT |
| BIEC930198 | AGCATAGCTTAATAATACACTTCATTTTCACATTTATTTACTTCAAAGTG |
| (SEQ ID | TATTTTCATGTTTTATGTACTTATCTATATTTTTGCTTTTAGCACAAAGAA |
| NO: 88) | CAGAAAAGTAAGATTACCACAGCATTCCAATATGTTAACAAAATTTGGA |
| | ATGAACTATGGATCAAACCTTGGGCACTGAAACTCTTCAGGGCCCTTTA |
| | A[A/G]GAACAGTTTGAAAAGGTTCTGTGAAGTTTNAATAGTTAGGCTAG |
| | AAGTCCCTCAAAGAAGAGGAATAGAACGCACAACCTCCCAAATAGCAG |
| | AGGCAAAAGAAGAGTGAGAAACCCACAGCAATCCTAAGGAGAacaaactaca |
| | tattttactgattttttttttcccatcttcctcaattagaatgtaatcgctaccaa |
| BIEC111585 | TTGATCTGAAGCTGTAAATTCCCCAGTAGCCAACCCTGTTGACTCACCTG |
| (SEQ ID | ATACTTCATGATCAGGCAGACTAACTTGCTTCCAGTCAAATAAACAGAA |
| NO: 89) | AGGAAATTATTTGAAAATCAGGAAAAAAATAATCTGGGATAGTCNGTGA |
| | TCTGGAGTTGTCAGTAAGAAAAACTAGCATTTTAGAAATGTTTAATTTAT |
| | CC[G/A]TTGACATTTTGTTCTGATTTTTCTGTGTGTGTTTGTTTGTTTTTCC |
| | TATTTAGGAGAAAAGGGAAGATGTAGCCATTTTAAGAAAAAATAGACCC |
| | ATTGAACAAATCTGAAAATATCAGTATGTATGCTGATGGCAGAAGAGTA |
| | TAAGAGAGACTCTGTATTTGTAAACATAATTGATGGTTAAGTAAAGTAC |
| | TCAATAA |
| BIEC122736 | TCTAGAGAGAGAAGTGTGCTTATCCTGAAAGTAAAAAAACCCAGGAGA |
| (SEQ ID | GAATAGGAAAGGACAAATATTTTTGGTAGAACCTGGTTGATCTGAACTC |
| NO: 90) | AGAATCTCCTGGAGATTCACGGGGTTCCCCGGCTTGGAAGGACAAAGGG |
| | AGAAGCCAGCACCTGGAGCAGAGTGACCGTCCTTTGTGATCTGTGTGCA |
| | CCACG[C/T]GTCAGAAATGTACCCAGCCAGCCACCGGATGTCACTGTTCT |
| | CTCGTGCTCCTCGTCTGGGCATCCCACAACGTCTCTGCGTGAATNTTCGG |
| | TGTCCTGCTTTGCAGCCTGACAGTGGCGCAGGCTCACCCGCACGGGCCT |
| | CGCAATGTGGGATCCCTGACTATCCCCACATAACCATGATTTTCGTTCCA |
| | GAGCAGCACA |
| BIEC134739 | CTATAACGGACTCCATGANCTGAGAGCTCATGTTTCAAACGGCAGGGNG |
| (SEQ ID | GGGGAGTGGGAAGTCACTAGATTGCTGGTTCTTAGACAGTCTTAGGGCC |
| NO: 91) | AGTTCTCATTCTCTCTCCTGCGCTGTAGGCTTCTGAAGACCTCCTCAAGG |
| | AACACTACAACGACCTGAAGGACCGTCCGTTCTTTGCCGGGCTGGTGAA |
| | ATA[C/T]ATGCACTCGGGGCCAGTGGTTGCCATGGTGAGTGTGCACGTGT |
| | GGGAAGTCACTGTGAATGGCTCAGTTGGGGGTGAAGGGTGGGTGTTGTG |
| | ATTCCTGCTTCTCATGCCGGGTCCTCATGGATGCGGAGCAAGCTGGGGCT |
| | GGGAGGGTTAGACANTTAGGATGTGTCAGCTCGCTGACGCACCAAAGAC |
| | AGGATGAAC |
| BIEC150644 | TAATAGGTTCAACAAAGTTGTGGCTGACTCCTTTAGTTGCTTATCCAAAA |
| (SEQ ID | TCCATTCCCTACTTCTTCCTCAATTTAACATTAATTTGATTTGGGACAGCA |
| NO: 92) | ATAGGTTTAGGTCCACAGTGATGGAATGGGACCAGCCTAAACTAATCAC |
| | AGCCTCCCTTGCAGCTACAGGTGGCCAAATCAATGCATATAGCCATAGA |
| | A[C/T]CTCTTCTTTCAGAGATCTCAGGCATACTTTTAGGAGAGTATGCTT |
| | TGAGACTATTGGACCTAGGCTAACTGACTTACAAATTAGATGAACGCAC |
| | TTCCTGACCAACCAAGCCAGAATGTATTCATCTTTTAGGCATTTCTCCTA |
| | TAAAACATTTACTCTGGTTTCTCTCATTAAATGAAAACCTATGCACTCAT |
| | TCATG |
| BIEC157008 | TCTTACTTTTATCCACAGAGCTTAAGAAGTTAGTCGGGCCAAAGACTGCC |
| (SEQ ID | CTCTACTTCTCCCTAGCTCACTATTACCCCTCAAGAAGACCTTGGGTGAA |
| NO: 93) | TGGCCCATCCAAGTTCTGCTCTGAGCAGAGAGACAAAATCCTTCCTTCAT |
| | TGATATGATCTTCCTTTAAGGAACTTTCCCCAGGATTGGCTCCCCTCACC |
| | [G/A]CTGACACTGAAGAAGTAAGGGACTTAGGGCCCAAGAAAAGCCTNC |
| | CTCTAGCTGAGACAAAGAAAATCTCTTCATAATCCAACATCAGGAGACC |
| | ACATGGAGGATGAAGCAAAAGCTTCAAAGACTGTCATAAAGATTGTGCA |
| | TTCACTCAGCTTTGTCCTTTGTTGGAGACCTCTCCAGGGCATGACTAAAG |
| | CAGGACA |
| BIEC161407 | TGTAATCAAAACTACAGGGCCTGACGAAGGAATTCAAATTCACAACCAA |
| (SEQ ID | CTTTTGTGACTTCTGGAAGGATAACCATAGTGACTTCCTGTAATAAAATC |
| NO: 94) | AGCCCCTGACCACCAAGAGAAGCCTGTGGAACCCCGAGACTTCAAGCCT |
| | GTAAGGACAGCTTCAGCCAGTCCCTTATCATTTAGTAGAAAACCCCTTA |
| | GGA[G/A]CTTCAGACATGTACTAGGACTTTTGGCCAATCTATACACTGAC |
| | TAGAAAACTTATGCATAAGTTCAGCACAACTATTGTTCTTGATATTTAGA |
| | GGGTCCTCAGGAAGGCCATGTGCTGGAAACAGAGTATGGCTAGGAAAG |
| | TTCTTTGGAGGGAAGGTGGACTTTCCACCCCCTGCTCAGGGGCAGTGTTC |
| | TCTGTTCCT |
| BIEC169520 | GACACTGGAATAACAGCAAGACCATGCGAAACATGGACGACACCGTCC |
| (SEQ ID | CTGCCCTCCTGGAGGTAACGACATGAGGAAGGGGTTATACGTACAGGCC |
| NO: 95) | ATGTGGTTACGAGTGTCCAGAGGTTGGCAAGGGGCTGCCCAGGATCCAG |
| | AACCCCCAGAAGCTGGGAAGCAAAGCAGATTCTCACACACCCTCACTCT |
| | CTCCC[T/G]GGCTCAGTGCAATAAGCTGTGAGTGCAGTCAGCTATTTCCT |
| | TAGAAGACTGATAAATTGATAATTACACAGAGATGTAACACATATAATT |
| | AAGCAATATTCTACAGGAACAGGAGTTTTGCTGCTTTTTTCCTTAATCAA |
| | ATTATTCCTTTCTAAAGGTCTCTGTTCTGAGGAAGAAGAGACAGGACAC |
| | CGATTCTAAGC |
| BIEC171790 | CAGGTAAGCCCCTTCTACTTTGTTCGGCCTCTTCTTTCCCTCCCTTATGCT |
| (SEQ ID | GTCAGTTCTCTCCCAGCTCTGCCCTCTCTTCCTGTAGACCTGGGTGAATG |
| NO: 96) | AAGGCTACTTCCCGGATGGTGTTTATTGCCGGAAGCTGGACCCGCCCGG |
| | TGGACAGTTCTACAACTCCAAACGTATTGACTTTGATCTCTACACCTGAG |
| | [C/G]CTGCTGAGGGCCCGGTTTGGTGGCCCCTTCTTTCCTGGACNCTGTG |
| | GAGGAGGCCCCACGTGCCTCAGGCAGTGAGGATATTGGGGGCCACTTTT |
| | CAGTCAATTTTCCTTTCCCAATAAAAGCCTTTAGTTGTGTATTATGGCCTT |
| | GGCTGTGCTGAGGGCCAAAAGCCTTCTTCACAGCTCCNGTGGACTCACC |
| | TCCAT |
| BIEC198778 | ttttagggaggtggtgggagctgggtggacaggagaagggaGACTTTTCTCAGTATAACTTACT |
| (SEQ ID | GCTATTTTAAAATTTTTGAACCATCCGAATGTATTACCTATTCAAAAAAA |
| NO: 97) | TTAGATGACAAAAGTCCCCACAAGGCTAGAATAAGAGCAAACTAAACA |
| | GATAAATTCCATGCTGGTGCAGTAAAATGGAGACAAGT[G/T]CCAATCTC |
| | AGAAGGTGACCCTGGCAACAAAGTTGTGGATGTAGGCTAGCTAGCCCTG |
| | GTCACTGTAAACCAATGAAAGAAATGTGTGGATGGAGGATGGGCATAC |
| | ACACATGCACACACAATGCCTTACCAGACACAAGCTGGAAAGGGTTCCC |
| | ACAATTGGAGATGTCTGCATGATCAGGCAAAGGCTAAGGGAGAGAG |
| BIEC219170 | GAGGAAGTAGAATTCAAGGAGGAAAAAAAATCCTAAGACGTTAGTTTG |
| (SEQ ID | TGAAACGTGAACAGAAGGCAAACTTGCTGCAACCCACGCCAGGCCAGCT |
| NO: 98) | CGCCCACCGAAACCGCGGCTCCAGGANGCGNGGACTCAAACACCCCTGC |
| | CTGGGCATCCTTGCCCNCGGTGGGAGAGGTGCCTGAAACTCAGAGGGGC |
| | GGTGG[A/G]GTGCTATGATCTGCGCTTCTCCCTCTGGGTCTATCCAGGTG |
| | TTTTGTACACGAAAAGATTTCAACACAGTGAAATAATCAATAACTTATA |
| | AGGCATGTCTATACTTGCAAAGTGAAATGTGGAATTGAGATGGTTTGGA |
| | GAGAAAAAGAGATTTTAAAAACAGACCCAAATAGTCCATNANAATAAA |
| | AAACTTATACTTT |
| BIEC238830 | AGAACCCTAGAGGCCCAATGTTCTACAGNGGCTGTTTCCAAGAGGGAGG |
| (SEQ ID | AGGCTACAGCNACTGCCACACTGGATACACTCAGAGGAGAAGGAAAGG |
| NO: 99) | GGAGGACCCCGAATGCCTCTCATGTATAAAAGCGCGGGATGGCAGGAG |
| | GCCTGTGTGGCCCAAGGAGCCAGAGGGAGCAAATTATGGGCAAAAGAA |
| | TTGCAAC[T/C]CAAGGCTGGAAGAGAAGAGCTCTGCCCTCAGCTGTGTCC |
| | CTGGCTCTCTGGCATCTGCTTCTAATGGGCAGGCGCCATTTTCNGAAGCA |
| | TCAGGCACTGGAAAGAAGACCTCTGCTGCTGGGATTGAAAGGAAGTAAC |
| | ANCCAGGAGGGGGAGATGCAAGGGCGAGGCTGGTCCCAGAAACAAGGC |
| | CTATAATGCAGACA |
| BIEC263072 | TAGGCCAAGGCCCTTCATCATCAATTCGGGAATCAGCTGTTCTGCTCATT |
| (SEQ ID | CATAATCCAAGTGGATAAAAGCGATGTTAATTCCCTTTCTAGTTCTCTCA |
| NO: 100) | ACTAGAAAGTTACGGAAGGTGCGTTATTATCTAGGCAGAAACATGTCTC |
| | CACTTAATATTGCGGGTTTGCTCCCTGGAAATGAAATGGTGAAGTGGGG |
| | CA[A/G]TATCTACGTATGTGCCACTCTTCTCTCCTCTAAGAAGACTGNGA |
| | GATCAGACTCAGGGTGAGAACCCTCACNCCAGCAAAGGCCAGGACACC |
| | CACAAAACTGTCTCCGTGAATACATGTCTCAGAATCACCATTTTTGTTCC |
| | ATCTTTCTCACCTCTACTTTTCTTTCTACAGTCTTCTCTTTAAGAACTAAG |
| | TGTCCTG |
| BIEC281414 | AACTATTTCTGAGGAATAGCAATAGCAATAGTCTCATGAGTTCGGTACTT |
| (SEQ ID | ANAGGGCAGCCAATCTAAGTCAGAAGGGGATAGGATCTACCCTGGGTCT |
| NO: 101) | CCCTGGACCACGATTGGATCCCATGTCTCCTGAGTTTGTGTGGCTGTGCT |
| | TCTTAAGCGCTCTGATCCTCGTGGATGCCAGCATGGTAGTCACTGTTCCT |
| | T[C/A]CCAGACAAGGCCTCTCTTCTGTAGCAACCCTCTCCAAAGGCACAG |
| | TTACCAACCTGCCTGCTCCTCCATTCAGGGTGGGCATATACAGGGGTGC |
| | AGGTGGTTTCGCTGCAGCTGCCCTGTCCCCAACTGCCCCTCAGGTGGGTG |
| | GGATGATCTATCTACCCTTGGCTTGTGTGGAAATCACATAGTAGCTGTGG |
| | AAGCAT |
| BIEC293770 | AACATGGAATGATTTTTCTTTGGAATTTTGCATCTGTCTTCTTCTATCCCT |
| (SEQ ID | CCTACCACAATCATAACTCAAACCATAATTCATTTATGCCTGGGCAAATA |
| NO: 102) | CTATTGTCTACCAGCTGAATTCTCATCCACCTCCTTCTAAACTTTCACACT |
| | GTTTCTTTGCTAAGTTTTTGTTTTCTTATTCTGTGCTTGAAAGCTTCA[C/T] |
| | ACGATGTCCCATTATCAACAGCATAGAATCCAGTCTCTTTACTTAGCATC |
| | AGATACCTTATAGTCCAGTCCTGACATTACTGGTTAATTTCATCTCTTCA |
| | ATGTGTACATTGATATACACGCTTCTATTTATCATACTTCCAGCATGCCC |
| | TATGATATTGGTTTTGCCTTACATTTGCTCTTCAGTCATGGAAATTTTTC |
| BIEC317564 | ggccactgctaaaaactgcccgctgttcctggccacacagcctgtcccaacatgttcacttgcttccttacaccagcaAA |
| (SEQ ID | CATTCTCTAGTGCGGTCTGCTAGGAAGATGGAGTCTTACATAGACAGAA |
| NO: 103) | CCTATCAAGAAAGTAGCATCCATCTCTTCACTATATCCTATTGGAGAGAA |
| | GAAAGCCACCCAGCCATGACG[C/T]ACCACTTGATTCCACGGCTGGACG |
| | GGAGGCTCACTGAGAGCGGCGGCGGAGAGGCTGAGCTGGACGCCAAGC |
| | TCCGCTCGCTCGCAGCCTCCTTCCCTGCCCCAGCTCCTCTGCTTCCGGGC |
| | CGCATCTGCAGGAGCACAAGCCCCGGGGCCGGTCCTGCTGCTCTTCCTG |
| | AGCCTCCCCTGCTGGTGCTACCTTGGGCC |
| BIEC324581 | GACCCCCACCATACCAGGGAACATGAAGTGTAGGCAGCCCTGAGGCCCC |
| (SEQ ID | TGACTGNGGAGATGCGCTCATGGGTCCATTAAGCTGGGAGGTCACACTG |
| NO: 104) | CACCGCTGGCTGGCCAGGACCTGCGCACAGGGTTTCTTCTGACTACATAT |
| | TTGTTTTTCAATTTGAATAGTTGTCCATGTTTAACCATCTCCAGATTTCTA |
| | G[C/G]TTATCTTGAAAAATGAGCCCACACAGGATCCAGGATTTCACATGA |
| | GCTGGGTCCCAACCCCTCCCAGTGGGGTGGGGTGGGCGTGCTTCAGGTG |
| | CCGCCACTCCCTGCTTCTCCTGAGTGGCCCACATTACTAATTTAGGTGAT |
| | TGCCTCGTCCCCGTGGGACCACGTGAGCAGCGCCTACTTCAATGTTCCTG |
| | ACACCA |
| BIEC328312 | attagattcagagagaggcggagtaacttgctcacactagtaagcagagtttaaactgaggttaaatgaatagaaagcct |
| (SEQ ID) | gagttctttccactAAAAATAATTCTGTGTAACTCAAAATTTTGTTCTTATTTATTT |
| NO: 105) | ATTTAGCATAAAAAAGTTTCTTANGCCTGGTATTGGAACTTGGTACTCTG |
| | ATAAAAGTATCAC[A/G]GCTTCTGAAAGCAGCCTAACCTTCCAAGAAAT |
| | ACAGTGAATCAGAAGCCTGTTGTCTCTATCGACTCCCCAAGAAAGCTCT |
| | AAATCTCACCTACATGCTTTCCAGAGTTGCTAAGTGCAGCCCTCCTTTCT |
| | TAGCAAGGGATTCACCCAATCACTAGTCCATGCACTATTGAATTGCAGTT |
| | TCAAATGCTGAGTGTAGAG |
| BIEC343880 | ATAACTAGAAAGGATGGGAAATGCCTTAGTGCTTACTATTTTTAAAAAG |
| (SEQ ID | CTACTTCCTCGATAGAAAGACTTTATTTACAAATAAATTGAAAAGGGTTT |
| NO: 106) | CTGAGGAAAACAACATATTACAAATACATTTTTGTTAACTTTNTTTNAAA |
| | AAGCATCACCACAACATATGTCTACTCAAAGAGCCTTCAAAACTCCATT |
| | TT[G/C]AGAAAAAGAGCAAAGAATCTTTATTTCCAAGCCAACAANCTAA |
| | AGGGNCAGTTTGGTCANACGCGTAACGACAAGGAACCTTAGCTTCCTGA |
| | TCCAGAGCAAACCCAGCAGTTCCTTTAAAGGTGACACAAGAAGGTAATG |
| | AGGAGATCTAGAGAGTGGAAACGCAAGGCCTGCTGAAAGTCTGCGCTTG |
| | CTTAAGCAGC |
| BIEC358651 | attcttttcatttctctgtaggtatttaccatatcccttcCTGGATTTGATCATTATAAATCTTATATC |
| (SEQ ID | ATGGTTATTTTAATTGATTCTGAGCTTTCTCCTGTCCTTACtttatttatttatttatttatt |
| NO: 107) | tattGAAGTACTTCATCCAGAAACAGCATCCTACACACGGGCTGCCATTGT |
| | CTGCTACTAGAGTTGCT[T/G]TAATTTAACTCTCCCCCTTCCCTGTCAAAA |
| | TTACATCCTAAGTATGCTCTGGGCTCAAACCAGAATGATCTTAATGTGGA |
| | TGCAATTGTATTTGAACCAAGGGTCAGCTTAGAGGCAGCTGGCAAAATG |
| | TTCAACAGACAGGACCAAAAGGAAGAATTGTAACAGAAAATGTGATTCT |
| | GTCTGGAGCAAAGTGAGAAAGT |
| BIEC425746 | CATAGGCTGAGTGGCAGAGGACCCTTACGAATCCCCCAATTCTTTGATA |
| (SEQ ID | CCTAAGGCAACTGTGTACAAGCTGATAATATAAATCTTGGGAAATAATA |
| NO: 108) | ACCGGAGAAATTCTAGGGCGCTCAGTTCTGGAAATAACTTTTGTAGAGC |
| | TCTGGATCTAAGGGCCTACCTTTCTTGGACAGCCTACACGGTCCCATGCA |
| | AGC[A/G]TACCACTGCTGGGATGGTCTAGTTCAAAGGAAGAAAACACCT |
| | TCCCTGCCTTTCTGCTTCAATTTGCCCTAACCATTTTCTGATTTGAGAAAA |
| | GCAACATAGCAAGGGGTACTAACACTTCTGTACTAATCGGCCAGGGTGA |
| | GGAGTGAACAATTAGAGTTGCTCTTTTAACTCTAATGGTTGTCAAGGCA |
| | GGGAGATGA |
| BIEC441654 | CTGTGTGGATTTATAATATTTCACTGTACTTTCCACTGACATTAATTGAA |
| (SEQ ID | TAAAATAATCTTATAGAGAGGTTTGGCTTCAGTTTAGTCACAATCTGATG |
| NO: 109) | ATTAAGTTTGATTTAATGCTATTTATTATTCTGTTTAAAAGGAGTCTTCA |
| | GAATTGTGTCATCTGGAGTCCAATGGGCTAGTTATCTGGTATGCTTATCC |
| | [G/A]TTCTCCAATTTTGATTATTTTCTTCAAATCATTTTAAAATGTTATTTT |
| | TCATGAACATTTCCTAACACTGCACTTGCATTTTCCATTTCACACCTCTCT |
| | AATCTATGTACAGTAAGGCTGGTATGACANCCTATTTAGTNATCCACAG |
| | TGTGGTNTTNATGAAGCTCATGTTACAGTTCCATTCCCTACACAGAACCT |
| | AA |
| BIEC455646 | AACAAGACCTTGATCATGTGCCACTGCGAGAGGAACTCGACGCGCCAGC |
| (SEQ ID | TCATGCGGCCGTGGCCTGAGTTGGGAAATGGCCAAGAGGAGTGAGCTGG |
| NO: 110) | AGCTGTGCTCATGGCAGTAGTGTCCGGTGGGATTCTGGTCAAGTCAGCT |
| | GGAGGTTCCGCCGGCAGGGTGGAGGCCAGAGGTATAGGGGTGTGGGTC |
| | AGAGG[C/A]CCCAGGGAATGTGCTGGAGTCATGGTGAGAGTAGCACCTG |
| | CCACAGGCTTTCCGCGTGGCTGCTGGGCTCTGGCGTATGCTGTGTTGCTC |
| | GCCTCCCCAGGGTAGCCTTGACATGATGAGCGATGGGAGCATCCCTTGG |
| | AAGACAGCCTCCCTGGGGAGCTGCGGGAGCCAGGAGGCACAAGCTGCA |
| | GCCGGGAGCAGAG |
| BIEC476263 | TATCCTCATCTAAAGCCAATGATAAGGNTTTTCAACATGTTGCTAAGAA |
| (SEQ ID | GAAGTCTATTGCAATAGATTGTTCCTTTCTAATTCTTTGTCACCTGGTTTC |
| NO: 111) | TTTTCTCCTTGAACATGGGTGGGAAAGATGGGCCACAGGTTTTTCTGCCC |
| | ACCTGGAAGGAATGACTCTACACCACCCCCTCTAGCAGGACTAGCACTG |
| | A[G/A]CTCTGACAAGATAGTCTTTGTCCAGGGTTGGTGATCTTGGGACCT |
| | GAGGCAATGAGATACAGGACAAGAAGGGATCATTGGAAAGGTCTAACA |
| | AAGGAAAGTGGGGAGCCACTTTCTTGGTATATTTGAGTCACAAGGCTTT |
| | GGATGCTCACCTTGCTATTTAATTTTTACAATTCCATTTTGTGATAATCAA |
| | NATCTGT |
| BIEC480445 | CAGGCAAGAAGCGTCTCAAGGGTTGAAAAAAATACAATCTGCCAATGTA |
| (SEQ ID | GACTTTCAAACAGATCCCTTTTTTCTTTGATTAAAAAAAAAAATGGAGTT |
| NO: 112) | ATTCCAAGATAGAAGTTACTGCAAGATAGAAGGTTGACAGTACTAAATG |
| | ACAGTCAAAAAACATATAACCTGAATAAGTAAAAAGAAATTAAAATGA |
| | AGTC[G/A]TTGGTATTTTTATGAATTTATGAAGTCAGCATGGTCTGTGGG |
| | TATAGATGCAGCTAAAACCTATGTACTCAAGTTTAAATTGCAGGTTGATT |
| | TTTCTACCCACACATATTTAAGTCAGTTGCTTTATTCTCATTTGGAGTTTA |
| | GCTCCCAACCTTGCACAAGATCTTGAACTTAATCTCATGTATTCTAGAAT |
| | TCAAGAT |
| BIEC500415 | TGCACATCCTGATAGCAGCAAAGACGAAAGTGTGNGAGGGGAAGGGAT |
| (SEQ ID | TNATCCCGAGGCAGCCAGCTCATCATCNGCAAAACTGGGATAGGAAAA |
| NO: 113) | AAGCTCGGGTCCTTCTCCCACAACTTAAGCTCGCATCTCCTAATTTTCAT |
| | AATTGAGTGATTTTCCCACTCTTTCCATCATTTTGGCTGGATCCTGCTGA |
| | GAAA[G/A]ATGGCTTTTTTTCAGAGCTGGAATAAAGACTCTTCAAGTTGA |
| | TATTGGGTTTAAGCCACAGATGCTAAGATGTCATCAAGTTCAAAGTCGG |
| | AATCTTCTAGAATCTTTGCCTGCAGACAGAGATGCTGAGCCAGCTGGCA |
| | GACGTGGTGGTGAGGACATGCAGAGCTCCCATACACTCCACTTGTCCAT |
| | GGAATTGTACG |
| BIEC514026 | TTTTGTGATAAAGGATTTCTTTGCATTTTTTCCTCTAGTCAAGTAAATTGC |
| (SEQ ID | TTGTGGGTTCTTCCTAAGAAAAATAATCCCTCTGGTGCTGCTTTTAATTT |
| NO: 114) | GATCAGGTTTAAAATGTTTTCAGAAGAGTTAAGCTTCCTTTACATTGGTG |
| | TCTGGTGTGGTCAGATGGAGGAATAGCTTTGGAATGAACTAGATTTTTT |
| | [A/C]GTGATGCACCGTTTGACCTTCCCACAGAAGGTTCAGTACAAGGAAT |
| | CAGTCAAAACAACAGCACCATTTTCACTTGACCTCGAGACATGTGGTGT |
| | ATACCCTTTACCCCGACAGATAGAACTTCCTAAGCATATTTTTCTTTGAC |
| | TCATGTTGTAAGAGTTTATGTTTCTTATGATATATATCCATTGTGTCCAAC |
| | TGTC |
| BIEC526317 | AAATTCTTTTTGAATGTTTACATTACTTTTCTGGTTAATAGTTTTAAAATT |
| (SEQ ID | CTGTGAAGGAGCATCTCTGAATTTATCTGAAATTTATAGATACTTTCCTT |
| NO: 115) | ATTCAAACAAAAACAAAACCACAACACAAACGCAAGGAAAAACAAGGG |
| | TCCAATAAAGTGGAAACTTCTGTTATGGTCTAACTTTTGGTCAGCAGTAT |
| | G[C/T]AAGCATAATTTTGGTTCAGGACTAACGCTAACGAGAGGCAAAGC |
| | TGAGGCTACGGCTACGGGATGATGGCTGAGGCTCATATTGTATTACTGG |
| | AGGGGCCCAGGGGGAAGTTAAAATGAGACACTAGCTCCTGTGCATCAG |
| | GACCGTCAGCTCTAGAGGTGTCAGGGGCCCCTGAGTTGGAGCAGTGAGG |
| | AATCCCCTCC |
| BIEC542390 | CCCCCAGCCCTCTGCTGGGTTCCTACCAGGCTCCAGCATATTGACCCCCT |
| (SEQ ID | GACTTCATGCCTCTGTTCAGACCAGGGTAGATGAACTGACAGCCGCCCA |
| NO: 116) | AGGAGCTGCCCCTTCCCCCCACCACCACCTAACCATGTCCCGCAGAGGA |
| | CACGCAAATAAAAGGGCCCTCTGAATGGACTTCAAATGCAAAGACAAAT |
| | TCT[C/A]AAAAGGCTGTGCATACAAAATGCACACATTGGTTGCCAGAGAT |
| | ACTAACGTTCATTAGTATTTATTAGAAATCGTGACACTGACACTTAGTTC |
| | GAGGGTCAGTCTCCGTGAAGGCGGCTGGCCGTGGCTGGGTGTGGCCAGC |
| | CAGCCCCCCTACTCCTCTCCTGGANGGAGATGGCCTGTGGGGAGCTGTG |
| | CCCCCAAGC |
| BIEC544278 | CCCACAGGACTCGGCTTCTAGGCGGCAGGGAGTGACTCCAGGACCAGAG |
| (SEQ ID | AGCAGGCAGCAGGAACCCTGAGGGACTGCAGGAAGCCAGGCTGCCCAC |
| NO: 117) | TCACTCAGTGGTTTGCAGGCAGAGGGAGCAGCTAGAAGCCCAGGAGAC |
| | CCTTGTCCACCAGCCGCCTCCTGGGCCCAACAGCCGCCCGCGGGCAGGC |
| | CCGGTG[G/A]GAATGCTCATCCGACCTGCGAAGGTCTCCATACTGCCAGT |
| | CTGGGCAGACTATGCGGGGCTGACAGTTGCCCCCAGATGTTTTACAGCA |
| | GCCGTGAAAGGGCCTCGAACTCCACAGATGGCGAGCGACTCGCAGCCAC |
| | TGGTTGTGGGTGTTCCTTGCTAACATCTGcacacacacacatgcacacgtgcacatgcatgc |
| BIEC555903 | AAAAGGTCACTTTCCAAACTGCTTTTGCTCCCAGGCTCTGCTCTGAATAA |
| (SEQ ID | TTCAAGTCATCCTCAGTAAGAGCAGCAGGCTTTGGGGTGATCTCCAGCC |
| NO: 118) | TGTTTGACAGGAACGGTGCTGACTTAAGCTAACAAGAGGTCATTGTCTG |
| | AGCAGAAGAATGGCATCCTAGCTTCTTGATGAGCAGAACCAGGCATGGG |
| | AAC[G/A]TAAAACAAGACCAACTGACTCATTGTAACTGAACCAGAGGCA |
| | GCCAAGTGCCTTCCCAAATTCCCTTCTTAGCAGGACCAAGCCCTTGAGG |
| | AGGAGGAGGCATTTATTGGGGATTGCTACAAATTCGGCTTTACTGCCAC |
| | CGCCTTTATAACCACAATATAAAGTAACTCCCACAACACAGTGAAGATA |
| | AGCTCTTTAAA |
| BIEC562394 | ACTGTGTGAGCAGCTGACCATCCTCTCTGGGTGGAGGTAGAGTGTAGAC |
| (SEQ ID | AGACAGATAAAAGGTTTTGTCTGGATGAAACCTTCATCATCTGTGACCT |
| NO: 119) | GCTTGAAATGTAGGAACTGCTACTCGGCAGTGAGGACACCCACCAGGCC |
| | CCTGTGACCTCCANGGGTGTGTTACCAGCACAGGCAGAGCACTCACAGT |
| | GATG[G/T]TCCTCTTCACAGAGGTGAGTCGGGGGAGATTCTTGGGCCACA |
| | TCTCTCTACAGTCTAGNCTTTGTAGTGTGTCCTGCATGTGGTGGTGTGGG |
| | GACTGATTCTGGTGGGGACCCCTTGCTGCATTAGGTCTCATTCACACCCT |
| | TATTTCAGTTTGTACTGTGGCTCCCATGCCTGGTTACCTGCAGTGCAGTG |
| | GTGACTCT |
| BIEC580675 | AATTACAATTTTAAGGAAAAAATCTATCTCCTTGTCATCACATTTCCCGA |
| (SEQ ID | CCTTCCTTTGAGAGAGATTCTAGAGTCCATTATTTCTGTGGAGATTGCTG |
| NO: 120) | AAAATATTTTTACAACTCTCTCCAATATATTTCTACTATTAAATATCTCCA |
| | TCTTATTTTACTAATTCATGTTTGTCTAAATTCTGAGGTTTTACAGGCT[T/ |
| | C]TTTGGTATTGCAATTCTCTTATCCAGCTCTCCATCTATGGAACATTAGG |
| | AAGTCTCTGAGGCCGGTCTGAATCTCATAATTTGGTAGTGAATCAGACC |
| | AACGATTTAAGAGGTGGTTTTCAGAGAAAGTCCAATTGTGTTTNAAAAC |
| | ATCTGCAAAGTACGTTTTTCCCCTCAGCATCTAGAAGGCACACATGAAG |
| | TTGA |
| BIEC590604 | TCTGTGGATTTTCAGTCTATATCCTGCTTCCTTTATGGGGCTCTGGCCTAA |
| (SEQ ID | GGGTGTGGTCACATGGTCACTCATGGCACCAGGCCAGCTTGGAGGGTGT |
| NO: 121) | GCTCTGCCCTTCTCTCTTTCCGTGACATTGGCATCTCATTGAGTCCTCCGT |
| | CTCCTTTCTCTGCTCTCTTTCCATTTCATTCCCTCCTCAAATCTTTGTT[T/G] |
| | TTCCCATTGAGGAGAGAAAGCCTCGCTTTCCAGTGGGGCTTGAGTATTT |
| | CCTACTCAGGTTGATGCGCCTCTTTTGGGAATATAAATTTTGCTTTCCTTT |
| | CAGTTTTCTCCTCTGTTTTCAGTTCAAGACAATCTTGTGTACATGGCCAA |
| | ATAAAACAGCGTGTTGTCAAATCCCGGGACAGGATGGTGATGGTTATTGA |
| BIEC600682 | CCACATGCTCTTTCCCACGCTGTCCTGAATTTATATACCATTATGTAATTT |
| (SEQ ID | TATTGAGAAAGATATAGGTATCAATACCTTGAGCAGTGAAATTAGCTTC |
| NO: 122) | TTCACTCTCTCATGCTTAAGCTAACGTTACAATGAGTCAGCATGCCCATC |
| | ATCCACAGTAACTCACNGAGGACGAACCAGTCTCGACTGGCACATGAGA |
| | T[G/T]ATGAATGGACTAGTGTAGGATTAAAAGTTTGCCTGATATATTAAA |
| | TGATGAAATCGTGAAAAATGCATACCTGGTAGGAAAAACTTAATGGGTA |
| | GGATGCATGATGAACTCAATATGAGGGAAGTAGGGGCCACTGCAGAAT |
| | ATGGAACTGAGGGGTGATTCTTAGTTATGGGGAAAACCCAAAGGTGTGC |
| | CTTTCCTCA |
| BIEC622581 | ACCACCTTTCTGAGTCCATGCCACTGGTGGAGGCCCTGCCAGCTGCCAG |
| (SEQ ID | ACCGCCGGGCAGGGTGCAGTCCAAGGGGCTCCAGCTGTGGTCTCTGCCC |
| NO: 123) | TCAGCTCAGTGGAGCTGAGGAAACATGCCATACAGCCAGACCTTACGTG |
| | GGGAGCAGCCCTGGCCAAGAATGAGATAAACGGGGGGTCGGCAAGGTC |
| | CCAGC[A/G]AGGGCATCTTGCACACCTGGATGGCCAGGGACCACAAGGG |
| | AGCCAAGTCATTGGGCTGAAGGGCAGGTCAGAAGGCAGCCGAGCCACT |
| | CACCAGCATCCTGGAAGCCACACAGGTGCTCCATNCGCTGGTCAACACA |
| | GCATGGGGTGGGCACCTCCTGCAGGGNCAtgggggcagacccaggccccagtgacttcac |
| | cag |
| BIEC633730 | TTCCATGCGACCAGGCTAAGGACACGAGCAACTCTCAGTAGCAAATATG |
| (SEQ ID | AAAATCCAAGCAAAAGAAAGAAAGACTACATTCAGCTCTTGTAAATCTC |
| NO: 124) | AGTCTCGCTCANCGCCAGGTGGACATGACAGTTCATTGNCGCGACCGTG |
| | GATGGCGGGTGTGTCCGGGTGCCTATGCTCTATAAAATACAGGAGGCAC |
| | CACA[T/C]GTCTGAGATTGTCGAATGTCTAGTGCTAATAAACGTCACACC |
| | CTGCAGCTTCTGAGGAGAAAGAAACTGTCCCAGTCCACGGCTGGCTCTG |
| | CTAATCAAGGCCAGCTGCTTGGGCCTGTGTCCAGAGAGTCAGGNAAGGG |
| | TGGCGGGGAGGGAAGAGGAGAAACAGGGGCCCCATCACCGCCACGGGG |
| | GGACTCCCCTGT |
| BIEC637205 | AAATGGAATAACAAAACAAAGAAAACAACAACTGTGTTTGTCGTTGTAG |
| (SEQ ID | CTGAGCAGGCATGGCCTTTTCATAGCGCATCTGAAAGTGGGAGAGAGTG |
| NO: 125) | TAGGATTTGTCCTGAGCATTGTTCCCGGGATTTGCCGTCACAGAACAATT |
| | CTGATTTCAGTGGGAGGGTAGGGCCAAGCAAGACTGCTTTTGCTCTCAG |
| | CCT[A/G]TTTAAAGAAAATGGTGAGCTTGCCTAGGAAATACCCAGTGTTC |
| | TGCAGGCCCACTGTGGCTTGTTGCATTAACCACCCAAACAAAAAAATGT |
| | TACTGTGCATCCTTTCTTAAAGAAATGGAACAATGAGACTGATGTTGGCT |
| | TCTTGAATGGAAATCTAGGGGCATAAGCCACCCATTCTCTACAAAACAA |
| | AACAACATG |
| BIEC687178 | AATCCTCCACACTAAAATCTAAGTCTAAAGACTGGAAGCCCTGATTTTTC |
| (SEQ ID | TGCCAGTTAGTCACTCATTTATTTATtcaataaatatctatcaagtatttactatgtgctaggtcctat |
| NO: 126) | tttaggtgttaggaaaacaaaaaTGATTAAGACACAAAATTTCTTGAGCTCTTCCCATA |
| | TGTCAGACACTCTCAAAAGGAA[T/G]TCATAACCCAGTGGAGGCATAGA |
| | AGGCAAATTGCCCAAATGACTGTTGTACAGTGTCCTATAATACAGGCTG |
| | GAAGAGGGTTAAAGGAGAGATCAATGAGATGAGAGAGATGTCGTACAA |
| | GCCCTATAAGACAGGAGTCAAAAAGGACCTCTAGAAAGAGCAGCAGCC |
| | TCATTTACAACCCACTATGGTCCAGGGTGCTA |
| BIEC706272 | TGATGGTGCNAAGATTTCATGATTGCTTTTCATCCTTGCTGGAAGGATCA |
| (SEQ ID | ATGAGACTGGGGATATCTCTGGAATCTGATTAAAATTTCTGGCAGGATC |
| NO: 127) | GAGTTGTGGGCACAGGAGAGAAGGTCACCATCTCTTTATCAACCCACCT |
| | CAGAGTCCTACCTCATGATCTAGGTCCTAAAAGGAAAGGCAGCACCACA |
| | GGC[A/G]AAAAATATTCTAACAATGGAAAACTGCTTCCACAGTCAGCTGT |
| | AGGAGTCAGCAGAGCCATGCTCATTTTGTGTAATCTGAAGGTCTTAGAA |
| | AGAAAGACGAGTGACAGAAATGTGATCCCACACCTCTACTCTCATCTCC |
| | TGCCAGGCTTTCTCCCATGACAGCTCCATTTCTCTAGGCCAAGCAATTCT |
| | TTCCTTCTA |
| BIEC942271 | TAAGGGCAAGAACTCTCTAGATTTCCCAAAATGAGGCATAAGCAGGAAC |
| (SEQ ID | CCTTGGTGGTAAGGAAACCCCAAAGTTTGCTTTCAACCTGAGTATGCTA |
| NO: 128) | AACAAATCCTGGATAATTTGAACTTTTGCACCGGGTATCATGGCAAGAA |
| | TTTAAAACCTATGACCTGTTCATGATGGGTAATCAAATGGGAAACTCCCT |
| | GCA[C/T]TAAAATAGAATTGCAAACTGTGAATGTGTAATAAACTTTGCTG |
| | TAGGGAAGGGAGACAGAGGGAAAATTACCCAACTCATTTTAGGCACTTG |
| | GTAGAAGTTCAAAAACAAACAAACAAATCAAAAAAACAAAAGAAAAAA |
| | GTAAAACCTCATCTGATAATTCTGGAAGGAAATATCAGACTCAAACAGG |
| | CTCTGGTTCCA |

In further embodiments, the present invention provides a panel comprising a plurality of assay compositions, wherein each assay composition is capable of identifying at least one of the nucleotide markers as set forth in Table 5 below:

TABLE 5
DOG SNP PANEL SEQUENCES (SET #1)

| SNP ID | Chr | Position | G | A | Discovery Breed |
|---|---|---|---|---|---|
| BICFG630J1290 | 1 | 9088016 | A | T | Alaskan Malamute, Boxer, German Shepherd, Poodle |
| BICFG630J5593 | 1 | 19444782 | A | G | Beagle, Boxer, Poodle, Rottweiler |
| BICFG630J227421 | 12 | 57771412 | C | T | Bedlington Terrier, Boxer, Poodle, Rottweiler |
| BICFG630J232150 | 12 | 64596878 | T | G | Beagle, Boxer, Poodle |
| BICFG630J235932 | 13 | 12459211 | A | G | Alaskan Malamute, Boxer, Italian Greyhound, Poodle |
| BICFG630J255886 | 13 | 43403946 | T | G | Beagle, Boxer, India Gray Wolf, Italian Greyhound, |
| | | | | | Poodle, Portuguese Water Dog |
| BICFG630J265884 | 14 | 12884975 | A | G | Boxer, German Shepherd, Poodle, Rottweiler |
| BICFG630J275606 | 14 | 44515618 | C | A | Boxer, German Shepherd, Labrador Retriever, Poodle |
| BICFG630J278829 | 15 | 16039028 | T | G | Alaska Gray Wolf, Bedlington Terrier, Boxer, |
| | | | | | Labrador Retriever, Poodle |
| BICFG630J282369 | 15 | 24063852 | T | G | Alaskan Malamute, Boxer, English Shepherd, |
| | | | | | German Shepherd, Poodle |
| BICFG630J304928 | 16 | 3886095 | G | C | Beagle, Boxer, German Shepherd, Poodle |
| BICFG630J319569 | 16 | 43009172 | T | G | Bedlington Terrier, Boxer, China Gray Wolf, |
| | | | | | Italian Greyhound, Labrador Retriever, Poodle, |
| | | | | | Rottweiler |
| BICFG630J331636 | 17 | 14160564 | G | A | Bedlington Terrier, Boxer, English Shepherd, Poodle |
| BICFG630J346559 | 17 | 48612703 | C | T | Boxer, Labrador Retriever, Poodle, Rottweiler |
| BICFG630J356853 | 18 | 22596913 | T | C | Beagle, Boxer, Poodle, Rottweiler |
| BICFG630J358084 | 18 | 44102666 | A | T | Bedlington Terrier, Boxer, Labrador Retriever, Poodle |
| BICFG630J373954 | 19 | 21194807 | C | T | Beagle, Boxer, Labrador Retriever, Poodle |
| BICFG630J391832 | 19 | 56281961 | T | C | Bedlington Terrier, Boxer, Italian Greyhound, Poodle |
| BICFG630J402866 | 20 | 58705091 | G | A | Boxer, German Shepherd, Labrador Retriever, Poodle |
| BICFG630J399661 | 20 | 39886765 | C | G | Alaskan Malamute, Bedlington Terrier, Boxer, |
| | | | | | Italian Greyhound, Poodle |
| BICFG630J414309 | 21 | 28639360 | A | G | Alaskan Malamute, Boxer, German Shepherd, Poodle |
| BICFG630J421119 | 21 | 49794475 | T | G | Boxer, German Shepherd, Poodle |
| BICFG630J431948 | 22 | 21464933 | C | T | Beagle, Boxer, Labrador Retriever, Poodle |
| BICFG630J425382 | 22 | 6210670 | T | C | Boxer, Labrador Retriever, Poodle |
| BICFG630J457850 | 23 | 13946934 | G | T | Beagle, Boxer, Labrador Retriever, Poodle |
| BICFG630J473226 | 23 | 36806100 | A | G | Beagle, Boxer, Labrador Retriever, Poodle |
| BICFG630J484553 | 24 | 8851728 | A | C | Boxer, German Shepherd, Labrador Retriever |
| BICFG630J497958 | 24 | 29602886 | T | C | Bedlington Terrier, Boxer, German Shepherd, Poodle |
| BICFG630J503647 | 25 | 3274907 | A | G | Bedlington Terrier, Boxer, German Shepherd, Poodle, |
| | | | | | Rottweiler |
| BICFG630J525153 | 25 | 52605143 | A | G | Beagle, Bedlington Terrier, Boxer, Poodle |
| BICFG630J533364 | 26 | 21482093 | A | G | Boxer, German Shepherd, Poodle, Rottweiler |
| BICFG630J537466 | 26 | 28425454 | G | A | Alaskan Malamute, Beagle, Boxer, Poodle, Rottweiler |
| BICFG630J548189 | 27 | 5814598 | A | G | Boxer, German Shepherd, Poodle, Rottweiler |
| BICFG630J553154 | 27 | 16146331 | G | C | Bedlington Terrier, Boxer, German Shepherd, |
| | | | | | Poodle, Rottweiler |
| BICFG630J566667 | 28 | 11501579 | T | C | Bedlington Terrier, Boxer, English Shepherd, |
| | | | | | Poodle |
| BICFG630J573029 | 28 | 23791787 | T | C | Boxer, German Shepherd, Poodle |
| BICFG630J585149 | 29 | 15036709 | G | C | Bedlington Terrier, Boxer, Poodle |
| BICFG630J597522 | 29 | 41369057 | T | C | Bedlington Terrier, Boxer, Italian Greyhound, Poodle |
| BICFG630J608671 | 30 | 28455073 | G | A | Alaskan Malamute, Beagle, Boxer, Poodle, Rottweiler |
| BICFG630J613547 | 30 | 39085959 | C | T | Boxer, German Shepherd, Poodle |
| BICFG630J630348 | 31 | 30276777 | A | G | Beagle, Boxer, German Shepherd, Poodle |
| BICFG630J635046 | 31 | 41099916 | G | A | Boxer, German Shepherd, Labrador Retriever, Poodle |
| BICFG630J638804 | 32 | 14056351 | G | A | Boxer, German Shepherd, Poodle, Rottweiler |
| BICFG630J636447 | 32 | 7803442 | G | A | Beagle, Boxer, German Shepherd, Poodle |
| BICFG630J654194 | 33 | 11445001 | A | G | Beagle, Boxer, Poodle, Portuguese Water Dog |
| BICFG630J660369 | 33 | 26075493 | C | T | Alaskan Malamute, Boxer, Labrador Retriever, Poodle |
| BICFG630J667882 | 34 | 30670918 | G | C | Bedlington Terrier, Boxer, German Shepherd, Poodle, |
| | | | | | Portuguese Water Dog |
| BICFG630J676160 | 34 | 40730781 | T | C | Boxer, English Shepherd, Poodle |
| BICFG630J689381 | 35 | 25937791 | A | C | Bedlington Terrier, Boxer, English Shepherd, Poodle |
| BICFG630J678332 | 35 | 6882284 | A | T | Alaska Gray Wolf, Alaskan Malamute, Boxer, Poodle |
| BICFG630J693521 | 36 | 7667844 | A | C | Beagle, Boxer, Labrador Retriever, Poodle |
| BICFG630J695147 | 36 | 11554366 | T | C | Beagle, Boxer, Labrador Retriever, Poodle |
| BICFG630J707814 | 37 | 12867303 | G | T | Boxer, German Shepherd, Poodle |
| BICFG630J715531 | 37 | 33363432 | T | C | Beagle, Bedlington Terrier, Boxer, Poodle |
| BICFG630J719405 | 38 | 19640071 | A | G | Alaskan Malamute, Boxer, German Shepherd, Poodle |
| BICFG630J724770 | 38 | 26352306 | A | G | Beagle, Boxer, Poodle, Rottweiler |
| BICFG630J729876 | X | 4043645 | T | C | Boxer, German Shepherd, Labrador Retriever, Poodle, |
| | | | | | Portuguese Water Dog |
| BICFG630J749105 | X | 98054740 | A | G | Beagle, Bedlington Terrier, Boxer, Poodle |
| BICFG630J745699 | X | 88286773 | A | T | Beagle, Boxer, Italian Greyhound, Poodle |

G = genomic allele; A = alternative allele; O = Other SNP within 30 bp of genomic/alternate allele: P = percent repeat.

The nucleic acid sequences of the markers of Table 5 are provided in Table 6 below, where the position of the polymorphic site (e.g., the single nucleotide polymorphism (SNP), insertion and/or deletion) is bracketed and indicated in bold:

TABLE 6
DOG SNP PANEL (SET #1) NUCLEOTIDE MARKER SEQUENCES

| | |
|---|---|
| BICFG630J1290 | GATTAGACCTTTAATGTTACAGCAAATATGGTTTATGATTCTTTT |
| (SEQ ID NO: 129) | TTAAAATTTCAAATAAAACTTTATGTTGAGAGCTATGACTGCAG |
| | TTCTTTCTCTTGTCCTCCCTTACCTAATGCCCCAAATTACTTTGGT |
| | TGTCTTCTACTGAAGTTTTTATTTCTTAAAAATCCGCAACATATA |
| | GGTCTAGGTGTTGTCTCAGA[A/T]GCCATGTAGGATTTAAACATC |
| | CCAACAGAGTGAAATGCTATTTCAGGAAATACGGTGCACGCTTG |
| | CCACCTAGTGGTGAGTGTGGAAACAAGTGAGGATTTCAAAGCA |
| | ATCCCAAAGAACGTGAATTCNNAGAAANACTAAGTTCACTAGTT |
| | ATNTTCAAATATAGTAAGGTATAAGTGTTATGTGAAAACTATTA |
| | TTTT |
| BICFG630J5593 | ACTCAGCCCCAGCCCTCAATGTGCACGTTATCTCATGGGGGAAG |
| (SEQ ID NO: 130) | GAAACACATGGACAAGTGGGGGGTGTCAGAGGTACTCAATGGT |
| | GGTACATAGGGACAAGATTGGCATGCAGTGAGCAGGGGCAAAT |
| | CCCCCCAAGTGTGGGTAGGTGAATGTGCTGGAAGCAGGTTGGAG |
| | GGGAGGAGTCAGGAAGATATGCAAGA[A/G]GAAGTATCCCAAC |
| | CAGTTCCAGCTCCCAGCTTGACCCATAGAAGGGGGAAGACATAA |
| | TTAAACTGCCTGGGGGCATCCAGGAAAATACTGGTGAAGACTCA |
| | GCAAGGTTTCTCCATCCTTCAGTCAGTGCACTGAAGAAGTGCAG |
| | CTGAGGAAAGAGCAGTAAGTTAGTGGACAATGACCACACACAC |
| | CAAGGTGTGCGG |
| BICFG630J24664 | CCTTCCCTCAGCACAGCCCCTGGCTCTTCACGGTCACTTGGAGGC |
| (SEQ ID NO: 131) | TGCCTCATGGCTCCCTTGGAGCTGTGCTTGCCTGGGCAATGGGCT |
| | AGTTCCTTCAGGGTTCAGAGGGCTGGAATGAGACCCTACTTGCT |
| | GTTGGCTTAGTAGACTCTACCCTGGAGCTGACAAGGGGAGGTGG |
| | CTCCACGGGCAGCCCTGCTCTC[A/G]CTGCCCGACTACCTGTGGA |
| | CACGTGTGGACACCGGCGTGCGAGTGGCCCTGGGGCCCCTGGAC |
| | CTAGCATTCTTCCCAGCCTCCACTTCAGAACTGGGATCTCTTAAC |
| | ACCTCTCCCCACGTCTGCCTCTGGCATCTGCTCTTCGGGCCCTCC |
| | CCCGGGGGAGGGGGCGGGGGGGAGTGGGGGGAATGTTGCTCTT |
| | GCTA |
| BICFG630J27518 | TGTGAATAATCTCTTATAAAAGCAGTAAAGATCATGCCATTATA |
| (SEQ ID NO: 132) | CCTGTTGAATTTGCTGCAGTTTTAGTTCTATTTTAAACAAGGTGT |
| | CATGAAAAGCACAGACTTACCTGTACGGTAGACAAAGTTGCCTT |
| | CGGTTTCTGATGATGAGGGAGACACCAATTCTTCCTCAAATTCA |
| | TTGGAACTAAAAGATCCCGAATG[G/A]TTTCTTTGCCTTGTCTTT |
| | CCCATCATGGCAGCATTTGTGGCCATGACATGTCTCAAGGAGTC |
| | GTTAAGGTAACCGAATTCAAATAAAGCTGCTCTTGTATTNGGGG |
| | GGGTGAATACGTAGTCCTCACTGGCCTGTGACTCTGGCCTCACT |
| | CCAGCTTTTATGACTGAGCTTTCACTTTTANTCACAGGATGATGA |
| | ACTGG |
| BICFG630J34588 | GGATAATTGCAAGTCATAAAAGAATTAAGACATTTTCTTCCTGA |
| (SEQ ID NO: 133) | AAAGACTAATTGAAACTCTAAGAAATGTGAGTTACATAGAACAT |
| | GCTGGCCACCATTTCAGCCATTTTTGTCTTTATTGAAAGGGCTGA |
| | TATTTTATTTCCAAGGAATTGCAAGTGTAGTTTTTAAAATACATG |
| | GTTGAAAATATGATAGACGTTA[C/G]AATGCTGAATTAGAGAAT |
| | GACTGATTTGAAAAGAGGTGCCATAAAGCTGTTACATTAACCCT |
| | TCGTTGAACATCATATGTTTGATGGTCAAAGTCTCCACGAAGAT |
| | AGACCGCCAATCTCATAAGGCACACTAGGGCGCTAGGTGAAGCT |
| | CACAGATGATCTCATGAGCTGGAGCCTGCAGGAGGAAGCGTTG |
| | GTGGGCA |
| BICFG630J36601 | CTTCCTGCCTATAATTTCCATAGACCAAAAGTCTTCTTTCCCCTT |
| (SEQ ID NO: 134) | AAACTAGAATAATTTCTTCTTTTCTCAATTCAGTTTTCCTATTAG |
| | AACAGACTANAAGGGAGGTTTTTTTTAAGATTCTGGGCTCTCAA |
| | CTTTTTTTTTTAAGGCAACAGAGACATCTTTTGGCCAATTANTGC |
| | AACTGCATGGTGATAGTAATG[A/C]AAAGTTAATACACTATGAG |
| | CTGCATTGGTGAGCCATTTTCTATGATCTGTTCAGTGATTCCTCA |
| | GTCCNGTGACGTTTCAAAGCTGATACAGCATTGGCCCACTGACC |
| | ACAATAGGAAGTTTTTCTGATAAAGAAAGGCAAGAGTCAGGAT |
| | CTGGATCCACTACCTGAAATGCAGTTCGATCTGAATGGATCCTT |
| | GGGTG |
| BICFG630J39325 | CACATCACGGACACATTTCCTATGATCTCTACTCCCNCTCTTTTG |
| (SEQ ID NO: 135) | TCCTGTAAAGTAGGAAGCAGTAAAAGGCTATAATCTGGGACAC |
| | ATTCTTCTATGGATGGATTTGGGGAAAATAAAACTTTTTCACTTT |
| | TTCTCAGGTATAGTGCTATTACACTATGTTATAATTAAACATAAA |
| | TTGCAAATATCACGAACATAAC[C/T]GTGATACCTTATTGAATTC |
| | AGGAATTAGCCTTCTCGTGAATCTTCATGGTTTGTGTAATGAAA |
| | GCTGGGGCAGTAGGGAACATTGTTGCTTCAGTGTGGTCTCCTTCT |
| | GGCTGTATGGCTGCTGTCCCATTTCACTTCAAGCATTATTTATCA |
| | GGTAGTTTCAGCCTCAAGATCTTTATGAGACCCTTTTAAAATATG |
| | TT |
| BICFG630J54631 | ACATGCACAAAACAGGAAAACTGGTTGAAACTCACTGGTGGCA |
| (SEQ ID NO: 136) | CCTGGGCAGTCACTTTATGGGCCTACTGAATGTTTCCATGGAAG |
| | TAGCCAAGGGAGACACACACTGCAGAGCNTCGTAAGTTGGCTCC |
| | TGACCACAGTTTGGCAAGGTGGAAGCCATATTATGGGACATCTA |
| | GGAGGAANCCCCTTGGGAGCAAGGT[A/G]GTGAGGGTCTCAAAA |
| | GACACAAAGTGTTCTAGGGCTTACTTATCTTTTTTAATGGTTTGT |
| | Gtggatttgagaaaatagtcaaaatgaaggataatagagggatgaaactgtcctacagagcaagagaccc |
| | caccagtggaacaaaactacacacaagatattagatattaggtataagatagaacaagagtatacttccc |
| BICFG630J64739 | GGTAATACCAATAAAACATCTATTGGTAACCTACTTCTTCCCTAT |
| (SEQ ID NO: 137) | TCAAATGGGCGCATGAACCAGATGCAACAGGGAGATGGAAACA |
| | ATTTGCCTGACTAGTTGCTTTTCAGGAGAGTAGGGTGAAAGTTC |
| | TAGTTATCCTGTGGGGTTCTGGGGCTTTAACTCTACTGCCTGTAC |
| | ATTTAATGTGAATGAACCTATTC[A/G]GTTGTTAGAATTTAAAAT |
| | ATGTAGAAGTTGTTTATAGTTTGCTATATTTCTTTCCTAACGTTG |
| | CGGGTTTTTTAAGAGAATGATTAGTAGGTAGAACTTTAAAACGT |
| | TCATCTGGATCTGAACCGAATCCTATTTTATAAATCCCTTGCTTT |
| | GCTGAAATAGGTGCAGGAAAGGTACGCTACACTTGATTTTAAAT |
| | TAAG |
| BICFG630J74970 | TTGTGCCCACACAGGACCTCCAGGGCTCCCCTTAGCCTGCTCTAT |
| (SEQ ID NO: 138) | ACATCAGTCAGATGGCCTCAGGTTTTTAGCTATTTAAAAAGTAA |
| | TCATCTAAAAAATAAGATTTGTACGGTGTAGTTGTTACCATCATT |
| | TTGGCGACATTTATAAAGCTGCATCACTGGTAATGTAGGGCTTT |
| | CCACCTACATTTATCTTTAACC[T/G]CATGCAAATTGGAATCAAG |
| | AACAGCTGATGCAGCCTAGTAAACCCATGGTAGAAGTTTCCAAA |
| | AGAAAGGAAATAAGTACACGGTCATAAATGCACTCTTATTTTTA |
| | TCAATAACATTTAAACATTAAATGCTAATTATGTAAAAACTCCC |
| | ATCAATAAAACCCCATTTATAATTTGCAAGGATCACTAGAAGTT |
| | GGATT |
| BICFG630J79584 | AGAACTCCTCTGTTCTTTCTTGTCCTCAAATGGTGGTGACTGTTT |
| (SEQ ID NO: 139) | TCATCAACCTATGCCTCCCCGCTACAGGCTTCTCAAGTTTGCAAA |
| | TACTGCTGAGTTTATAAACGGTTACACACAAGCTGTACCTACCA |
| | TGGTGACAATGAGCACGAGGAGCTTCAGTAAATACTGACAGGTT |
| | TTGGTGGGAGGCCCACCCTTCT[G/A]TCCTATTTCACACTCAAGG |
| | AACCTGCCCCATCAATCCTGGGGCTTCCTCCCTTCCTCAGGCACC |
| | TGGACCCTGCATCTTCCCTCTAGCCAAACGAGACATTCCTGCCC |
| | AAGGACAGCGAGGCAACTTGTTTCTGCACTTGCAGCACTTTGCA |
| | GATCACAAGGCCTTCCGAGAGTGGGAATCAGTGAACACCCAGA |
| | GATCC |
| BICFG630J89999 | GATCTCAAAACAGGTCCACCCTGGCTCATGCAATCTAGCCGAGT |
| (SEQ ID NO: 140) | TGGGGTGACACCAGCTCTGATCACTTGAGGGGCGCAGCCAGCAG |
| | TATTGAGCCTCTCTATGTACTGGGCACTGTGTGTGCGTTTACACC |
| | AGTTCTCACAATTCAGTGACACACATGCAGGAGGGCGGAGGGG |
| | AATAGTAATAAAAGAAGTTTCCAG[A/G]AATAGTAAGACCAACT |
| | TTTAACAGCGGGTAGATAGGGAATAAAAAAACGTTTTAAAATTG |
| | TCAAACCATTTCCTTTCTATTTCTCAACGTAGGGCATTGCCGGGA |
| | GGGGCACGGATCNAAGAACNAAGTCCAGGCCTGCCTCGTTGGT |
| | GTCGGAGNACAGCCCTAGAAAACAACGTGACTCTGGGGATGTA |
| | CGTCAGGGA |
| BICFG630J98358 | Acaccgggctccctgcgtggagcctgcttctccctctgcttgtgtctctgcctttctctctctgagtctctcat |
| (SEQ ID NO: 141) | gaatcaataaataaaatactttaaaaaGTAAAATAAAAAAGAAACAGTGCTCTTC |
| | CTACATAGGGAGACTAAATGATAGCTGCTGTTTGGGGTGAGTCT |
| | CCAGACCAGAACCAGACCAGGGTTG[C/T]CCAATCTAATATAGA |
| | TTGAGGAGTGGGCCAGATTTGCAGAGGCAGAGGGGAGGAGGGA |
| | CAGACAGGGACTCATGGGACctgtgtgaagcctgttacgcacattatgtcattcaagcgt |
| | aaacagacctgtgggtaccggtgctagaattacctccatttcacagatgtggaaagtgagactcagaCCC |
| | CAAGAGCTCGTT |
| BICFG630J101630 | TCTTCCCAGTAGGCCAATGTCAGTGGCACCATCTCAACCATAAG |
| (SEQ ID NO: 142) | GGAAGTTAAAGGATCCCTGTCCNCTGCTCCATTTCACTCCCAGG |
| | AGAGAAGAACTTTGATGAAAATCAAGAGGAGATACAGTGGTGC |
| | TCCTGTCTTAGCAGGCAGAGCTAAGCTGTGAGAAACCTCTGCTG |
| | GAGATGACCACNCTCATCTGGATTG[T/C]TTATTTGGCCAATCTT |
| | TTATGATCTTTGCTCTCAGGTAGTATCTGGGCTGCCTTCTGCTGA |
| | GGAGGGCCTCCTTCCCTGAGATTCCAAGCTGGATTGTCAGAGGG |
| | ACCAGTGGAGAGACNGAGTTGAGGGGACCGCACAGACTGGGTC |
| | TGTGGCCTCAGCGGAGACTAGCTTGTTTCCCTAGCTTAGTACGCC |
| | TGTGCAC |
| BICFG630J111559 | ACTGCACTGCTTTCTCCTTACAAAAATTTCACGATTTTTGTGCTC |
| (SEQ ID NO: 143) | TCCTTGCTTACATCTTCTAAGTCTTCTGGGTCCAGTCTTTATTTCA |
| | ATCATGGGCCAAAGCAGTTCATAGTGAGTGACTAGGTTCTATGT |
| | CTCCTACGAAAGGCTGCCACTCCCAGGGTGAGTTAAGATGACTC |
| | TGNTAAGGCCGGTTTTGAATG[T/C]TCCGTGGTGAACTCAAGACT |
| | GCTGCTCCAAAGCAAGAACTTGGGCTCATAAAACAATAGTGTTA |
| | TTACGATCGACGCAAAAAGGTGTCTTATTAGTTAAGTTGCTGCC |
| | CTGCTGCCAAGAGCAGTGATCTAGCTTCTAATTTTCCTTTTTCAA |
| | GATGATCAGGATGAGACTCATCAGATGGTTTTGCTCAGATTAAA |
| | GGA |
| BICFG630J113042 | GCCACCTCTCTGAATCTTTTCTGGTGTTCTCACTATTTCCTTTAAC |
| (SEQ ID NO: 144) | TTTTTGTCACATTCACACATTGCTTAAAAAGTGTGGAAAAGGTA |
| | CCTCTTTTGGAGAAAGAGCTTAAGAGTGAACACTCAGCTACTCT |
| | GTCTCTTGCTTTACTCTGGGTTGGGAAGCATACCTGGCCCAACTT |
| | GGTGCCAGTGCCCACCAAAAC[A/G]AGGACATCCCTGGCCCAGT |
| | TCATGTCAGGCNTCAAGAGCAGGAAGACGTGAGGGAAAGAAAG |
| | GGACATGGAGGTTAGAGCTATTAGAGCAAATCACCCTGTGCTTC |
| | CTAGGGGTCTGTGCTGATCCTTTTCCACCTCCTGAGGGTCAGGGT |
| | ACTTTCACTTGGGATGTGCTCTACGACAGGCAGCATAcaaacacaccca |
| BICFG630J120171 | CTCTTGGTGTGCATGAGGGGGGAAGAGATATTTGTGTACATGAA |
| (SEQ ID NO: 145) | GGCCACACCTGGGCCAGNTTCCATGAACTCCTGTGCTGACTCCT |
| | GAGCTAAGATCTCCTGCCTCCCTGTACCTCCCTGGGCAGCTCTCT |
| | TGAAAGCAGCAATTCCCTGATGGCCCCAGTTTCTTGGTCGAGGG |
| | CCTAGTGGCCGCTCTTCCCTGCT[G/T]GGNCCTGATGGGTCAGAC |
| | ATCGGCTCCCCCTTGCCAGAATAATGGTTCGGCCGCAGAAACCC |
| | ACACAATCTCCCTGCGAGACAGGTCTTTGTTGTTCTGATGTCCGT |
| | GGTCCCAGACATTCAGCAGCTTGCACACTCAGGCTCAGTGTACA |
| | CACTTANACTGTCTTGTCTGGAATCGCTAACTGAGCCTGACNCCT |
| | TAGG |
| BICFG630J132438 | TAGTAAGTTAATGAGCCCCTGACATATTTCCAGGAAATGTTCAT |
| (SEQ ID NO: 146) | CTTCACCAAAAATGAGCCATTTCTCTTCACTCAAGAGTTACTCTT |
| | CATTCTACTGTTTTCATGGTTTTCAGATACTTTATACATTACTTGA |
| | GNCTCGCCACGATCCTCAGAAATGCACAGGAACCCATCAGTTGG |
| | CGTCCTCCGCAGACGAAGCCA[C/G]GGTTCAGAGGCGTCAAACA |
| | GCACGTCTGGAGCCTCGTGGTTAATCAGCAGTGGATTTGGGTTA |
| | AACCCAGGCCCTTGTTCTCCAGACCTGTTCACGCTCAGGCTGAC |
| | ACGCTCAGGCTCCGGGACCACGGTCACTGTCACAATGCTTCCCT |
| | CCCTTGCTCATCATTTTCAACTATTTAAAAGGAAACTCAAGGGG |
| | NTGGA |
| BICFG630J137139 | GGCTTCCTCTGTCCTACAGCTCATCCCAGAGCAATGAATCTGCCT |
| (SEQ ID NO: 147) | GAATCTGTTGGTGACAAGCTAAGGGCATGGGCTTCTCTGGGCAA |
| | TTTGTGGTAGGATAAGACTGGTGCTGGAGACAGGAGAAATTGCG |
| | GGAACCCTTTCTGCCTTTGAGCCACTTCATTCCCAGTTCCNGAGG |
| | AGAAGGCTAGGGGTGGGGGTAG[C/T]TTAGCCCAGGGCCTCCTC |
| | CTGGGGGTGGAGTTGACCTTGAGAGGAGCAGTGGCATCTACCAT |
| | CCCTTCCTCTTGGGGCTCTTCAGTGCCAATCTGAACAGTCTGGAA |
| | ATGGAATCTCTGGGACCCCTCCCCATCACTATTATTATAATAAAC |
| | TCCAGGATTGTATCTGCGGAGCCTCAGGCTTCAGGAATGCAGCC |
| | TGTA |
| BICFG630J145174 | GTGTAGAAATGGAATGGAAAATTCAGTTGAAACACACACACAA |
| (SEQ ID NO: 148) | TGTCAAAATTCAGTTGGTTTTACCATAGGAGATTAAATTAGCCA |
| | AACAAGGGTTCTCAGTCTCTATTTTAGGTCAATTTTGAGGTTGAT |
| | TATGGTTTGTGAATATTTAGTGTACTGTCAGTTTCATAAAAATAA |
| | AAGGTAAAACTTTTCTCCCTGAT[T/A]GTCTGAAGTGATGAGAAT |
| | TTATATATTAGCAGGCAACCCCAGGAAGCAGTGTCTCTAGTGGT |
| | ATATCAAAGGCCAACATTAAAGTATTAACTTCCCTAAACTTAGG |
| | TTTATTAGGTTTTATATCTGGTATCAACAGACTCTTATGCTTCTA |
| | GTCAGAAGATTTTTAAGAGGATAGACATTCTAAACAATGCCAGG |
| | ATCAA |
| BICFG630J156161 | GTGTGAGCGCGGATGCATAAGGATGGCGCAGAGCTCTGGACTC |
| (SEQ ID NO: 149) | AAGCAGATGAAACAGGGTGGGAGTGAACACTGGACGCTGGAAG |
| | GACAGGCTCAGGCAGGAGCAGTGGGGAAGCACGCCCTCCCCGG |
| | TGCTGCTATCTTTCCGTGTCAGGACACAGCCGCACAGTGGCTTTT |
| | GCTCATGCACGTGCAGCTATGTGTGT[T/C]GACAATTCCAGCTAG |
| | AAGGGTGCAGAAACTAAGCAGAACTTGACTGAGTAGGACAGCG |
| | GGCAGCAGGAGGGCCGCCCCCGTCACCGGGAGGGTCACACGTG |
| | CAGCTCCAGCCAGAGGAAGCTGGGGCACGTCGGTCCGGACCTCC |
| | GCAGTATGTCCGCGGTTTGTCCACGGCATCAGGGGACACCGAGG |
| | CAGGGTAGCCA |
| BICFG630J156875 | Ctagatcctcttttacaggtgtcatccactctcttcttgatcacatccaatgagattatgatacttactcagctctc |
| (SEQ ID NO: 150) | gcaNgccaagagagctgagtcagtatcatttctttgttctattgttttagtNgctagaaagttcttAACAG |
| | TAAGGGATATCTATAATTTTCACACATTTTCCAAGTTTTCACCCA |
| | TCNAAAG[G/A]GGACCAAAGGTTTGAAGAAAGTTTCCAAGTGTG |
| | TCTTAATTGTTTTACTCCNGGTGAAATATCCAAGATCTTTCAGCN |
| | ATAGATAATAAAGTGTACAATTAGAANATTATTATCTACTTCAA |
| | TCAGGGCACTTTATTTCTGCAAATGGGAACAACATTNAGCACTA |
| | TTACCTTCTTTAGCAGCTCTGACCACTTGATTGT |
| BICFG630J160536 | AAATCAAGTTCAAGGAAATATTATTTCCTGCACGCCAGGTAGAT |
| (SEQ ID NO: 151) | GTCAGGCACTCTGCTGGTGCTCTGTGTGGGCGATCCCCTTTCCTC |
| | CTCCCCATATTCTGAATGAGAGGGACTGTCCCATCCTTTCCTAGG |
| | AGGAGGAGCCTTAGGTTCTGTGGGGTGAGGGACCAAGTCTACTT |
| | TCACACCCATGCTCCTTCCTCT[T/C]GTTGACACCTTCTTGAAAAT |
| | ACAACAATCCACATTTCGAGTGCTATCGTACCAGGTCAGACAGC |
| | CCACACACCCTTAAAAATATTTCCTTCTCCCTCCAGTCTTTTCCT |
| | CAGAGTAAAAGCTTTCGGGAAAGGCCCAGATGTGCTATAAGGG |
| | AAACTCAGCAATTATGCAAGTGAGAagcacagcccaatggttagagcg |
| BICFG630J164406 | GCCGTGGGATCACGTGATGCAGTTCCTGGGAAGTGGTTGGTGAG |
| (SEQ ID NO: 152) | TCAGTTTCGTGCTCCTTGCCCAGCATTGGTAGTTTGGNTGCATGC |
| | AGACTGGGAGTATCCCTGAGGTGAAGGGAGCTTGATGGTTATAT |
| | GTTCATAGTGATGAATTGCAATCATGCTGGAGCCCGAGTCTGTT |
| | TCCCCAAAGTGCCTCATTCCAGA[C/G]GCCCGCAGGCATCTGTCT |
| | CTTGGAGCACCCTACCNCGTTTGCATAGGGCAGTGCATTCAAAG |
| | ANACTTGGGTGGAGGACATCTGGCATCATGTCTTCCATATGTCA |
| | AGGCCGAATCNAGCAGGTGGATAGAAACAGGACTAAANCAGCA |
| | TCCNTGANCATACTTCCTNAGTGACCAGCAGTCCTGCTCTGATTT |
| | ACCCAG |
| BICFG630J168764 | TGGGCATAACTGCCCACAGTGGGCAGCACATGGCCCCAGTCAAG |
| (SEQ ID NO: 153) | TAAAAGACTCCTTACCACCTTCCCTGAATCTTTCTCCCCAGTCCC |
| | TGTATATTGGAGTAGAAGAGACACAGGAAGAGGAGGATGTATC |
| | CCCAGAATGAAGGAGCAGACCACAGCCCAACTCCCCACTAAGG |
| | AACCTAGAGCCATTGGAAAGAGCTA[T/C]ACTGGAGGGAANGGA |
| | GGAGAAAGGCAGGGCCAAATTTACTNGAAGTTTCACTGCATCAG |
| | ACTGAACCAGGCTTTGTGAACCTAAGTGGACATANGGGTCTGAG |
| | ATTTGCCCACGATATGATCAAGGGGTGGGGAAAGGGGCTTTAAT |
| | GGCCAGTAAGTGAGGGAAAGTATTATATGCTTATATTTTTCCTG |
| | CTAAGTCAG |
| BICFG630J178333 | AAGAGCAGCTCTGCCTAGTGGTAAGGCAAGGGAGGTTGACATTT |
| (SEQ ID NO: 154) | GGATGGGTATCTGCCTGGTCCCTGCTGGCAGTAGCCCAAAGAGC |
| | ACTACTGTTCATGGGGATGGCTTCCATGACCTGAGCCAGGGGTT |
| | GGATGGCAGCCTTAGCTCCCAGGGTCTGCTGGTGACCTCTGCGT |
| | CAAGGGGGTGATAACTGGGTCCCA[T/C]AGTGCCTTGTCCACCTC |
| | TTTTACACATTAGGTTGGCCTTCTTGTAGAAGCAGAGTGACTTCT |
| | CGGGCCATGGAATGTGGCTCTTTTCCCAGGAGGCCAGACTAGGT |
| | CTGGGACAAAAGCTTTGGGCCAGGGGTAGAGCTAGCTTTGGAGT |
| | GAGCACAAATATGCACGTGTGTGTGCACGATGTGTGTGTGTACC |
| | TGTGTG |
| BICFG630J182918 | TTTAAGAGGCTGGTCTTCTGAGGAAGAAATCACAAAAATTATAA |
| (SEQ ID NO: 155) | ATCATAAGATGGCATTACTGACATGAGAGTGAATCACACTGGTA |
| | CATNCACAAGTGACGGGCTGGTGCAAGAGACTTTAAAATAAAT |
| | GTTTGAAATAATCAAAGACANAAATGAAGGACTAAGTGCATCCT |
| | GCAAGAAGAGGGCAGTTTGAGAATG[T/G]CTGCTTTGCAAACAA |
| | TGACAACAACAAAACAGATTTTAGAATCTAAATAAATAGTATTT |
| | TAAAAAATGTACAATAGAAAAAAATGGATCCTGATGATAATTTT |
| | GAGGTTTCTTTCTGGCAATAACTGATATAAGAAGCTCCAGAATT |
| | CTGAGTACATAGTGTGTGTGTAGGTGCACACACATGCACGTGCA |
| | CATGTCTGT |
| BICFG630J190167 | ggactcgatcccaaacttcaggatcatgacccaagccgaaggcagatgcttaactgactgagccacccag |
| (SEQ ID NO: 156) | gtgNcccTAGGCATGACTGTAAGACTTTTGATTATCCTCATGCTGTA |
| | GAGGTGGGTAGTATATATATCAGTGCCATGTGATTCATTTGAGC |
| | AACTTCCTACTGAGAATTTATTTACAAGGATGTGTATAT[C/G]CT |
| | CCTGGTTGAGAGCTCTGAAAATGATGTAAGGCAACATGAAGATC |
| | CTAGAGAATATTTTATTTACACCCCCTAGAAACTTTTAATGTCAG |
| | CAGATGCTAGCTAAGGTGTACTTAGCGTCTGCTTGTGTACCCGA |
| | TGTGCTAGGTAActctgtgcctctgtttcccattgacaaggtaggaatagtaagagtgtctacat |
| BICFG630J209785 | GTTTGGGATCTGGCTCTTGGATGTTACCAAGTATCTGGGGACAA |
| (SEQ ID NO: 157) | AACTCAGAATAACTAACTTTCTTGCTTCTCCTCGAAATGGGAAC |
| | ATAGCTTCTTTGGCTTATTCAAAGGCAAAATATGGGTCGTGNGT |
| | AAACAGGGCCTAGTGGAAAAACAGTAGAGATGGAGCCTTTCTA |
| | AAATGGATTCCCTTCAGTTTCTCTA[T/C]GTCATGAAGGCTCACT |
| | GCCACCGCCTCTCAAGCAGAGCCACAGTTTCACTGGAGTGGCCG |
| | CCTGGCGTGGCCATNGGGCCAGACACCAAATGCAAGAAGCACA |
| | AATGCGTGACAGAAAGAGCTTCCCCTTTCTGCTCCCTTCTGTGGA |
| | TCGCAGATGCTGCATTTGAGTTTTGGGACAGCTGTTTTCCCCCGG |
| | GGGGCTC |
| BICFG630J215562 | ACCGGCTTGTAACTGGCAGANCCCANTAGGCATTTTCTNAAGGA |
| (SEQ ID NO: 158) | TACTGGCACTGGGACATCCACCANTCTAANAGGGAAGGTATTAT |
| | CTAGTGCTGCTTGATNNTATTTCNGGATATGCCTGCTTCCTTTTC |
| | TGCTTACAGAGTTCCTATGTCCCTTCTTCTTGTTGTTGTACCGTGT |
| | GCTATGACTGCCATCACTTCA[C/A]TCTGCTGGTTGTTTGGAGGT |
| | TTGTTTTNTCTCGACAGAACTCAGAGATACAGGGTAGGAGATGG |
| | GATCGGTCGTGTGCACCCTCAGCCTGACAGGCACATANGCACNG |
| | GCATCGAGGAGGACTATGGGTTGACTTCTCATGAGAGTAACAGA |
| | ATCCTGAGGAGAAGAGTCATACGATCCACTCTTTCATCAGATTC |
| | TCTT |
| BICFG630J227421 | TTTATTAAATAAGATATCCCTTCAACATTGGTCTGTAATGCTTCC |
| (SEQ ID NO: 159) | ATTAGCATGTAGTTAATTCAGAGACACATCNATGCATATTCCAT |
| | TACATTTTAATGCACATCAATTATTTTGTCAGTACCACACTGTCA |
| | ATTGTCAAAGCTCTAATGAAGTGACATATGATGTTATTCCATTAC |
| | AGTATCTCAAGATGTGAACTA[C/T]CAGTTCTGGGTCATCTTACC |
| | TTTCCTTGCAACCATCCCCCNCGCCCNCCCCCCNCACACACACA |
| | CTTTATTTGTGNAGGATAATTCCGAATTAAAAATAGCAAAACTC |
| | TACACTGTCCTGCNAGATGTACAATTAAAANGATGAAGAAAACC |
| | AAAGACCCAGTTTTGTCTTCCCCAGCTACCAGAGTGAGCCCAAA |
| | AATA |
| BICFG630J232150 | Aaatggcatttccatgtcacattcctatgtccccaaatcaggatttgaagccggttgttctgacttacggccca |
| (SEQ ID NO: 160) | aaactcgtttcaccacaacaGACGTACAAGAAGAAAGACTAACAAATCCAT |
| | TTTATGAAGTAAAGGATCTAACTACAATTTTTGTTCCAAAGCTTA |
| | TTGACACCAGGACCCAGTGGGCTGGGACAC[T/G]GATATATTTT |
| | ATCTTCCTGTACNTCACAGCTGTCCAAATCTTGATCTCTTCAATA |
| | GTGACCCATTACACAGtctcatccagtcttctggatttaaataccatgtatattactaataattata |
| | cattttatctctaacgtgacNctttNcattagataactaacatttcaatattattcatccaaaatcgaggtactga |
| | ta |
| BICFG630J235932 | TGGTCACAATTGTTGACACTTCCAGCCCCAGTTCCTCCTAAAAG |
| (SEQ ID NO: 161) | GGATAAAAGAAAGGAAAGGATATCTAGTGGCAGGAAAGTATGA |
| | AAAGGGCAAATCCTCTGACTTAGCAGAGGGACTGACACNGAGA |
| | AACTTAGGTCAGGGTAGAGGTAGAAAAGGAGCACAGAGGAAGT |
| | AGCAGTGTCTCTGAAGGAAGCATTGCC[A/G]TCACCAAGCCAAA |
| | TTTATCCAAGGACTCACACATTTCTGTGACAGGATCCCTCAAAT |
| | AAGAAGGCAAGTTTCCTGTAGAGAGACACAAATGAGAAAGGCA |
| | GGGACCTTTTTTAGCAGAGCTGAGATTTTCTGGAAAACCTGGGG |
| | AAGCACACACTTCTCAACAATTCAGTTAAATTCTTTACACTATCT |
| | TTAGTTCAGAA |
| BICFG630J255886 | CTCCCACGCCGCCTCTCTTTCTATCCCAACACCTCCCATACTACC |
| (SEQ ID NO: 162) | TTGAGAAGCAGGTTCCTGCTCTGGGGATTGTCCTGGGAGCACAG |
| | TTTTTCAAATGCTTGAATCTTCTTCCTGAGGAGAGAGAAAGAAG |
| | GCAGGACGGATGTTGGAACACTAGGNTCGGGTTGAGGGCAGAC |
| | CCTGTGATCTGAGACCTCGAAGGA[T/G]TTCAACTGCTGGATCAG |
| | GGTTTCCTTGGGTTCCTCTGAGCACTCAGGTCGTTGAAGGACAC |
| | GGGAGGGAGCTCTTTGGAGGGTCATCCGGTGCATCCGNTGCCCT |
| | ATTAATTGAACNGCTCTGCTTTGGTCAGTTTTGGTTTCAATTGCG |
| | AAGAGACTCTAGTTGCTGTCATGTTCGCATCCAAAACCTTGTAC |
| | CTCAGC |
| BICFG630J265884 | CAACACAAATTATTTATACCATAGTGAGTTGAATATAAGAATAT |
| (SEQ ID NO: 163) | GGAGAGAAAAACAACTCAAAATGTATGTTTGACAATAGTGTATT |
| | TTAATGAATTTAGTGATATACAAAGTAAGATAATTGCTTGTTACT |
| | ACAAAGAGTTATTTTGATATAAAGAACCAACTACACAAAATTAT |
| | AGTTTTATGTGTATTTTAGTCAA[A/G]AAATCATCTTGACAAGTT |
| | TAATCTTATCAAGGGTTAAAATAGATATATTACAAATTGATATA |
| | AAGACCTATATTTCATATAGANGTAATATACAgggatgcctgggtagctca |
| | gcgattgagcatctgactttggctcagagcatgatcccaggtctggggattgagtcccacatcagggttcct |
| | gcaagaagtctgctt |
| BICFG630J275606 | ATTGTATTTGCATAGCCCCTCTGACGACTTGCATTAGCTCGATTC |
| (SEQ ID NO: 164) | CATACAAACCCGTGTGCCCCAGTTTCATAAAGCCTTCTCTCCTTG |
| | GCCAAATGAAATCAGCCTCTCCAAGTGACCCTCAACTTTAACAC |
| | TTCAAAGTAAAGCACAGAGTTACTTTGATTATTACCACAGTACT |
| | TGACCACAATCCAGAGAAAGTC[C/A]ATGAAAACCAGGACCAGA |
| | TAGAGTTAATGCTTTCATAGAAACAAAATGCCGCCTGTGGATGC |
| | TGAGTGCCAGCACATCATTAAGGGAAGGATAGGAATAAGGCCT |
| | TCTTAAGAGCTGACATTAAAAATTGAAATCCATTCTGTAAAAGA |
| | CAGGCCTTGTGTATTTTTTAAAGCCCAGAGCTATAGCAGCTGAA |
| | GGGTAAT |
| BICFG630J278829 | attagcaagaaaattgcacggggaggtggcatgggaaggaaagaccagagccacagcctccctgcaag |
| (SEQ ID NO: 165) | ggctcttgcctCCGCTCTCATCTCCCNGGGGCAGAGATCTCGGTCCCCT |
| | TCCCGGGCAGANTCTCCCTGGGGCAACCTTGCCAGAGAGAAAAT |
| | GCTTCCTCCGGGTGGTGCTCAGACATGCCNTCTAGAACG[T/G]CC |
| | CACCGGTGTGTCTCAGCAGGTACGCGGATGGGATGGCAGTGATG |
| | GGGAGCCTNGGAGATTCCCTGCAGACNATCCAGACAGGAGCTG |
| | AGGCTGTGCAAGGGACACTGAGCCTGCCCTCTGACCGCCACTGA |
| | CTCCTGGTTCCCATTGCATCCTCTGGGTGCTGCCTCCTGTGCACC |
| | CTGTCCTATTTCCCTGAAGTAA |
| BICFG630J282369 | GAGGCCTTGGAGATACTCGTCTCAGAGGTATTTGGAGAATAACC |
| (SEQ ID NO: 166) | TTGCAAGAACTCACTGGCTGGTGACAGTAATTCAGTTAGTTCTA |
| | CATCTTTCTAGTGGAATCTAGAGTAAAATATACAGAGCCAGGTG |
| | ATAAATTTTGAAAAAGGCCCTGATATGGCAGTGCCATGCATTTT |
| | AAGTGTACATCAAGCTTTCTGGAG[T/G]CAGCTTGAGATGGATGT |
| | TCAGTGTATTGGTGTCACATTTTTAGACATGTTCGCAAGGTCTCA |
| | TTTTTTTTCTGTCTCTTCATTCTTGCATTTCAGCAGCTAGAAATGG |
| | CTTTGCTGCTTCAAAACTCTGTGATATCTCTTTATGATAAATTTA |
| | GATTTTAAATGCCATTGTCACTTGCTGAATGCATTTGTAAGAAAT |
| | GT |
| BICFG630J304928 | TGGCTACCTTGGCGGAAAGCTCTGGTCCTNGCACAGGTGGCTTC |
| (SEQ ID NO: 167) | ACCCAGGCCCTGCCACTGCCTGCGGCNCCCTAGTGAGGCAGGGT |
| | CCCATCCCTGCAGGTGCGGCCCNCGATGCCAGTGGATGCTCCTT |
| | CTAGAACAGCTCACCCAACACGTGCCTTTGCCTTTTCCATGTTTT |
| | TTCAATGTATCTCTGCCTCTTCC[G/C]TGCAAGATTTCTCTTGTTC |
| | TAGAGATGNGATTGTACACCGAGCGGAAAGGGGTGGATCTGGC |
| | GGGACCNGAGGGCACCCCACCCCCGTGTGCCTCACCCTGCCTTC |
| | TGNACACCCCTCTGTGAGCAGGACCAGAGCTGCGGGCGCTGGG |
| | GTTCCCAAGTCTGTGCGTCCATCCCGAGACCTATTCTGCAAAAG |
| | GGGGATT |
| BICFG630J319569 | CCACGACCCGAGCCACAGCCCAGAGTCAGAGGCTAAGTGACTG |
| (SEQ ID NO: 168) | AGCCACAGGCATCAAGGCCCGGGTTCCTGCCGTGGGCACTCCTG |
| | CCCCCCACGGCCCCCGAGAGCTCGGGCTGCGGGCTGCGCTCCCA |
| | CCCCAGGCCTCCCGGCGGCTCCATGCACGTCCCCTCTGTCCCAA |
| | CTCAGGGTGCAAGGGCCTCGGCCGG[T/G]AAGGCGCCTCCCATC |
| | TGCCCGACGAAGCCCAGCCGGACGGAGCTGCTGGAGCAGGAAT |
| | TCCAACAACTGCTGCCCGCCTTGCTGCGCAGGGATGTCATCTCC |
| | GTTTTCATCTTTTTAGACAACTGTCATGGATTTGCCACCACCGAC |
| | GAGGTGCTGGATCTGCTGTTTACGAGAGTGAGCACCTGNGCCTC |
| | CGCAGCCCA |
| BICFG630J331636 | ATGTAAGAATAATCAGAAGGAAGTGAAATATATAAAAAACAAC |
| (SEQ ID NO: 169) | TTACTGGTGAAAAATCTCCAGTACAGGTTTTTTTATTCATTAAAT |
| | CCTCTCTTGGTAAGGGTAAACTTACCTGTGGTAAGGCAAGGAGA |
| | TAAGCAAGAGCCAATGTCATGTCATTTGGTAAAGCATCACTTGC |
| | CAGCTGCAAGAGAACTGAAGGCAG[G/A]GGGAAAAAAATGCAT |
| | TATTATTCTTGAGGATCTATATGACATTTGTAGTTACTGAGTGCC |
| | ATGTTTTTAATCTTGCATGATTACCACAGATAATTAGCTTCAGAG |
| | GACTCTTTAAGACCTTTTACAAATGCCTCTTAGTACCATCCAAAT |
| | ACACATCATAGGAAAAATTGTTATTAAATAGTAACCCTGTCTTA |
| | ATTCAG |
| BICFG630J346559 | GAGAGAAGTCTTCTAAGCGCATATCTGGCCTTTCATAGGGATGT |
| (SEQ ID NO: 170) | ATAAAAGGGAAAAAAACATTGATAGCATACAAAANGACATTTA |
| | ATGATTTGCTTCTCTGAGATCTTAACTGTATCNGGCTTCTTTTTTA |
| | CATTCTATATCCCTGACCTCTTCACCCAAATGCCTAGAGTTCTTC |
| | CAGCTTCTAAATAAACAGTATG[C/T]ACTTGTTGCCATATTAAAC |
| | CTTGATTCAGATCCTGCTTAATTTCCATAATCTTTTTAGACCCCT |
| | ACATCTCACTTAGATAATGACCCTTTAGCTTGAATTAAACTATGT |
| | ACCATTGATCATTTACTTGTTTTCTCTTGGAGTTTAATTTAATCAT |
| | TCTAGTTTAGTTCAAAAGGACAAATAGCTTTCCACACAATGTTTG |
| BICFG630J356853 | CATAACTCCACCCCAATCCTCACACAAATGATTCCTCCTAAAGT |
| (SEQ ID NO: 171) | CACCAATGACACCCTTGCCACAGAATCAGAGGATCCTTTTCAGC |
| | CTTCCTCTGTCATAAAGTCTCAAGAGCAAATCATACCTTCACTGT |
| | TNTCCTTACATAAAAGATTATCTTACCCCAGAACCTCCTGATTTT |
| | CTCCTTCTTCCATCCTGATCTC[T/C]ACTTCTCAATTTCCTTATGG |
| | GCTCACTCTCCTCACCACAGCTTTTAATTCCTGGGAGCTTCAGTC |
| | CTTTATGTCCTCCCTTTGCACACTCCCGATAAGATATTTAATATA |
| | CTACAGCACAATCCTAAGGGTCTAAGAATAATTTTTAACACATT |
| | CCTTTCCCTCATATCACTCTGTATCCACTTCCATTACGTAGCTCTA |
| BICFG630J358084 | ATTGTAAGCTTGCTAACCGAAGTGAGCCATCCTTCTCAGGAGAG |
| (SEQ ID NO: 172) | GGAAGACAGCAAGNAGGCCTTGCCTGAGGGAAATAAACTTAGT |
| | CCAGTAGGTACGTTCTGTGAGAATTTGGCAACCCTCTGATGTGG |
| | ACACACAGTGCCTATGGACGAAGTACTTAGTGACAGTTGCATAA |
| | GTGATTGGAAAAGGCACTCCACACC[A/T]GTCCACCCACAGAGG |
| | CCCCTTGGGCTTCCAGTTTCTACTCTGCTTTGGAGAAGAGATTCC |
| | ACTGTGGTGAGACATGTCCATTCAGGGGACCTATTNTCATGCAT |
| | CTCTCCACTTGTGGGGAGTTGAAATGGCCATGGTCTTTAGACCT |
| | GGAGATCATCCAGGGACAATTTCTCATCATCAACTGGACTCCTT |
| | CTACCATT |
| BICFG630J373954 | cttactttcaattatgaagaaaacacattcccttttcggtttaatccagtttcacccaatcacttttaatccaaaga |
| (SEQ ID NO: 173) | aatttggctaatacaTATCATTGCCACCACCACACATTATAAATGTGTAAG |
| | TATTTACAGCCACTTTTCAGGAAGAGATTTGTTTTCAAGGAAATT |
| | TCTCTTGCTAGCTTTCCAAATGTTTAA[C/T]ACCTACTTTGAAAA |
| | GTAAGAGACAGAAAGTGCAACCTGCTCTTGCAAATGTGCCCTCT |
| | GACATGCTAAGCCTAGTCAGTCCCAGTAGAATCAGCTAATCAGA |
| | ACTGTGCAAGACCTGGTCCTCAGTTGCCAGGAGGGAAGGGGTA |
| | GTTTGCCTTCACAACTCACGGAAATAGGGCAGTAGAAATTGACG |
| | AGGCCTTAGGT |
| BICFG630J391832 | TCAGGCCACCCCTGCAAGCACCTGTCTCCCCCTCTGATTTAAGCG |
| (SEQ ID NO: 174) | ACACGCTTTCAATCCCACCCCACGGAGGGCCTCCCGTACTTTCA |
| | AGCGAGAGCTGGCNGGATGCCTCTCTTCTTTCCTTGGATTTCCCA |
| | CCTTCCTCGCCTCCGCAGATCCCCGAGCATCTCAGGCTGCTGGCC |
| | ACCACTGAGTCACCAGGAGTA[T/C]AGCACTGCAGTACCTAGCT |
| | CTACCCGCCATCAACTACCGCCAGAGTCAGAGCACTGCACCCCG |
| | CACTTCCCACCCCACAGGAAAGCTCGGCTGTCCTACGGGGCTGG |
| | ACCAAAAGGGGGNAAAAAATGTTTTTGTACTTCTAATGGCTCCC |
| | CTCTGAACCAGTGCAGCTGAAATCCCCACAGTTCTAGAGAACGA |
| | GGTNC |
| BICFG630J402866 | TGCTGGAGCGTCAGGCAGGAGAGGCCCAGTGGTTAGAGACACA |
| (SEQ ID NO: 175) | AAGCAGGCTGTGCCCCAAGCCTCCTGCTCCCCTGTCTGCCTCCTG |
| | AGCCAGGCTTTTCNTGCCCCGGCCCCCAGGCCTTGTCGTGACTCC |
| | CCGTGTGCCTCCCNGCGTCACCACCAGGCAGGAGGGAGCAGAT |
| | ACTGTGTGGGNGGCCCTGCCGAGC[G/A]GCTCCCTGAGCTGTGG |
| | CCATGAAGCACAATGTGCTTGCTGCCCTCAGGAGGCTTCTGGCC |
| | TCTAAGAGAGCAGCCAGTGGGACTGCAGGAGCAAGGAGGAGCT |
| | TTGAACTGATCAGGGGTAGGGGTTATGATGGGGCAAGTGGGGT |
| | GGGGAGCAGTCTTCAGCCAGGTGTGCAGGGAGGGCCTCTCTCTG |
| | GGGAGGAGAC |
| BICFG630J399661 | TCCTCCCAACAGTTGTCCAGCAGCCCCTCGGAGCCCCGTGGCTG |
| (SEQ ID NO: 176) | CCTGTGGAGCTTCCCTGGCCCTTTCCTCTACGTGCCTCCTCGTCC |
| | CCGATTACTTGGAGTTTGGTGGGAGAGACAACTCTACCCAGGGC |
| | TTCTGCGGCTGCTCAACAGGCACTGGAGGGACAGCTGGGGACTC |
| | GGAGGGACCNAGAGTCACGTGCA[C/G]GGTGGCNAGTGAGATG |
| | GAATCTAAGAGCTCTTCGGGCTCACAGCCTTTCCGCTCTGCAGG |
| | AGGGAAGGCCTGTCTCTCGGGGCAGCAACCAGCCATCCCGCAGT |
| | CCCNGGCTCTCCCTCCCTTACAACGCCCAGGAGGCTCCAGTGGG |
| | TGCTTGGGCTCTGAAACGCTGTTCTTCCCCCCTCTTTACACCCCC |
| | CCTCCCC |
| BICFG630J414309 | TGTTCCATTACCTTTCTTTTTGATCCCTGTGGCTCAGCCAAGTTA |
| (SEQ ID NO: 177) | AAAGAGTGAGAAACATGGACCTTTTTTGTCTCTGTTCCGGCTGTC |
| | CAAGGACAAGGCCTGTTCCTGGGATGCGAAAGCTCTGAGGCAGT |
| | CAAGGCTGACTTCTCCTTCCTCCTTTATCTTGCCAGAGGCTGGCC |
| | CAACTGTGTCTCTCCCACCTT[A/G]TGAGCTCATGTACCCCTTGG |
| | GCTCTCGCTACTCCCAACCCAGTATTCAAATTTGTTTTTTTGGGG |
| | TTTGCTTATTTTGCTTTGTTTTAATATGGAAGGNGGTGCTCTCCC |
| | TACTATGCCAGAGTTGTCCTTGNNGATGGGGGCGAGACTACACT |
| | TGACCTCTTGACCTACTGTGANCACTTTGCAGAGTCTCTGGTCCTT |
| BICFG630J421119 | CAGACTAAGTAGATACATAAAGAAATAGAGTTCTATTCTTTTAC |
| (SEQ ID NO: 178) | TTACTATGACACATGGCCTGCTAGGGAAATACGAATTTAACTTA |
| | AACCCGAGTGACAAGAGGAATATAGCAAAATATGGGCAGTTGA |
| | ATAATGATATACTTTAGGAAATCTGTAAAACATTAAGAACTGTC |
| | TTATTTATGGAAATCCTATACAGTT[T/G]TCCAGAGTCTTGAATG |
| | TAGATCTGTTTTAAAAGGAACTCGTGGACCATTTCAACGTCGTTT |
| | CTAGTGGCCTGACTTGTCACAAGTCATTTCATTCCTGGGGAACCC |
| | CTGGTGGCAATTAGAAGTGAGGGAATTGGATAAGGAAAAATTA |
| | AGCCTAATTAAAATCCTTGTACAAGAAGTCAGGAAAGAATAAA |
| | CAAACTTG |
| BICFG630J431948 | TGTTGCTTGAAGATATCTGGAAGTGTAACAAGTTTAAATAAAAT |
| (SEQ ID NO: 179) | GAGTTCTGTTGGAATTAGGAAAAAGAATAAAATGTCTNTGTTGA |
| | GTAAAACATTTTTAAGAGGGGCTACTTTCCTCTTTCTGTGTGCAT |
| | TGTTATTAATTGCTCAGGAAAATGGTTCCATGTAAAAATCTCAT |
| | GTGTAACTATTNCTTATCTATAT[C/T]TCACAATCTAATACACTTT |
| | TCTAGAAAGTCTTAAGTTAATTTTTTGTTTCGATGCCTAAATTAA |
| | AATAAAAGATTAAAACCTTGGATGCAATTAGCAAACATTTTTGT |
| | AGTTGTGCAGAGTAAATTAAAGGACATTTGTGTGCTTATTTTCA |
| | ANTCTAATGGAGAGCAAATTACATTNTtttttgtttttttNaaattta |
| BICFG630J425382 | AGTGCCGGGAAGGAAATCAAAAAACAAATGTGGCTGGCTTGGG |
| (SEQ ID NO: 180) | ACACTTAGATTTCTGAAAGCTATAAATGTGAAGTCTCATGTTTTC |
| | AGCCTTAATAGCCAGAGGACTTTGCCTTGTGTTCAAACAATGTT |
| | CTAGCTAATAGAATCACAGTCGTGGAAAAAGACTTCAAGAAGTT |
| | CTGATAGTTCTTTTTCTGCCTCGT[T/C]CAAGAACAGATCGCAAT |
| | TCAGTCTAATGGGAAGGTCATGCTCATTTAGACAGACTAATTTTT |
| | AAAGGCCACTATGAAATTATTTTCTTTATGATCATTAAACAAAA |
| | ATATTCGAAAATCAAAGAAAAAGCTGAATGATTCTGGTCTCCTG |
| | CAGGACTGTGGGCTGTGGAACTGTGGTCAAAGATCACTACAGTG |
| | ACCTTT |
| BICFG630J457850 | ccatagggagcctgcttctccctctgcctgtgtctctgcccctctgtgtgtgtctctcatgaataaataaaaagt |
| (SEQ ID NO: 181) | aaatcttaaaaaaaaaAAAAAAAAAAAGCCCACAGTGGTAAATACATACA |
| | CAAGAATGATCTGGCCNCTCAGGCCACCAGGATAGAAGGTGCC |
| | CTGTGCTCTGGACAGTTTCACGAAGCCATTGT[G/T]TAGACTGCT |
| | CTATAGAACACATAGACCTGTGCAGCCCCCTTCTCTCCTCACAA |
| | AAAGCCAAAACAAAACAAACCCTCtcaaataaggtcaggaaacttttgcctaagca |
| | aaatttaaaaagattatttcaaagcacaaaactcaagaggctttaatatgccaatctgcactgtgaatttctaag |
| | aagCAGTAGACTGTTG |
| BICFG630J473226 | AATCCCCCAGAGCAGCAGTTCCAATCGATGAGGAGCTGGCAAC |
| (SEQ ID NO: 182) | ATCCGGGCTGGGCATGGAAAGGTGAACAAACATTGTCCATTACC |
| | CTGCCAATCGCCAGTCCCCTAATTCTGTTATTTTTTTTCCTTGGGT |
| | AAATTTGGTTTCCTGAAATTAATTATTCAACAGGAGGTGACAGC |
| | CGGTGTGTAGCAGCACTGTTGGA[A/G]CAGAGAATAAAGAGGCA |
| | CATTGGACACAGCAGCTGCACCTCCCAGACCCTGAAATTTAAGA |
| | TCTTTATAAATGATCTGTTAAAACTATAGTGACGATAAGCTTATG |
| | AATCATGATCTATATTAATCAGGGCTGCTGATATGGAAAGATTA |
| | ATTGAAACGTGCAGTTCTACACAAATGATAAAGTGGTAACAATT |
| | TAATAT |
| BICFG630J484553 | AGGAAGCCGTGAGATTAGAACATAAGCTTCTGCATCCAGGGGA |
| (SEQ ID NO: 183) | AATTTCCACAGAGGGAAATTGTGGCCCTGGTGCTCACTTATACC |
| | TGATTCTTGCCTCTCTTTCACACGGGAATCATGGGTTGGGTTTGA |
| | AAAAACTGCAGAACTGTATAAAACCTCTCCTTCCTTTCTTTTTCA |
| | AGCTAGGAGAACACAGTGTTCAC[A/C]ATATAGCCTCCCACTCA |
| | CTTCACAGAATTGACAAGGGAAAACAGTGTGCTTGTGGGCCTGA |
| | GCACGACTTAAGCAGGGTGAAGTCTGGGACAAGACTGCACCAG |
| | GATCCTTCCTCCCCTCCCTTTAGGTTCTTTGCCTATAGGATTCTA |
| | AAGGCTCAAGGCCATGGGGGCAGTGACACTTGCTTAGGGAGAC |
| | CCAGCCAC |
| BICFG630J497958 | GGTTGATAAAATCAGGGCTTCATTGTCTTTTGCCAGCCTCAGTTT |
| (SEQ ID NO: 184) | GGCCACTTGAGAAATGACAACATTGGACCAAATAATGAATTTCT |
| | GATGCTTCTAGAGTCTGTGATTTCCACATGCTGTGACTGTAAGA |
| | GCAGAGTCATCAAGGCTTGGTTTTCTGACAAACAATTCCAGGGA |
| | AATAGAGCTGGTGGGGGAGGGGA[T/C]CCCAGCGCTCACCCCAC |
| | CGCAGCCCCCACAGAGGGCTTCCCGAGCTGCCACCCAGCTGGTT |
| | GACCCCCAAAGGAGCAATTTGCACTTTCTGCTTTCCTGGCCTAA |
| | GATAAAAATACCCCTGTCACATTGGATTAGCATCTCCCCTTTCNC |
| | TGAGAATCTTCTCACGGATGCAGCCCCCTTGCTTTGTCAATATTT |
| | TCAGA |
| BICFG630J503647 | AAACTAAATGCTCACAANGGCAAAAAGCAATGTGAGNNGACCT |
| (SEQ ID NO: 185) | TGCAGGGGCAGGGCGAGTCATGGAGCGATTGAAAAAGAAAGAA |
| | AAAAAAGCAACAATTTTTAATAGAATTCNGAAAGTCTGCTGCCT |
| | CGTCTGGTTTACAAATAGGCATTGTTNGAGGAGACAGAATAAAT |
| | AAGAGCTAACTACAGCATGNATTACC[A/C]AACACTAANCCCAT |
| | CAACGAGTCCCGGTGGCAGCACAGATCACTCAGGCACGCCTTGG |
| | TCACTCTCCNCATATTTATTTATTAAGAAGACAGTGGAGTCTGGC |
| | TAATGCGATACAAAATTAATATCANCTGTAAAGAAACATAACCC |
| | ATACATTCAAAGCGATAACTCTACCGACACCCTCCCCCCCAACT |
| | CAATCAAGT |
| BICFG630J525153 | CTAGAGCCAGGAATGTTCCGATGTCACCGGCAACTCACGGTACC |
| (SEQ ID NO: 186) | ACCACGTCCAAGGCTGCTTCCTATTCCACGTGCGGCAGAGGCTG |
| | CCCGCCTGCCTCCCCCCTCCCAGGGGCTGCCTCCCCACCTCAGGT |
| | GGCACCGTCCTCAGAACTGGGGCACAGAGGATGCAAGCCAAGC |
| | TCATCAAATCCTCTCCCCGAGACC[A/G]CCGTCTGCTGTAGAACA |
| | CNGCCGCCCAGAGACATGTACNAGAACCCCTAACCGGCTCGTGT |
| | CGGCCCGTGTGTCTATGGAGGCGTCATGGATGAGCTCTTACACA |
| | CTCGCCCGTGACTCCACCATCACAAAGTAGAAACAAACCAAGA |
| | ACGCTGTAACGATGGAAAATCTACTGACCCTGACCCCCTACCCC |
| | TCCCCGCT |
| BICFG630J533364 | GCNCAGGGACTGCCACCGAAGAGCCCTGAGCATCTTCTGACCAG |
| (SEQ ID NO: 187) | TCCAGGCTGATGTGGCTTCACTCCTGTTGCTGCACATCTCACCAT |
| | TCTCTCTTGCTGTTGGCCAGTCTTCTGCCCTCACTTTGTGCTTTCC |
| | CATGTCAACATGGAATTGGAGCTGCTCAGACTTGAGCTCAGGGT |
| | ATCGATGGTCCTAGAAATGGA[A/G]TGTCAGGAACANGTGAGTG |
| | GGTTTATGTGTTTTCCCCAGGAAATCAAGAACTGATGGTAAACA |
| | GAAGCANGAAACACCATTTGAGTTACTGGCGTGTTAGTGGAAAA |
| | CCAGTACATCACCCCTGCCAGTGCAGGTAGGTAGCTGACCCACA |
| | TAGTTCCGTATCACCTTCTTGTTGAAATTAGTATCTTGCATCTAT |
| | TCTT |
| BICFG630J537466 | ACAGAACATGGCTCCTCACATGGGANCAGCCTCACTNACCCACA |
| (SEQ ID NO: 188) | ACATTTCAGATAAGGAGGAAGTGAGACAAAAACCCCTGGGTCC |
| | TTTTATCTGGTCCTCTTCATTGAGAAGCTTCTGTGGAGCTTACAG |
| | TCAGGATCAGATCTGGTGTTGACCAGCAGGCTCTTACCATGGAG |
| | AAGGTCCACAGGGAGTTACCAGTG[G/A]TGGCAGGGTCACTTGT |
| | TGCCTATCCTGGGACCTGCAGCTGTCAAGTTCCGGAATNATTCTT |
| | TTTCNTTCCTGGTTCCTGCCCTGAGACCCTCATGAGAGGCTCTGA |
| | GTTGTGTGTTTCACACAGAATAAGAGGTGGCTTTGACGTCCAGT |
| | CCCCTAGATCTGTCAGCCATGAGGTTTGCACATGCACATTTACA |
| | CGTGTC |
| BICFG630J548189 | GTACGCACACACGTATATACGTACACACGCATACGTGTATATAA |
| (SEQ ID NO: 189) | CACGCAACAGACNTATGAAGACCCGCACAGAGATTAGAACGCG |
| | ATTAGAGATCAGAGAGCGAACCTCAAGGGGCCTGGCCCGGAAA |
| | CTAATANATGTGGAAAGTCACTGAGGGGCTGACGGAGGCTGGA |
| | CNTTCAGACGAAAGCCAGACTAAAGGG[A/G]CGAGAACTGGTCT |
| | GTAAGCAGCCTCTATGTAACGGTGCCCGGACCAGCCCTGCCGAT |
| | GACTGGACACCCCAACTTCCGCCGAAGGCCAGACAGTCACGCCG |
| | ACAGGAGTGGAGAGGGTTTTAAATCCCCCAGAGAAAAAAGAGC |
| | TGAAAGCCCCAGGTAGGGCAAGNGGGAGAGAACGAGGCCACGG |
| | GGGCAGCCACACA |
| BICFG630J553154 | TCCAGAGGGCAGCGGCAACACANGGTGAGTCTCCAGGGGTGTG |
| (SEQ ID NO: 190) | GCACGGGAGACCCCACAGACCAAGACCACGGCGGGCTCCGCCT |
| | TANNGCATCAATCCNCNGAGAGCGGTCCCGNGCCACGTTGCCTC |
| | TCTGGAGCATGATGCAAAGGCANACGNCTGCTGCCNGAGACTCT |
| | CAGGGCTGACAAGTCTCCAGCCAAGG[G/C]CTCACATGTCCTTG |
| | GCNTGTCAACCGTGATCGCGAGCAGCAGCNTGGCCCGATGCCCG |
| | TCNCTTCNTTGTGGNAGCCAGACTGGCTTCGCAACTCNACTCAC |
| | CTGCTACGCGCCCAGGACTGNTGAAGCCGGGGCCCCCTGCGGTC |
| | CTGCCCACNCTGCNAGCTTCCAGGGTCGTGGCAANCGGACTCCC |
| | GCCAACACCT |
| BICFG630J566667 | AGCCTTCAGAGGGGGCTCCGGCCATCAGGTGGGCCGCAAATCTC |
| (SEQ ID NO: 191) | CGCACAATGGGATGGTAATGTCTCTGAAGGACAAACAGGACAA |
| | CCACATTTTGCACGTCAGTAATTTATAAAAGACCAACCACAGCA |
| | AAGATATTCTTTAATAAACACTATTTCTTAAAATCACAGATACGT |
| | ATGTAATTCACAGTAATCACATAC[T/C]GAAAGAAAAAATACTC |
| | TTTGCTCACATGATTACATTAATCTGATGACCAAGTTACCAGTGA |
| | CATAGCTTAGTCTAGCTTTTACAGTATGAAAAGAGTAATCTAAA |
| | AGCAATTTCTCTTTTAGAGTGGAAAAAGTTAGCTTACAACAGGT |
| | TTCCTGAGACATATCTGCTATAAGTCTCCCTCTATGTCCACACTC |
| | AAGGAT |
| BICFG630J573029 | TTTGGTAAATCTCANATTTTGACATTTTATANTGCCTAAATTCCA |
| (SEQ ID NO: 192) | AGCTGTCTGTGtttttNttttttNtttttttttCTGGTCCTAAAATACGTAATTCT |
| | CCAACTCAGTTTTTCAACCTCCAGAAATATTTTAGCTCTCCTCCT |
| | CCTCTTCATCCTTCTCTTCCACATCTCTAATCCTCATGTCTGTGTT |
| | GCTCTTGA[T/C]CTTGACTTTGGAGCCAACCAATTCACTCTTTAG |
| | CCAACACTGGGCTGGNCCAGAGAAGGGAGCGTGGATAAAAAAC |
| | TAATGAATATTGTCAGTTATCCACATTCTGAAATTAATGGAATTA |
| | TTGCCAACAACTTGGAAGACCTGCCAGAGAGGGAAAGTGACAG |
| | ACTCCTGGCAAAGACAATGATAGGATGAGCAGTCTT |
| BICFG630J585149 | GTGACCAAGCACTGTGGTGTGCCCCTCACTCCTTTATCGTCCGTT |
| (SEQ ID NO: 193) | GACTCTAAGCATCAGGACATCATGGAAAAGACGTGTGAGATTCC |
| | TCTGCCCTTTCCCCTCTGCTATTCTCTGATAATTTTTCCTCCTCCC |
| | AAACTGCCTGGGAGCCCTGCTTCGCTCTGCTACACATCCTGGCC |
| | ACAAAGGAAAAGCAAGACTTG[G/C]AGAGTGTGGTTATCTGGGG |
| | TCCTTTCCCCCTGGCTTTTTCTCCCTGGCCTGTCAACGATGCAGG |
| | CCTGGGGATTTTCAGCCTGGGCTATGCCATGGACTCNGAGCTAA |
| | AATGCTTTCCATGGCTGAGGCTCAGAAAGCAGGTAAGAAGTCCT |
| | GGTTTAGAGGCAAAATCTTCTTTTCTCATCCACAGAAAGCCCCCT |
| | TGT |
| BICFG630J597522 | tttAACTAAAGCTCCAGTCCCCCATCCCCNTCCCCCATCCCTGCCC |
| (SEQ ID NO: 194) | CGGAGCCTCCAGCATCTAACCGGCATTTACGAAGAAGAGGTGG |
| | ACGGTCGCTCCTCCCCTCGGATAGTGTGGGTTTAGGGCTTCGGG |
| | GTCCAGTACANCACGGCCTGCACGCAGTCTGGCTCTCTCAGGGC |
| | CTTGCGACCTGATCTGGGCCTGT[T/C]GTCATCACTGCACATCCC |
| | CCGGGCGCCCACCTGGCAGGTGGCAGGCTCCCCCNGCAGGTGGC |
| | AGGCTCACCTGGCAGGTGACAGGTGACGTGCTGCCCACCTGGGC |
| | GCGGCAGGTGGAGGAGCACCCAGCACCATCCCCGTAAGTGGGC |
| | GCAGTCGGCCCTGGGGTTTCGCGGGGCCAGTGACTCAGCCAGTG |
| | GCCACTC |
| BICFG630J608671 | GAAATGGTAGTAAAAGGGTGCACGCCTTATAATTTAGGCCAGGC |
| (SEQ ID NO: 195) | CTGCATGACCTCAAAGCACCCAAGCAACTCATTGAACAGAAGA |
| | ATCAATCAAGTTTGATACCGGTTGACAAACGAATAATAGGACAA |
| | GATCATTTGCTTGCTTTGCCAGTGCCCCCCAGGTGTGCTCATGGG |
| | GCATGAGCTTTAGTGCAAGTGCCA[G/A]CGAAAGAACCTGTATT |
| | TCCTGTTAGCCTGGTGGTCTCTTCAGAGGGCAAACTTCAATAAT |
| | ACTGATGGTGTGGATTACTCGAATTTGCCATTTGCTACTTGACAC |
| | AGTACCCTTAAAATAGCCCGTCAGCCAAGCAGCCGTGATTGTGT |
| | TTCTCATGCCACCGTCCTTTGCAAAGTAGGTTTGTGGATGGTATT |
| | TCGGGG |
| BICFG630J613547 | TTTTCTTTCCTCAAATTCCTACAAAGGCCAGACTTATTTTACCAG |
| (SEQ ID NO: 196) | GATGCCTTCCAATTGAACTGGCTATAGGCCCAGCCTTTCAAGAA |
| | ACCAACAGCAAATGCTGGCCTCTGAAGAGGTCAGTATTGAAAG |
| | GTAAATCTTATATTCACCTAGGACTTCTAGGGTGTTGTCCTCTCA |
| | GAGGCCAGAATTACCTCTCAGGG[C/T]CATGAGAATGGCCTTTTG |
| | GGGACCAGGATTCTGATGGCAAGAGCCTGGGCTCCAATGAGCTT |
| | CAAACTGATCTTCTTTTCTATCCATTAACCACTGGTATTTCTGAA |
| | AGTCAGCCCTGAATTTCTAATCTCTACTTGGGAGTTAATTACCAC |
| | CTATAAAGACAGTGGCTGCAAAAAAAAAAAAATCTCTATTTTCC |
| | ACCC |
| BICFG630J630348 | GGAGGGGATAAGGCAACCCCCTTGGGCCCTACTTTGGAGAAGA |
| (SEQ ID NO: 197) | CTTTGTAGAGATGAACAACTGTCCGCAGGCCTAGGATGCAAGTG |
| | TCAAGGCCGGACCTTCCCTGGGTTCCTCAGCCAGGTCCACTGTG |
| | GAGTCTCCCACGCATGGCCTTAAATGGCCACGCCCGGGCCTGAC |
| | AGGGCAGTCACGATCCCGGAACTAC[A/G]GTCACTCGGCTTTAA |
| | GAAGCCCATTGTAAGCCTGACCACCAGGGAAGAGTTGGCCAAA |
| | CTCCATCCCAAGACTGGACGGTAGCCCGGGAGATTAAATCCTAA |
| | ATAAATACTCCAACTAAATGCCTTGACTAAGAAGCCATGCTGGT |
| | CTCTAGTTGGAAATAAGGCAAGAAAGAGCNGTGATAACATCAA |
| | CCACACAAGGG |
| BICFG630J635046 | TGGTGACCCNGTGGNGGGGAGGCGGGCGGGGAGGCAGGTGGGG |
| (SEQ ID NO: 198) | GTCCATGTGGGCACTTCCCGGCTTGGGCCTGTCCTTCAGCGGGA |
| | GCAGAGACCAGAGCCGNGCCGGGGGCCACGCGGAGCCTCACCG |
| | GGGTGGGCGTCCGGGATCCCGCCTAGGAGGGGGTTGGCGGGCA |
| | GGGCCCATCCCNGCCCTGCCCCGTGCC[G/A]CTCGCCCGGGTTCT |
| | GCAGGGCCTGGCGCTCATTTCTGCGCCTCTGCGGCAGCGGAGCT |
| | CCCCGAGCCCCAACCGTGGTGTCTCCGGAGCCCCCNCGGGAGAC |
| | ACGACCACGTTCTCCCGGGAGCACCTACAGNGGCCCCCNNAGG |
| | AGCAGCCCTTCCAGCTTGGTGTCTGGGCTCCGTGCCCTTGCACCA |
| | GAAGTTTCCA |
| BICFG630J638804 | TCTTGAAACATGGACAAGGCAAAATCAAGAACAAAATCATCCTT |
| (SEQ ID NO: 199) | TTGACAGACAACACAGTAATTGAGAAGCACCTAGAGAAGTATG |
| | GTGTTATGTGCTGGAAGACCTCATTCATGAAATTGCCTTTCTGGG |
| | GAAGAATTTCCAGGCAATTTCTGAGTTCTTAATGCCTTTCCATCT |
| | CTCATGCTACCAAGAAGAGAGTG[G/A]GCCTTCTCAAAGAGGTG |
| | GGCTTACTTGGCTATGGAGATGAATGCACCAATCAACTCATTTG |
| | GCTGCTGAACTAAACTGAGAACTCTGAAAGCACAGTGCAGTGG |
| | AGGCATGTGTTTTGTTTTTTGGAATTGTTATCCAGTATCTTCAGA |
| | AAAGATTATTTTCTGCTATATCTTCAACAACTAGATAGAAGGGT |
| | CAGGAAA |
| BICFG630J636447 | AGGAAACTGACACCACTGATACCCCCTGGGGTTGGCATGCACCT |
| (SEQ ID NO: 200) | CAAAACTTatagataaatacataaataagtaaataaataaataaCaattaaaataataaaataaaaaa |
| | GAAATTTCTAGCAAAGATAGTTTAGAAGTAATATGTGTCTTTCA |
| | TATTACTAATTCTCCCCACAAAGGAGAGCTATTTCTTTTAACTG |
| | [G/A]TCAATACAACCTCAGTTATTTCACATCTTNACCTCACATAAA |
| | TTTTTTTTAATGTCATATTGTATTATTTTGAATCCTGTTTNCATGT |
| | AAGTTTTGTGTGTNTTCCCAAGTAAACTGTAAATTTTCTTCTCTA |
| | TATTGTGGGTGCCAACCCTGAAACCTGACNGGAGTGGTCCTTCT |
| | TTGTGNGCCTAGGCCTTCTAGCT |
| BICFG630J654194 | gatcgtaggatagttttatttttagttttttgaggaacctccctgctcttctccagagtggctgctctagtttgcatt |
| (SEQ ID NO: 201) | cccatcaacggttcaagagggtatgcctttatccgAGGATGTTAATTTCTTGCTCTCA |
| | ATTTTGTTATCATTTGGAAACCATTTCTTTGAGTAACTTAATGGT |
| | TAGGGGTGCAGAGGGAAGA[A/G]CAATTACTTTGCTGACTGGAT |
| | GAAGTGTTTTGAGGCAGCGTGTACATACCTATGATGGGCAGCGT |
| | GACAACGGACACCTCAGGGATGCTGATTTTTCCAGTTGGTGAGT |
| | GAGTNTGTCTTCATGTTTGAATGAACTGGGCTGGCATGCAGAGT |
| | ATATAGCANTATGACTAGTCTCAGTTTATTGGTAATGTGAATGT |
| | AAG |
| BICFG630J660369 | TGCTGGCCTCCCTTGCTGGTGTCCCTTGTCTCCTAGCTCACCCTC |
| (SEQ ID NO: 202) | ACCTCCTATGTGGTTTGTCATACTGGCCCCCTCTCCAGCCCCCGC |
| | CCTGCCCTTGTGGCCTAATTCCTTGGCAGAGCTCCTGGCTGACCA |
| | GACACTTGCATGGCTGTTGATTCCTTTGGCTGGATTCCCTTAATC |
| | CCTGCAAGCTCTCTTCCTCG[C/T]TTCAGACCCTCAGCAGAACCC |
| | GGACTCAACTTCTGGAAACAACCAAATGGTAGCTCTCAGCTCCA |
| | TGGCGGTACATTCTTGACAAAGGCCACATTTTTCTATGATTGGG |
| | AGCTCCAGCCTTAGGGCTGTGCAGTCCCCTAAAGTCATGATTTCT |
| | GGGCCCTGCCAGTCAGCCATCTGCAGGGAGGTAATTtatcagttac |
| BICFG630J667882 | GTGTCTGCGTGTGTGTCCGTGTCTCTGTTTCTCGGTCAGTGTGCG |
| (SEQ ID NO: 203) | TGGACACACCACATGATTTCAGTTCAGTCGGGGTCAGGCTGGAG |
| | AGTCAGGGCCTCCTGTCTACGTTCAGTTCTNCGACTGANGGCAG |
| | GTTTCCTAGGGCCCAGGTCAGGGCGACCCACTGGGGGCACCGCC |
| | GTNCACAGCTCACCCAGGGGAAA[G/C]GAGGGAAGGGCCCGGA |
| | ACGCACGACAGGCAGGGGTTCNGGATGAGGACGGCCTCACCCC |
| | TTCTAGGCCAAGACCCCGACACCTTCTGTAGGGTTTGCCTTGGAT |
| | TCAGGAGCCTAGGATCGTGGGGAGCTATTGCCCATCCCTGCCCN |
| | GGGGGGACTGTCATCTTCTGGGCCTCCAGAATGAGGGTACCGGT |
| | GCCCNtgt |
| BICFG630J676160 | ATTTAAATAAGTTGCTCCCAATATTATAATAGACACAAAGTGAA |
| (SEQ ID NO: 204) | GCTTATTGGTAATTACTCATTATGAGACAAAATGATTAAGAACC |
| | CCANGGAAAAGAAGAGAACAGATTTGAATCATTTTTTATGTATA |
| | GGTAGGACATACTCCCAGGTTTTATGAGCAATGTGGCTTGAGGT |
| | TACTAATACCTTAAGAAAATTAAG[T/C]GCTAAGTGTGTCTTAAC |
| | TGCATCAATAATTTAAAGTGTCCTAAACACTATGGTAAAAATGC |
| | TCCCAAGATTCCTACTCAAACATTTTGAGACCTTAAGCTAATAG |
| | GTATTACTGTCCTCCCCCACTCCCTGCATAGAACACGGGCTACC |
| | AGGCAGATGTCGAATCTGAAGAAAAGAGGATATTAGGGCCCAA |
| | TAATCAGA |
| BICFG630J689381 | GCTAATTAATCCATCCCACTGGGAGGCACACTTATCATTAAAAG |
| (SEQ ID NO: 205) | GAGGCAGCTGATTTCAAAGCTTCATACCCTCCCTTCCCAGCTCA |
| | GCTTTCTATTTGGTTTCCAGAGTAATACGGGTTGCCTGAGAGCTG |
| | GGAAAAAAGAAACCTCATGTTCNTTTTCTGTAGTGGGTTTTGAT |
| | CTTGTAGCTGCTTATTTCTTAAG[A/C]ATTAGGAAAAGGGTACTT |
| | TACAGCTGGAATGGGAGATTGTAGACTGGAATGTGTAAAAAGG |
| | TGATATGAATCTTCAGGCTGCATTAGCTCTAGGAAGACCTCTCA |
| | GTTTAAAGAATGATGTTCATCTTCAAGAGAGAGATTAGAAAGCC |
| | NGTAGCTGTATTTGCTTGAGGATGCAAGTGAGATTCAGTGATCT |
| | GGAAATG |
| BICFG630J678332 | GCAGCTGTCTCAAGCACAGCATGTGCATCCTTGGGCTGCAGGTG |
| (SEQ ID NO: 206) | ATTGTCACTGAAAGAAAAATCTTTAGCTTCCCTAAGTTAATAAA |
| | CTTTTAACTAGGTTAAAAGGTAAACGAAGTGTGATGAGCACAGC |
| | CAGGAGTCTGATCCCGTCAGAGACTTGTCAGGGCCTCCTGCCTG |
| | GACAAGTGACAAGTCTGTGATTGG[A/T]TGGGACAGTCTGCCTG |
| | CCCAGAAACCTAGCTGTAGGAAGTTAGTGCAAAATGGAAACCA |
| | GTANAGGTATTGTATAATGACTCCATTGTGATTTAGAAATTCCN |
| | GATCATTTCATATGACATTTCTTTTTAATCACTTAATGTGAATAT |
| | ATAAAGAGTTTGACATTTGTTTAGATATTTTTCTCCTTTTGATGC |
| | TATCTCT |
| BICFG630J693521 | ATTTACTCGAGCTTTAGTTCATTTCTTTTGTGTGTCATACAATTAT |
| (SEQ ID NO: 207) | TCTAGTTTGTCAGTTGGTAATTGACATTGAACAAGCTAGAGAGA |
| | TTTTGGTTTTGGGGAGAAGTAAGGGAAACTGAGTATTTGTAAAG |
| | AAATGCTTCAGTAGCTCGGGGCTCCCCAGCCTGTCCTGTCAAGA |
| | ACTCTGTTATCTTTGCATCATC[A/C]TATCAGATAATACAGTCAT |
| | CATTTTAATGCCAAATGTCACTTTTGTCTCTTTAAAGAAACTAAC |
| | ATGTTGTTATCACTACTGATGTCAGATCAGCTGGTATTTATCTTC |
| | AACTTGATAAAAATGTGCAGTGGTTCCCTGTTCTCACATTAAGA |
| | CCCAGAAAGATTTAATGAAAAGTATTGTGTGGCAGCCTTACTTG |
| | GTC |
| BICFG630J695147 | gtaagctctctctccctcatgaaaataaataaattaattaattaaattaaataaataaaaTAAAAATCTT |
| (SEQ ID NO: 208) | AAGAAAAAGATGTAAGGAATAAGTAGGAGAAAGATAAGCAAA |
| | GAAGATAGTTCTAAGGTCCATAGTCTGGATTAGGACCCCATCTC |
| | TATTTCTGTCTAGGACAGGATGCTAGCTACTGGGAGGCAATGGC |
| | [T/C]TATTCTTTAGGTTACATAAGTGTATTTGTAAGCCCTTGAGGG |
| | AGCACCAAAGACTAAGAACATTTTCTTGAGTACTGAAGTATAGC |
| | CTAAGAGTCTTGGGTAAGCTTGTGCTACTCATGGAGAAATTAAA |
| | AGGATACCACTTTCCACTCTCAACCTCCATGAAACCCCAAGAAA |
| | CTAACATGAATATCCAAATCTGCTTC |
| BICFG630J707814 | AATAGCCCAGCAACTCCTGAGTGGATTAGGATGACTTGTGTGGA |
| (SEQ ID NO: 209) | TCAGTTTACTGACATGGTAAGAGTAGTGGAGGGATGTCCCAGCT |
| | ATAACCACCGCTTAATGGATTTTCATGTTCTTAGTATGGATTTGG |
| | TAATCATGACAAAATCACAGGGCAGGTTCATCTTTTCCAATGAA |
| | AATTCTCACTCTGCCTNTATACT[G/T]CAAGCAACTGCAAACCTC |
| | CTCAAATGATGGCTTGTTTCAGACTGCAAAGAAAAGCAGACTCA |
| | GTTGAGCCANTGCTAGGAGCGAATCAACATGGTAGCTAACTTCT |
| | TGAAACATTCTGTCAAAATGTAGTTGATGTGGTATTTTAATCACC |
| | TTAATAACCAAATTAGATTAAATAGATATCTGATCTGGTCAATA |
| | ATTCA |
| BICFG630J715531 | GAAGGTGACTGCCCCATGGGAGATGCTTGAGCTTCTCTCCCACG |
| (SEQ ID NO: 210) | TTACTAGGTCCCCCGGTGACAACAGCTATGGGTGCGGGGCATCT |
| | CCCTTCACGGTGCTGCCACATGGGTGCCTCACTGGTCCCGTTGCC |
| | TTGGGACAGAATCACTTTCTCTTGTTGTGGCAGCGGGGTTTGAG |
| | GCAAAACCGAAGTAACAAGATGA[T/C]AAGGAGATGACCCGGG |
| | CTCTTGTACCCTGACGGGGAGGGGCATGGCGGGTGGGTCTGCTC |
| | TTGTCACCATGCAGGAGGAGCGAAGCACCACGCAGTTTCAAGA |
| | GGCAGAACTCGCCTGTGCAAGAAGATGAGCTCTGTACAGTAGCC |
| | CCGTCTCGGGATTAGACCAGATGACCCCGAAAGCCCCATCGTTA |
| | GGGTTGAAG |
| BICFG630J719405 | ACAATAGTAACTACCTGCCCTGGTTGAGATAGAACATCTCTCAG |
| (SEQ ID NO: 211) | GGTGATGATTTTTTTTTTTTCAACACAATACAATAATTGAGGTAG |
| | GAGCCTGAGTTTCAGATGGGTTCCTGTGTTGTGGGACCTGTGAT |
| | AAAGCATATACCTTCCAATGATACTAGCATTTTCTAAAACGTGA |
| | CTGGCCTCTTGAACCCTGTCTGA[A/G]GACAGGGACAGACCAAG |
| | GACACGACACTGTCAATACGGACTCCTAATCCTGCCTGTTGCTC |
| | CTACATACAGTGGCTTTATCTTCTCTTACACACAAGAGCCACCCC |
| | TTTANTCTTCTGTTATCTTGAAAAGATACCTGAAAAGAGCCCTGC |
| | CTCAGGATCTTGGACTTAAATCTCAGTTGTTCTTCCAACCATTTA |
| | TGGG |
| BICFG630J724770 | AGTTCCCTCTTAGTCCCCTGAATGGACCATTCCTTGTTGTTGAAT |
| (SEQ ID NO: 212) | TAACTACTATGTGCCCTNGACTTTTCTAGCAGTCACAAAGGCAG |
| | ACTGGGACATTTATTGGATCTTAGGTCTCTTATTAGCCAAGGTCC |
| | TTTTCAGCCTAAGCTGTTGAACAAATCAGGTTACTACCTCAGCC |
| | AGACAGAAATGGTGAGAGCTTA[A/G]TGCAGGCAGAAATGTTAG |
| | TAGAAGTGCATACATTTCTGCCCTTAGCAGGATAGCACCAGCCT |
| | TCTCTCTGGGATGTGAAGTNTAGGAGTAGACAGAGGAGGTGAG |
| | AGCTGCTTCCTCCACCCCTGCAGGGAGGGTTGGAGAGCAATGAC |
| | TCTCTGGTACTTACTCTCCTTGCAACGGCCTTGGGCTTCCTGGCT |
| | TCCTTC |
| BICFG630J729876 | TCCCATGCTAACACTCACTGTGGTCATGTCAAATGCACGTACCC |
| (SEQ ID NO: 213) | CTTCCAGATTAAGTGTTGTAAGTCCCAACGCGTGGTTCCGCCTCA |
| | AACTGTTGTTGAGGGCTTATGTGAGAAAATGAAGATATTAAAAT |
| | CCATGTTGACCTTAGCATGAGCAACGAAATGTCTAGAAGCCCAA |
| | GACCAAAAGTAGATTCCCTTTCA[T/C]GTGGGCCCTTCACCATTG |
| | AATTGATGAACCTGTTGAGCAACGCGCTCAGTAACAATGACCCT |
| | CCACTCAATCTTCCAGATTACAGGATGCAACTTCATCTTCCAGAT |
| | GCCAGGATTACAACTTCAACACCGCTAGATCTGAGGTGTCACTC |
| | TCCACAAATGAGCTTATCCTGACTATAAAGAGTAACCATATCAC |
| | CACAG |
| BICFG630J749105 | TGTTGCATTAATGGCTTATTTTGCATATCTGTTTGATTGTACCAT |
| (SEQ 1D NO: 214) | CTTAAAGATTCATTTAAACCTGGGGAGGACAGCATGACTCCTAC |
| | CTGCTTCTCATTAATAACATGCTTTTCAGTGAGTGGATAATGAAT |
| | GACCTGGATAGAAATTAGCAGAAAATGCAGATTGCCATTAGGTG |
| | TAGAAGTGGGGAGGGAGTCCAC[A/G]TTTTCCACCATACCGACA |
| | AACAATTTCAAGTCAGAAGATATTGAAAACAAGCCTCAAAGAG |
| | CCATAACTGTCTAGGAGGATTTTTAATTAAATATCCTGTTGTCTT |
| | TACATGTAGAACTGTGAAGGAAAATGCATCCTAATAAAAATCAA |
| | AATTTGCAAGTGACTTAAAAAATCTTGGGTTAATAGAAACAAGC |
| | TATCTA |
| BICFG630J745699 | ACTCAAAGGAATGTACTGAGGTTTCTGAGGCATGAGAGCAAAA |
| (SEQ ID NO: 215) | GGGTCTAGGTGACAGACAACACTCAAAGTCTGATATGGGTTGTC |
| | ATCCTGGTTCTCAGTTATTAGTCTTATAATAAGAACTCTGACCAT |
| | ATCTGGAAATTCCATAACCCAGAACTCAACTTCCTGAGAAGGAA |
| | CTTGTTAGATCTAGGCAGACAGAC[A/T]AGATAGTCTTCATTTGC |
| | ACCAAGAAACTGAGGCAGAAGTTCAATCATCTAGACTGAATCAC |
| | CATGGGTTAAGGGACAGAAAGGCCACAGGGACATAAGTCCAGG |
| | GGTCACTCCAGGCTCACTGGACACCTTGCATGGGGAAGAATAAC |
| | TAAGACCAAACCTATTAATTGGAAGAGATAAAGCTCCTAATACA |
| | CTCCCAGC |
| Agouti A82S | CGAGACAGACGTGAGGACAGGTGGGGTGGACGTGGCCGGCTTG |
| (SEQ ID NO: 216) | GGCAGCCCTGGCGTTTCCCTGCAGAAAAAGGCTTCGATGAAGAA |
| | CGTG[G/T]CTCNTCCCCGGCCCCCGCCACCCACCCCCTGCGTGGC |
| | CACTCGCAACAGCTGCAAGTCCCCGGCGCCCGCCTGCTGTGACC |
| | CCTGCGCCTCCTGCCAGTGCCGCTTCTTCCGCAGCGCCTGCACCT |
| | GCCGCGTTCTCAGTCCCAGATGCTGAGCGCGCCCAGCGGCCTCC |
| | AGGGGGTTGGCTGAT |
| AGOUTI-R83H | CGAGACAGACGTGAGGACAGGTGGGGTGGACGTGGCCGGCTTG |
| (SEQ ID NO: 217) | GGCAGCCCTGGCGTTTCCCTGCAGAAAAAGGCTTCGATGAAGAA |
| | CGTGNCTC[G/A]TCCCCGGCCCCCGCCACCCACCCCCTGCGTGGC |
| | CACTCGCAACAGCTGCAAGTCCCCGGCGCCCGCCTGCTGTGACC |
| | CCTGCGCCTCCTGCCAGTGCCGCTTCTTCCGCAGCGCCTGCACCT |
| | GCCGCGTTCTCAGTCCCAGATGCTGAGCGCGCCCAGCGGCCTCC |
| | AGGGGGTTGGCTGATTATCTAAGAA |
| AGOUTI-R96C | CGAGACAGACGTGAGGACAGGTGGGGTGGACGTGGCCGGCTTG |
| (SEQ ID NO: 218) | GGCAGCCCTGGCGTTTCCCTGCAGAAAAAGGCTTCGATGAAGAA |
| | CGTGNCTCNTCCCCGGCCCCCGCCACCCACCCCCTGCGTGGCCA |
| | CT[C/T]GCAACAGCTGCAAGTCCCCGGCGCCCGCCTGCTGTGACC |
| | CCTGCGCCTCCTGCCAGTGCCGCTTCTTCCGCAGCGCCTGCACCT |
| | GCCGCGTTCTCAGTCCCAGATGCTGAGCGCGCCCAGCGGCCTCC |
| | AGGGGGTTGGCTGATTATCTAAGAA |
| MLPN-DILUTE | GGAGGTAGATGAGCCTCTGGGGACGCCCCCCTCCTGCTGCCCAG |
| COLOR | GGCCGAGGGGCCCCCGGTCCTCTCTGTGAGGCTGACTCTGACTC |
| (SEQ ID NO: 219) | TCCTCCTCTTGCCCCTGCCTGCACCTGTGAAGAAAAAGC[G/A]CC |
| | TCTCCTTCCACGACTTGGACTTTGAGGCAGACTCTGACGACTCCA |
| | CTTGGTCTGGAAGTCACCCCCCCCACTCGTCCCCAGTCTCAGTGG |
| | CCACAGACAGCCTGCAGGTCAGTGGGCTCATTTCTGGCCCCCCA |
| | GCCTTCCCGGGATAACCTGAGCGACAGGTACGTGGGCCCCAGGT |
| | GGGGGACGGGGCGCTCTGGGAAGGAGTCCGATGGCCATATCAA |
| | GCTTCGGGG |
| MASK | ATGGTCTGGCAGGGCCCCCAGAGAAGGCTGCTGGGCTCTCTCAA |
| (SEQ ID NO: 220) | TGGCACCTCCCCAGCCACCCCTCACTTCGAGCTGGCTGCCAACC |
| | AGACCGGGCCCCGGTGCCTGGAGGTGTCCATTCCCAACGGGCTG |
| | TTCCTCAGCCTGGGGCTGGTGAGCGTTGTGGAAAATGTGCTGGT |
| | GGTGGCCGCCATTGCCAAGAACCGCAACCTGCACTCGCCCATGT |
| | ATTACTTCATCGGTTGCCTGGCTGTGTCCGACCTGCTGGTGAGCG |
| | TGACGAATGTGCTGGAGACGGCCGTCATGCTGCTGGTGGAGGCA |
| | GGCGCCTTGGCTGCGCAGGCTGCTGTGGTGCAGCAGCTGGACGA |
| | CATCATTGACGTGCTCATCTGTGGTTCCATGGTATCCAGCCTCTG |
| | CTTCCTGGGCGCCATCGCCGTGGACCGCTACCTCTCCATCTTCTA |
| | CGCGCTGCGATACCACAGCATCGTCACACTCCCGCGGGCGTGGC |
| | GGGCCATCTCCGCTATCTGGGTGGCTAGCGTCCTCTCCAGCACG |
| | CTCTTCATTGCCTACTACAATCACACGGCCGTCCTGCTTTGTCTT |
| | GTCAGCTTCTTTGTAGCCATGCTGGTGCTCATGGCAGTGCTGTAC |
| | GTCCACATGCTTGCCCGCGCCCGCCAGCACGCCCGAGGTATTGC |
| | CCGGCTCCGTAAGCGGCAGCACTCCGTCCACCAGGGCTTTGGCC |
| | TCAAGGGCGCTGCCACACTCACTATCCTGCTGGGCATTTTCTTTC |
| | TCTGCTGGGGCCCCTTCTTCTTGCACCTCTCACTC[A/G]TGGTCCT |
| | CTGCCCTCAACACCCCATCTGTGGCTGCGTCTTTCAGAACTTCAA |
| | CCTCTTCCTCACCCTCATCATCTGCAACTCCATCATTGACCCCTT |
| | CATCTACGCCTTCCGCAGCCAGGAGCTCCGAAAGACTCTCCAAG |
| | AGGTAGTGCTATGTTCCTGGTGA |
| MC1R_YELLOW | CATTTTCTTTCTCTGCTGGGGCCCCTTCTTCTTGCACCTCTCACTC |
| (SEQ ID NO: 221) | ATGGTCCTCTGCCCTCAACACCCCATCTGTGGCTGCGTCTTTCAG |
| | AACTTCAACCTCTTCCTCACCCTCATCATCTGCAACTCCATCATT |
| | GACCCCTTCATCTACGCCTTCCGCAGCCAGGAGCTC[C/T]GAAAG |
| | ACTCTCCAAGAGNTAGTGCTATGTTCCTGGTGAGGCTGCAGGCT |
| | TGAGGCCAGGGTGCTGGCCAGAGGGGGGTGGTGATTGATACCC |
| | ATGTGACTGGGGCAGTCACTTGCAGAAAAGGACAGATGAGCTG |
| | ATCTGTGGTGTGGTGGATGCATGGACCCTCTGGGGCCAGAGAAA |
| | GGAATAAACAAAAATCTCCAGGAGTTGCTGTGGAGAATGGAGC |
| | AGGCTGAGGAGATGGTGGGGCCACA |
| TYRP1_345P | TACATATCCCATCCTTTTCCCAGGTACTGAGGGTGGGCCAATTA |
| (SEQ ID NO: 222) | GGAGAAATCCAGCTGGAAATGTGGCTAGACCAATGGTGCAACG |
| | TCTTCCTGAACCANAGGATGTCGCTCAGTGNTTGGAAGTTGGTT |
| | TATTTGACACA[CCT/*]CCTTTTTACTCCAATTCTACTAACAGTTT |
| | CCGAAACACAGTGGAAGGTAAGTAAAAGAAATCAGTGCTTTGA |
| | ATTCACAGTTAACTGAACTATTCACATTCAGATCTCTTTGAAAAA |
| | TCTTTGAAAAACCATATAGATCCTGTGAATTTACATGAATGCTG |
| | CCTCCAGTTATGATGTAGTCACAATTCTCTGCTCGAGAAAGAAC |
| | TTCTTAAAGAAAAGTGTCAGACCGTGAAACTCTTTTTAATTATCA |
| | TAGAGGAGAAGTGCTTAGAAATTAT |
| TYRP1-MC1R | ATGAAAGCTCATAAACTCCTCTCTCTGGGAAGCATCTTCTTGTTC |
| (SEQ ID NO: 223) | CTGCTTTTTTTCCATCAGACCTGGGCTCAATTCCCAAGAGAGTGT |
| | GCCACTGTTGAGGCCTTGAGAAATGGTGTG[T/C]GTTGCCCAGAC |
| | CTGTCCCCAGTGTCTGGGCCTGGGACTGACCCCTGTGGCTNCTC |
| | ATCAGGGCGGGGGAGGTGTGAGGCAGTGATAGCAGACTCCAGA |
| | CCCCACAGCCACCATTACCCNCATGATGGCAGAGATGATCGGGA |
| | GGTTTGGCCCACACGGTTCTTCAACAGGACCTGCCACTGCAATG |
| | GCAATTTCTCAGGACACAACTGTGGGACTTGCCGTCCAGGATGG |
| | AGAGGAGCTGCCTGTGATCAGAAGGTTCTCACAGTCAGGAGAA |
| | ACCTCCTGGCCTTGAATACAGAAGAGAAGAACCACTTTGTCCAG |
| | GCCTTGGATATGGCAAAGCGCACAATTCACCCTCAGTTTGTC |
| TYRP1_EX5 | TCTTGCTATGTGTAAAAATTAAAGGGCAAAGATCAGATCTCTAA |
| (SEQ ID NO: 224) | GTATCCTATAAATATTTACATATCCCATCCTTTTCCCAGGTACTG |
| | AGGGTGGGCCAATTAGGAGAAATCCAGCTGGAAATGTGGCTAG |
| | ACCAATGGTGCAACGTCTTCCTGAACCA[C/T]AGGATGTCGCTCA |
| | GTGNTTGGAAGTTGGTTTATTTGACACANNNCCTTTTTACTCCAA |
| | TTCTACTAACAGTTTCCGAAACACAGTGGAAGGTAAGTAAAAGA |
| | AATCAGTGCTTTGAATTCACAGTTAACTGAACTATTCACATTCAG |
| | ATCTCTTTGAAAAATCTTTGAAAAACCATATAGATCCTGTGAATT |
| | TACATGA |
| Myotonia | CCATTCCTCTCTGCCTCCCTTCCCTGCCCCTCCCATCTCTCTGTCT |
| congenital | CTCTCTCCCCTAGTAGCAGCCATACTATTACACTGACATGCTGA |
| (SEQ ID NO: 225) | [C/T]GGTGGGCTGTGCTGTAGGAGTTGGCTGTTGTTTTGGGACGC |
| | CACTTGGAGGCAAGTGATTTACCCCTCCTACATCAGTCCGCTGCT |
| | TGGGCTTGCTCCCCAGCCAGGTTTTGTCAGCATCCCCAAGTGTG |
| | ACATTACCAGTTACAACAA |
| CLAD_1 | GCCGCGTGGGGTCGGCCCGCGTCAGGCCACCTCTCACGGAGCTG |
| (SEQ ID NO: 226) | CCTCCTCCTGCCGCCAGCGTCCTGCCAGGAGTGCACCAAGTACA |
| | AAGTGAGCACGTGCCGGGACT[G/C]TGTGGAGTCGGGGCCCGGC |
| | TGCGCCTGGTGCCAGAAGCTGGTAAGAGCCCCCCCCCAGGGACC |
| | TCGCGCCCGTCCTGCCCGTCCCGCGTTCCCGTCCCCGTTCCTGTC |
| | CCCACGCCCTCCCTCTGCCTCT |
| CLN2-TPP1 | GGAAAATACCTGACCCTAGAGGATGTGGCTGAACTGGTCCGGCC |
| (SEQ ID NO: 227) | ATCACCACTGACCTTCCGCACAGTCCAAAAATGGCTCTCAGCAG |
| | CTGGAGCC[C/*]GGAACTGCCACTCGGTGACCACACAAGACTTTC |
| | TGACTTGCTGGCTGAGTGTCCGA |
| CLN5_B_C | CTAGGAAACACATTTAACCAAATGGCAAAGTGGGTAAAGCGGG |
| (SEQ ID NO: 228) | ACAATGAAACAGGAATTTATTACGAGACGTGGACTGTT[C/T]AA |
| | GCCAGCCCAACAAAGGGGGCTGAGACATGGTTTGAATCCTATGA |
| | TTGTTCTAAATTCGTGTTAAGGACATACAAGAAGTTGGCTGAAC |
| | TTGGAGCAGAGTTCAAGAAGATAGAAACCAACTATACAAGAAT |
| | ATTTCTTTACAGTGGAGAACCTACC |
| CLN8_DOG | GAGAACGTAGCAGTTCACCTGTCCAATGTGCTCTTCCGGACATT |
| (SEQ ID NO: 229) | TGACTTGTTTTTGGCCATCCACCATCTCTTCGCCTTTCTGGGATTT |
| | CTTGGCTCCGTGGTCAACCTCGGAGCCGGCCACTATCTGGCTAT |
| | GAGCACGC[T/C]CGCTTCTGGAGGCGAGCACTCCCTTCACCTGCA |
| | TTTCCTGGATGCTCCTAAAGG |
| TFT_CH | ACGCCCACCCTCCAACCCGCACGGTGCTCGGTGCTGCTCCCCGG |
| (SEQ ID NO: 230) | CTCCTGCCCCCACAGTCCGCTCGAGAGCTTAGCGGTTGCTTCTGA |
| | GTGACCCACTTTATGTCCCCCCTTGTGCCATAGAGACCTCAGCA |
| | AAAGGGGGCTCCCTTCCCCGTCGCAGCTTCTGTCTTTTTCCAAAC |
| | TCCCCGAGCCGACCAGCCGAGCCGTCTCCCGAGCGGCGGAGATC |
| | ATGGAAGCCTCCGTGCAGGCCGTGAGGACACGGGTCTATGGGA |
| | AGCTGGGG[C/T]GATCTTGGCCTCTCACCGGTAAGCGCCCCGAG |
| | GGGCAGGCCCCAGGGCCTCGACCCAGGACAGCATGGCCAAGGG |
| | AAGGTATCTGGGTTCCCCTGGGAGGTCCTGCAGCCCCCTCCGGA |
| | CGTGGGGAAACGGCTCAAGCCCCCAGGCAGCCCCGTCCTGGCAT |
| | CAGAGAGTGTGGGGGTGTTGGCGCGGCCCGGACGGAAGGTGGG |
| CYST_NEWFOUN | AGGTATTCAAGAGAAACTGGACTACATCACAACTTTAAATATAA |
| (SEQ ID NO: 231) | AAACCATTTGGATTACTTCATTTTACAAATCATCCCTTAAAGATT |
| | TCCGATATGGTATCGAAGACTTC[C/T]GAGACATTGATCCTATTT |
| | TTGGAACAATGAAAGATTTTGAGAATCTGCTTGCAGCCATACAC |
| | GATAAAGGT |
| Dach_narco | GTCCGGCACCAAACTGGAGGACTCCCCCCCTTGTCGCAACTGGT |
| (SEQ ID NO: 232) | CATCTGCTCCGGAGCTGAATGAAACTCAAGAGCCCTTTTTAAAC |
| | CCCACCGACTATGACGACGAGGAATTCCTGCGGTACCTGTGGAG |
| | GGAATACCTACACCCGAAAGAATAT[G/A]AGTGGGTCCTGATCG |
| | CTGGCTACATCATCGTGTTCGTGGTGGCTCTCGTGGGCAACGTCC |
| | TGGGTGAGTCTGGCCCCGGGCAGCCCTCCCGAGGGCTGTCACGG |
| | CCCCTCTGCGCGGGCGGGGCTGCCGGGGCTCTGAAGAC |
| DYSTROPHIN | ATAAAGAGTAACACTCTTAAGGAATGATGGGCATGGGTTGTCAA |
| (SEQ ID NO: 233) | TTAAAAATCAGAAATGAAGTGAATCTTGTGAAATATTGTAAATT |
| | GATTTATATTTATTTTTATGTGTGTGTGTTTCAG[GCCAG/*]ACCT |
| | GTTTGATTGGAATAGTGTGGTTTGCCAGCAGTCAGCCACACAAC |
| | GCCTGGAACATGCATTCAACATTGCCAAATATCAATTAGGCATA |
| | GAGAAACTGCTTGATCCTGAAGGTCGGTACATTTCTGGACTACC |
| | ATAGTTTTTAGTATAGTTTAATATTTATAATCTCAGA |
| globoid cell | GGGTTGCCATGGTCATTTCCTGGATGGATAGGAAAAGGTTTCAA |
| leucodystrophy | CTGGCCTTACGTGAATCTTCAGCTGACTGCCTACT[A/C]TATCAT |
| (SEQ ID NO: 234) | GACCTGGATTGTGGGTGCCAAGCATTATCATGATTTGGACATTG |
| | ATTATATCGGG |
| GM-gangliosidosis | ATCGACTCTATCTCCTGTGGCTCTTGCTTGTCACCATTGCCTATA |
| (SEQ ID NO: 235) | ACTGGAACTGCTGGCTTATACCACTACGCCTCGTCTTTCCATATC |
| | AAACACCAGACAACACACACTACTGGTTTATTACAGACATCACA |
| | TGTGATATCATCTACCTTTGT[G/A]ATATGCTATTAATCCAGCCC |
| | AGACTCCAGTTTATAAAAGGAGGAGACATAAT |
| GM-gangliosidosis | ATGCTTCCCAGAGGACATTCACAATTGACTACAGCCACAACCGC |
| (SEQ ID NO: 236) | TTCCTGAAGGACGGCCAGCCCTTCCGCTACATTTCGGGAAGCAT |
| | TCACTATTCCCC[G/A]TGCCCCGCTTCTACTGGAAGGACCGGCTG |
| | CTGAAGATGAAGATGGCTGGGCTGAATGCCATCCAGACGTAAGT |
| | AAGAGGGCGCTGGGCTCTCACCTGGGCCTAGACACCCATACCTG |
| | GAGAGAGAGAGCAGCTGGATC |
| Hemophilia B | GGTGCCTAAGGTGGCTGGCACTGACTTGCCGTACCCTCCCCATG |
| (SEQ ID NO: 237) | TCTCCTTGTGTCTGCAGTGGGTGAATGGGGTCCATGTGGCAGAG |
| | CACGAGGGGGGTCACCTCCCCTTCGAAGCTGACATCAGCAAGTT |
| | GGTCCAGAGCGGGCCCCTGTCCTCCTGCC[G/A]TATTACCCTTGC |
| | CATCAACAACACGCTCACCCCCCACACTCTGCCGCCAGGGACCA |
| | TCGTCTACAAGACAGACGCTTCCAAGTGAGCAGCACTCTGCTCC |
| | CCTGCCCCCCCTGCCCCCCACCCACTGGGCTTCCGACT |
| hereditary cataracts | CCGAGCCACGTGCCTTCGGTCCACGAAGTTCACCATTTATAACA |
| (SEQ ID NO: 238) | ACATGTTCTGTGCTGGCTTCCATGAGGGAGGTAAAGATTCATGC |
| | CAGGGCGATAGTGGGGGACCCCATGTCACCGAAGTAGAAGGCA |
| | TAAGTTTCTTAACTG[G/A]GATTATTAGCTGGGGTGAAGAGTGTG |
| | CGATGAAAGGGAAGTATGGAATATATACCAAGGTGTCCCGGTAT |
| | GTCAACTGGATTAAAGAAAAGACGAAGCTCACCTAAAGAATAA |
| | TGTATTTCCAAGGTTGACACGTTTAGGGTAGAAAATGGACAAGG |
| | TCCTTTACTAACTAATCACTTTTTTTATCTCTTTAGATTTGACTAT |
| | ATACATTCTC |
| hereditary cataracts | GTGCAGGGAGAAGGGCCTGGCACTGCTCAAAGAAGAGCCGGCC |
| (SEQ ID NO: 239) | AGCCCAGGGGGGGAAGGCGAGGCCGGGCTGGCCCTGGCCCCAA |
| | ACGAGTGTGATTTTTGCGTGACAGCCCCCCCCCC[*/C]ACTGTCC |
| | GTGGCTGTGGTGCAGGCCATCCTGGAAGGGAAGGGGAACTTCA |
| | GCCCCGAGGGGCCCAGGAATGCCCAACAGCCTGAACCAAGGGG |
| | TCCCAGGGAGGTACCTGACAGGTGAGC |
| PRA | AGGAGTTTTCCCGTTTCCACGAAGAGATCCTGCCCATGTTCGAC |
| (SEQ ID NO: 240) | GACTGCAGAACAACAGGAAGGAAT[G/A]GAAGGCCTTGGCTGA |
| | TGAGTACGAGGCCAAGCTGAAGGCCCTGGAGGAGGAGAAGCAG |
| | CAACAAGAGGACAGGACGACAGCCAAGAAAG |
| PHOSPHOFRUCTOKINASE | GTGTTCTGGGGATGCGTAAGAGGGCTCTGGTCTTTCAACCAGTG |
| DEFICIENCY | ACTGAGCTGAAGGACCAGACAGATTTTGAGTGAGTACATCTGCT |
| (SEQ ID NO: 241) | TCCCTGGTAGTTTCAGGGTCTGCTCTTCCCAGCCTGTGTGCTGCC |
| | TTCAATCCTCTCATCCTAGGACTAACACCGTCATCACACCTATTT |
| | CAGATCTTAACCCCGTGCCCTAAAATCCGGCCTCTTCTACTCAAC |
| | TTCTTTCCATAAGCTTTGGATAGAAGTCAGTTGGGTTGCTAAAA |
| | GCTGAAATCATCATCTCTCTCATTTCTCTGTAGTCACCGCATCCC |
| | CAAGGAACAGT[G/A]GTGGCTGAAGCTGAGGCCCATCCTCAAAA |
| | TCCTAGCCAAGTACGAGATTGACTTGGACACCACAGAGCACGCC |
| | CACCTGGAGCACATCAGTCGGAAGCGATCTGGAGAAACTTCTAT |
| | CTAACCCTCTTTGGAGTGAGGGTCATGGATTGTCTGATCATGGTC |
| | AGCTCACCCCCTGATAGATCCAAGTCCATGTATCCCCAAGTATTT |
| | TAGCTCATTTTTCTTTAGGTTTCCTTTTATTCTGCAACTGTAGCCA |
| | TGACCAGCTCTGGCCAGGGAGCTGGGGCAGCGGGCAGTGAGTA |
| | GAGGCTCCTTTTAGGTGGAATTTATCAACTTCTACCCCAGCTTCA |
| | TCTGTCACACAAGACTGGGCTCCTCTAGTGCTACTGCTAGATTTC |
| | AGCTACTCGGTTAGAATTTTCCTGAAAATAAGCTTTATTTATTTC |
| | TTTGTGATAACAAAGTCTTGGTTCCTCTATTACTTTTACTGCAGT |
| | GACAAACAATAGCTACACTAATAAATGCCAACTGGTCACTGTGC |
| | TTTTGGTTCTCCTGTTGTCACTTTCACAAGTGAATGTCATCCTGT |
| | CAACC |
| PRA | CAGAGCCTGAAGTCGTCCTGCCGGAGCCCTGGGTGGCCAAGCTC |
| (SEQ ID NO: 242) | AGGCCTCAGCAGCACTCTTNGGACTGAGCCGCCCACGGGGCAGC |
| | CGCCAGGACCGCAGCCATGAACGGGA[C/G]GGAGGGCCCGAAC |
| | TTCTACGTGCCCTTCTCCAACAAGACGGGTGTGGTGCGCAGCCC |
| | CTTCGAGTACCCACAGTACTACCTGGCTGAGCCATGGCAGTTCT |
| | CCATGCTGGCTGCCTACATGTTTCTGCTGATCGTGCTCGGCTTCC |
| | CCATCAACTTCCTCACGCTCTAC |
| Thrombasthenic | GCGGCACGACTTGCTGGTGGGCGCGCCACTGTTCATGGAGAGCC |
| thrombopathia | GCGCGGACCGCAAGCTGGCCGAGGTGGGGCGCGTGTACTTGTTC |
| (SEQ ID NO: 243) | CTGCAGCCTCGAGGTCACCAGGCGCTGGGCGCCCCCAGCCTCCT |
| | GCTGACTGGCACACAGCTCTATGGGCGATTCGGCTCGGCCATCG |
| | CATCTCTGGGCGACCTC[G/C]ACCGGGACGGCTACAACGGTAAG |
| | GGGCAGAGAGGAGCACCGCTTGCTTCAGACTGGTTAACAGCCA |
| | GAACCAAGACCGCCGATTTGACCAGAGGGCAGCCAGAGCGGGG |
| | AAGGGCTTTTCTCTGGAAGAGTTGAATGGGACCAGTTTGTTTGC |
| | ATTGGTCCAGGC |
| SCID | GATCTTTGGAAGATATTTGATTACCTAACCTTGGTAATTGTTTTA |
| (SEQ ID NO: 244) | TAGGATTAAAACTAAGTTGGATCTAGGAGGAGTGATTCAAGATT |
| | TTATTAGTGCCCTAGAACAGCTCTCTAATCCTGAAATGCTCTTTA |
| | AGGTAATGTAATAGCTTCTAACTCATAAAACATAGAATTTGGAT |
| | TGAACTTACTTGCAGTCAACTTGGTTTTTCCCTCTCTCTCTCTTTT |
| | TTTTTTTTTTTTTTGCACAGGATTGGACTGATGATATGAAAGCCG |
| | AACTGGCAAAAAACCCTGTTAATAAAAAAAACATTGAAAAGAT |
| | GTAT[G/T]AAAGAATGTATGCAGCTTTGGGAGATCTAAGGGCTC |
| | CAGGGCTTGGGGCTTTCAGAAGGAGGTTTATTCAGGTAGGGATA |
| | GGTGGCAGCCTGCCTATATAATAATGGAATCATTGTAACAATCA |
| | GTAGTTATATTTTCTGGCTTGTTAATAATCCTGG |
| WELSH_SCID | CCTTCAGGATCCTAACTTGTTCAGGCCAGGGGAATGACCACACA |
| (SEQ ID NO: 245) | CACACACATATCTCCAGTGATCCCCTGGGCTCCGGAGAACCTAA |
| | CCCTTCACAACCTGAGCGAATCCCAGCTAGAACTGAGCTGGAGC |
| | AACAGACACTTGGACCACTGTTTGGAGCATGTTGTGCAGTACCG |
| | GAGTGACTGGGACC |
| | [*/C]GCAGCTGGACTGTGAGTGACTTGGGTCATGAAGGTGGCAG |
| | CAAAGGCCAAGCAAATAGGGATAAAGGATTCAATCAGC |
| SCID_X | GTTTCTAAGGTTCTTTCCACCGGAAACTATGACAGAAGGAAATG |
| (SEQ ID NO: 246) | TGTGGGTGGGGAGGGGTAATGGGTGAGGGGCCCAGGTTCCTGA |
| | CAGTCTACACCCAGGGAACGAAGAGCAAGCGCCATGTTGAAGC |
| | CACCATTGCCACTCAGATC[CCTC/*]TTATTCCTGCAGCTGTCTCT |
| | GCTGGGGGTGGGGCTGAACTCCACGGTCCCCATGCCCAATGGGA |
| | ATGAAGACATCACACCTGGTGGGAAACATGGGACTGGAAGGGG |
| | TTGGTGAGAGGGGAGCCTGTGGGAAGGGGTCGCATAGAAATCT |
| | TGAACCTGCCATGGGGCATTAGAAGGATGTGGGCAGAGTTTAAG |
| | AGTGCTGTGGAGA |
| SCN_DOG | GGAAGGCTAAGTGGAGCAAATAAATGTTTGTTCTGAAACATTAA |
| (SEQ ID NO: 247) | GAATTACTTCATTGACTTTTTAACAGAATATGCAATAAATTAAAT |
| | ATTTCTTATCTATAGGAGAAAGAAAAAAAAA[*/A]CAAAGGAAG |
| | ATAGAAATCTTACCAAAGATGTTTCACTTCTAGACCTGGATGAT |
| | TGTAAGTGTTGAAATTTAAATTTTTTCTTCTCTTTTTAGTAGTAG |
| retinal dystrophy | TTAGCCCTTTTCTTTCACAGCTTGAAGGTTACTGGACTGAAAAAC |
| (SEQ ID NO: 248) | TCCGTTTGCTTCTGTAGGTTTTTTTCTTACTTCCGAGGAGTGGAG |
| | GTCACTGACAATGCCCTTGTTAACGTCTACCCAGTAGGGGAAGA |
| | TTACTANGCCTGCACGGAGACCAACTTCATTACA[AAGA/*]TTAA |
| | TCCTGAGACCCTGGAGACAATTAAGCAGGTAGGACGAAATGCTC |
| | AGGCGACGTTGCTCAAGAATTTAGAATTTGCAGTTTAGATTTAA |
| | CTGCAATTTTGGGGAAAGCTCATGAGGGCCAAATAGATTGTCTC |
| | GCTGCCTTGCTTTGTCATCAACTACTAGCCATGTGACACGAGGC |
| | ACTCTTTA |
| type-2 von | GGGATATCCGATACCGGGGTGGCAACAGGACCAACACTGGACT |
| Willerbrand's | GGCCCTGCAATACCTGTCCGAACACAGCTTCTCGGTCAGCCAGG |
| (SEQ ID NO: 249) | GGGACCGGGAGCAGGTACCTAACCTGGTCTACATGGTCACAGG |
| | AAACCCCGCTTCTGATGAGATCAAGCGGATGCCTGGAGACATCC |
| | AGGTGGTGCCCATCGGGGTGGGTCCACATGCCAATGTGCAGGAG |
| | CTGGAGAAGATTGGCTGGCCCA[A/G]TGCCCCCATCCTCATCCAT |
| | GACTTTGAGATGCTCCCTCGAGAGGCTCCTGATCTGGTGCTACA |
| | GAGGTGCTGCTCTGGAGAGGGGCTGCAGATCCCCACCCTCTCCC |
| | CCACCCCAG |
| Type III von | TGTCGCTCCCTCTCTTACCCGGAGGAGGACTGCAATGAGGTCTG |
| Willebrand | CTTGGAAGGCTGCTTCTGCCCCCCAGGGCTGTACCTGGATGAGA |
| (SEQ ID NO: 250) | GGGGAGATTGTGTGCCCAAGGCTCAGTGTCCCTGTTACTATGAT |
| | GGTGAGATCTTTCAGCCCGAAGACATCTTCTCAGACCATCACAC |
| | CATGTG[G/A]TAAGTGCGAGCAGCATGACCAGGGACCTCAGGAA |
| | TGGCGGAGCTTGTAAGGAAAATGGTCTTCTGGGTCCTTCATTTC |
| | ACGGTTGGGAAACTGAGGCCCAGGAAGGGAAGTGACTTGCCCT |
| | GAGTTGCACAGCTCGAATGATTTCCTTACATCGCTGGAAACTAG |
| | AGCAGACTGCCA |
| Type III von | GAAGGGAAAAATGAGTGAGTAAATTATATTTTGGGGAAGATTTT |
| Willebrand | TTTGTTGTTGTTCATTTGTTACGTCCTTGGGGAGAGTTCTCCATG |
| (SEQ ID NO: 252) | AGATGGGATTAATGATGTACATCAGATGATTAGAGGTAAATATC |
| | CCGGCTTTTTTGGTAATAATCATAGTTACTGACTCTTTTCTCTTTC |
| | AGGGGGTTTCCAAAATGGCAAAAGAGTGAGCCTCTC[C/*]GTGT |
| | ATCTCGGAGAATTTTTCGACATTCATTTGTTTGTCAATGGTACCA |
| | TGCTGCAGGGGACCCAAAGGTAAGTC |

The present invention is not limited to species such as horses and dogs, but can be used in a variety of species. For example, the following tables demonstrate sequences that may be used determined genetic characteristics, such as parentage, identity, sex, genotype and/or phenotype and breed determination in cats. Thus, in further embodiments, the present invention provides a panel comprising a plurality of assay compositions, wherein each assay composition is capable of identifying at least one of the nucleotide markers as set forth in Tables 7 and 8 provided below:

TABLE 7
CAT SNP PANEL SEQUENCES

| | Cat Genomic | | | |
|---|---|---|---|---|
| SEQ ID NO | Location | SNP CONTIG | SNP | Description |
| 253 | Un: 51,831,052 | c200902194.Contig1 41887716 | A/G | Many |
| 254 | c2: 703,930 | c201102843.Contig1 52683485 | A/G | Many |
| | | 42085143 | | |
| 255 | c2: 703,930 | c201102843.Contig1 52683485 | A/G | Many |
| | | 42085143 | | |
| 256 | E2: 64,720,639 | c209402154.Contig1 40390026 | A/G | Many |
| 257 | D4: 812,589 | c210302384.Contig1 51478757 | C/T | Many |
| | | 46990850 | | |
| 258 | B4: 147,961,464 | c214001733.Contig1b00 | A/G | Many |
| | | 40834831 41883837 | | |
| 259 | B1: 156,143,186 | c216702119.Contig1 50170968 | A/G | Many |
| 260 | F2: 77,518,182 | c217102268.Contig1 51882103 | T/G | Many |
| 261 | A2: 17,611,273 | c218902205.Contig1 43673924 | C/T | Many |
| 262 | B3: 107,303,663 | c220002309.Contig1 41798812 | A/C | Many |
| 263 | D2: 74,626,676 | c221302563.Contig1 39163914 | C/T | Many |
| 264 | A3: 88,919,777 | c221802646.Contig1 38897465 | C/T | Many |
| 265 | A1: 151,473,414 | c222902793.Contig1 42602082 | C/G | Many |
| 266 | B1: 178,757,633 | c223102384.Contig1 51610716 | C/T | Many |
| 267 | B4: 19,612,127 | c225702363.Contig1 44291991 | A/C | Many |
| 268 | E2: 11,112,283 | c226102304.Contig1 45346791 | C/T | Many |
| 269 | A1: 15,263,737 | c228202754.Contig1 41061200 | C/T | Many |
| 270 | A3: 40,227,427 | c229902453.Contig1 51587423 | A/G | Many |
| 271 | C2: 150,072,397 | c230302478.Contig1 47807293 | A/G | Many |
| 272 | B2: 43,290,061 | c231602346.Contig1 42950909 | A/G | Many |
| 273 | D1: 124,939,879 | c232702561.Contig1 50699305 | A/G | Many |
| | | 43049735 | | |
| 274 | C1: 123,746,252 | c233302605.Contig1 45945358 | C/T | Many |
| 275 | F2: 75,210,562 | c237202594.Contig1 39922895 | C/T | Many |
| 276 | A3: 14,410,638 | c238102323.Contig1 51345702 | A/G | Many |
| 277 | F1: 33,007,663 | c238602943.Contig1 43762371 | A/G | Many |
| | | 42805370 45085530 | | |
| 278 | A1: 208,380,043 | c239502892.Contig1 39703120 | A/G | Many |
| | | 39628806 | | |
| 279 | E2: 35,480,527 | c379002760.Contig1 38992791 | C/G | Many |
| 280 | A3: 118,999,155 | c246003822.Contig1 42533812 | C/G | Many |
| 281 | A2: 10,913,767 | c248603449.Contig1 43067711 | A/G | Many |
| 282 | B2: 47,659,161 | c248803703.Contig1 41077986 | A/C | Many |
| 283 | D2: 89,706,040 | c249103480.Contig1 51530567 | C/T | Many |
| 284 | Un12: 7,317,515 | c252004127.Contig1 39641583 | C/T | Many |
| 285 | Un2: 523,114 | c253404131.Contig1 37960459 | A/G | Many |
| 286 | B2: 156,308,475 | c256404084.Contig1 54345379 | A/G | Many |
| | | 43778944 | | |
| 287 | A2: 25,685,296 | c259703305.Contig1 41812011 | C/T | Many |
| 288 | A2: 2,129,037 | c261103489.Contig1 51387364 | C/T | Many |
| 289 | A2: 161,801,210 | c263503219.Contig1 39442596 | A/C | Many |
| 290 | D3: 7,290,581 | c265103456.Contig1 43475101 | A/G | Many |
| 291 | F1: 19,516,618 | c267903188.Contig1 39678411 | A/G | Many |
| 292 | D2: 82,189,281 | c278503306.Contig1 42981906 | C/T | Many |
| | | 51016912 | | |
| 293 | D1: 36,295,835 | c281903151.Contig1 52096151 | A/G | Many |
| 294 | D3: 33,258,191 | c288803295.Contig1 44646770 | A/G | Many |
| 295 | C2: 63,676,887 | c293703365.Contig1 40266370 | C/T | Many |
| 296 | A3: 48,181,817 | c372702909.Contig1 52632612 | C/T | Many |
| 297 | A3: 11,904,341 | c297603245.Contig1 44279919 | A/G | Many |
| 298 | E3: 63,458,569 | c298202957.Contig1 40800988 | A/C | Many |
| 299 | B2: 112,716,268 | c302302970.Contig1 40606850 | A/T | Many |
| 300 | B3: 149,673,110 | c307303163.Contig1 40903035 | C/T | Many |
| 301 | C1: 125,311,520 | c314603195.Contig1 42747048 | A/G | Many |
| 302 | B1: 19,312,704 | c315703075.Contig1 38246147 | C/T | Many |
| 303 | B2: 120,276,458 | c315703352.Contig1 53559241 | C/G | Many |
| 304 | B2: 159,389,942 | c332003111.Contig1 52844210 | A/G | Many |
| 305 | D4: 39,362,745 | c337003053.Contig1 41695419 | C/T | Many |
| 306 | B1: 172,534,764 | c354102993.Contig1 39140016 | A/G | Many |
| 307 | POINTED1 | | G/A | Many |
| 308 | POINTED2 | | G/T | Many |
| 309 | ALBINO | | C/* | Many |
| 310 | CHOCOLATE | | G/A | Many |
| 311 | CINNAMON | | C/T | Many |
| 312 | Mucopolysaccharidosis | | T/C | Many |
| | Type VI | | | |
| 313 | Mucopolysaccharidosis | | G/A | Many |
| | Type VI MILD | | | |
| 314 | Polycystic Kidney | | C/A | MANY |
| | Disease | | | |
| 315 | Hypertrophic | | G/C | MAIN COON |
| | cardiomyopathy | | | |
| | MC | | | |
| 316 | Hypertrophic | | C/T | RAGDOLL |
| | cardiomyopathy | | | |
| | RG | | | |

The nucleic acid sequences of the markers of Table 7 are provided in Table 8 below, where some polymorphic sites (e.g., the single nucleotide polymorphism (SNP), insertion and/or deletion) are bracketed and indicated in bold; however, those skilled in the art can readily identify other polymorphic sites by researching the particular sequence in corresponding cat registries or databases. Many sites may be identified.

TABLE 8
CAT SNP PANEL NUCLEOTIDE MARKER SEQUENCES

| | Cat Genomic Location | |
|---|---|---|
| SEQ ID NO | and/or Description | Sequence |
| 253 | Un: 51,831,052 | TAGTCAGTCTTGGATACATTCGGCCACAGAGTCCTTC |
| | | AAAAATTGCCTTTCAGTCCTATGTTGACAAAGGTAAGT |
| | | CCAGGGCATTTCAAGGTGCCCAACARGAGTGCTAAT |
| | | GTGTAGTCAGGGTCAGAGATATTGGGAGGGAGCTAT |
| | | CCTCACTTATGGGACAAGAGGAACATGGAGTTACACA |
| | | CATAGGATAAATGAAAA |
| 254 | c2: 703,930 | GGATGTGGAGAGATCGGAAGCCGTCCGCCCCGGTG |
| | | GTGGGATTGCCAAACGGTATAGCTGTTCTAGAACACA |
| | | GCCCGCGGTCCCCGGAAAAGTTACTATARGACTGTTA |
| | | CGTGTCCCCGCAAGCCCACCTCCGACGCCCGACAGG |
| | | ACTGACAGCAGGGTCCCGGAGAGAGGCACCTACGTC |
| | | CGCAAAGGTAAGTGCGGGAG |
| 255 | c2: 703,930 | GGATGTGGAGAGATCGGAAGCCGTCCGCCCCGGTG |
| | | GTGGGATTGCCAAACGGTATAGCTGTTCTAGAACACA |
| | | GCCCGCGGTCCCCGGAAAAGTTACTATARGACTGTTA |
| | | CGTGTCCCCGCAAGCCCACCTCCGACGCCCGACAGG |
| | | ACTGACAGCAGGGTCCCGGAGAGAGGCACCTACGTC |
| | | CGCAAAGGTAAGTGCGGGAG |
| 256 | E2: 64,720,639 | TAACACCTCTGAGCTGCATTTCCCTTCATTTGGGGCT |
| | | GAATGACGAGAGGTGCAGAATGTTCTTTCCAAGGTTT |
| | | TGGAGAGAATTCAGTGAGACAGTGGCRAACGGTGCC |
| | | CGATACAGTAAGTGCTCAATAAAATACTAAAGCGGAA |
| | | TCTAGTGGAAACTGCTCAACACCACCAGCGGTTTGGG |
| | | GAGCTAAGAAGGCAACA |
| 257 | D4: 812,589 | AAGTTCCCAGGATAGCTGCACACCAGGTACAGCGAG |
| | | AAGACTGGGTCAGATCAAGAGGCTCTGGGGAGACAG |
| | | TCTTCAGGGGCAGACAAGGATATACTGTCYGATGCAT |
| | | CTGAACCAATCAGACATGGTGACAGGCTTCTTCACCT |
| | | GATAAGAAGATTCAACTGGCAAGAAGCACACAGACAA |
| | | CCAAGTTAGCAAAGCAGA |
| 258 | B4: 147,961,464 | AGGGAGGAAATAAAGATGTTTGATTTATTACTGATAAC |
| | | CCCGAGGTTTGAGTGTGCACCCAAAGGGATGTGCTG |
| | | TGAATCTCCGCTTCTGAATGAGACACRCTCAACAGCC |
| | | AGGACACTGGTACAGCTGGCAAACCACAAGCTACCC |
| | | CTGTAGGAACAGGCGCCTTGCTGCATGGCGGAAAGC |
| | | TAACCGGAAACCCCCACT |
| 259 | B1: 156,143,186 | GTGTAGAGTGAGCTTGAGTACTTTGGCTTTTGTGTTTT |
| | | GTACTCAAACCCACGGTCCTCTGGTTTCAAATCTGTG |
| | | GTAGAGAACTACTCTTCCTTAGGTCRTTCTTGGATTCA |
| | | CGCCAACCCTTCTCTCTAGTCCTTACCCATTGTCTTGT |
| | | TCTCCCTTGATGATATCAGGAATCTTCTCCTATCTCAG |
| | | GGTCTGAGTGTT |
| 260 | F2: 77,518,182 | AAGCCTATGATAGAAACGAAGAGCCCATTCCAACCTA |
| | | ACAGGTACCCTCAAATCACCCTGGTGCAAGGGCAAAA |
| | | ACTCGGGAAGCAGGTCAGTCGTGGTTKGAAACCCATT |
| | | TCTGTTGCTTTCTAGCAATTGTTTTTTGAAACTTTCTG |
| | | CACCTCGGTTGCCTTACAGCGCTGAAGTGAGGGTCA |
| | | CACGATTGAGGGTCTA |
| 261 | A2: 17,611,273 | GTGAGCAAAAGTGGGTCAGGGTACAGACAGGCAGGG |
| | | GGGTTCCAAGCAAAGCAAAACCCACATGAAGGCTCA |
| | | GGGCTGTGCCTACCCCATCTGCACAATGYTAAAAATC |
| | | TCACCTAATGTATTTAAACATCCCTTTTGTCTAGACCA |
| | | TTCTCATATAGGTGTCAGGACGACCCCTAAAACAATC |
| | | AGAATGTATCACTATAT |
| 262 | B3: 107,303,663 | CTTGGAAAAATAGAATTTTACAGTTGGAAGGACTGTA |
| | | CAGTCTTCCAAATCACTCACTTTCATTACTTGTTCCAC |
| | | TGACAGCTTTCACTAAAAGATTACTMTGGAAAAACAG |
| | | CTTCCCTTCTCATCCCTGAAGTGAGTCAAATTGCCTTT |
| | | TCATGTATCTGTTCCAGAGGGCCATCTGCTCTGCCCC |
| | | CAGGAGAAGCTTCT |
| 263 | D2: 74,626,676 | GAATTTCTGCCCTTGTGTTCTGTGGCATCCCTCTCCTT |
| | | GAATTTCTGACTTTTCACTTCTTTTATATCATTTAATTC |
| | | TCATAACAGTCCTGGGAGGTAGGYGAGCAGAGATTAT |
| | | TAACGTTTTTGAAGATGAGCACACTGAGGCCACATTA |
| | | TTTAGCTTCTCTAAACGTTTCTCATCTGTTAAACGAGG |
| | | AGCAGGACAAGA |
| 264 | A3: 88,919,777 | GGTCCCAGTCTTACTTACTGTAAACCAGGAATAGCAC |
| | | TACCTGCACCTTTGCATTGCATTGCAAACAACAATGA |
| | | CTGACATTAAGAAAATCCTCAGTAAAYGTTGGCATTTT |
| | | TTGTTAAATTCTTGACCCTATCATTTACTAGCTAAGGG |
| | | AAGTCCGTGTAAGAGACTTCATTCTCTTCACACCACA |
| | | GTTCTCCTCTATGC |
| 265 | A1: 151,473,414 | AAAAGTGGAAATGTGTATTACAGAGGCAGTCCCAGGC |
| | | ATGGCAGGCTCTGACAGGGTGTTGGAGATGATGGGT |
| | | GGGCTTGAATACCCTGCCTGTGGGGGASGGGGGTGC |
| | | TGGGAGAAGCACAAGGACCCAGGGGAGATGGCACC |
| | | CTGCATGTCTGGGCCTGGGTGGGGGATTTAAGTGGT |
| | | CCACGTCCCTATCTAGGAAGC |
| 266 | B1: 178,757,633 | TTATCACTGGCCTCTTACTGTGGCCAGCCCCCACGAG |
| | | AACTCCAGTGAGACAGCAGGCAATAGCTCATGAAGTG |
| | | AAATCATTCAATGCCAAAAGGACTTCYGGGCCCCCGC |
| | | CATGTGCAGGGAGCCACTTACACCCCAGCCACACGG |
| | | AGGGAAGCAGGAGTGCTACACCGGTGGCAGAGAAGA |
| | | GCCACCCCCCCAGGCCGT |
| 267 | B4: 19,612,127 | TCCCTTGAGGGCACCCGCCTTAGATCACACCTTCTCT |
| | | CAAGGTGCACGTGACAGGGCAAATCTTTTGCTTCCAG |
| | | CCCAAGCTTGGTAGCTTAAGTGAATGMATTTAGTTTTA |
| | | TGTAGATTCTGGTCTCCTGACCAGAAATCACTAGGAA |
| | | GGAACAGGTTTGTCTAACATAGCTTGTAAGTGCCGGG |
| | | TCCCTGCTGGCCATT |
| 268 | E2: 11,112,283 | GGTCACAGACCCCAGGCCACCACCCACCTGGATGCC |
| | | GGAAGGCTGGGCACTCCTGGAATGGCCGGGTCCAG |
| | | CCTGGTTATTCCCTCGCCCGCAGCCAAGTYCATCCCT |
| | | CCCGGTGGGGTCCACACCATCTTTCTTCCAAACCCCA |
| | | CAGGTGCAAAGGGCCTCTTAGCAGCAACTACTTCCG |
| | | GGGAGGGAGCAGGTGACAGC |
| 269 | A1: 15,263,737 | TGATTCTTCAACAAACTCACAATCCACTTTAGTAATGG |
| | | AAGCAGCTCTACCTTTGGCAAACAAAAGCAGAAAAAG |
| | | TACAACCATGGCTGTGTAGAAGTCCYATATATCACTG |
| | | CATGCTTAAACTTTACTCAGCAAAACTTTTAATTCTTTG |
| | | GGGAAGGCAAGAGAGAAATAGTACCTGAAAACCAAG |
| | | TATTAGTATTCTCA |
| 270 | A3: 40,227,427 | TGGACTCTAGTTCTACCAATGGTTACTCTGAGATAATG |
| | | CTTATCCTATCTTATGCTGGGAGGAAGCTGGTCAAGT |
| | | AGGTATGAGCCTGTACAACTGCTGCRGGATCAGAGC |
| | | TGCCTCAGGCCTCTGGTTTTAGAGGCCTCGTGATTTC |
| | | CAAGGGAGGAAAAGGCCAGCATTGCTTGTCCTGTCA |
| | | GCCTCCCTCCTGGTTTC |
| 271 | C2: 150,072,397 | CATCCAGAAGTATTTAAAAACAATCTTTTTGCATGTCA |
| | | TGTTTATTTATTTTTGAGAGAGACAGAGCACAAGCAG |
| | | GGGAGGTGCAGAGAGAGACGGGGAGRCACAGGATT |
| | | CGAAGCAGGCTCCAGGCTCCGAGCTGTCAGCACAGA |
| | | GCCTGATGTGGGGCTGGAACTCAGACTGTGAGATCA |
| | | TGACCTGAGCCAAAGTCGA |
| 272 | B2: 43,290,061 | CCCAGGCATGAGGAGGGCAAGAGGGTAGGGCTGTA |
| | | GTTGTCAGTGGGGCGAGCCCTGTCCCCCTGAGCCCT |
| | | TGGTGGGGCTCTGACTCCCTAAAACTTTARAGGGAAG |
| | | ATAACCTACCCTCAAATAGTGAGTGTTTTTCGCCCTTC |
| | | CTCCTCAACTCTGAAACATTTGCAGTCAAGGGTAGTA |
| | | GGGGACCCTAACCACAGG |
| 273 | D1: 124,939,879 | ATCGGGCTCCACGCTGACAACGTGGAGCCTGCTTGG |
| | | GATTCTCTGTCTCCCTCTCTCTGCCTCTCTTTCTCTCT |
| | | CAAAAATAAATAAATAAACATTAAAGRAAGAAAAGAAA |
| | | AAGAAATGGATTTGAGGAAGTATATCAAGCAAACAAA |
| | | AACACGGGTTGGCGCAGGAGTAACAAAGTGGCAAAG |
| | | TGTCGCCTAAATAGCA |
| 274 | C1: 123,746,252 | GCTAAACATTCTACAACGTACACAACAAGGAGTAGTC |
| | | ATTCCTGGTCCAAAATATTGATTGTGTTGAGCCTGAG |
| | | AAACTCCTATTTAAATAACTGAGTTCYCTTTTCATTTAG |
| | | TACAAGTATTTCTCACACTATTGTACAATTCCTACAATT |
| | | AAAACTATACAATATTCCTGATCACCCTGCTAACTTCA |
| | | CCCATCTTTCT |
| 275 | F2: 75,210,562 | ACGCAGACAGAGTTACTGGGCCCCAAAGCCACAACC |
| | | CGGCTTCCCTTCCTTCTCCCTTTGCCTTCTGTCAAGTT |
| | | TTAATTCAATTAAATTAGGGAGAAACYGGGGCACCTG |
| | | GCCTAGCTCAGTCGGTCAAGGGTCCCGCTTCGGCTC |
| | | CGGTCACGATGTTACGGCGACATACACAGGGCTGGC |
| | | AAAGGTGAGCCTTCCCGC |
| 276 | A3: 14,410,638 | AATCATTAAAAATGATGAAATTGGAGAATACTTCATGA |
| | | CACAGGAAAATGCAAAATCATCTTAATTGAAAAAGCAT |
| | | TTCTTCAATATATATGTGTAGTTCRTAATCAGGGGAAA |
| | | AAACCAGTCCCATAAAATTCTCCTATTCCAAAAGAAAC |
| | | ATCACATCATAGAACATAGGGCCCTTCCCTCTTTTCCT |
| | | GAGAGCTTCAA |
| 277 | F1: 33,007,663 | CCATTCATTTCCTCTTCAGTGCAGGGAACCACCCAAT |
| | | CAGACAGTTGCACAATGAAAAGAAGGTCTGAGAATGC |
| | | CAAGGTGTGTCACTGGTTGTGGTCCCRCAGCCAGAA |
| | | AGGGCAAAGGTGGATTGCCAAACCAGGGCTACGTAA |
| | | CTTCAGCTGAGAAACCCTGCTGCCTTCAGGCCTGCAA |
| | | TTTCTCTAGACCTCAGTT |
| 278 | A1: 208,380,043 | TTTCAGCAAGCATTTACTAAGTGTCTGTGATGTAAGG |
| | | CTGGTTAGGTTCTGAGTGTAGGAAATAAACTGGACGT |
| | | GGCATCCTTAAGGAACTCATGATCCARGGTATCATTA |
| | | ATCCCCAAAGCAGTAAGAAAGAGGCCATTGCAGAATA |
| | | GGGGGTGGGGGTGGGCAACACCTGGAAGTTGAAGA |
| | | CCAGGTCAGGAAAGGTCA |
| 279 | E2: 35,480,527 | TTCCGACTCACATCATCCTCCACTGATGTCCTAATAGA |
| | | GGGCACTGTGCTTTGGTCATACATGAGTTTTGATCAA |
| | | GAGTTATATTTTCTAGTTAAAATGASAAACTGTAAAAC |
| | | TGGATATGAGGCCTTCAGCTGTATTTACTAATTAATCA |
| | | TACTGAGTTTTGATCCAGATGTGGGAGGAACTGAAAA |
| | | TTCCCCTGTGTAA |
| 280 | A3: 118,999,155 | ATAGTGGTAATAATATTATTAATCTTGTAGAATGCTTAA |
| | | GTAGATTGGTTTTTTTGTTTTCTTGTTTTTGTTTTTGTT |
| | | TTTGTTTTACATACATAAGCCTSTTAGCATAGTACCTG |
| | | GTATACATTTCTGTATTCAAGGAATCATGTCAATTGTC |
| | | TATTTATGTATAGTAACATAGACTCTGGGCCCATCCCT |
| | | TCTCTTTCA |
| 281 | A2: 10,913,767 | CTCTGCCACAGCACGGATGAGTCTTGAGGACGCGGT |
| | | GCCGAGTGCAGGAAGCCTCGGAAAGGTGCTTGCCAG |
| | | GGGCTGGAGGCGGGGGAGGTGGGGAGTGRCTAATC |
| | | AGCATCCCTCAAGTTTCGGCCAAGCAAGATGAATGAG |
| | | CTCTAGAGACGCGCTATATACAGCACTGTGCCTGGAG |
| | | TCGACGGTAATGCTTTGTGC |
| 282 | B2: 47,659,161 | ATTCCAGTGGCTGGTGTCATTATAGGAGGCCACGCG |
| | | AAGACACAGAGACAGAGAAGACCACCATGTGACCAC |
| | | GGAGGCAGCAGTTGGAGCTCCCTGCAACMAGTCAAG |
| | | GAACGTCAGAAACCACCAGAAGCTGGAAGAGACAGG |
| | | AAGGATTCTTAGAGCCTCCGGAGGGAGTGTGGCCCT |
| | | GCCAACATCTTGATGTCAGAA |
| 283 | D2: 89,706,040 | ATCCGTGTCACGTTCAGTCCTCATGACCGCTTTGGTC |
| | | TGCCCTCAGCCTCGCTCCCACCTTTGGGCTCTGAACA |
| | | CCCATCAGGAGGCTCTGCTCGGATGGYGTGAGTGTT |
| | | CCGAGATTTGAGGCATCATCAAAACGGTCAATTACAC |
| | | AAGTCTGGTAAGAACGCAGCTGTTTGCTTTACTTTCAA |
| | | AAGTCTTTATTAGGGG |
| 284 | Un12: 7,317,515 | TAGCCTTGACTCCTGGTTATTTTCACATAGGCTGCCT |
| | | GCATTTGATATTATCCTCAGAAAGTCTCGCTTTTACAT |
| | | TTTCGCACATAGATATCATCCCTTCYCATTAAAGTGCT |
| | | CTGATGACACTTCTGTGTTTGTTTATTAGAGCTCAGCA |
| | | GGAATTATGAGAGAAGTCGTTTTAAGAAAAGAAAAAA |
| | | AAAACAACCTTTT |
| 285 | Un2: 523,114 | CCGGAATGAGGCAAGCCTGGTACTGAGAGCAGATCC |
| | | CTGAAGCCTGGATGGGCAGAGCTTGGTGTAAACAAAT |
| | | TAAGTAGTGAAAGTCTGTGGAGCACTGRTTCTTATAG |
| | | GTGGATGGACAAATGTTTATGCTGGGAGGCTGGGGA |
| | | GGAAAATGCCACCTGACAGCTCCTTTGTTCCTGGAGG |
| | | GGTCTCCCAGTTATCTCT |
| 286 | B2: 156,308,475 | AGCGGCTGAATAATAGGTGTTTTTTTATGGATCATTGA |
| | | AGGTAGGGGCTGCTGATGCCAGGGTAGACCGAGGTT |
| | | TGTTTGAAGCCAATGTTCTGGAACATRTTTGGGATCT |
| | | GACTCTTCTGAGATGTGATCAGGTGTTTGGGAGCCTA |
| | | GGACAAAAACTCAGAGAATGATGAACTTTCTTGCTTC |
| | | CCTCTTAACAGTGGGA |
| 287 | A2: 25,685,296 | TGTTCTCTCTTCTGTCCACATATCGATCCAGGACTATG |
| | | GTAGGAGTCGACTCATTGCCTTCTCAAACAGGGGTGT |
| | | CACGGTCAGGATTTGGAATGACAGTYCAAGGGGCCT |
| | | CCAGCTTTGCCATTGCTGCAGGTTAGGTCGGGAAGC |
| | | TGCGGACTCTGTGACTGAGATTACCATTCAGGACATT |
| | | TAATAGGGGGTGATTTG |
| 288 | A2: 2,129,037 | TATCCGGGGTGTCTGGGAGAGGCGTCTTTGAAGAAG |
| | | TTTACATTGGAGATGAGATCTGGAGGATGCGAGGGT |
| | | GGGGAGAGGAACAGAGATCCTGAGGATAYGGATTTG |
| | | AGTGTGTGTGTGTGTGTGTTGGAGTATTCAATGGCTC |
| | | CCTATTGTCCTCACAATAACACCCAAACTCCTCCCTCT |
| | | TTCCAGGGACAGAGGGAC |
| 289 | A2: 161,801,210 | TTAATAAATTGAGTCAAATAATTCTCCCTCTCTTGTCT |
| | | GAGCCAGTGCTTTTCTGCTTGAGGAATGAGTAGCTTA |
| | | GATGATTGATAACAGAATCCATAACMTTTCCCCTCCA |
| | | AGTCACCAGCTTGAACCCAACCTAGTTGAGCAATGAG |
| | | AGACATTTGTTTCCCCAGGCAGCTTATGAGAGGTTTG |
| | | CATGAAATGAATGGG |
| 290 | D3: 7,290,581 | CCAACCACGATATAATTGCACTCATCCAAAAACAGAA |
| | | GTGACCGGGGCCACAGTAAATGTGCCTCTTTTGCAAC |
| | | TCTCATTCCTCTTAAATCTCAAATAARGATTAAAAATGT |
| | | GCATTTGAGGAGGGCCACCGTGGTGGCTCAGTCGGT |
| | | TAAGTATCTGACTCTTGATATCGACTCAGGTCATAATC |
| | | TCATGGTTTGTGAG |
| 291 | F1: 19,516,618 | AGCACAGCTGGGGATCTTCCATCCCGGTGCTGTTTCC |
| | | AGCAGCTCGGTGCGGAAGCACCACCTTTCTGGCTTG |
| | | TAAACTGAGAATACTGATCCAGCCCCCRTGAAGAGAC |
| | | ATTACCTAAGAAACCTCCCTTATGAAGCCTTCAAGGT |
| | | GAGAGTATTTTACAGGGAAATCCACAGGCTAAAAATA |
| | | AAAACACAACTATACCT |
| 292 | D2: 82,189,281 | AGGAAAAGGAGAACATTAACATGATTCTTGGAGGTTC |
| | | AACGGTGTTAAGTCCAACCCCTCACAGGACTCCACGC |
| | | ATCCCTTAGCAGGAGTTAAGGGAGAAYGGTAACCCTC |
| | | ACAGTAGGACAATCCCCCTGCAGACCTCCTCCATCAT |
| | | CAAGGTCAAGTGGGAACTCCAGAACTCTGAGTGTTTT |
| | | GGCAAGATGCTCCCTC |
| 293 | C1: 36,295,835 | GAGATGAATGACTGCCCTGAGTGCCAACAACAGAAA |
| | | GTGCATAGGATAAAGAGAGAGTTGACACAGGAACTCA |
| | | TACCCCTGAGGTAACATACCATACACCRAAAGTTAGC |
| | | AGTGGACAATAAGTAGCTTAGAGCCCTGAACTTGTCC |
| | | TGTCATATATCCAATGTCAGCACCACTCTGGAAACAC |
| | | AATCCCACATAATCCCC |
| 294 | D3: 33,258,191 | ACACCAAAGGACAAAGGCCAAGGGCAGGCTTACTGG |
| | | CTTGGATGAGTGATGGAGGGCTGCTCTTAGGGAACA |
| | | CGGGGCTGGGGCGAGGGCCGGTGACACARTGTGTG |
| | | ATCAGGCGGGGCTTTCCAGCAGCCTCAATGCTGAGG |
| | | GGGGCAGGAGGCCAAAGGCAGCGCCTTAAGAAGCA |
| | | GGCCAAACAGGGGCATCTGGGTG |
| 295 | C2: 63,676,887 | ACTAAATCATGTGCTTCTTCTATGAGGACTCCAAATGA |
| | | GTTCATAGCTTACTCATCACTAACATGAGCACCACACT |
| | | AGGTAGTTCGTATACTGTTTCATTYGACTCTAATGGCC |
| | | TTATAAAGTAGATGTAATCATTATCCCACTTTGCAGAG |
| | | GGAAAAAACAGAAACTTAGCTTAAGCAATTTGCCTGTT |
| | | TACATTACTAG |
| 296 | A3: 48,181,817 | CCCTCCCCCACTCTCGCTATACGTGTGTGTCTCTCTC |
| | | TCAAAAATAAACACTAAAAAAATGTTTTAAAAACACAA |
| | | CGTGGTTAATTCATGTGGAATTCCTYATGTTACAAGTT |
| | | ATTTGCAAAATTTTTTTCTTCTTCTTCTCATTCAGTTTT |
| | | ACCTGGGACTGGACGGAGCCAGACATTTGTGATCCA |
| | | AGCTTCTACTACA |
| 297 | A3: 11,904,341 | TTTCCCATGAGACTCATGCTCTAAAAGGAGATGCAGA |
| | | CCCAAGTGTGAGGAATGGAATGACGAAGACTGAAGC |
| | | ACCCTTTTTTTGAGAGAGAGAAAGGGCRGGGGGGGG |
| | | GTGTACTTTAAGCAGTTTCCATGCCCAGCACAGACCC |
| | | CAACGCGGGGTTCAATCCCCTGACCTTGAGATCATGA |
| | | CCTGAGACAAAACCAAGA |
| 298 | E3: 63,458,569 | GTTCTTTGCTGAGCATAAAGGATTGGCTATGGGGAAT |
| | | TTTCTTTTATCTTTGAAAATCTGTTGCGTATCTTTTAGA |
| | | AATAGTTTTTACCTGGTTTTCTCTMTTTTTGTTTAATTT |
| | | TTTTTTCTTTTGGGGGGAATTATATGGGGCGGAAGTT |
| | | TTATACCAAGAGGCACACAGCATATTCTATGTTAGCAT |
| | | TGACTCCTCTT |
| 299 | B2: 112,716,268 | TTGTTTCTATCTAATTTTTTATCTTCTTCCCTGATACTT |
| | | AATTTTTATTTGTCTTTTATCTCCAACTGTCTGTAATAG |
| | | TTACTTCCAAAAAAAAAATCAAWCATGTTTAAAACAGA |
| | | ATTCACAGCCCACTAACCCCAAATAACCCCAAAACAT |
| | | AGCCCTCAACTGTTTCATTAAGAAGATGTTCTCTGGA |
| | | GTCAAAAAGAA |
| 300 | B3: 149,673,110 | ACAGCGGGCTCTGGGGCTCGAGCTGGCTCCCTTGGT |
| | | CACACGCAGCTTTCCATGATGCTTCCAGTTCTCCAGA |
| | | ACTCTCCGATTGATCCTGCCCTCCCCAYGGGGCAGA |
| | | GCGTCCACTCCTGGTGACTCCTGGTCCCCTGTATCTG |
| | | TGCCAACGGGGGCCGGTGGCGGGGGGGGAAGCGG |
| | | CGTGGCTTGGCTGGAGGGGTA |
| 301 | C1: 125,311,520 | GGGTTTTGGGCTTGTCATGGGTAAACAAGGGAGGCA |
| | | TCTAAGGTGGTTCTGTGCAGTAAACCATTTCCAGGAA |
| | | CACAAAAGGGCGGGGGGAGTTTTTTAGRAAAAATAAT |
| | | TAATGTTTATTTTTCAGAGAGAGAGACAGAGAGACAG |
| | | AGGGTGAGTGGGGGAGGGGCAGAGAGAGAAGGAGA |
| | | TACAGATCTGAAGCAGGCT |
| 302 | B1: 19,312,704 | GACCCTGTGTGAGGTTTTAAGCCTGGTTTCTCTACTC |
| | | ACTAGTTGTGTGACCTTGAGAAACCAACTTAATCTCAA |
| | | CCGCATATGGTTGTAGAGAGAATTAYGTAAGATAATA |
| | | AAAAATTGAGAGCACTGCCGGGCATGTAAAAGCTCAA |
| | | TAATATTAAATGTTGTCATTGCTATTGTCATTAATACTG |
| | | GCCAGGATCCAGC |
| 303 | B2: 120,276,458 | GGGAATCAAGGAAAACCCAAAGTACTAAATGGATATT |
| | | ATAAATATAGGAGATGAATTTTTTTTAGTAGATATATAT |
| | | AGAGAGCTGATAAATGGCAAGGGTSTAAAAGAACCAA |
| | | TGAGTATAATAAAAAATATTTCTTAGCAAATAAAGTATT |
| | | TAGATGTTTGAACAGTGCTTTTCAATTTTTTATTGATTA |
| | | ATTTGACAAT |
| 304 | B2: 159,389,942 | AATGACCCCTTTCTATTTGACAGAATTCACATCAAAAG |
| | | CCAATGAGATGAGGCCAGGAGTTTGCTTTCCCTGTTG |
| | | TGAAGGTGGGAGGGGAACCAGCAGCRGTGATAAGTG |
| | | GCTGTGATCCCTCCAACCTTTGCCAAGGTGAACCTCC |
| | | ACCCACCCCCCTCACCGTGTCTCAGATAGAAATGCTT |
| | | GTTTCTGATGTTTTTC |
| 305 | D4: 39,362,745 | ACAAATTTCATTGCATTTGAGAAAAGCGCTGTGCTGG |
| | | GGAAGACCTTTTTGTTTTTGGAACACACTACTAGCATG |
| | | GTGAGCCTCACGGAGTCTTTGCTTAYGAACGTATAAA |
| | | TATGCTTGTAGGTCAATGGCATCATACCAGAATACAC |
| | | TGCAATAGAAGCACATCTTTCCTCGTATTAAAAGGATA |
| | | GGTATCTGTGCATA |
| 306 | B1: 172,534,764 | AAAATAATCAAAATCTGGGGCGCCTGGGTGGCGCAG |
| | | TCGGTTAAGCGTCCGACTTCAGCCAGGTCAGAATCTT |
| | | GAGGTCCGTGAGTTCGAGCCCCGCGTCRGGCTCTGG |
| | | GCTGATGGCTCAGAGCCTGGAGCCTGTTTCCGATTCT |
| | | GTGTCTCCCTCTCTCTCTGCCCCTCCCCCGTTCATGC |
| | | TCTGTCTCTCTCTGTCCC |
| 307 | POINTED1 | TTAGCCGATTGGAGGAGTACAATAGCCGTCAGGCTTT |
| | | ATGTGATGGAACTCCAGAGGGACCATTACTGCGCAAT |
| | | CCC[G/A]GAAACCATGACAAAGCCAGGACCCCAAGGC |
| | | TCCCCTCCTCTGCTGATGTGGAATTTTGCCTAAGTCT |
| | | GACACAATATGAATCGGGTTCCATGGATAAAGCTGCA |
| 308 | POINTED2 | ACACTGCTTGGAGGGTCTGAAATCTGGAAAGACATTG |
| | | ATTTTGCTCATGAAGCCCCTGGTTTCCTGCCTTGGCA |
| | | CAGACTCTTCTTGTTGCTGTGGGAACAAGAAATCCAG |
| | | AAGCTGACC[G/T]GGGATGAGAACTTCACTATTCCATA |
| | | TTGGGATTGGCGAGATGCTAAAAGCTGTGACA |
| 309 | ALBINO | TTAGCCGATTGGAGGAGTACAATAGCCGTCAGGCTTT |
| | | ATGTGATGGAACTCCAGAGGGACCATTACTGCGCAAT |
| | | CCCGGAAACCATGACAAAGCCAGGACCCCAAGGCTC |
| | | CC[C/*]TCCTCTGCTGATGTGGAATTTTGCCTAAGTCT |
| | | GACACAATATGAATCGGGTTCCATGGATAAAGCTGCA |
| 310 | CHOCOLATE | TGACCCTGCTATTCGAAGCCTTCACAATTTGGCTCAT |
| | | CTATTCCTGAATGGAACAGGGGGACAAACCCATTTAT |
| | | CTCCAAACGATCCTATTTTTGTCCTCCTGCACACTTTC |
| | | ACTGACGCAGTCTTTGATGAATGGCTGAGGAGATATA |
| | | ATGCTGGTGA[G/A]ACATTTCCTATGTTAACAAGATGT |
| | | CTTTGGCATATTTTAGATGTATCCACATTTCCATTGGA |
| | | AAATGCCCCTATTGGACATAATAGGCAATACAATATG |
| | | GTGCCATTCTGGCCTCCAGTTACCAACATAGAAATGT |
| | | TTGTTACTGCTCCAGACAAACTGGGATATACTTATGAA |
| | | GTTCAATGGCCAAGTGAGTATTGAAAATGTATCTTTTC |
| | | TGTGGAAATTACCAAAACTACATTTGCTACCTTTTAAG |
| | | GTAATGACAG |
| 311 | CINNAMON | CAGGTGTGAGGCAGTGACTGCAGACTCACGACCCCA |
| | | CAGCCTCCATTACCCGCATGATGGCAGAGATGATCG |
| | | GGAGGCTTGGCCCACGAGGTTCTTCAACAGGACATG |
| | | C[C/T]GATGCAATGGCAATTTCTCAGGACACAACTGTG |
| | | GGACTTGCCGTCCTGGATGGAAAGGAGCTGCTTGTG |
| | | ACCAGAGAGTTCTCATAGGTAAGTGGGGATCTGCATG |
| | | TACATACAGTTCTTCATGAGACTCTATGCATTTAATAG |
| | | GAACCTAAATCATTTGAACTGGAAGCACATCTGAAAAT |
| | | CATACAAC |
| 312 | Mucopolysaccharidosis | GCTGTGGCTGTTGGTTTCCTCCGCCGTCTCCATACAA |
| | Type VI | CGATTCTGCGATACCCTCATCAGACCCACCGACCAAG |
| | | ACCCTCTGGC[T/C]CTTTGATATTGATCAGGACCCAGA |
| | | AGAAAGACATGACCTGTCAAGAGACTATCCCCATATT |
| | | GTCGAGCAGCTCCTTTCCCGCCTCCAGTTCTACCACA |
| | | AACATTCAGTGCCTGTGCATTTCCCGGCACAGGACCC |
| | | CCGCTGTGACCCCAAG |
| 313 | Mucopolysaccharidosis | CATGACCTGTCAAGAGACTATCCCCATATTGTCGAGC |
| | Type VI MILD | AGCTCCTTTCCCGCCTCCAGTTCTACCACAAACATTC |
| | | AGTGCCTGTGCATTTCCCGGCACAG[G/A]ACCCCCGC |
| | | TGTGACCCCAAGGGCACTGGGGCCTGGGGCCCTTG |
| | | GGTATAG |
| 314 | Polycystic Kidney | TTCTTCCTGGTCAACGACTGGCTGTCGGTGGAGACTG |
| | Disease | AGGCCAATGGCGGCCTCGTGGAGAAGGAGGTGCTG |
| | | GCAGCAAGTAAGGGCCTGGGCCCGTCCCTGCCCGG |
| | | GCTGGCCGAGGGGTGGCCTGTGCCACTGGCCTCCT |
| | | GAAGCCAGCTGTGCCCTTTCTGCAGGCGACGCGGCT |
| | | GTGCGGCGGTTCCGGCGCCTCCTGGTGGCCGAGCT |
| | | GCAGCGTGGCTTTTTTGACAAGCATCTCTGGCTCTCC |
| | | CTCTGGGACCGGCCTCCTCGGAGCCGCTTCACCCGC |
| | | GTCCAGCGGGCCACCTGTTG[C/A]GTCCTCCTCGTCT |
| | | GCCTCTTCCTGGGCGCCAATGCTGTGTGGTACGGGG |
| | | TCGTGGGAGACGCCGCCTACAGGTGGGTGCCCGAG |
| | | GGGGGCCCGATGATCTCCTCCTGCCCGACCCCTCCT |
| | | ACCCCCCACAGCCTCTCCCAGCCCGGGTCTCTCTCC |
| | | TCTCCTGCCACACAGCGCGGGGCCCGTGTCCGGTCT |
| | | GATCCCGCTGAGTGCCGACACAGTTGCCGTCGGCCT |
| | | GGTGTCCAGTGTGGTCGTCTATCCCGTCTACCTG |
| 315 | Hypertrophic | CTCAGCCTTCAGCAAGAAGCCAAGGTCAGTGGAAGT |
| | cardiomyopathy MC | GGCAGCCAGCAGCTCTGCTGTGTTCGAG[G/C]CCGA |
| | | GACAGAGCGGTCAGGAGTAAAGGTGCGCTGGCAGC |
| | | GGGGGGGCAGTGACATCAGCGCCAGTGACAAGTATG |
| | | GCCTAGCAGCCGAGGGCACGAGGCACACTCTGACAG |
| | | TGCGGGACGTGGGCCCCGCCGACCAGGGACCCTAC |
| | | GCAGTCATCGCTGGCTCCTCCAAGGTCAAGTTTGACC |
| | | TCAAGGTCATAGAAG |
| 316 | Hypertrophic | GGCTACATCCTGGAGCGCAAGAAGAAGAAGAGCTTC |
| | cardiomyopathy RG | CGGTGGATG[C/T]GGCTGAACTTTGACCTGCTGCAGG |
| | | AGCTGAGCCACGAGGCACGGCGCATGATTGAGGGC |
| | | GTGGTGTATGAGATGCGAGTCTACGCGGTCAATGCC |
| | | ATCGGCATGTCCAGGCCCAGCCCTGCCTCCCAGCCC |
| | | TTC |
| 382 | MLPH DILUTION | atggggaaaaaactggatctttccaagctcacggacgacgaggccaagcaatctggg |
| | | aagtggttcagcgggactttgatctgagaaggaaagaagaggaaaggctggggggat |
| | | tgaaggacaggattaagaaagagagctcccagagggagctgctctcggatgcggccc |
| | | acctgaatgagacccactgcgcccgctgcctgcagccctaccggctcctcgtggccccc |
| | | aagaggcaatgcctggactgtcacctcttcacctgccaagactgtagccacgcccaccc |
| | | ggaggaggagggctggctctgtgacccctgccacctggccagggttgtgaagatgggc |
| | | tcactggagtggtactacgggcacctgagagcccgcttcaagcggtttgggagcgcca |
| | | aagtgatccggtccctgtgcgggcggctgcagggtggaggtgggcctgagccaagcc |
| | | ctggagagggaagtggagacagtgagcagacagaagaggatggagaactggaca |
| | | cagtggcccaggcccaaccccttgggagcaaaaaaaagcgcctctccattcacggctt |
| | | ggactttgatgcagactctgatggctcgactcagtccggcggtcaccccccatatctgtcc |
| | | ccggtccccatggccacagacagcctgcaggccctcacaggtgaatcccgtgccaag |
| | | gacacctcccaggaggccgtggtcctggaagaggctgatgtcggtgcccctggactcc |
| | | accctcatccagaagagcagacagacagcctctcagctgccagacaggacaccctca |
| | | ctgagccccgcttccccagacagtcctgcacaacagccctggggttggctgtcacaccc |
| | | ggtccaggcgtcatcagcagtagtgagcggctctcctcccggtacccggctgacgaag |
| | | gcacctccgatgacgaggacaccggggctgacggtgtggcctcccagagcctcacgt |
| | | ggagggactgcgccccggctgagagccagcatctcaccggccaccagcccacagac |
| | | gccgacagagaagaagagaccctaaagaggaagctggaggagatgaccagccac |
| | | atcagtgaccagggggcctcgtccgaggaggaggggagcaaggaggaagaggca |
| | | ggactgaacaggaaaacctccatcgaggacctccccggggcagccccagaggtgct |
| | | cgtggcttcgggccaaacgtccagacaggaaacaagtccccggggtcctcaggaact |
| | | catgcagcccggcagaaccacggaccaggagctgctggagctggaagacagagtg |
| | | gccgtgacggcctctgaggttcagcaggtggagagtgaggtttctaacatcaagtccaa |
| | | gattgccgccttgcaggctgccgggctctcggtgagaccctcgggaaagccccagcgg |
| | | aggtccaacctcccgatatttcttccccgactcgttgggagattgggccagacccctaag |
| | | gatccaaacgcagagccttcggatgaggtcaaggtgatgactgcaccctaccttctgag |
| | | aaggaagttcagtaatcccccaaaaagtcaagataaggctggcgactcctttgaccgg |
| | | cagtcagcgtaccgcggatccctgacgcagagaaaccccaacagcaggaagggagt |
| | | ggccaaccacagctttgcaaaacccgtgatgacccagcggccctga |

In further embodiments, the present invention may be used to identify characteristics associated with cattle, multi-breed and the like. For example, the following tables demonstrate sequences that may be used determined genetic characteristics, such as parentage, identity, sex, genotype and/or phenotype and breed determination in cattle and the like. Thus, in further embodiments, the present invention provides a panel comprising a plurality of assay compositions, wherein each assay composition is capable of identifying at least one of the nucleotide markers as set forth in Tables 9-11 provided below. Further information for sequences provided herein may be identified by searching appropriate genetic databases. Table 9 provides allele variations between allele 1 and allele 2 to assist those skilled in the present art and the approximate location in centiMorgans of the centromere as used by those skilled in the present art.

TABLE 9
CATTLE AND MULTI-BREED SNP PANEL SEQUENCES
T

| | | | Approx. | | | |
|---|---|---|---|---|---|---|
| SEQ ID | SNP ID* | Chr | location^{%} | Allele 1 | Allele 2 | Description |
| 317 | MBS042-1 | 2 | 28.6 | G | A | SNP Marker in many breeds |
| 318 | MBS029-1 | 2 | 107.3 | A | G | SNP Marker in many breeds |
| 319 | MBS048-1 | 4 | 80.7 | G | C | SNP Marker in many breeds |
| 320 | MBS007-1 | 5 | 41 | C | A | SNP Marker in many breeds |
| 321 | MBS030-1 | 5 | 108.3 | G | A | SNP Marker in many breeds |
| 322 | MSB043-1 | 5 | 129.2 | G | T | SNP Marker in many breeds |
| 323 | AH2-5 | 6 | 83.5 | T | C | SNP Marker in many breeds |
| 324 | MBS044-1 | 7 | 28.2 | C | T | SNP Marker in many breeds |
| 325 | MBS014-1 | 8 | 73.6 | T | C | SNP Marker in many breeds |
| 326 | MBS031-1 | 10 | 33.5 | C | T | SNP Marker in many breeds |
| 327 | AH8-4 | 11 | 56.6 | G | A | SNP Marker in many breeds |
| 328 | MBS015-1 | 11 | 130.5 | T | C | SNP Marker in many breeds |
| 329 | AH25-1 | 13 | 50.7 | G | A | SNP Marker in many breeds |
| 330 | MBS046-1 | 13 | 94.6 | T | C | SNP Marker in many breeds |
| 331 | MBS047-1 | 16 | 78.9 | G | T | SNP Marker in many breeds |
| 332 | MBS018-1 | 17 | 78 | G | T | SNP Marker in many breeds |
| 333 | MBS020-1 | 17 | 106.4 | G | A | SNP Marker in many breeds |
| 334 | MBS033-1 | 18 | 55 | C | T | SNP Marker in many breeds |
| 335 | MBS021-1 | 18 | 62 | T | G | SNP Marker in many breeds |
| 336 | AH13-4 | 19 | 34 | A | G | SNP Marker in many breeds |
| 337 | MBS034-1 | 19 | 45.3 | T | C | SNP Marker in many breeds |
| 338 | MBS054-1 | 19 | 67.8 | C | A | SNP Marker in many breeds |
| 339 | MBS049-1 | 21 | 38.7 | C | G | SNP Marker in many breeds |
| 340 | MBS025-1 | 23 | 8 | C | T | SNP Marker in many breeds |
| 341 | MBS039-1 | 23 | 11.5 | A | T | SNP Marker in many breeds |
| 342 | MBS035-1 | 23 | 37.5 | A | G | SNP Marker in many breeds |
| 343 | MBS028-1 | 24 | 69.8 | G | A | SNP Marker in many breeds |
| 344 | MBS040-1 | 25 | 16.1 | T | C | SNP Marker in many breeds |
| 345 | MBS051-1 | 25 | 56.8 | T | C | SNP Marker in many breeds |
| 346 | MBS041-1 | 29 | 56 | C | T | SNP Marker in many breeds |
| 347 | 421_10 | 1 | | C | G | SNP Marker in many breeds |
| 348 | 423_24 | 10 | | G | A | SNP Marker in many breeds |
| 349 | 425_2 | 9 | | A | G | SNP Marker in many breeds |
| 350 | 431_a2 | 5 | | G | A | SNP Marker in many breeds |
| 351 | 487_67 | 14 | | G | A | SNP Marker in many breeds |
| 352 | 448_67 | 2 | | T | C | SNP Marker in many breeds |
| 353 | 16_2 | 7 | | G | A | SNP Marker in many breeds |
| 354 | 417_16 | 4 | | G | A | SNP Marker in many breeds |
| 355 | 486_67 | 3 | | C | T | SNP Marker in many breeds |
| 356 | 436_C10 | 4 | | C | T | SNP Marker in many breeds |
| 357 | 454_g11 | 17 | | C | G | SNP Marker in many breeds |
| 358 | 013.SP3 | 6 | | T | C | SNP Marker in many breeds |
| 359 | 018.SP6 | 3 | | C | T | SNP Marker in many breeds |

*As designated by from Heaton et al (2002) Selection and use of SNP markers for animal identification and Paternity analysis in U.S. beef cattle
^{%}Location in centiMorgans from centromere

TABLE 10
CATTLE DISEASES AND TRAITS

| SEQ ID | SNP | DISEASE/TRAIT | Allele 1 | Allele 2 | BREED |
|---|---|---|---|---|---|
| 360 | BLAD | (BLAD) Bovein Lymphocyte | A | G | Holstein |
| | | Adhesion Deficiency | | | |
| 361 | DUMPS | (DUMPS)—Def. of Uridine | C | T | Holstein |
| | | Monophosphate Synthetase | | | |
| 362 | CHONDR | Dwarfism/chondrodysplasia | * | GGCA | Dexter |
| 363 | PROTO | Protoporphyria | G | T | Limousin |
| 364 | HYPOTR | Hypotrichosis | C | T | Red Angus/Charolais |
| 365 | SYNDAC | Syndactyly | CG | AT | Holstein Angus |
| 366 | CITRUL | Citrullinemia | C | T | Holstein-Freisian |
| 367 | CVM | Complex Vertebral | G | T | Holstein |
| | | Malformation | | | |
| | | COLOR | | | |
| 368 | E+ | Black coat color | T | C | many |
| 369 | RED | Red coat color | G | * | many |
| 370 | DUN | Dun coat color | C | T | Dexter |
| 371 | ALBINO | Albinism | * | C | Braunvich, Brown |
| | | | | | Swiss |
| 372 | ROAN | Roan coat color | C | A | Shorthair, Belgian |
| | | | | | Blue |
| | | QTL | | | |
| 373 | Ucalp | u-calpain SNP | G | C | many |
| 374 | CALPA | calpastatin SNP | G | C | many |
| 375 | MYOS | myostatin increased muscling | C | A | many |
| | | mass | | | |
| 376 | ABCG2 | ABCG2 | A | C | Holstein |
| 377 | Kcasein | kappa-casein | A | C | Holstein |
| 378 | zfxy1 | Zinc Fingers X + Y | T | C | Many |
| 379 | zfxy2 | Zinc Fingers X + Y 2 | T | A | Many |
| 380 | GHR | GHR gene | A | G | Angus, Charolais |
| 381 | Bcasein | Beta casein | C | A | Many |

TABLE 11
CATTLE SNP PANEL NUCLEOTIDE MARKER SEQUENCES

| | | |
|---|---|---|
| SEQ ID | SNP ID | SEQUENCE |

| | | |
|---|---|---|
| 317 | MBS042-1 | Ctggagtgcgtttcaaaatggaacagataaaaaactagtaagtacataagtacatatctactgg |
| | | cctttgatctgactagttccccagtctcaggtct[g/a]tttgctgttaatcaccagtgagagaaggtc |
| | | ctaccctatct |
| 318 | MBS029-1 | Tctgggagaggtacacggggtgggggaggggcgagtggctgcctcgggaggcacgggaga |
| | | ggtgaaaagcagctgagggatcacggatgctttgaacNgggctgcaa[a/g]tagttgatacga |
| | | agtcaccgtgattgctttcgaccggtatgatttgtacaaaccagctcaacccttg |
| 319 | MBS048-1 | aaccgtgacggcatcatctgcaagtcggaccttagagagacctactcccagctcggtgagggc |
| | | acccgtctcctgcctggcccagcccctctcacaccag[g/c]gacccggctcagagctgctgcct |
| | | gNcccNgcctgtactcctgtcctggctcacaccacccacccacccctagaacacattccttccN |
| | | cttcacttcccccacccagtga |
| 320 | MBS007-1 | ggaagaggggcaagggagagctaaaggcctNgacgggatcagtgagagaccagccagct |
| | | gagtgacttag[c/a]aggggaggatggagccacctccaggagagttggctNgaaaggatttct |
| | | tctcNgcccatcgatttcctgcctcactcct |
| 321 | MBS030-1 | Tcttgaaaggttgctctgccacctgctgcttaaccttctcagcccctgtggtgtttccaaagggctgg |
| | | tcac[g/a]gtcctcaggcttgggtgtggcctgggtcttggagagggatggtgctgcgggcaagtc |
| | | tgtgtccatg |
| 322 | MSB043-1 | gagaggggaggggaggggagggttgccctcctacacctggccccacctgtacctccttcNgt |
| | | [g/t]agcctttgttctagctagaagggcccctgaattctccaggtaacccctgagagggaaggaa |
| | | atgcct |
| 323 | AH2-5 | tgtcagataaaatacaggatgtccagttatatttgaatattaagtaaaacaatgaatattttttcagtg |
| | | t[t/c]gcatgtacattgttccatgtaggacatgcttatattttagaaattattcattgtatactgtaaattc |
| | | caatcga |
| 324 | MBS044-1 | Cggtccatgccatgttactgtctgtaaccctgtgggcactgctagaacctcacttctgaccataact |
| | | gaagcccaggg[C/T]gatgagaggtgatggagctctgactattaggccgcccagctctggtct |
| | | gggttcttaaccacttctcaaga |
| 325 | MBS014-1 | gcagactcagcagccactaacaaggggctgcgagccatcaaaggggtccgtggaaaagact |
| | | gtagagagca[t/c]gggaaaggagccactatgcaggaagtcacaggaagctttgcagaaaa |
| | | actaacatttgagctggctccag |
| 326 | MBS031-1 | gtgccactccaaggtggtgaagaacaatctgaaccccagctgggagccgttccgtctgtccctg |
| | | cactccctgtg[c/t]agctgtgaNgtccaccggcctctcaaggtgaggccccacgagtaaggg |
| | | cagcctggtagagcagccagcctctg |
| 327 | AH8-4 | gacttcagaatggaaaccctctctccctaaagaaagccatacccagggagtccacNtgggctg |
| | | aataacccc[g/a]aggactggcagaagggaagggaagaatgtagctgcagcctgaacttca |
| | | ctgttgtctNatccatgcccNactgcctt |
| 328 | MBS015-1 | gtcagagcccaggctggtccgaggccgcacccgctggcctccctgccccgtgagagggggag |
| | | gcaggaacatcccatc[t/c]ggaagtagccgctcttccaagtctggaatcaggaggagctcagt |
| | | aaatgctggttgaatgaatgaatgaat |
| 329 | AH25-1 | tgacctggtcactttctatgtggcttcctgtattccctttgttgtctaatgtcagaaactataactatcta |
| | | [g/a]ttcacactaggttctctataaattatttgctgaacaaaatatttcttcttttgaaaataagagaaa |
| | | catagagtttac |
| 330 | MBS046-1 | ctgggagtggagagtggattgggaagcctggggaactgtggacctgtgggcaatcccttagc[t/ |
| | | c]tttctgagcctcagtttctccatctgtacaaaaggggcaatcataccNatttcacagtcaggtga |
| | | actgtgcag |
| 331 | MBS047-1 | gccgtctacacatgcatatctgaaaggaatgaccctcctaggcagaggaggagaaagaatcat |
| | | ttaaatgtctttacagtaatgctc[g/t]aaaatttttggctggccagcatggctgctttattacatctctc |
| | | caaagagcacaNtacctgccatcaagtgcagatacgcat |
| 332 | MBS018-1 | atgcttgttctcgcttgtgcagaaaacattgttccagattcaatcgactgggttcatgtcccctcacat |
| | | agtttttaaggttatttatttaaa[g/t]gtctaatgtattttattgtaacagacattgttttgccaacattgc |
| | | ctatttca |
| 333 | MBS020-1 | gcaggccttcgagtccatgctgcgctaNatctactatggcgaggtcaacatgccgcccgaggac |
| | | tccctgcatcctagcctcagggcttcc[g/a]gccacgcccggcagccttggaccccgccagcc |
| | | aggggctgggcNgtttcccaaggattcNtggtggccccgtctcccctgagctctct |
| 334 | MBS033-1 | gagcgcctgatggaagagggtctgggggcctgggttcttgggtccagaggatgaagtggcagg |
| | | ggacgccgattctt[c/t]ggtcccaagagaggaagggcctgagtcttgctgaaggaggagact |
| | | gggacctcaatttctgggtcccaaaagagaaatgttg |
| 335 | MBS021-1 | tgtcttccactttctactgcgattNgtcacttcttcatccatgggagggagaggagagctcttctcag |
| | | attgcctgatttcc[t/g]attcctttcatcctcagccggctcttcccagacaaggagagcatgcttga |
| | | tgcggctttcaccttggcagctgagatttccagcaag |
| 336 | AH13-4 | ctttctgacttgaaaccatttNgaggagacaggggggatctttaagaggtaacttcagtcttcgag |
| | | gttagggtccccactttgtagaggggatgagaa[a/g]ttggttttgcagctgatgggtccagtgt |
| 337 | MBS034-1 | cctactcccagtccaagcagtttttactggactcaggtgagacccagagctgagccctcagctcc |
| | | cagctagtctggaccagcttcgagctgattgccacc[t/c]cctgctccacctcccccaggctactt |
| | | ctctgacaacatcc |
| 338 | MBS054-1 | tccgtgcctgccctcagcctgcccagcggggaagctctggtgggtgtggaatggtggtggcaga |
| | | aagggt[c/a]ccgcgggtctcccattNgtcttcccctgagtccctgcccagccgggactgcctgg |
| | | atctgagaggtgggacaaggaggtggcttNgccgcaggtcaccg |
| 339 | MBS049-1 | ttctcatacaaaaagactttgttctgacagctgctcactgcagaggaaggtgaggggcagtagcc |
| | | agcctaccctacctcaggcct[c/g]gacagggacccgtcctctccccaggagctgagcccagg |
| | | ggtacccctccttcaccccaggatcctggaaatcaggaccacaagagctcatcag |
| 340 | MBS025-1 | aagcagaggaggtccagagaagcctctgccccacccctcaggcctctgttggctccctggccc |
| | | cactctcaaaatctgagacttgta[c/t]tcaacccttttctttcccaggaggttctagatctcctcaga |
| | | ttccttcaacagcctcttcctgtaggaatgacccctcctgc |
| 341 | MBS039-1 | ccccagcacagccccttgccaaggtatacatctatggatatctttggggtaatgaggaggaaaa |
| | | ggtcttaaatggggtgg[a/t]tccagagtaaaaggcacctgtcttttctccacagaaaaaggggg |
| | | taaagcggaaagcagatactaccacccctacacc |
| 342 | MBS035-1 | ttgaacacttactatatacttggcattgttctaggttctgtgaatacagttNtgaacaaaacagagca |
| | | aaaatccttgccttcatggg[a/g]tagggaatggggagacagacaatatacataataaataact |
| | | aaatgtattttttgtgttaagaggtaataaatacagtggaa |
| 343 | MBS028-1 | ttctacccacctaaatgagacattccttttatgtctagccctcactcccagtgtaaacctgttgtgtttcc |
| | | ctgtattttcctgca[g/a]tgttgtcacagaggaagtggggtagtgtagttctgccttgatcacgctN |
| | | tctctrtcttagctgttrtttaaaattttattgt |
| 344 | MBS040-1 | aatcagttgacccttaaagacacttgtatgtatttcagaagttttctcatgccaagctaagagcagttt |
| | | cacgtagtata[t/c]ttcagggtcaggaaggcNgcccataaagggcattgtttgtgatcctttgag |
| | | gacgttgaggtgctgt |
| 345 | MBS051-1 | tggagcagcaccccagctgccggtacgatgctctggagatctttgccgggtctggaacctcggg |
| | | ccagcaactcggacgcttctg[t/c]gggaccttccgacccgcgcctgtagtcgcctccggcaac |
| | | caggtgaccctgaggatgagatcggacgaggg |
| 346 | MBS041-1 | gccttgtcagtgggtggggtgNggttgttgatggcagcggtggtgaagtgataacaaacagcca |
| | | tctactgcctttttctcctggtaac[c/t]gttagcatttcctcttgtcctcactagacttccctgg |
| 347 | 421_10 | cttcttttaatttccaggtctcagtctgggagcccNgggaactgtgcccctgtttctgact[C/G]ttgt |
| | | tgccttgagggttttggaggttgacatggtcagcaacccgaagaagcacctcagatggtctgtga |
| | | gtttcagacacagtgataatcacagctggcccacagccagaggaggaggcatcctactaacaa |
| | | gagcagtggatgctttggttatgcaaatgta |
| 348 | 423_24 | tgaagtgggagtagggtgccggcaaggcaaggcatagagcatgggaaaggaggcagggttt |
| | | ggaaagcggaggcaggctggacagagacaagggttagtctctggaatgttgacatatgtggag |
| | | cccaggggaggagggcctcatgtgccatgctaggc[G/A]atacaagattctcttcaagtctgg |
| | | aggaactgctcaatgcagggggcttagagggccctacctgggtccttttctcatctctgccctgca |
| | | agctNcccatgtgttctttaccccaaggtgtcggccatcacccatcccatggctatcaatctttcttcc |
| | | tggctttgag |
| 349 | 425_2 | gagagcaccctctcatccctccttcagcttgagcagaccatcatcttccagtctctccagggcaag |
| | | acaatctgggaattaccctttacaagagaaaagcttaaaatattatgaaacttaaaataattagtg |
| | | acactttctaaaaatgtaataaatctgcaaagactattttaactctagagagattctttaatttacaatt |
| | | tgttgagcttcataatcctattagaagtctgcccacaaaatactcaaaaatcccattcaaagcaga |
| | | acagagaactgttcaacatctttccaacaggacagaagtcaacctgggaacagctgttctctctg |
| | | cggctcaggtagtga[A/G]atgcacaggacctgtcctgggtactgtccctgtgagtattcaagct |
| | | ggactcattagctgcagttactttgctgccagtcatgattccatacatacacacacacacatgcac |
| | | gcacatgcaca |
| 350 | 431_a2 | TatctgtgcactcatcatgttcataNaaggattatttataatagacaaaattgtagaagaaaccca |
| | | aaatgtccattgacgtttggaataaaaaatgcatgattggattcacaggtgaatgaataaatgaa |
| | | aagtagtttatacatatgatggagtattattagccttaaaaaaaatctgacacgtgtgacaacatgg |
| | | atgaatcttgaggacattatattaagtgaa[G/A]taagccaatcgtaaaagaaaaatgctatatg |
| | | gtttcacttatatgaggtatccagaacagtcaaactcatagaaacagaaagtataatggcagttg |
| | | ccaggggctggtggggaggtaaagtgaggagttaatgggactaaaatttcagtttttcaagataa |
| | | aaaagttctgaagattagatgcacaaaatccgagtgcacttaacactgctgaatattcacttaaac |
| | | ggtaataaagtttatgccatgtatcctttgcg |
| 351 | 487_67 | aaaattgtgagtaaacataaaaggcaaaaagttatgacactgaaagatgagccacccaagtc |
| | | agaaggtgtccagtatgctgctggggaagagtggaagacaactctaatgtggctgggccaaag |
| | | cagaaataacattcagttgtggatgt[G/A]tctggtggtgaaagtaaaatctgatgctataagca |
| | | aaatgaactgagaactacctctactttagaactacctctagaaacatctacctgtttcattgacaact |
| | | ctaaagactttgacaatgtggatcatgccaagctgtgaaaaactcttaaagagatgggaatacc |
| | | agNccatcttacctgtctcctgagaaacctgtatgcaagacaagttgcaacagttgtatagaaca |
| | | actgtatagaacaactgactggttcagaattgag |
| 352 | 448_67 | ttctatagacatagacttagaagagaggacatttgtttccctcctaggttcttaaaggaaaatagctt |
| | | tcaaaa[T/C]ttaatttttattatgtttttgcaaacttgctaaacctagagaaaagcaatgttgttagg |
| | | gggaggggaagtaagtttacataatacttataaatatttccttgatatctataaatatttgctca |
| 353 | 16_2 | caagctttaagagccactctctgccccctcataatctggtctcccccaccacccagacctgtctcc |
| | | gccgggccgctcagttcccctcctcctacagactcactgaac[G/A]tgctgcctgactccctggt |
| | | gtttccccccaccccccacagtactgtgcagccctggactccctgatcagcatctccaactgcagt |
| | | gtcatccaaaggaccaagaggatgctgaatgcactctgtcctcacaagccctcagctaaggtaa |
| | | ggcccccctgtcctttgacctggcacccacttctgccagccctgggctcaccctggga |
| 354 | 417_16 | actttggggtNaaacttccagaataaatatttgcaggggaagggtcactcagcccagcccctcc |
| | | actgcctgggcatctgcattcttggaaggaaggaggtgatgtagggaggagggagagtgagga |
| | | tgccacgtggagccNagctgcatttcac[G/A]tcacagttgatgtcaccacttggcagatgtccc |
| | | agggcatggggggaatggtagactgtcaaagcaggtgtgaaacactgaactaaaacagacat |
| | | ttagcaaatcaaacaaggagaaagtggttaagaNccagctgaagacactggcaaggcaagtt |
| | | ctgatgctggc |
| 355 | 486_67 | ttactattatgcatttctgatcaccgacctaaactggggtttcaactcttattagcatttttagtatgatctt |
| | | acttgtttctgttgNgacctcattctcctacttactgtaaaactaNNtctatcttctctattg[C/T]tca |
| | | gagtcttcattgaactaaactggtaaacaaaccacacagatgtgaaaataaatttctaatctatta |
| | | agttaaattttgctatataaaaacacttgttaaaatttaaaaaatagaactaccattt |
| 356 | 436_C10 | aactgtagaatctatatgcttagcttgtttagtggttcacagggtaatcaatagtaaaagtgggttaa |
| | | attcaatgtaatccagacaactgcaaggtatgtatatatattatacata[C/T]ataatttaaatgttg |
| | | atcataaaactcacaaagaaatttatattaaaaaatagaatgctatcatgcattattttatttagacct |
| | | tttagtcaccaagaggaggtaatttttccttagtctgtaaaattcaggaaattgaccatttaaag |
| 357 | 454_g11 | cagaNagttataaatgctaatttaagagtataatgccatccaa[C/G]tgcctaattgagaacaat |
| | | gtaagaataaatttgaggaaaataaatacttgcaaactgtgctaattatatatttgatcagaaaatt |
| | | cacatagaaaatattttaaattggtgtttttccacagtggatg |
| 358 | 013.SP3 | gattggagtgttgcatgtataagccagggaacaccagcaattgtcagcaacaccagaagctaa |
| | | gagaaagacatggaacagattct[T/C]tcctggacccttcagggaaagcatgatcttgttaaca |
| | | ttttgaactaatagcNtccagaactgtgagaaactgggagtttctattgtttaaagtgaaagtgaag |
| | | ttgctcagtcgtatccaactcttt |
| 359 | 018.SP6 | accacgctcaaagctcaggtcctgagaatatgaccctccccaccaggccccagattctgcagc |
| | | caatgtgaccttgattcttctgggaactactcaaaggcccaaggctc[C/T]tctacccaagggtt |
| | | gctgaaaatccatcaagagcccagcaagagggagaggcagggtgtgggtctgagctccaac |
| | | ccacaggcaacaagtcttttNgggggagagaggg |
| 360 | BLAD | Ctatgtcagaacgtgtgcttgcctgaaatggaatctgaataggcatcctgcatcatatccaccagc |
| | | ataagagaatggggagagtcctgaggttctgaggcctgacaagatgccataagtgcccatgaa |
| | | ccccccccacccccagaccagatagtacaccctgactatctcccaaatcctggcaggtcaggc |
| | | agttgcgttcaacgtgaccttccggagggccaagggctaccccatcg[a/g]cctgtactacctga |
| | | tggacctctcctactccatggtggatgacctcgtcaacgtcaagaagctggggggtgacctgctc |
| | | cgggccctcaatggcatcaccgagtcgggccgcattggtga |
| 361 | DUMPS | GTGCAAATGGCTGAAGAACATTCTGAATTTGTGATTGGTTTTATTT |
| | | CTGGCTCC[C/t]GAGTAAGCATGAAACCAGAATTTCTTCACTTGAC |
| | | TCCAGGAGTTCAGTTAGAAGCAGGAGGT |
| 362 | CHONDR | CTTATTAATTAAGGACAAACGTCATTTCACTGGACTGGGTAGGGG |
| | | TTGGAAGTCCAAGGACTAGCAAGATTTGTGCACAAGCCTGGCCC |
| | | ACCTCATGGGCCCCTGGGTCCCCTGAAGAAGGGCCAGCCTGGG |
| | | GTGGGTCACTGGTACAGGAGCCCCCAGCCCTCACCANACATGTC |
| | | CTCTTTAGGTGTTTCAGCGGCGCCCTCTCCAGAAGAAGAGGAGG |
| | | GTAGTGCACCCACAGCAGGCCCTGACGTGGAGGAGTGGATGGT |
| | | GACACAAGTGGGGCCTGGCGTGGCTGCTGTCCCCATCGGGGAG |
| | | GAGACGACTGCAATCCCAGGCTTCACCGTTGAGCCAGAAAACAA |
| | | GACGGAATGGGAACTTGCCTACACCCCAGCGGGCACTTTGCCAC |
| | | TAC[*/GGCA]CAGGTCCGTCCGGGCTCTCCTGCATGTCCTGCTGC |
| | | CTCCCTGGGCCAGGGTGTGGCCTGGAAGGGGGGAGGAGGAAGT |
| | | GTTCTCTCCCTGGGACCCGTGATCTGTTCCCCAGCCCTGACCCC |
| | | CAGCCCTGATTTTATTTAAGTGTGCTGCCATGGGTAACTTCAGCC |
| | | ACCTCAGCAGGCATCCAGGACCAATCCTA |
| 363 | PROTO | cggtgtctgcgcttctgaccgtctgtccttcccgtctgtcctgcaggccctggccgacctggtccact |
| | | cacacctccagtccaaggagcgctgctccacacggctgactctgagctgtccgctctgcgtgaa |
| | | ccccacctgcagggagaccaaatccttcttcaccagccagcagctgt[g/t]accctggcggcac |
| | | gccgctgggaggtgcgcgtgcccgcctcccgacacctccgaggaggaggagggcgcatccg |
| | | gccgttagggaggaggttacatccg |
| 364 | HYPOTR | Tccctgcagctctgagtcctggaaaataaggctcagttgatgcttggcaaaaggctcagactga |
| | | gcctggcttggctcatacagggagcaaaagctcagtgccattggctgcctagatgaagaggaa |
| | | agcaagtagacagagcatgccctctgactggcctgtcctattttgcaggtgctgctgataaagctg |
| | | gaact[c/t]gagaaaaccaggttggctgggaattgctgtcttgctgggaaggagggaaggcca |
| | | caggcctgagccacccttgagtttgctccctgctaagttttctgaggctttcttttgtgaggagaccct |
| | | ggaggttc |
| 365 | SYNDAC | Ctgttaataaccaagacgatcagagccaccggagagccacgttcacatctgaagctcttagtcc |
| | | ttttcggccccctgttgttatatccctccttcctgccccactgtccctgggagtcagggagccctgaat |
| | | ctcctccatttggagaagcgtcaaactgggagacttgattctgccccaggccagatgttcatttgctt |
| | | tgctcatagggaccaacgcttgtggcgtgaacaa[cg/at]gcggatgcactcacctctgctttgc |
| | | cagaacctcggactttgtgtgtgcctgtcctgatgagcccgacggccggccctgctccctcggtga |
| | | gttggactgacgggcccccctgcaacagcgga |
| 366 | CITRUL | TGGTCACCCGTCACGATGTCCGGCAAAGGCTCCGTGGTTCTGGC |
| | | CTACAGTGGGGGCCTGGACACCTCCTGCATCCTCGTGTGGCTGA |
| | | AGGAGCAAGGCTATGACGTCATTGCCTACCTGGCCAACATCGGC |
| | | CAGAAAGAAGACTTTGAGGAAGCCAGGAAGAAGGCGCTGAAGCT |
| | | TGGGGCCAAAAAGGTGTTCATTGAGGACATCAGCAAGGAGTTTG |
| | | TGGAGGAGTTCATCTGGCCGGCCATCCAGTCCAGCGCACTGTAC |
| | | GAGGAC[C/t]GATACCTCCTGGGCACCTCTCTCGCCAGGCCCTGC |
| | | ATCGCCCGCAAGCAGGTGGAGATCGCCCAGCGAGAAGGAGCCA |
| | | AGTATGTGTCT |
| 367 | CVM | tttttaaaattatagattgtaaaggcaatatcactatgggaaaaaaaaatgattctaaggttttttcaa |
| | | aagctctcctctgtaatccccaggaatggaaatggttgcatttttaccttaaggtctaagagtgggct |
| | | ctaaacatgtattttgtaaaatattataggaattaaacttgtgttgtttctttttgttcagtggccctcagat |
| | | tctcaagagcttaattctaaggaactttcagctggctcacaatttgtaggtctcatggca[g/t]ttctc |
| | | acagcatgtttttccagtggctttgctggggtttactttgagaaaatcttaaaagaaaccaaacaatc |
| | | agtgtggataagaaacattcaacttggtaagttttaaatgttttctaacattacttttaaagtgattatat |
| | | tgttatatttaaagatttctatgtatctttaattaaataaaccttataaaaactgcttgttgttg |
| 368 | E+ | GGGGAGCCATGAGTTGAGCAGGACCCTGAGAGCAAGCACCCCT |
| | | TCCTGCTCCCTGCGGGACGATGCCTGCACTTGGCTCCCAGAGGC |
| | | GGCTGCTGGGTTCCCTTAACTGCACGCCCCCAGCCACCCTCCCC |
| | | TTCACCCTGGCCCCCAACCGGACGGGGCCCCAGTGCCTGGAGG |
| | | TGTCCATCCCTGANGGGCTCTTTCTCAGCCTGGGGCTGGTGAGT |
| | | CTCGTGGAGAACGTGCTGGTAGTGGCTGCCATTGCCAAGAACCG |
| | | CAACCTGCACTCCCCCATGTACTACTTTATCTGCTGCCTGGCTGT |
| | | GTCTGACTTGCTGGTGAGCGTCAGCAACGTGCTGGAGANGGCAG |
| | | TCATGC[T/C]GCTGCTGGAGGCCNGTGTCCTGGCCACCCAGGCG |
| | | GCCGTGGNGCAGCAGCTGGACAATGTCATCGACGTGCTCATCTG |
| | | CGGATCCATGGTGTCCAGCCTCTGCTTCCTGGGTGCCATTGNTG |
| | | TGGACCGCTACATCTCCATCTTCTACGCCCTGCGGTACCACNGT |
| | | GTTGTGACACTGCCCCGAGCGTGGAGGATCATTGCGGCCATCTG |
| | | GGTGGCCAGCATCCTCACCAGCCTGCTCTTCATCACCTACTACAA |
| | | CCACAAGGTCATCCTGCTGTGCCTCGTTGGCCTCTTCATAG |
| 369 | RED | GGGGAGCCATGAGTTGAGCAGGACCCTGAGAGCAAGCACCCCT |
| | | TCCTGCTCCCTGCGGGACGATGCCTGCACTTGGCTCCCAGAGGC |
| | | GGCTGCTGGGTTCCCTTAACTGCACGCCCCCAGCCACCCTCCCC |
| | | TTCACCCTGGCCCCCAACCGGACGGGGCCCCAGTGCCTGGAGG |
| | | TGTCCATCCCTGANGGGCTCTTTCTCAGCCTGGGGCTGGTGAGT |
| | | CTCGTGGAGAACGTGCTGGTAGTGGCTGCCATTGCCAAGAACCG |
| | | CAACCTGCACTCCCCCATGTACTACTTTATCTGCTGCCTGGCTGT |
| | | GTCTGACTTGCTGGTGAGCGTCAGCAACGTGCTGGAGANGGCAG |
| | | TCATGCNGCTGCTGGAGGCC[G/*]GTGTCCTGGCCACCCAGGCG |
| | | GCCGTGGNGCAGCAGCTGGACAATGTCATCGACGTGCTCATCTG |
| | | CGGATCCATGGTGTCCAGCCTCTGCTTCCTGGGTGCCATTGNTG |
| | | TGGACCGCTACATCTCCATCTTCTACGCCCTGCGGTACCACNGT |
| | | GTTGTGACACTGCCCCGAGCGTGGAGGATCATTGCGGCCATCTG |
| | | GGTGGCCAGCATCCTCACCAGCCTGCTCTTCATCACCTACTACAA |
| | | CCACAAGGTCATCCTGCTGTGCCTCGTTGGCCTCTTCATAG |
| 370 | DUN | TATAAAATATGAAAGAACTTTTATTGTTACCCTTAAACATTTTAAGT |
| | | CACCTTCAGAACATAATAATATATTAATACAAACTGATTATGTCTAT |
| | | TAACAAGGTGTCTTTGACATATTTTAGATATATCCACATATCCACT |
| | | GGAAAATGCCCCTATTGGA[C/T]ATAACAGACAATACAATATGGTA |
| | | CCATTTTGGCCTCCAGTTACCAACATAGAAATGTTTGTTACTGCTC |
| | | CAGACAACCTGGGCTATACTTANGAAGTTCAATGGCCAAGTGAGT |
| | | ACTGAAAATGTATTTTTACTGTGGAAATTTCCAAAATCAAACTTGT |
| | | TACCTTTAAAGTAATCTCAGTTTTCTGAGATAAAGTAACC |
| 371 | ALBINO | gcagatcgtctgcagcagactggaggagtacaacagtcgccaggctttatgcaacgggacgtc |
| | | tgagagaccattactgcgcaatcctggaaaccacgacaaagccaggacccc[*/c]gaggctc |
| | | ccctcctcggctgatgtggagttttgcctgagtttgacccagtatgaatcaggttccatggataaag |
| | | ctgccaatttcagctttaga |
| 372 | ROAN | gaaggcctcaaattccattgaagattccagcctacaatgggcag[c/a]cgtagcattgccagca |
| | | ttcttttctcttgtgatcgggtttgcttttggggccttttactggaag |
| 373 | Ucalp | agcatcctcggggcgtctgagctggccctcataagataacccctgggactggggtctctggactt |
| | | gcccttgtggaggcctcctgacctgggccagggaaggacaggccccagggatagaggctggg |
| | | caggtcagtggccgccagcccctggcagtgccgttttcctacagctcctcggagtggaacg[g/c] |
| | | cgtggacccttacatgcgggagcagctccgggtcaagatggaggatggggagttctggtgagc |
| | | agccccctcctcagtctgagtgggcaccccagctcccaaccccacccccctgaaaaccagctg |
| | | tgccatgtctcttgatgcctcgactgggcatcctggttcactctc |
| 374 | CALPA | attttgaactctcatctttcaacacttaagtcctacctagaatggcagttatttgtttttctgttaaaacgg |
| | | cacctctgtgtggcatcagcaggtattgcaatttgcttgtgtgattcttgctgaatttggaaggaagg |
| | | aattgcattgtttcaaatttt[g/c]tacccaaagtgaaatttgtcacatgtaaatcatactaatttaaat |
| | | tctcacaattgactacataaaacacaagtgttatgaattgctttctactcctcagagaaaagtagca |
| | | atatgtgtcatattattaaccccatg |
| 375 | MYOS | ggaaaatgtggaaaaagaggggctgtgtaatgcatgtttgtggagggaaaacactacatcctca |
| | | agactagaagccataaaaatccaaatcctcagtaaacttcgcctggaaacagctcctaacatca |
| | | gcaaagatgctatcagacaacttttgcccaaggctcctccactcctggaactgattgatcagtt[c/ |
| | | a]gatgtccagagagatgccagcagtgacggctccttggaagacgatgactaccacgccagg |
| | | acggaaacggtcattaccatgcccacggagtgtgagtagtcctgctggtgcagagcaacgactc |
| | | tgctgactgctgttctagtgttcatgagaaaccgatctatttt |
| 376 | ABCG2 | agtattcacgagactgtcagggacttaaagaggctatttgctagacggcaccagatctgattcttg |
| | | gtatttgttttttgtagatattttcagggctgttggtaaatctcaaaaccgtcgtgccttggttgtcatggc |
| | | ttcaatacttgagcattcctcgatacggct[a/c]tgcggtatgttctccttatctgtcaccgtgctggtt |
| | | cattgtccccatgctggaaacagccagaataaagcctctcatatccttggccatgagctgtgcaa |
| | | gttttaggacaatgaaggagagtttcctattaagccttgggtcaagttgataatcacctgggatttct |
| | | ctagtcaccttgttgtctgagg |
| 377 | Kcasein | GCCCAAATTCTTCAATGGCAAGTTTTGTCAAATACTGTGCCTGCC |
| | | AAGTCCTGCCAAGCCCAGCCAACTACCATGGCACGTCACCCACA |
| | | CCCACATTTATCATTTATGGCCATTCCACCAAAGAAAAATCAGGAT |
| | | AAAACAGAAATCCCTACCATCAATACCATTGCTAGTGGTGAGCCT |
| | | ACAAGTACACCTACCA[c/T]CGAAGCAGTAGAGAGCACTGTAGCT |
| | | ACTCTAGAAG[a/C]TTCTCCAGAAGTTATTGAGAGCCCACCTGAGA |
| | | TCAACACAGTCCAAGTTACTTCAACTGCGGTCTAAATACTCTAAG |
| | | GAGACATCAAAGAAGACAACGCAGGTAAATAAGCAAAATGAATAA |
| | | CAGC |
| 378 | zfxy1 | AGTAGAGGCAGAAATCGTCACTGATCCTCTGACAGCCGA[t/c]GTA |
| | | GTGTCAGAAGAAGTATTGGTAGCAGATTGTGCCTCAGAAGCAGT |
| | | CATAGATGCCAACG |
| 379 | zfxy2 | atgtggctgcccacaagggtaaaaaaatgcaccagtgtagacattgtgactttaagattgcagat |
| | | cc[t/a]tttgttctaagtcgccatattctctcagttca |
| 380 | GHR | Caccaagtgccgttcacctgaactggagactttctcatgtcactggacagatggggctaatcaca |
| | | gtttacagagcccaggatctgtacagatgttctatatcagaaggtatgggcttcatgcttttctgatttc |
| | | t[c/g]tccatgaattttctgatgaaaatccattgagtgtcatgcagt[a/g]gtgggaatggaaata |
| | | atcttctttggtgatctaaatgcattcacccattcattcatttaaatatattagttaagcccttactatatgt |
| | | tggg |
| 381 | Bcasein | GATGAACTCCAGGATAAAATCCACCCCTTTGCCCAGACACAGTCT |
| | | CTAGTCTATCCCTTCCCTGGGCCCATCC[C/A]TAACAGCCTCCCA |
| | | CAAAACATCCCTCCTCTTACTCAAACCCCTGTGGTGGTGCCGCCT |
| | | TTCCTTCAGCCTGAAGTAATGGGAGTCTCCAAAGTGAAGGAGGC |
| | | TATGG |

### Methods of Simultaneously Identifying a Plurality of Polymorphisms for the Determination of at Least Two Characteristics in an Animal

The present invention provides for methods of simultaneously and efficiently identifying a plurality of nucleotide polymorphisms that correlate with at least two characteristics, wherein the characteristics include parentage, identity, sex, genotype and/or phenotype. Thus, profiles for individual animals or groups of animals may be formed for future use or to research animal history.

In one method, the presence of a plurality of nucleotide polymorphisms are detected by performing PCR assays using an assay plate or panel, wherein each assay plate contains over 3.000 assays. e.g., 3072. An example of such a plate or panel is OpenArray™. In certain embodiments, four plates each containing over 3,000 assays each for a total of over 12,000 assays can be performed simultaneously. In other embodiments, multiple machines, each having four assay plates, can simultaneously perform between about 24,000 assays to several hundreds of thousands of assays. Each assay on the plate or panel is capable of detecting the presence of a polymorphism contained within a nucleotide marker sequence as provided in Tables 1-11. In particular, each assay is capable of discriminating alleles of a polymorphic sequence by detection of either allele 1, allele 2, or allele 1 and allele 2 at the polymorphic site in a nucleic acid sample.

Each individual assay, according to the method above, contains a nucleic acid sample, sequence-specific forward and reverse primers to amplify the polymorphic sequence of interest, two modified oligonucleotide probes (e.g., TaqMan® probes) and a DNA polymerase. One oligonucleotide probe matches the Allele 1 sequence; the other oligonucleotide matches the Allele 2 sequence. Each modified oligonucleotide probe contains a reporter dye at the 5′ end of the probe (e.g., a VIC® dye, or a FAM™ dye). A nonfluorescent quencher is attached at the 3′ end of the probe. Oligonucleotide probes of the present invention are 25 to 35 nucleotides in length, but more preferably 30 nucleotides in length and perfectly complementary to a region within the nucleotide marker sequence referred to as the invariant region. The invariant region contains no further polymorphisms, other than the polymorphism utilized to discriminate allele 1 from allele 2.

In the present invention, according to the method above, the forward and reverse primers hybridize to a sequence of DNA within the nucleic acid sample that is either upstream or downstream of a sequence corresponding to the invariant region within the nucleotide marker. The sequence is then amplified by PCR. During the PCR reaction, each oligonucleotide probe anneals specifically to a region spanning the invariant sequence of the nucleotide marker. The DNA polymerase contained within the assay mix can cleave the oligonucleotide probe only if it specifically hybridizes to a PCR-amplified sequence present within the sample. Cleavage separates the reporter dye from the quencher dye, increasing fluorescence by the reporter. Thus, the fluorescence signal(s) generated by PCR amplification indicates the presence of a specific polymorphic allele within the nucleic acid sample.

Oligonucleotide probes used in allele discrimination are linear fluorescently-labeled probes used to monitor PCR product formation either during or after the amplification process. As the DNA polymerase extends the upstream primers and encounters the downstream probe, the 5′ to 3′ nuclease activity of the polymerase cleaves the probe. Following cleavage, the reporter fluorophore is released into the reaction solution and fluorescence is detected.

More specifically, an oligonucleotide probe, containing a fluorescent dye at the 5′ end, that matches the Allele 1 sequence will generate a fluorescence signal at the wavelength of that fluorescent reporter dye only if the Allele 1 sequence is present in the nucleic acid sample. Similarly, a second oligonucleotide probe, containing a fluorescent dye at the 5′ end, that matches the Allele 2 sequence will generate a fluorescence signal at the wavelength of that fluorescent reporter dye only if the Allele 2 sequence is present in the nucleic acid sample. In this way the presence of either Allele 1, Allele 2, or both Allele 1 and Allele 2 of a nucleotide marker sequence of the present invention can be identified from an isolated nucleic acid sample in the assay described above using two different fluorescent dyes for each probe. Fluorescent dyes can include VIC®, FAM™, and other dyes known those of ordinary skill in the art.

In certain embodiments, a polymorphism of the present invention can be identified in part, by its position within a 30 nucleotide invariant region using the polymerase chain reaction in combination with oligonucleotide probes. This position can be, for example, the position within brackets and in bold, as shown in Tables 2, 4 and 6 above.

The present invention provides for a method as described above, wherein a single plate comprises 64 assays for identification of the polymorphic sites within the nucleotide markers according to Table 2 and/or 64 assays for identification of the polymorphic sites within the nucleotide markers according to Table 4 and/or 128 assays for the identification of the polymorphic sites within the nucleotide markers according to Table 6. In other embodiments, nucleotide markers according to Table 7 or 9 and 10 are used to detect polymorphic sites within the nucleotide markers according to Tables 8 and 11 respectively. A single plate may be any available or offered to those in genetic screening arts and is thus nonlimiting.

PCR reactions are performed using assay plates according to the method above by simultaneously thermal cycling using a commercial flat-block thermal cycler. The fluorescence output is subsequently read using a computer-based imaging system. Each plate is capable of performing over 3000 assays simultaneously. One, two or three plates performing over 3000 assays can be performed simultaneously.

In this way, high-throughput cost-efficient analysis of over 3000, 6000 or 12,000 (e.g., 3072, 6344, 9216 or 12,288) polymorphic sites can be assayed simultaneously. The present invention therefore provides a rapid and powerful method to simultaneously determine at least two characteristics, such as parentage, identity and/or phenotype in a single animal, in more than one animal and/or in more than one species of animal at a much lower cost than previous systems.

A nucleic acid sample useful for practicing a method of the invention can be any isolated biological sample obtained from an animal, such as an equine, canine, feline, or human, that contains nucleic acid molecules, including portions of the gene sequences to be examined, or corresponding encoded polypeptides, depending on the particular method. As such, the sample can be a cell, tissue or organ sample, or can be a sample of a biological material such as blood, milk, semen, saliva, hair, tissue, and the like. A nucleic acid sample useful for practicing a method of the invention can be deoxyribonucleic (DNA) acid or ribonucleic acids (RNA). The nucleic acid sample generally is a deoxyribonucleic acid sample, particularly genomic DNA or an amplification product thereof. However, where heteronuclear ribonucleic acid, which includes unspliced mRNA precursor RNA molecules and non-coding regulatory molecules such as RNA, is available, a cDNA or amplification product thereof can be used.

In another aspect of the invention, the identification of a plurality of polymorphisms can be performed where the oligonucleotide markers are attached to the assay plate itself, and polymorphisms are detected by hybridization of an isolated nucleic sample to the oligonucleotide marker itself. In such a method, a plurality of nucleotide marker sequences is utilized, wherein each of said nucleotide marker sequences comprises a polymorphism, and wherein said plurality of nucleotide marker sequences correlates with at least two characteristics selected from the group consisting of: (i) parentage; (ii) identity; (iii) genotype (iv) phenotype; and wherein each of said nucleotide marker sequences is complementary to a nucleotide sequence derived from one or more animals.

In such a method, at least two characteristics of an animal are determined by: (a) contacting a nucleic acid sample with the composition comprising oligonucleotide markers; (b) hybridizing said nucleic sample to said plurality of nucleotide marker sequences in said composition; and (c) detecting oligonucleotide sequences within said nucleic sample that have hybridized to said plurality of nucleotide marker sequences, wherein each of said nucleotide marker sequences is complementary to an oligonucleotide sequence derived from one or more animals.

In certain embodiments, the nucleic sample is detectable labeled, and the hybridization of the nucleic acid sample with the nucleotide marker sequence results in fluorescence.

In certain other embodiments, the nucleotide marker sequences are attached to a substrate where the substrate can be, for example, a chip, wafer, slide, membrane, particle, bead, or any surface which would be compatible with the assay considered.

As used herein, the terms “bead,” “microsphere,” “microparticle,” and “particle” are used interchangeably. Bead composition may include, but is not limited to, plastics, ceramics, glass, polystyrene, methylstyrene, acrylic polymers, paramagnetic materials, carbon graphite, titanium dioxide, latex or cross-linked dextrans such as sepharose, cellulose, nylon, cross-linked micelles and polytetrafluoroethylene.

Beads may be associated with a physically or chemically distinguishable characteristic. For example, beads may be stained with sets of optically distinguishable tags, such as those containing one or more fluorophore or chromophore dyes distinguishable by excitation wavelength, emission wavelength, excited-state lifetime or emission intensity. Optically distinguishable dyes combined in certain molar ratios may be used to stain beads in accordance with methods known in the art. Combinatorial color codes for exterior and interior surfaces are disclosed in International Application No. PCT/US98/10719, incorporated herein by reference. Beads capable of being identified on the basis of a physically or chemically distinguishable characteristic are said to be “encoded.”

The detection of the chemically or physically distinguishable characteristic of each set of beads and the identification of optical signatures on such beads generated in the course of a genetic or other test (such as diagnostic or prognostic test) using such beads may be performed by respectively recording a decoding image and an assay image of a set or array of such beads and comparing the two images. For example, in certain embodiments, a system with an imaging detector and computerized image capture and analysis apparatus may be used. The decoding image is obtained to determine the chemical and/or physical distinguishable characteristic that uniquely identifies the probe displayed on the bead surface. In this way, the identity of the probe on each particle in the array is provided by the distinguishable characteristic. The assay image of the array is obtained to detect an optical signature produced in the assay as elaborated herein below.

In addition to being encoded, beads having specific oligonucleotide probes or primers may be spatially separated in a manner such that the bead location provides information about bead and hence about probe or primer identity. In one example, spatial encoding may be provided by placing beads in two or more spatially separate subarrays.

In a preferred embodiment, beads can be arranged in a planar array on a substrate before decoding and analysis. Bead arrays may be prepared by the methods disclosed in PCT/US01/20179, incorporated herein by reference in its entirety. Bead arrays also may be formed using the methods described in U.S. Pat. No. 6,251,691, incorporated herein by reference in its entirety. For example, light-controlled electrokinetic forces may be used to assemble an array of beads in a process known as “LEAPS”, as described in U.S. Pat. No. 6,251,691. Alternatively, if paramagnetic beads are used, arrays may be formed on a substrate surface by applying a magnetic field perpendicular to the surface. Bead arrays also may be formed by mechanically depositing the beads into an array of restraining structures (e.g., recesses) at the surface of the substrate. In certain embodiments, the bead arrays may be immobilized after they are formed by using physical means, such as, for example, by embedding the beads in a gel to form a gel-particle film.

A target that forms a hybridization complex with immobilized probes can be visualized by using detection methods previously described herein. For example, probes annealed to target strands can be elongated with labeled dNTPs, such that extension occurs when the probe perfectly matches the number of repeats in the target. Several other configurations for generating positive assay signals may be readily constructed.

As described for sequence-specific probes in general, parallel interrogation repeated sequences may be immobilized on solid supports via a linker moiety, use of which is well known in the art. As a general rule, probes should be sufficiently long to avoid annealing to unrelated DNA target sequences. The length of the probe may be about 10 to 50 bases, more preferably about 15 to 25 bases, and even more preferably 18 to 20 bases. In a multiplexed assay, one or more solution-borne targets are then allowed to contact a multiplicity of immobilized probes under conditions permitting annealing and elongation reactions.

The present invention offers advantages over the existing methods of analyzing polymorphisms in animals because of the combination of nucleotide marker sequences that can be simultaneously detected, and because of the efficient and cost-efficient method by which a large number of nucleotide markers can be assayed simultaneously. The present invention further offers advantages that at least two characteristics including parentage, identity and phenotype can be simultaneously determined in at least one, two, three or four and up to forty-eight different animals on one assay plate.

The present system also offers the advantage of simultaneously detecting polymorphisms of the marker sequences as set forth in Tables 1-11. In this way, the present invention can simultaneously detect different kinds of polymorphisms including, but not limited to single nucleotide polymorphisms (SNPs), insertions and/or deletions and other mutations.

In another aspect of the invention, a polymorphism within a nucleotide marker sequence can be detected based on the lack of incorporation of a specific nucleotide, for example a fluorescently-labeled or radiolabeled nucleotide.

Additional methods known in the art can be utilized for determining the presence of a plurality of polymorphisms in a sample.

For example, the identification can use microarray technology, which can be performed with PCR, for example using Affymetrix technologies and GenFlex Tag arrays (See e.g., Fan et al (2000) Genome Res. 10:853-860), or using a gene chip containing proprietary SNP oligonucleotides (See e.g., Chee et al (1996), Science 274:610-614; and Kennedy et al. (2003) Nature Biotech 21:1233-1237) or without PCR, or sequencing methods such as mass spectrometry, scanning electron microscopy, or methods in which a polynucleotide flows past a sorting device that can detect the sequence of the polynucleotide. The presence of a polymorphism can be identified using electrochemical detection devices such as the eSensor™ DNA detection system (Motorola, Inc., Yu, C. J. (2001) J. Am. Chem. Soc. 123:11155-11161). Other formats include melting curve analysis using fluorescently labeled hybridization probes, or intercalating dyes (Lohmann, S. (2000) Biochemica 4, 23-28, Herrmann, M. (2000) Clinical Chemistry 46: 425).

An oligonucleotide ligation assay (Grossman, P. D. et al. (1994) Nucleic Acids Research 22:4527-4534) also can be used to identify a polymorphic site within a nucleotide marker sequence, wherein a pair of probes that selectively hybridize upstream and adjacent to and downstream and adjacent to the site of the polymorphism, and wherein one of the probes includes a terminal nucleotide complementary to the polymorphism. Where the terminal nucleotide of the probe is complementary to the SNP, selective hybridization includes the terminal nucleotide such that, in the presence of a ligase, the upstream and downstream oligonucleotides are ligated. As such, the presence or absence of a ligation product is indicative of the presence of the polymorphism. An example of this type of assay is the SNPlex System (Applied Biosystems, Foster City, Calif.).

An oligonucleotide also can be useful as a primer, for example, for a primer extension reaction, wherein the product (or absence of a product) of the extension reaction is indicative of the polymorphism. In addition, a primer pair useful for amplifying a portion of the target polynucleotide including the polymorphic site can be useful, wherein the amplification product is examined to discriminate the alleles at a polymorphic site. Particularly useful methods include those that are readily adaptable to a high throughput format, to a multiplex format, or to both. The primer extension or amplification product can be detected directly or indirectly and/or can be sequenced using various methods known in the art. Amplification products which span a polymorphic site can be sequenced using traditional sequence methodologies (e.g., the “dideoxy-mediated chain termination method,” also known as the “Sanger Method” (Sanger, F., et al., J. Molec. Biol. 94:441 (1975); Prober et al. Science 238:336-340 (1987)) and the “chemical degradation method,” “also known as the “Maxam-Gilbert method” (Maxam, A. M. et al., Proc. Natl. Acad. Sci. (U.S.A.) 74:560 (1977)), both references herein incorporated by reference) to discriminate the alleles at the polymorphic site.

Other techniques including fluorescence spectroscopy, capillary electrophoresis (CE), and high performance liquid chromatography (HPLC) can be used for detection. The presence of a nucleotide marker polymorphisms can also be determined using microchip electrophoresis such as described in Schmalzing et al., Nucl. Acid. Res. 28:e43 (2000). In addition, the presence of a nucleotide marker polymorphism can be determined using denaturing HPLC such as described in Nairz K et al (2002) Proc. Natl. Acad. Sci. (U.S.A.) 99:10575-80, and the Transgenomic WAVE™ System (Transgenomic, Inc. Omaha, Nebr.).

Oliphant et al. report a method that utilizes BeadArray™ Technology that can be used in the methods of the present invention to determine the nucleotide occurrence of a SNP (supplement to Biotechniques, June 2002). Additionally, nucleotide occurrences for SNPs can be determined using a DNAMassARRAY system (SEQUENOM, San Diego, Calif.). This system combines proprietary SpectroChips™, microfluidics, nanodispensing, biochemistry, and MALDI-TOF MS (matrix-assisted laser desorption ionization time of flight mass spectrometry).

As another example, the presence of a nucleotide marker polymorphism in a sample can be determined using the SNP-ITT™ method (Beckman Coulter, Fullerton, Calif.). In general, SNP-ITT™ is a 3-step primer extension reaction. In the first step a target polynucleotide is isolated from a sample by hybridization to a capture primer, which provides a first level of specificity. In a second step the capture primer is extended from a terminating nucleotide triphosphate at the target polymorphic site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument (Beckman Coulter, Fullerton, Calif.). Reactions can also be analyzed by binding to Luminex biospheres (Luminex Corporation, Austin, Tex. Cai. H. (2000) Genomics 66(2):135-43).

Other formats for nucleotide marker polymorphism detection include TaqMan™ (Applied Biosystems, Foster City, Calif.). Rolling circle (Hatch et al (1999) Genet. Anal. 15: 35-40, Qi et al (2001) Nucleic Acids Research Vol. 29 e116), fluorescence polarization (Chen, X., et al. (1999) Genome Research 9:492-498), SNaPShot (Applied Biosystems, Foster City, Calif.) (Makridakis, N. M. et al. (2001) Biotechniques 31:1374-80), oligo-ligation assay (Grossman, P. D., et al. (1994) Nucleic Acids Research 22:4527-4534), locked nucleic acids (LNATM, Link, Technologies LTD, Lanarkshire, Scotland, EP patent 1013661, U.S. Pat. No. 6,268,490), Invader Assay (Aclara Biosciences, Wilkinson, D. (1999) The Scientist 13:16), padlock probes (Nilsson et al. Science (1994), 265: 2085), Sequence-tagged molecular inversion probes (similar to padlock probes) from ParAllele Bioscience (South San Francisco, Calif.; Hardenbol, P. et al. (2003) Nature Biotechnology 21:673-678), Molecular Beacons (Marras, S. A. et al. (1999 Genet Anal. 14:151-156), the READIT™ SNP Genotyping System from Promega (Madison, Wis.) (Rhodes R. B. et al. (2001) Mol. Diagn. 6:55-61), Dynamic Allele-Specific Hybridization (DASH) (Prince, J. A. et al. (2001) Genome Research 11: 152-162), the Qbead™, system (quantum dot encoded microspheres conjugated to allele-specific oligonucleotides) (Xu H. et al. (2003) Nucleic Acids Research 31:e43), Scorpion primers (similar to molecular beacons except unimolecular) (Thelwell, N. et al. (2000) Nucleic Acids Research 28:3752-3761), and Magiprobe (a novel fluorescence quenching-based oligonucleotide probe carrying a fluorophore and an intercalator) (Yamane A. (2002) Nucleic Acids Research 30:e97).

In addition, Rao, K. V. N. et al. ((2003) Nucleic Acids Research. 31:e66), recently reported a microsphere-based genotyping assay that detects SNPs directly from human genomic DNA. The assay involves a structure-specific cleavage reaction, which generates fluorescent signal on the surface of microspheres, followed by flow cytometry of the microspheres. With a slightly different twist on the Sequenom technology (MALDI), Sauer et al. ((2003) Nucleic Acids Research 31:e63) generate charge-tagged DNA (post PCR and primer extension), using a photocleavable linker.

A method for identifying a nucleotide marker polymorphism also can be performed using a specific binding pair member. As used herein, the term “specific binding pair member” refers to a molecule that specifically binds or selectively hybridizes to another member of a specific binding pair. Specific binding pair members include, for example, probes, primers, polynucleotides, antibodies, etc. For example, a specific binding pair member includes a primer or a probe that selectively hybridizes to a target polynucleotide that includes a polymorphic site or that hybridizes to an amplification product generated using the target polynucleotide as a template.

As used herein, the term “specific interaction,” or “specifically binds” or the like means that two molecules form a complex that is relatively stable under physiologic conditions. The term is used herein in reference to various interactions, including, for example, the interaction of an antibody that binds a polynucleotide that includes a polymorphic site or the interaction of an antibody that binds a polypeptide that includes an amino acid that is encoded by a codon that includes a polymorphic site. According to methods of the invention, an antibody can selectively bind to a polypeptide that includes a particular amino acid encoded by a codon that includes a polymorphic site. Alternatively, an antibody may preferentially bind a particular modified nucleotide that is incorporated into a polymorphic site for particular allelic differences at the polymorphic site, for example, using a primer extension assay.

A specific interaction can be characterized by a dissociation constant of at least about 1×10-6 M, generally at least about 1×10-7 M, usually at least about 1×10-8 M, and particularly at least about 1×10-9 M or 1×10-10 M or less. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

The system can be a microfluidic device. Numerous microfluidic devices are known that include solid supports with microchannels (See e.g., U.S. Pat. Nos. 5,304,487, 5,110,745, 5,681,484, and 5,593,838).

To facilitate detection, hybridization complexes can be modified to contain one or more labels. These labels can be incorporated by any of a number of means well known to those skilled in the art. Detectable labels suitable for use in the present invention include any composition detectable by spectroscopic, photochemical, biochemical, immunochemical, electrical, optical, or chemical means. Useful labels in the present invention include high affinity binding labels such as biotin for staining with labeled streptavidin or its conjugate, magnetic beads, fluorescent dyes (for example, fluorescein, Texas red, rhodamine, green fluorescent protein, and the like), radiolabels (for example 3H, 125I, 35S, 14C, or 32P), enzymes (for example horseradish peroxidase, alkaline phosphatase and others commonly used in an ELISA), epitope labels, and calorimetric labels such as colloidal gold, colored glass or plastic beads (for example polystyrene, polypropylene, latex, and the like). Means of detecting such labels are well known to those of skill in the art. Thus, for example, radiolabels can be detected using photographic film or scintillation counters, and fluorescent markers can be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. One method uses colloidal gold as a label that can be detected by measuring light scattered from the gold. The label can be added to the amplification products prior to or after the hybridization.

“Direct labels” are detectable labels that are directly attached to, or incorporated into, the nucleic acids prior to hybridization. In contrast, “indirect labels” are affixed to, or incorporated into the hybridization complex following hybridization. Often, the indirect label is attached to a binding moiety that has been attached to the amplified nucleic acid prior to hybridization. Thus, for example, the amplified nucleic acid can be biotinylated before hybridization. After hybridization, an avidin or streptavidin conjugated fluorophore will bind the biotin-bearing hybrid duplexes, providing a label that is easily detected.

Means for detecting labeled nucleic acids hybridized to probes in an array are known to those skilled in the art. For example, when a colorimetric label is used, simple visualization of the label is sufficient. When radiolabeled probes are used, detection of the radiation (for example, with photographic film or a solid state detector) is sufficient. Detection of fluorescently labeled target nucleic acids can be accomplished by means of fluorescence microscopy. An array of hybridization complexes can be excited with a light source at the excitation wavelength of the particular fluorescent label of choice and the resulting fluorescence at the emission wavelength detected. The excitation light source can be, for example, a laser appropriate for the excitation of the fluorescent label.

In a preferred embodiment, the hybridized nucleic acids are detected by detecting one or more labels attached to the sample nucleic acids. The labels may be incorporated by any of a number of means well known to those of skill in the art. However, in a preferred embodiment, the label is simultaneously incorporated during the amplification step in the preparation of the sample nucleic acids. Thus, for example, polymerase chain reaction (PCR) with labeled primers or labeled nucleotides will provide a labeled amplification product. In a preferred embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and coloimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

An oligonucleotide probe array complementary to the reference sequence or subsequence thereof is immobilized on a solid support using one of the display strategies described below. For the purposes of clarity, much of the following description of the invention will use probe arrays where the reference sequence or subsequene thereof is selected from any one of the oligonucleotide marker sequences of Tables 2, 4 and/or 6 derived from horse or dog; however it should be recognized, as described previously, that probe arrays derived from other animal genomes may also be used, depending on the phenotypic trait being monitored, the availability of suitable primers and the like.

The methods of this invention employ oligonucleotide arrays which comprise probes exhibiting complementarity to one or more selected reference sequences whose sequence is known. Typically, these arrays are immobilized in a high density array (“DNA on chip”) on a solid surface as described in U.S. Pat. No. 5,143,854 and PCT patent publication Nos. WO 90/15070, WO 92/10092 and WO 95/11995, each of which is incorporated herein by reference.

In another embodiment, the present invention provides an isolated vector that includes a polynucleotide or oligonucleotide disclosed herein. The term “vector” refers to a plasmid, virus or other vehicle known in the art that has been manipulated by insertion or incorporation of a nucleic acid sequence.

Methods that are well known in the art can be used to construct vectors, including in vitro recombinant DNA techniques, synthetic techniques, and in vivo recombination/genetic techniques (See, for example, the techniques described in Maniatis et al. 1989 Molecular Cloning A Laboratory Manual, Cold Spring Harbor Laboratory, N.Y., incorporated herein in its entirety by reference).

### Systems for Determining Multiple Characteristics in Animals Using the Simultaneous Identification of Polymorphisms in Biological Samples

The present invention provides for systems to order and display the fluorescence and/or hybridization pattern, for example, of the assay plate utilized to detect a plurality of oligonucleotide marker polymorphisms.

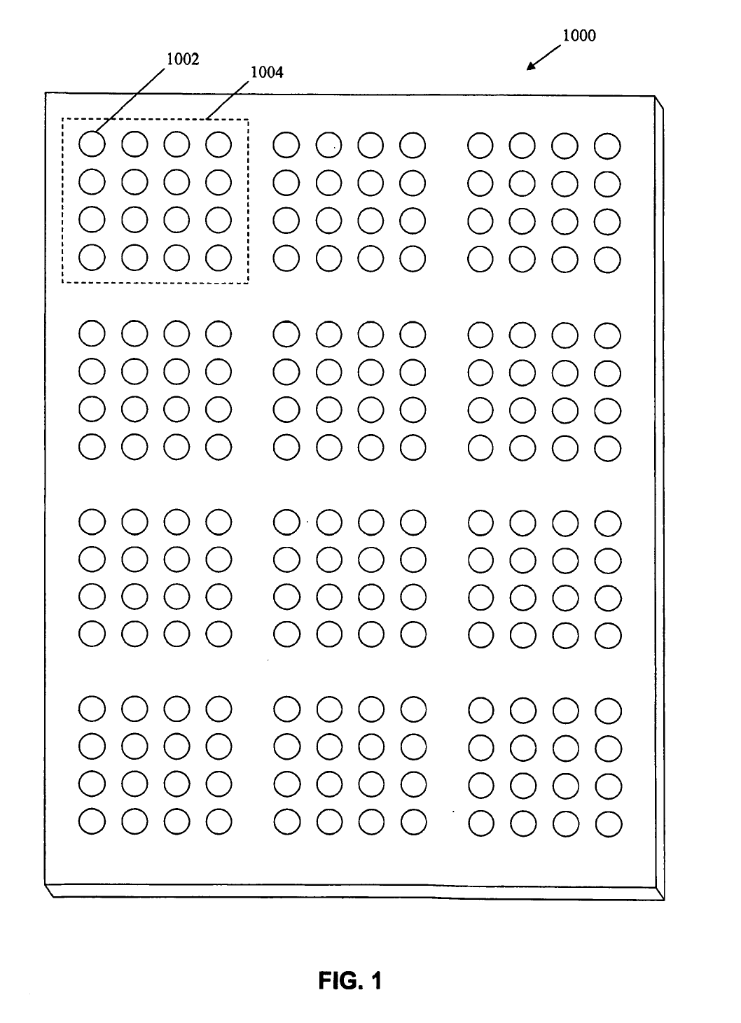

FIG. 1 is an exemplary reaction plate or panel 1000 upon which a plurality of samples or assays may be stored for processing in accordance with any of the techniques described above. In FIG. 1, panel 1000 includes an array of recesses 1002, which may be implemented as wells or through-holes. A well is defined as a recess that extends partially through panel 1000. For instance, a well does not form a hole through panel 1000. A through-hole, on the other hand, is defined as a recess that extends entirely through panel 1000 from one opposing surface to another, thereby forming a hole through panel 1000.

In the embodiment of FIG. 1, recesses 1002 are grouped into a plurality of subarrays 1004. Each subarray 1004 is shown to include a matrix of recesses 1002 having four rows and four columns for illustrative purposes. However, persons skilled in the art will recognize that subarrays 1004 can have any number of rows and columns or some other configuration. In fact, recesses 1002 need not be grouped into subarrays at all.

Referring to FIG. 1, samples are placed in respective recesses 1002 of panel 1000. Each sample may include a primer sequence pair, an oligonucleotide probe, a nucleic acid sample and/or a nucleotide marker sequence, to provide some examples. According to a first embodiment, each sample includes a respective primer sequence pair and a respective probe. Each of the primer sequences is capable of hybridizing to a sequence that is about 30 to 60 nucleotides upstream or downstream of a polymorphism present within a nucleotide marker sequence. In this embodiment, each of the primer sequence pairs flanks a polymorphism present within a nucleotide marker sequence. Moreover, each of the oligonucleotide probes is capable of hybridizing to a region that spans the polymorphism present within the nucleotide marker sequence. The plurality of primer sequence pairs and the plurality of probes is capable of detecting polymorphisms present within a plurality of nucleotide marker sequences. In this embodiment, the polymorphisms present within the plurality of nucleotide marker sequences correlate with at least two characteristics of an animal, such as parentage, identity, breed, sex, genotype and/or phenotype.

According to a second embodiment, each sample includes a respective nucleotide marker sequence. Each of the nucleotide marker sequences includes a polymorphism and correlates with at least two characteristics, such as parentage, identity, breed, sex, genotype and/or phenotype. In this embodiment, each of the nucleotide marker sequences is complementary to a nucleotide sequence derived from one or more animals.

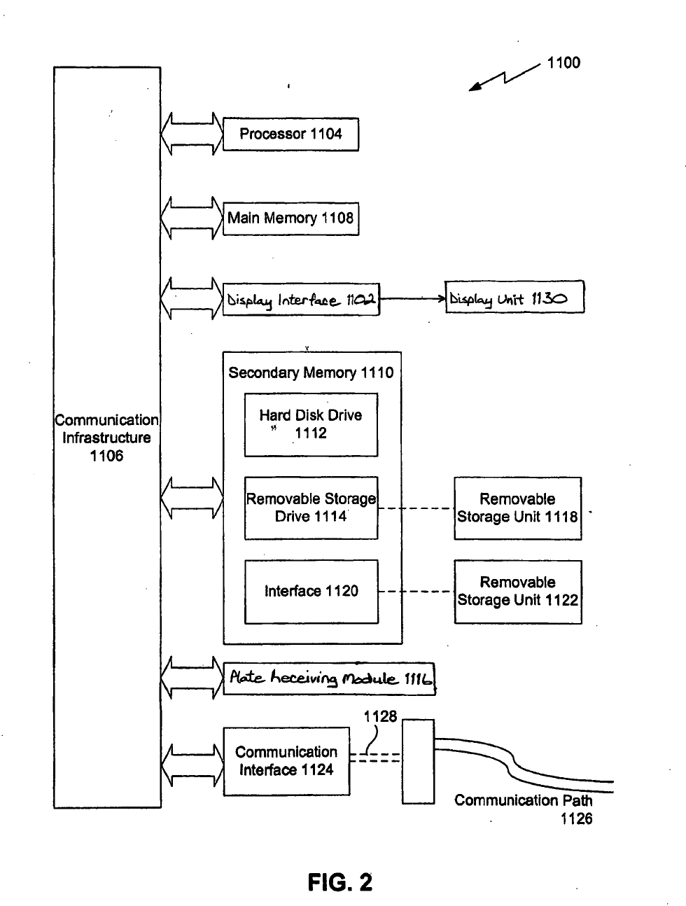

FIG. 2 illustrates an exemplary processor-based system 1100, which may be used to process samples according to an embodiment of the present invention. One or more aspects of the present invention may be implemented as programmable code. The programmable code may be provided in any of a variety of formats, including but not limited to C, C++, Java, and Visual Basic. Various embodiments of the invention are described in terms of exemplary processor-based system 1100. After reading this description, it will become apparent to a person skilled in the art(s) how to implement the invention using other processor-based systems and/or computer architectures.

FIG. 2 will be described with continued reference to reaction plate 1000 shown in FIG. 1 for illustrative purposes. However, the scope of the present invention is not limited to the use of reaction plate 1000. Any object capable of storing samples may be used in lieu of reaction plate 1000.

Referring now to FIG. 2, reaction plate 1000 is provided to plate receiving module 1116, which secures reaction plate 1000 using a securing element. Samples may be provided to reaction plate 1000 before providing reaction plate 1000 to plate receiving module 1116. Alternatively, plate receiving module 1116 may be used to manually or automatically provide the samples to reaction plate 1000.

Once the samples are loaded in plate receiving module, the samples may be processed in accordance with any of the techniques described above. For example, processor-based system 1100 may process the samples to identify characteristics, such as parentage, breed, identity, and/or phenotype, associated therewith. In another example, processor-based system 1100 may process the samples to identify SNPs therein.

Processor-based system 1100 includes one or more processors, such as processor 1104, to facilitate processing the samples. Processor 1104 may be any type of processor, including but not limited to a special purpose or a general purpose digital signal processor. Processor 1104 is connected to a communication infrastructure 1106 (for example, a bus or a network).

Processor-based system 1100 also includes a main memory 1108, preferably random access memory (RAM), and may also include a secondary memory 1110. Secondary memory 1110 may include, for example, a hard disk drive 1112 and/or a removable storage drive 1114, representing a floppy disk drive, a magnetic tape drive, an optical disk drive, etc.

Removable storage drive 1114 reads from and/or writes to a removable storage unit 1118 in a well known manner. Removable storage unit 1118 represents a floppy disk, magnetic tape, optical disk, etc. As will be appreciated, removable storage unit 1118 includes a computer usable storage medium having stored therein computer software and/or data.

In alternative implementations, secondary memory 1110 may include other similar means for allowing computer programs or other instructions to be loaded into processor-based system 1100. Such means may include, for example, a removable storage unit 1122 and an interface 1120. Examples of such means may include a program cartridge and cartridge interface (such as that found in video game devices), a removable memory chip (such as an EPROM or a PROM) and associated socket, and other removable storage units 1122 and interfaces 1120 which allow software and data to be transferred from removable storage unit 1122 to processor-based system 1100.

In FIG. 2, an optional communication interface 1124 allows software and data to be transferred between processor-based system 1100 and external devices. Examples of communication interface 1124 include but are not limited to a modem, a network interface (such as an Ethernet card), a communication port, a Personal Computer Memory Card International Association (PCMCIA) slot and card, etc. Software and data transferred via communication interface 1124 are in the form of signals 1128 which may be electronic, electromagnetic, optical, or other signals capable of being received by communication interface 1124. These signals 1128 are provided to communication interface 1124 via a communication path 1126. Communication path 1126 carries signals 1128 and may be implemented using wire or cable, fiber optics, a phone line, a cellular phone link, a radio frequency link, or any other suitable communication channel. For instance, communication path 1126 may be implemented using a combination of channels.

In the embodiment of FIG. 2, processor-based system 1100 further includes a display interface 1102 that forwards graphics, text, and/or other information from communication infrastructure 1106 (or from a frame buffer not shown) for display on display unit 1130. For instance, display unit 1130 may provide a graphical or textual representation of the results of processing the samples. Display unit may be a printer or a computer monitor, to provide some examples.

In this document, the terms “computer program medium” and “computer usable medium” are used generally to refer to media such as removable storage unit 1118, a hard disk installed in hard disk drive 1112, and signals 1128. These computer program products are means for providing software to processor-based system 1100.

Computer programs (also called computer control logic) are stored in main memory 1108 and/or secondary memory 1110. Computer programs may also be received via communication interface 1124. Such computer programs, when executed, enable processor-based system 1100 to implement the present invention as discussed herein. Accordingly, such computer programs represent controllers of processor-based system 1100. Where the invention is implemented using software, the software may be stored in a computer program product and loaded into processor-based system 1100 using removable storage drive 1114, hard disk drive 1112, or communication interface 1124, to provide some examples.

In alternative embodiments, the invention can be implemented as control logic in hardware, firmware, or software or any combination thereof.

The Examples provided herein illustrates the use of genotyping analysis to identify SNPs that can be used to determine parentage, identity, and/or phenotype of an animal (see Examples, infra). Information related to allele frequencies are utilized to correlate the presence of SNPS with a particular characteristic. The identification of particular SNPs in a target nucleic acid sequence. In some embodiments, forward oligonucleotide primers and reverse oligonucleotide primers were used to amplify specific target sequences prior to extension.

The identification of a plurality of nucleotide marker polymorphisms, for example, can establish a “record” for individual animals, such that the unique set of nucleotide marker polymorphisms detected in an individual nucleic acid sample isolated from an animal can be used to link a genetic profile to that individual animal's identity. This information can be obtained by on-chip genetic testing and can be linked to a concurrently recorded biochemical ID marker which in turn can be cross-referenced with existing veterinary records to ensure authenticity.

Many software programs for the analysis of nucleotide marker polymorphisms have been developed. Software programs to be used in the present invention include: The present disclosure incorporates the use of all of the software disclosed above used to classify animals into populations based on DNA polymorphisms as well as other software known in the art.

The genetic profiling of animals plays an increasingly important role, not only in basic and applied clinical research, but also in the diagnosis of disease and in the assessment of predisposition to disease. A safe, reliable genetic testing protocol preferably will incorporate all relevant information relating to patient identification within individual tests. The present invention provides methods and compositions for linking the genetic profile obtained from the analysis of a patient's sample to a patient's identity. This correlation between a patient's genetic profile and identity is established concurrently with the genetic test or any diagnostic or prognostic test, on the basis of recording a genetic fingerprint or molecular identifier (ID).

### Methods of Determining Diagnosis and Diseases

The invention further provides a diagnostic method useful during diagnosis of a disease, e.g., which involves detecting the presence of a nucleotide marker polymorphisms in tissue or other cells or body fluid from an individual animal and comparing the measured presence with a standard nucleotide marker containing a polymorphism in normal tissue or body fluid, whereby the presence of a nucleotide containing a polymorphism compared to the standard is indicative of a disorder.

By “assaying the presence of single nucleotide polymorphisms (SNPs) or polymorphism” is intended qualitatively or quantitatively measuring or estimating the present of SNPs, insertions, deletions, inversions and/or other mutations in a first biological sample either directly (e.g., by determining or estimating absolute presence of nucleotide containing a SNP) or relatively (e.g., by comparing to the disease associated with the presence of a nucleotide containing a SNP in a second biological sample). Preferably, the presence of a nucleotide containing a SNP in the first biological sample is measured or estimated and compared to a standard nucleotide marker containing a SNP, the standard being taken from a second biological sample obtained from an individual animal not having the disorder or being determined by averaging levels from a population of animals not having the disorder. As will be appreciated in the art, once the “standard” nucleotide marker containing a SNP is known, it can be used repeatedly as a standard for comparison.

The method, compositions and systems according to the present invention provide for detection and diagnosis of diseases as further described below.

Hyperkalemic periodic paralysis (HYPP) is an inherited disease of the muscle, which is caused by a genetic defect. In the muscle of affected horses, a point mutation exists in the sodium channel gene and is passed on to offspring. Sodium channels are “pores” in the muscle cell membrane which control contraction of the muscle fibers. When the defective sodium channel gene is present, the channel becomes “leaky” and makes the muscle overly excitable and contract involuntarily. The channel becomes “leaky” when potassium levels fluctuate in the blood. This may occur with fasting followed by consumption of a high potassium feed such as alfalfa. Hyperkalemia, which is an excessive amount of potassium in the blood, causes the muscles in the horse to contract more readily than normal. This makes the horse susceptible to sporadic episodes of muscle tremors or paralysis.

This genetic defect has been identified in descendents of the American Quarter Horse sire, Impressive. The original genetic defect causing HYPP was a natural mutation that occurred as part of the evolutionary process. The majority of such mutations, which are constantly occurring, are not compatible with survival. However, the genetic mutation causing HYPP produced a functional, yet altered, sodium ion channel. This gene mutation is not a product of inbreeding. The gene mutation causing HYPP inadvertently became widespread when breeders sought to produce horses with heavy musculature. To date, confirmed cases of HYPP have been restricted to descendants of this horse.

Severe Combined Immunodeficiency Disease (SCID) is an inherited disease specifically seen in pure and part-bred Arab horses. Foals afflicted with this condition have an enhanced susceptibility to infection and first show signs of disease at between two days and eight weeks of age. Clinical diagnosis of the disease is not straightforward as the symptoms, such as raised temperature, respiratory complications and diaharrea, are typical of new-born foals with a range of infections. SCID affected foals always die within the first six months of life, regardless of the level of veterinary care administered. SCID is therefore a distressing condition both for the animals involved and the owners and carers of the horses, and results in financial loss due to dead foals and veterinary expenses.

Junctional epidermolysis bullosa (JEB) is an inherited disease that causes moderate to severe blistering of the skin and mouth epithelia, and sloughing of hooves in newborn foals. This condition is also known as red foot disease. Affected foals are typically born alive, but soon develop skin lesions at pressure points. The condition worsens with time and the foal eventually succumbs from severe infection or has to be euthanized.

JEB in Belgian Draft horses has been shown to be the result of a specific mutation in a gene that affects the production of normal and healthy skin (F. Spirito et. al., J Invest Dermatol 119:684-691, 2002). To date, this mutation has been found only in Belgian Draft horses and derivatives of that breed. JEB is inherited as a recessive trait. Animals that carry two copies of the mutated gene (homozygous recessive) will develop the disease. Animals that carry one copy of the mutated gene and one copy of the normal gene (heterozygous) are carriers of JEB. Carriers do not develop the disease and have normal epithelium, but they have a 50% chance of passing on the mutation to their offspring. If N is used to represent the normal gene and J the mutated gene, an affected animal is designated J/J, a carrier animal is N/J and a normal animal is N/N.

Comparative biochemical and histopathological evidence suggests that a deficiency in the glycogen branching enzyme, encoded by the GBE1 gene, is responsible for a recently identified recessive fatal fetal and neonatal glycogen storage disease (GSD) in American Quarter Horses termed GSD IV. In the GBE1 cDNA sequences for control horses and affected foals, a C to A substitution at base 102 has been identified that results in a tyrosine (Y) to stop (X) mutation in codon 34 of exon 1. All 11 affected foals were homozygous for the X34 allele, their 11 available dams and sires were heterozygous, and all 16 control horses were homozygous for the Y34 allele. The previous findings of poorly branched glycogen, abnormal polysaccharide accumulation, lack of measurable GBE1 enzyme activity and immunodetectable GBE1 protein, coupled with the present observation of abundant GBE1 mRNA in affected foals, are all consistent with the nonsense mutation in the 699 amino acid GBE1 protein. The affected foal pedigrees have a common ancestor and contain prolific stallions that are likely carriers of the recessive X34 allele. Defining the molecular basis of equine GSD IV will allow for accurate DNA testing and the ability to prevent occurrence of this devastating disease affecting American Quarter Horses and related breeds. See e.g., Ward et al., Mammalian Genome 15(7): 570-577 (2004).

Lethal White Overo (LWO) syndrome occurs when a horse is homozygous (OO) for the frame overo gene. This genetic disorder causes the intestinal system not to develop properly (involving aganglionosis of the bowel). The foal will die within the first 72 hours after birth when its first meals cannot be digested properly. The lethal white foal will be born almost pure white. This genetic abnormality is caused by a dinucleotide TC-->AG mutation, which changes isoleucine to lysine of the EDNRB protein.

Horses that do not have LWO syndrome can still be carriers of the LWO gene. When they are carriers of this gene, they are said to be heterozygous (nO) for the LWO gene and may pass it on to offspring. The heterozygous LWO gene in a horse occurs when the diploid (one copy from mother and one from father) of the LWO gene contains one frame overo copy and one non-frame overo copy and is often referred to as positive for frame overo. Since frame overo is a desirable quality and requires one frame overo copy, proper mating must be done to avoid possible loss due to lethal white overo while still achieving a high probability for the frame overo pattern. The way to avoid this problem is to avoid breeding frame overo to frame overo.

In additional embodiments, the disease is selected from the group consisting of congenital myotonia, muscular dystrophy, globoid cell leucodystrophy, GM-gangliosidosis, Hemophilia B, hereditary cataracts, phosphofructokinase deficiency, thrombasthenic thrombopathia, retinal dystrophy, type-2 von Willerbrand's disease, and Type III von Willebrand. In certain other embodiments, the disease is selected from the group consisting of hypertrophic cardiomyopathy, polycystic kidney disease and mucopolysaccharidosis.

Further information regarding disease may be identified by searching genetic databases or consulting periodicals or texts used in the vertinary industries and genetic testing industries. Thus, the diseases and sequences provided herein are intended to be nonlimiting with respect to scope.

### Kits and Uses

The invention also relates to kits, which can be used, for example, to perform a method of the invention. Thus, in one embodiment, the invention provides a kit for identifying a plurality of polymorphisms. Such a kit can contain, for example, an oligonucleotide probe(s), primer, or primer pair, or combinations thereof for identifying the nucleotide polymorphisms according to the present invention, following hybridization, primer extension, cleavage of the probe and fluorescence detection. Such oligonucleotides being useful, for example, to identify a polymorphism as disclosed herein; or can contain one or more nucleotide marker sequences corresponding to a characteristic selected from the group consisting of identity, parentage, breed, sex, genotype and phenotype.

In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease). The primers or probes can be included in a kit in a labeled form, for example with a label such as biotin or an antibody. In one embodiment, a kit of the invention provides a plurality of oligonucleotides of the invention, including one or more oligonucleotide probes or one or more primers, including forward and/or reverse primers, or a combination of such probes and primers or primer pairs. Such a kit also can contain probes and/or primers that conveniently allow a method of the invention to be performed using an assay plate or another substrate according to the invention.

The kit can also include instructions for using the probes or primers to determine a plurality of nucleotide marker polymorphisms.

The methods of the present invention are useful in the prevention of mishandling, mislabeling and switching of samples in the course of genetic testing. This invention prevents or corrects identification errors associated with mishandling, mislabeling and switching of samples by incorporating a genetic fingerprint or molecular identifier into the record of the genetic or other test, obtained, for example in the form of an image. In this way, an unambiguous link between that record and the animal's identity is established. The molecular identifier may serve to track and to confirm the identity of the sample, thereby providing a means for authentication. The methods of the present invention provide compositions and methods to create a genetic ID, also referred to herein as an ID, concurrently with the completion of a polymorphic genetic analysis.

It will be understood by one of ordinary skill in the art that the compositions, methods and systems of the present invention can be utilized for cost-efficient and rapid analysis of a plurality of polymorphisms in other species of animals, including but not limited to humans, birds, reptiles, and amphibians. One of ordinary skill in the art can also utilize the present invention to detect other polymorphisms, such as SNPs, deletions, insertions and other mutations that are linked to diseases and/or phenotypes associated with the animals according to the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example. Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed. (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc. N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor. N.Y., (1986); and in Ausubel et al., Current Protocols in Molecular Biology, John Wiley and Sons, Baltimore, Md. (1989).

All of the references cited above, as well as all references cited herein, are incorporated herein by reference in their entireties.

### EXAMPLES

### Example 1

### Simultaneous Identification of Multiple Characteristics Using 64 Horse Nucleotide Marker Sequences

A nucleic acid sample isolated from an individual horse was analyzed to determine the presence of a plurality of nucleotide marker polymorphisms using an assay plate according to methods of the invention. On a single plate, 64 separate assays were simultaneously performed to determine the presence of a plurality of nucleotide marker polymorphisms, where the nucleotide marker polymorphisms comprise those as set forth in Table 2.

In each assay, sequence-specific forward and reverse primers were hybridized to the nucleic sample according to the methods of the present invention. In addition, two modified oligonucleotide probes, a first oligonucleotide probe matching Allele 1 of the nucleotide marker sequence and a second oligonucleotide probe matching Allele 2 of the nucleotide marker sequence was combined with the nucleic acid sample. Each modified oligonucleotide probe contains a reporter dye at the 5′ end of the probe (e.g., a VIC® dye, or a FAM™ dye). A nonfluorescent quencher was attached at the 3′ end of the probe. Each of the first and second oligonucleotide probes were perfectly complementary to the invariant region of Allele 1 and Allele 2 of a nucleotide marker sequence according to Table 2. Finally, a DNA polymerase was added to the reaction in order that the oligonucleotide probe would be cleaved and its fluorescent reporter dye released upon matching with Allele 1 or Allele 2. The DNA polymerase contained within the assay mix can cleaved the oligonucleotide probe when it specifically hybridized to a PCR-amplified sequence present within the sample.

The forward and reverse primers were hybridized to the nucleic acid sample. The nucleic acid sample was then amplified by PCR. Cleavage separates the reporter dye from the quencher dye, increasing fluorescence by the reporter. Thus, the fluorescence signal(s) generated by PCR amplification indicates the presence of a specific polymorphic allele within the nucleic acid sample.

PCR reactions were performed using assay plates by thermal cycling using a commercial flat-block thermal cycler. Examples of the concentrations and amounts of reagents for the PCR reaction include but are not limited to those listed in Table 12. In this example, the concentration of DNA in the 5 μl sample was 30.3 ng/μl giving 1 ng of DNA in each well of the 64 well loading plate. The starting DNA stock solution can be modified based on the amount of DNA added to the sample. For example, if 1 μl of DNA is added to the sample, a 150 ng/μl stock solution would be required to obtain a final DNA concentration of 30 ng/μl. If 2 μl of DNA is added to the sample, a 75 ng/μl stock solution would be required to obtain a final DNA concentration of 30 ng/μl. Additional concentrations and amounts of reagents and DNA can be used in the methods of the present invention.

TABLE 12

| | Stock | | Final | Volume | Master |
|---|---|---|---|---|---|
| Reagent | conc. | Units | conc | (ul) | Mix Tube |
| ABI Taqman | 2 | x | 1 | 2.5 | 1584.00 |
| Master Mix | | | | | |
| BSA | 10 | mg/ml | 0.05 | 0.025 | 15.84 |
| Pluronic | 20 | % | 1 | 0.25 | 158.40 |
| F38 | | | | | |
| Glycerol | 15 | % | 0.5 | 0.16666667 | 105.60 |
| H20 | — | — | — | 0.06 | 36.96 |

| | | |
|---|---|---|
| Master Mix total volume in each well of Black | 3 | 1900.80 |
| MatriCal loading plate | | |
| Add 2 uL DNA at 75 ng/uL | 2 | |
| Total volume in Black MatriCal loading plate: | 5.00 | |

The fluorescence output was subsequently read using a computer-based imaging system. The fluorescent output measurements were utilized to determine which particular alleles were present at the polymorphic position of each nucleotide marker sequence. Results of the assays listing the determination of both alleles for each nucleotide marker sequences are provided below in Table 13, where the assays were performed using individual samples isolated from 10 different animals.

TABLE 13

| Sample. | | | | | | | | | | |
|---|---|---|---|---|---|---|---|---|---|---|
| SampleID | 16317 | 13306 | 11986 | 13218 | 11987 | 13219 | 16317 | 13306 | 11986 | 13218 |
| ECA1_1- | C C | C C | C C | C C | C C | C C | C C | C C | C C | DS* |
| 001.Genotype | | | | | | | | | | |
| ECA1_2- | A G | A G | A G | A G | A G | A G | DS | A G | A G | DS |
| 002.Genotype | | | | | | | | | | |
| ECA1_3- | A G | A G | A A | A A | A A | A A | A G | A G | A A | DS |
| 003.Genotype | | | | | | | | | | |
| ECA2_1- | DS | DS | DS | DS | C C | C C | DS | C C | DS | DS |
| 004.Genotype | | | | | | | | | | |
| ECA2_2- | G A | G A | A A | G G | G A | G A | G A | G A | A A | DS |
| 005.Genotype | | | | | | | | | | |
| ECA2_3- | T T | T T | T G | T T | T T | T G | T T | T T | T G | DS |
| 006.Genotype | | | | | | | | | | |
| ECA3_1- | C C | T C | T T | T T | T T | T C | C C | T C | T T | DS |
| 007.Genotype | | | | | | | | | | |
| ECA3_2- | A A | DS | A A | A A | A A | A A | A A | A A | A A | DS |
| 008.Genotype | | | | | | | | | | |
| ECA4_1- | A G | A A | A A | A G | A A | A G | A G | A A | A A | DS |
| 009.Genotype | | | | | | | | | | |
| ECA4_2- | G G | DS | G G | G G | G G | G G | G G | G G | G G | DS |
| 010.Genotype | | | | | | | | | | |
| ECA5_1- | A G | A A | A G | A A | A A | A A | A G | A A | A G | DS |
| 011.Genotype | | | | | | | | | | |
| ECA5_2- | A G | DS | A G | A G | A G | A G | A G | A G | A G | A G |
| 012.Genotype | | | | | | | | | | |
| ECA5_3- | T C | C C | T C | T C | T C | T C | T C | C C | T C | DS |
| 013.Genotype | | | | | | | | | | |
| ECA6_1- | T G | T T | T T | T T | T T | T T | T G | T T | T T | DS |
| 014.Genotype | | | | | | | | | | |
| ECA6_2- | G G | A A | G A | G G | G G | G A | G G | A A | G A | DS |
| 015.Genotype | | | | | | | | | | |
| ECA7_1- | C C | C T | C T | C C | T T | T T | C C | C T | C T | DS |
| 016.Genotype | | | | | | | | | | |
| ECA7_2- | C C | C C | C C | C C | C C | C C | C C | C C | C C | DS |
| 017.Genotype | | | | | | | | | | |
| ECA8_1- | T T | DS | T T | T T | T T | T T | T T | C C | T T | DS |
| 018.Genotype | | | | | | | | | | |
| ECA8_2- | C T | C T | C C | C T | C C | C C | C T | C T | C C | DS |
| 019.Genotype | | | | | | | | | | |
| ECA9_1- | T T | C T | C C | C C | C C | C C | T T | C T | C C | DS |
| 020.Genotype | | | | | | | | | | |
| ECA9_2- | T T | T T | T T | T T | T T | T T | T T | T T | T T | DS |
| 021.Genotype | | | | | | | | | | |
| ECA10_1- | A A | A A | A A | A A | A A | A C | A A | A A | A A | DS |
| 022.Genotype | | | | | | | | | | |
| ECA10_2- | T T | T T | C C | C T | C C | C C | T T | T T | C C | DS |
| 023.Genotype | | | | | | | | | | |
| ECA11_1- | T T | T T | T T | T T | T T | T T | T T | T T | T T | T T |
| 024.Genotype | | | | | | | | | | |
| ECA11_2- | T T | T T | T T | T T | T T | T T | T T | T T | T T | DS |
| 025.Genotype | | | | | | | | | | |
| ECA12_1- | T C | T T | T C | T C | DS | DS | T C | DS | DS | C C |
| 026.Genotype | | | | | | | | | | |
| ECA12_2- | T T | T T | T C | T C | T T | T T | T T | T T | T C | T C |
| 027.Genotype | | | | | | | | | | |
| ECA13_1- | A A | DS | A A | A A | A A | A A | A A | A G | A A | DS |
| 028.Genotype | | | | | | | | | | |
| ECA13_2- | G G | DS | A A | A A | G G | A A | G G | A A | A A | G A |
| 029.Genotype | | | | | | | | | | |
| ECA14_1- | G G | G G | G G | G G | G G | G G | G G | G G | G G | DS |
| 030.Genotype | | | | | | | | | | |
| ECA14_2- | G G | C C | G G | C G | C G | G G | G G | C C | G G | DS |
| 031.Genotype | | | | | | | | | | |
| ECA15_1- | A G | A A | G G | A A | G G | A A | A G | A A | G G | DS |
| 032.Genotype | | | | | | | | | | |
| ECA15_2- | G G | G G | G G | A G | G G | G G | G G | G G | G G | DS |
| 033.Genotype | | | | | | | | | | |
| ECA16_1- | A A | DS | A G | A A | A G | A G | A A | G G | A G | DS |
| 034.Genotype | | | | | | | | | | |
| ECA16_2- | T T | T T | T C | T C | T C | T C | T T | T T | T C | DS |
| 035.Genotype | | | | | | | | | | |
| ECA17_1- | A A | A A | A A | A A | A G | A A | A A | A A | A A | DS |
| 036.Genotype | | | | | | | | | | |
| ECA17_2- | T T | T T | T T | T T | T T | T T | T T | T T | T T | DS |
| 037.Genotype | | | | | | | | | | |
| ECA18_1- | A G | A A | A A | A A | A A | A A | A G | A A | A A | DS |
| 038.Genotype | | | | | | | | | | |
| ECA19_1- | T T | T C | C C | C C | C C | C C | T T | T C | C C | DS |
| 039.Genotype | | | | | | | | | | |
| ECA20_1- | T T | T T | T T | T T | T T | T T | T T | T T | T T | DS |
| 040.Genotype | | | | | | | | | | |
| ECA21_1- | DS | A T | A T | A T | A T | A T | A T | A T | A T | DS |
| 041.Genotype | | | | | | | | | | |
| ECA22_1- | T T | DS | T T | T T | T T | T T | T T | T C | T T | DS |
| 042.Genotype | | | | | | | | | | |
| ECA23_1- | C C | C C | C C | C C | C C | C C | C C | C C | C C | DS |
| 043.Genotype | | | | | | | | | | |
| ECA24_1- | T C | T T | T T | T T | T T | T T | T C | T T | T T | DS |
| 044.Genotype | | | | | | | | | | |
| ECA25_1- | T T | T T | T C | T T | T T | T T | T T | T T | T C | DS |
| 045.Genotype | | | | | | | | | | |
| ECA26_1- | C C | C C | C T | C T | C T | C T | C C | C C | C T | DS |
| 046.Genotype | | | | | | | | | | |
| ECA27_1- | C T | T T | T T | C T | C T | T T | C T | T T | T T | DS |
| 047.Genotype | | | | | | | | | | |
| ECA28_1- | C C | C C | C C | C C | C C | C C | C C | C C | C C | DS |
| 048.Genotype | | | | | | | | | | |
| ECA29_REPL- | C C | C T | T T | C T | T T | T T | C C | C T | T T | DS |
| 049.Genotype | | | | | | | | | | |
| ECA30_1- | A G | DS | A A | A A | A A | A A | A G | A A | A A | DS |
| 050.Genotype | | | | | | | | | | |
| ECA31_1- | C T | T T | C T | T T | C T | T T | C T | T T | C T | C C |
| 051.Genotype | | | | | | | | | | |
| ECA31_2- | A A | G G | G A | A A | G A | G G | A A | G G | G A | DS |
| 052.Genotype | | | | | | | | | | |
| ECAX_1- | A A | A A | A A | A A | A A | A A | A A | A A | A A | DS |
| 053.Genotype | | | | | | | | | | |
| ECA1_4- | T T | T T | T T | T T | T C | T T | T T | T T | T T | DS |
| 065.Genotype | | | | | | | | | | |
| E_AGOUTI_10. | GAAA | * * | GAAA | GAAA | GAAA | * * | GAAA | * * | GAAA | DS |
| Genotype | AGAA | | AGAA | AGAA | AGAA | | AGAA | | AGAA | |
| | GCA* | | GCA* | GCA* | GCA* | | GCA* | | GCA* | |
| CREAM- | G G | G G | G G | G G | G G | G G | G G | G G | G G | G G |
| CRE.Genotype | | | | | | | | | | |
| HORSE_RED- | C C | T T | C C | C C | C C | C T | C C | T T | C C | C C |
| MC1R.Genotype | | | | | | | | | | |
| SABINO- | DS | DS | T T | T T | T T | T T | T T | T T | T T | T A |
| SABI.Genotype | | | | | | | | | | |
| SILVERH- | C C | C C | C C | C C | C C | C C | C C | C C | C C | DS |
| SILH.Genotype | | | | | | | | | | |
| TOBIANO- | C C | C C | C C | C C | C C | C C | C C | C C | C C | DS |
| TOB.Genotype | | | | | | | | | | |
| HYPP_NEW- | C C | C C | C C | C C | C C | C C | C C | C C | C C | DS |
| HYP.Genotype | | | | | | | | | | |
| HORSE_LWO- | TC | TC | TC | TC | TC | TC | TC | TC | TC | DS |
| LWO.Genotype | TC | TC | TC | TC | TC | TC | TC | TC | TC | |
| HORSE_JEB- | * C | * C | * C | * C | * C | * C | * C | * C | * C | DS |
| JEB.Genotype | | | | | | | | | | |
| HORSE_GBE1- | C C | C C | DS | C C | C C | C C | C C | C C | C C | C A |
| GBE1.Genotype | | | | | | | | | | |

*DS: Polymorphic alleles were read under different stringency conditions with reliable results.

Each sample was tested against the 64 markers listed in Table 2. The two oligonucleotide probe contained VIC® and FAM™, respectively, at the 5′ end of the probes. The control was no template.

Assays as described above were performed using additional samples isolated from 10 other animals. Results of the additional assays listing the determination of both alleles for each nucleotide marker sequences are provided below in Table 14:

TABLE 14

| Sample. | | | | | | | | | | |
|---|---|---|---|---|---|---|---|---|---|---|
| SampleID | 11987 | 13219 | 10740 | 15849 | 15051 | 15850 | 16297 | 16298 | 10740 | NTC |
| ECA1_1- | C C | C C | C C | C C | C C | C C | C C | C T | C C | DS |
| 001.Genotype | | | | | | | | | | |
| ECA1_2- | A G | A G | A G | A G | A G | A G | A G | A G | A G | DS |
| 002.Genotype | | | | | | | | | | |
| ECA1_3- | A A | A A | A A | A G | A G | A G | A A | A A | A A | DS |
| 003.Genotype | | | | | | | | | | |
| ECA2_1- | DS | C C | C C | G C | DS | DS | C C | C C | C C | DS |
| 004.Genotype | | | | | | | | | | |
| ECA2_2- | G A | G A | A A | G G | G A | G G | A A | A A | A A | DS |
| 005.Genotype | | | | | | | | | | |
| ECA2_3- | T T | T G | T T | T T | T T | T T | T G | T T | T T | DS |
| 006.Genotype | | | | | | | | | | |
| ECA3_1- | T T | T C | T C | T T | T C | T T | T T | T C | T C | DS |
| 007.Genotype | | | | | | | | | | |
| ECA3_2- | A A | A A | A A | A G | A A | A A | A A | A A | A A | DS |
| 008.Genotype | | | | | | | | | | |
| ECA4_1- | A A | A G | G G | A A | A A | A A | A A | A A | G G | DS |
| 009.Genotype | | | | | | | | | | |
| ECA4_2- | G G | G G | G G | G G | G A | DS | G G | G A | G G | DS |
| 010.Genotype | | | | | | | | | | |
| ECA5_1- | A A | A A | A A | A A | A A | A A | A A | A A | A A | DS |
| 011.Genotype | | | | | | | | | | |
| ECA5_2- | A G | A G | A G | A G | DS | A G | A G | A G | A G | A G |
| 012.Genotype | | | | | | | | | | |
| ECA5_3- | T C | T C | T C | C C | T T | T C | T C | T C | T C | DS |
| 013.Genotype | | | | | | | | | | |
| ECA6_1- | T T | T T | G G | T T | T T | T T | T T | T T | G G | DS |
| 014.Genotype | | | | | | | | | | |
| ECA6_2- | G G | G A | G G | G G | G G | G G | G G | G G | G G | DS |
| 015.Genotype | | | | | | | | | | |
| ECA7_1- | T T | T T | C C | T T | C T | C T | C T | T T | C C | DS |
| 016.Genotype | | | | | | | | | | |
| ECA7_2- | C C | C C | C C | C C | C C | C C | C C | C C | C C | DS |
| 017.Genotype | | | | | | | | | | |
| ECA8_1- | T T | T T | T T | T T | T T | T T | T T | T T | T T | DS |
| 018.Genotype | | | | | | | | | | |
| ECA8_2- | C C | C C | C C | C T | C C | C T | C C | C C | C C | DS |
| 019.Genotype | | | | | | | | | | |
| ECA9_1- | C C | C C | C C | C C | C C | C C | C T | C T | C C | DS |
| 020.Genotype | | | | | | | | | | |
| ECA9_2- | T T | T T | T T | T T | T T | T T | T T | T T | T T | DS |
| 021.Genotype | | | | | | | | | | |
| ECA10_1- | A A | A C | A A | A A | A A | A A | A A | A A | A A | DS |
| 022.Genotype | | | | | | | | | | |
| ECA10_2- | C C | C C | T T | C T | C C | C C | C T | C T | T T | DS |
| 023.Genotype | | | | | | | | | | |
| ECA11_1- | T T | T T | T C | T C | T C | T C | T C | T C | T C | DS |
| 024.Genotype | | | | | | | | | | |
| ECA11-2- | T T | T T | T T | T T | T T | T T | T T | T T | T T | DS |
| 025.Genotype | | | | | | | | | | |
| ECA12_1- | T C | DS | DS | T C | T C | T C | T C | DS | T C | C C |
| 026.Genotype | | | | | | | | | | |
| ECA12_2- | T T | T T | T T | T T | T C | T T | T C | T T | T T | DS |
| 027.Genotype | | | | | | | | | | |
| ECA13_1- | A A | A A | A A | A A | A A | A A | A A | A A | A A | DS |
| 028.Genotype | | | | | | | | | | |
| ECA13_2- | G G | G A | A A | G A | G G | A A | A A | A A | A A | G A |
| 029.Genotype | | | | | | | | | | |
| ECA14_1- | G G | G G | G G | G G | G G | G G | G G | G G | G G | DS |
| 030.Genotype | | | | | | | | | | |
| ECA14_2- | C G | G G | G G | G G | C G | G G | G G | C C | G G | DS |
| 031.Genotype | | | | | | | | | | |
| ECA15_1- | G G | A A | A G | A A | A A | A A | A A | A A | A G | DS |
| 032.Genotype | | | | | | | | | | |
| ECA15_2- | G G | G G | G G | G G | A G | G G | G G | G G | G G | DS |
| 033.Genotype | | | | | | | | | | |
| ECA16_1- | A G | A G | G G | A A | G G | G G | A G | A G | G G | DS |
| 034.Genotype | | | | | | | | | | |
| ECA16_2- | T C | T C | T T | T C | T T | T T | T C | T C | T T | DS |
| 035.Genotype | | | | | | | | | | |
| ECA17_1- | A G | A A | G G | A A | A A | A A | A A | A A | G G | DS |
| 036.Genotype | | | | | | | | | | |
| ECA17_2- | T T | T T | T C | T T | T T | T T | T T | T T | T C | DS |
| 037.Genotype | | | | | | | | | | |
| ECA18_1- | A A | A A | A A | A A | A A | A A | A A | A A | A A | DS |
| 038.Genotype | | | | | | | | | | |
| ECA19_1- | C C | C C | T T | C C | C C | T C | C C | C C | T T | DS |
| 039.Genotype | | | | | | | | | | |
| ECA20_1- | T T | T T | T T | T T | T T | T T | T T | T T | T T | DS |
| 040.Genotype | | | | | | | | | | |
| ECA21_1- | A T | A T | A A | A T | A T | A T | A T | A T | A T | DS |
| 041.Genotype | | | | | | | | | | |
| ECA22_1- | T T | T T | T T | T T | T T | T T | T T | T T | T T | DS |
| 042.Genotype | | | | | | | | | | |
| ECA23_1- | C C | C C | C C | C C | C C | C C | C C | C C | C C | DS |
| 043.Genotype | | | | | | | | | | |
| ECA24_1- | T T | T T | T T | T T | T T | T C | T T | T T | T T | DS |
| 044.Genotype | | | | | | | | | | |
| ECA25_1- | T T | T T | T T | T T | T C | T C | C C | T C | T T | DS |
| 045.Genotype | | | | | | | | | | |
| ECA26_1- | C T | C T | C C | C T | DS | C T | C T | C T | C C | DS |
| 046.Genotype | | | | | | | | | | |
| ECA27_1- | C T | T T | T T | T T | T T | T T | T T | T T | T T | DS |
| 047.Genotype | | | | | | | | | | |
| ECA28_1- | C C | C C | C C | C T | C C | C C | C C | C C | C C | DS |
| 048.Genotype | | | | | | | | | | |
| ECA29_REPL- | T T | T T | C T | T T | T T | T T | T T | T T | C T | DS |
| 049.Genotype | | | | | | | | | | |
| ECA30_1- | A A | A A | A G | A A | A A | A A | A A | A A | A G | DS |
| 050.Genotype | | | | | | | | | | |
| ECA31_1- | C T | T T | C T | C T | C T | C T | T T | C T | C T | DS |
| 051.Genotype | | | | | | | | | | |
| ECA31_2- | G A | G G | G A | G G | G A | G A | G G | G G | G A | DS |
| 052.Genotype | | | | | | | | | | |
| ECAX_1- | A A | A A | A A | A A | A A | A A | A A | A A | A A | DS |
| 053.Genotype | | | | | | | | | | |
| ECA1_4- | T C | T T | T T | T T | DS | T T | T T | T C | T T | DS |
| 065.Genotype | | | | | | | | | | |
| E_AGOUTI_10. | GAAA | * * | GAAA | GAAA | GAAA | * * | GAAA | * * | GAAA | DS |
| Genotype | AGAA | | AGAA | AGAA | AGAA | | AGAA | | AGAA | |
| | GCA* | | GCA* | GCA* | GCA* | | GCA* | | GCA* | |
| CREAM- | G G | G G | G G | G G | G G | G G | G G | G G | G G | DS |
| CRE.Genotype | | | | | | | | | | |
| HORSE_RED- | C C | C T | C C | C T | C C | T T | C T | T T | C C | DS |
| MC1R.Genotype | | | | | | | | | | |
| SABINO- | T T | T T | T T | T T | T T | DS | T T | T T | T T | T A |
| SABI.Genotype | | | | | | | | | | |
| SILVERH- | C C | C C | C C | C C | C T | C C | C C | C C | C C | DS |
| SILH.Genotype | | | | | | | | | | |
| TOBIANO- | C C | C C | C C | C C | C C | C C | C C | C C | C C | DS |
| TOB.Genotype | | | | | | | | | | |
| HYPP_NEW- | C C | C C | C C | C C | C C | C C | C C | C C | C C | DS |
| HYP.Genotype | | | | | | | | | | |
| HORSE_LWO- | TC | TC | TC | TC | DS | TC | TC | TC | TC | DS |
| LWO.Genotype | TC | TC | TC | TC | | TC | TC | TC | TC | |
| HORSE_JEB- | * C | * C | * C | * C | * C | * C | * C | * C | * C | DS |
| JEB.Genotype | | | | | | | | | | |
| HORSE_GBE1- | DS | C C | C C | C C | C C | C C | C C | C C | C C | DS |
| GBE1.Genotype | | | | | | | | | | |

***

Again, each sample was tested against the 64 markers listed in Table 2. The two oligonucleotide probe contained VIC® and FAM™, respectively, at the 5′ end of the probes. The control was no template.

The presence of particular alleles as disclosed in Tables 8 and 9 were utilized to determine the presence of at least two characteristics selected from the group consisting of parentage, identity and/or phenotype using information available to one of ordinary skill in the art.

### Example 2

### Simultaneous Identification of Multiple Characteristics Using 128 Horse Nucleotide Marker Sequences

A nucleic acid sample isolated from an individual horse is analyzed to determine the presence of a plurality of nucleotide marker polymorphisms using an assay plate according to methods of the invention. On a single plate, 128 separate assays are simultaneously performed to determine the presence of a plurality of nucleotide marker polymorphisms, where the nucleotide marker polymorphisms comprise those as set forth in Tables 2 and 4.

The assay is performed according to the methods described in Example 1 above. Results of the assays as measured by fluorescent output are tabulated.

### Example 3

### Simultaneous Identification of Multiple Characteristics Using Horse and Dog Nucleotide Marker Sequences

A nucleic acid sample isolated from individual horses, cattle, cats and dogs are analyzed to determine the presence of a plurality of nucleotide marker polymorphisms using an assay plate according to methods of the invention for each individual animal. On a single plate, up to 3000 separate assays are simultaneously performed to determine the presence of a plurality of nucleotide marker polymorphisms, where the nucleotide marker polymorphisms comprise those as set forth in Tables 2, 4, 6 and 8.

The assay is performed according to the methods described in Example 1 above. Results of the assays as measured by fluorescent output are tabulated.

### Example 4

### Raw Data Plots Showing Examples of Markers for Parentage, Identity, Sex, Phenotype and/or Genotype and Breed Determination

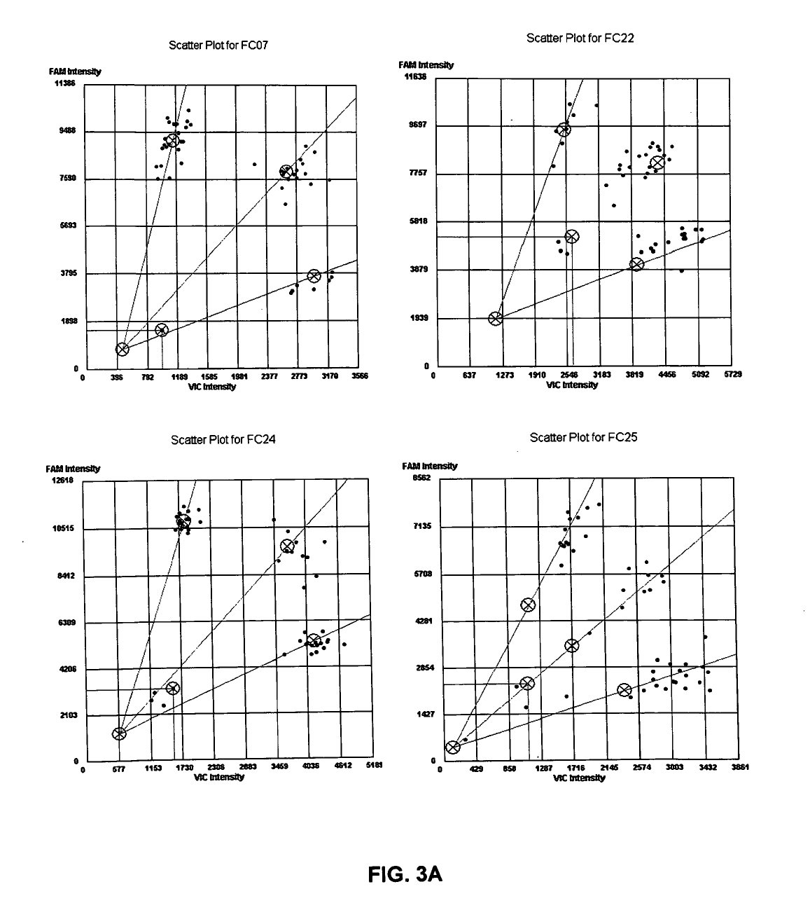

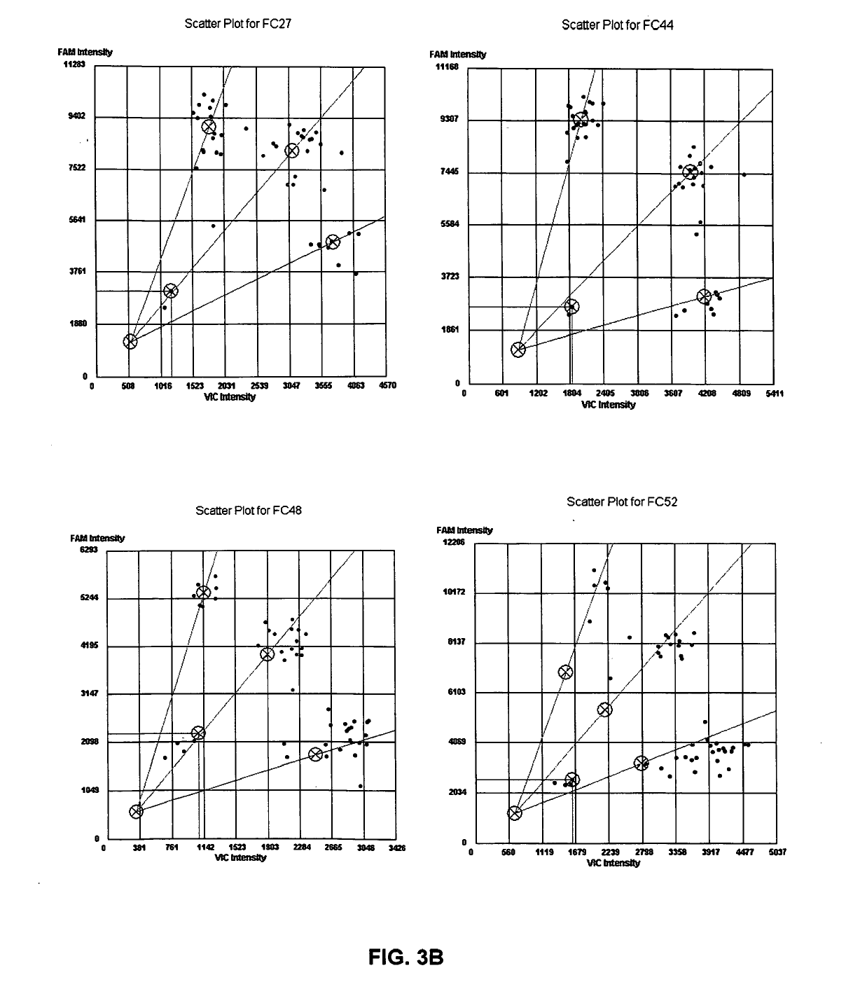

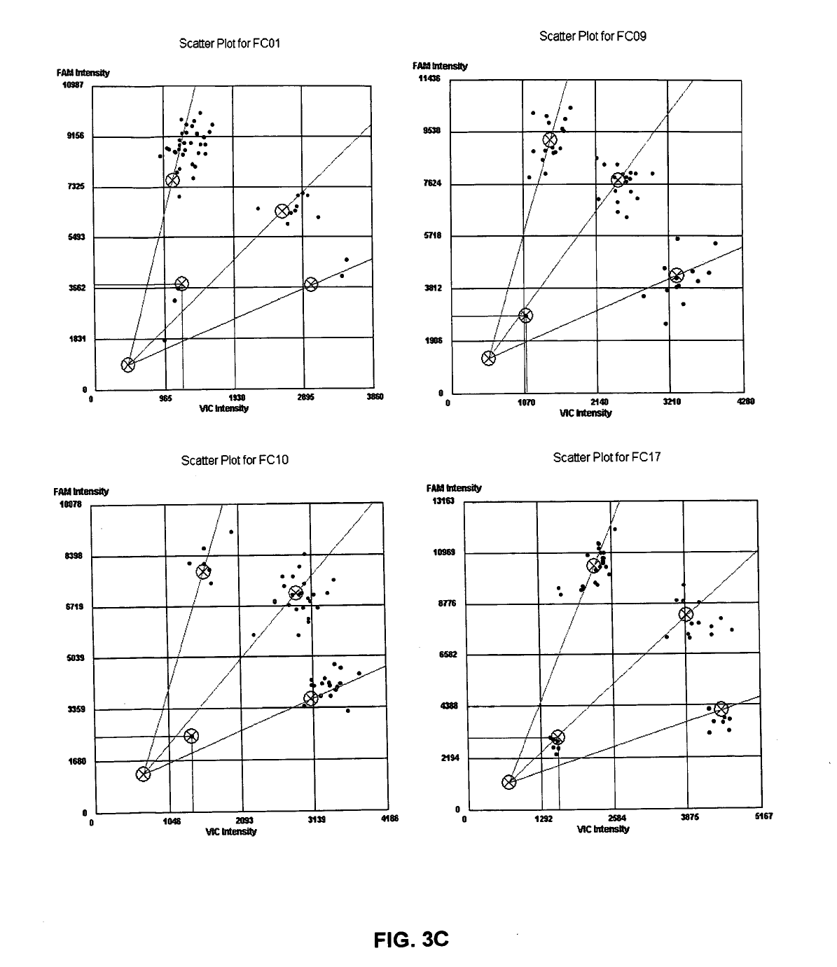

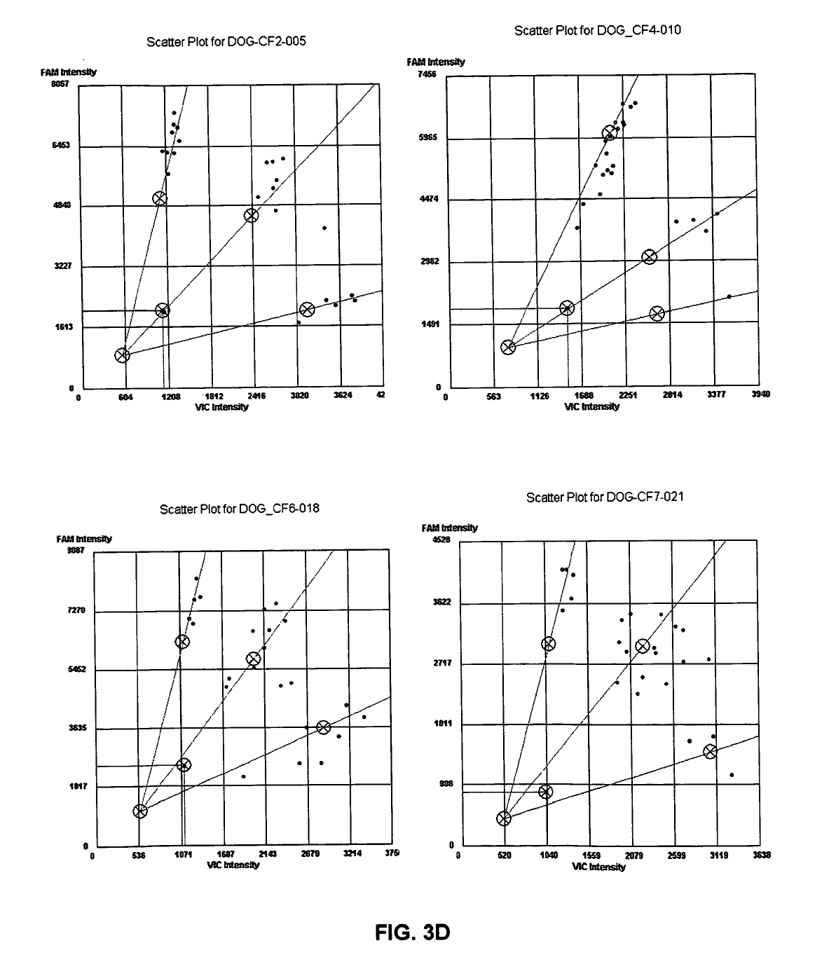

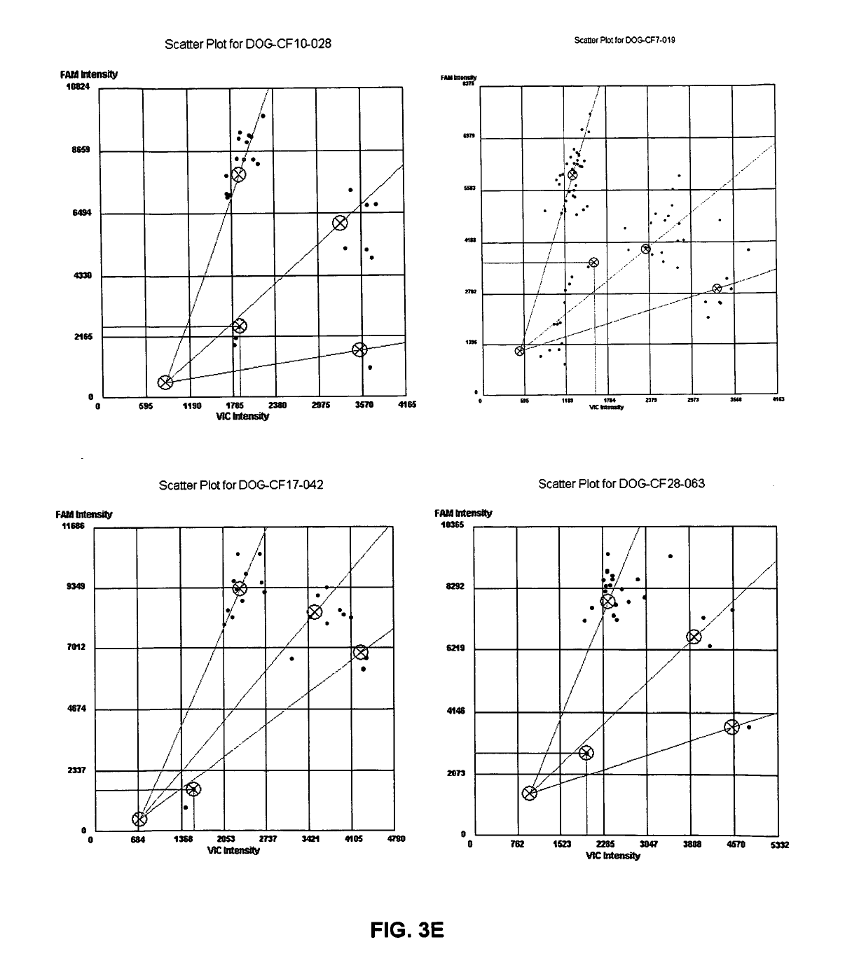

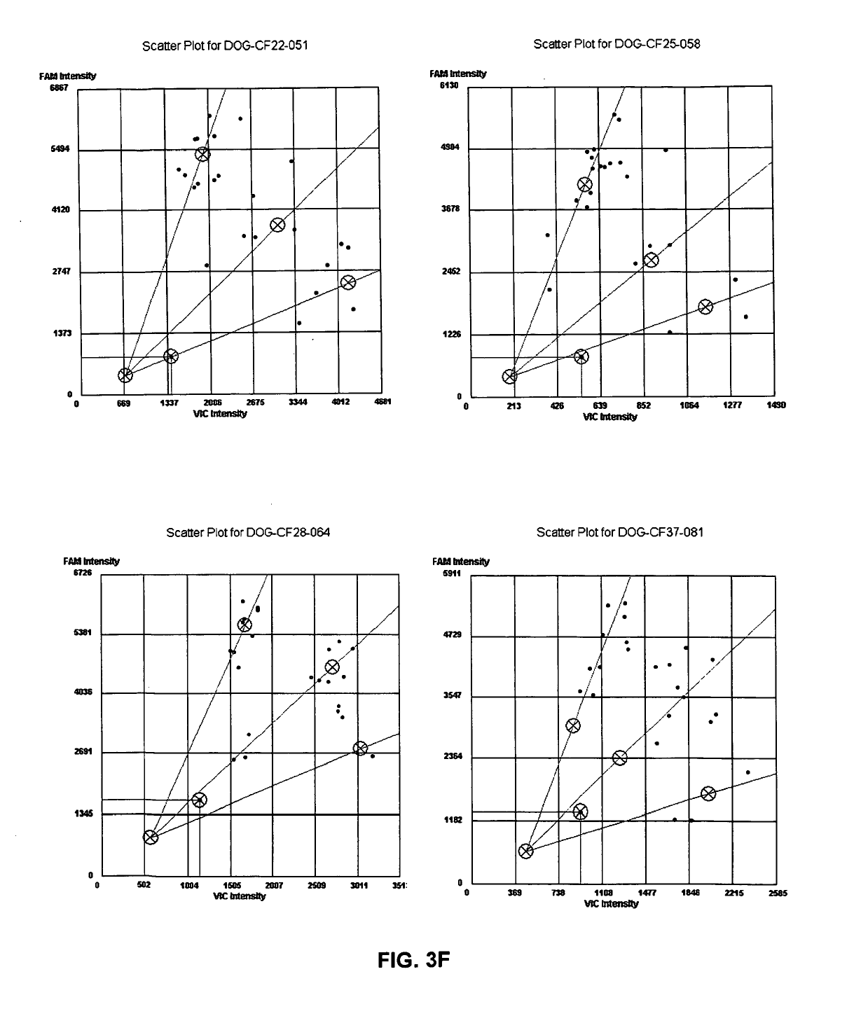

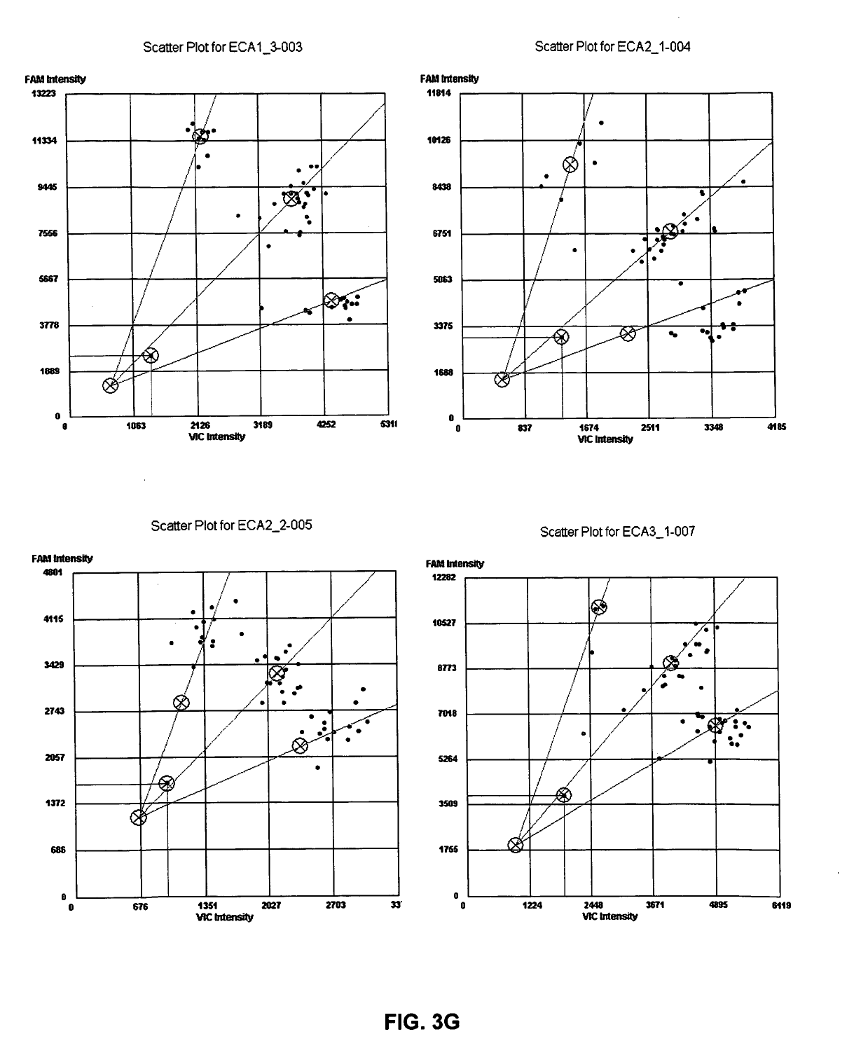

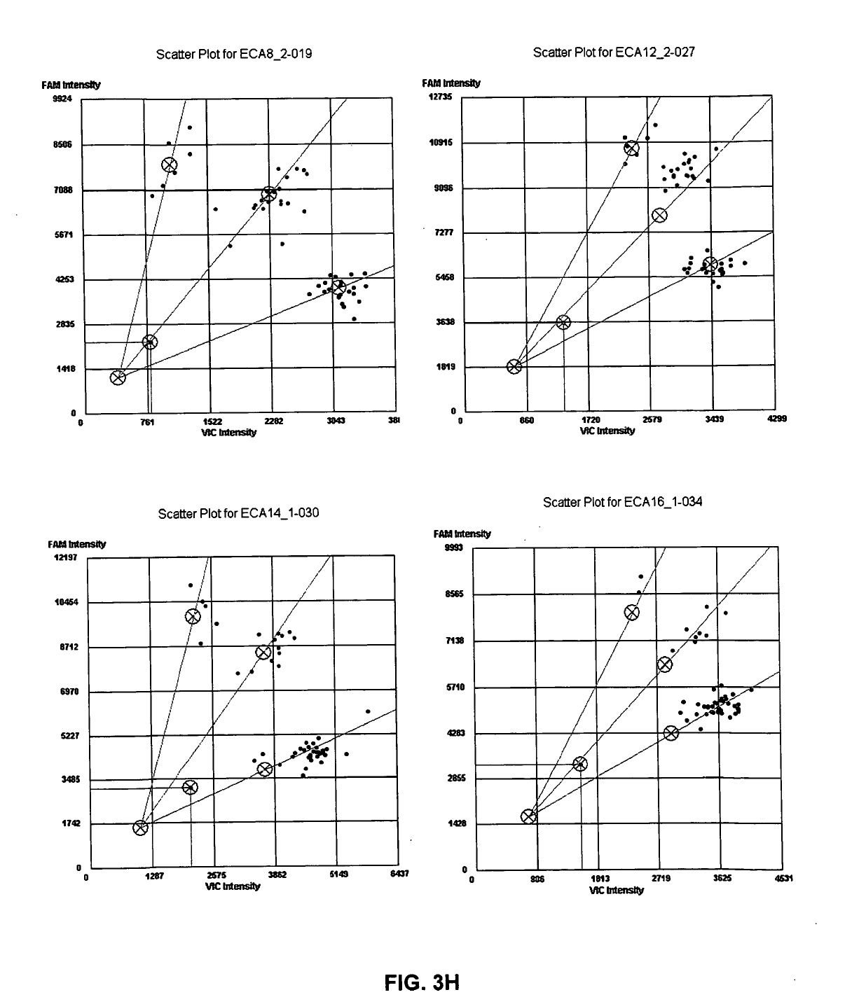

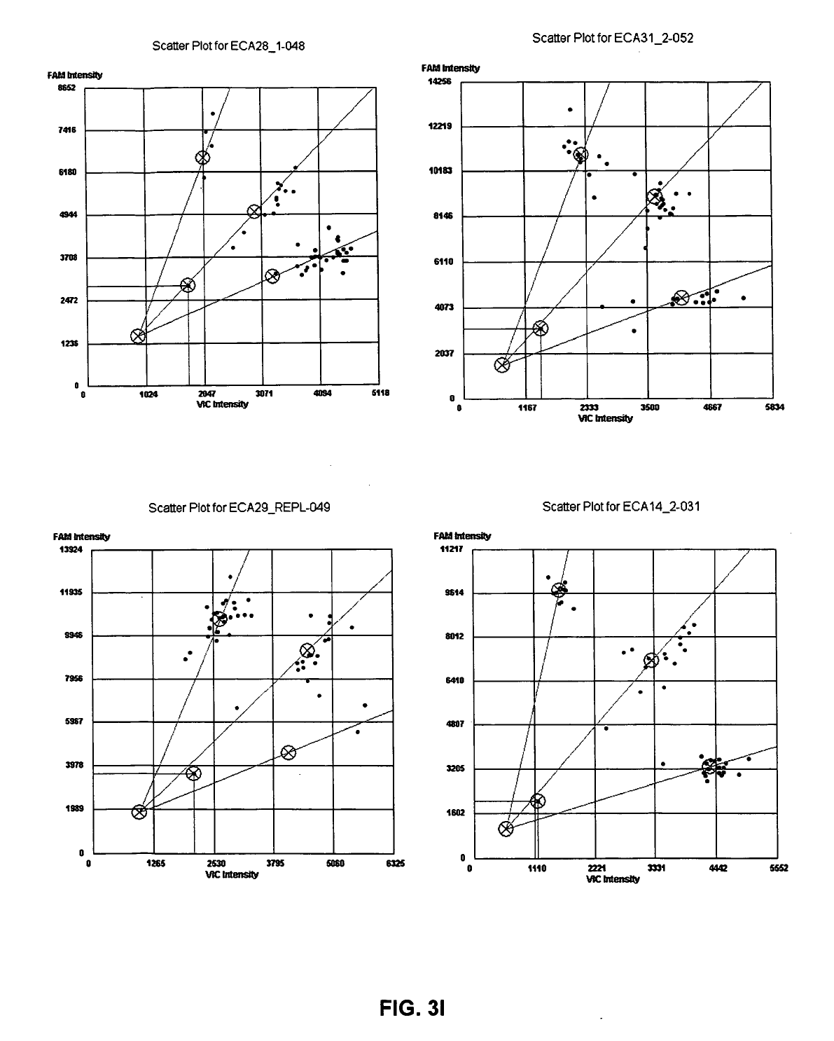

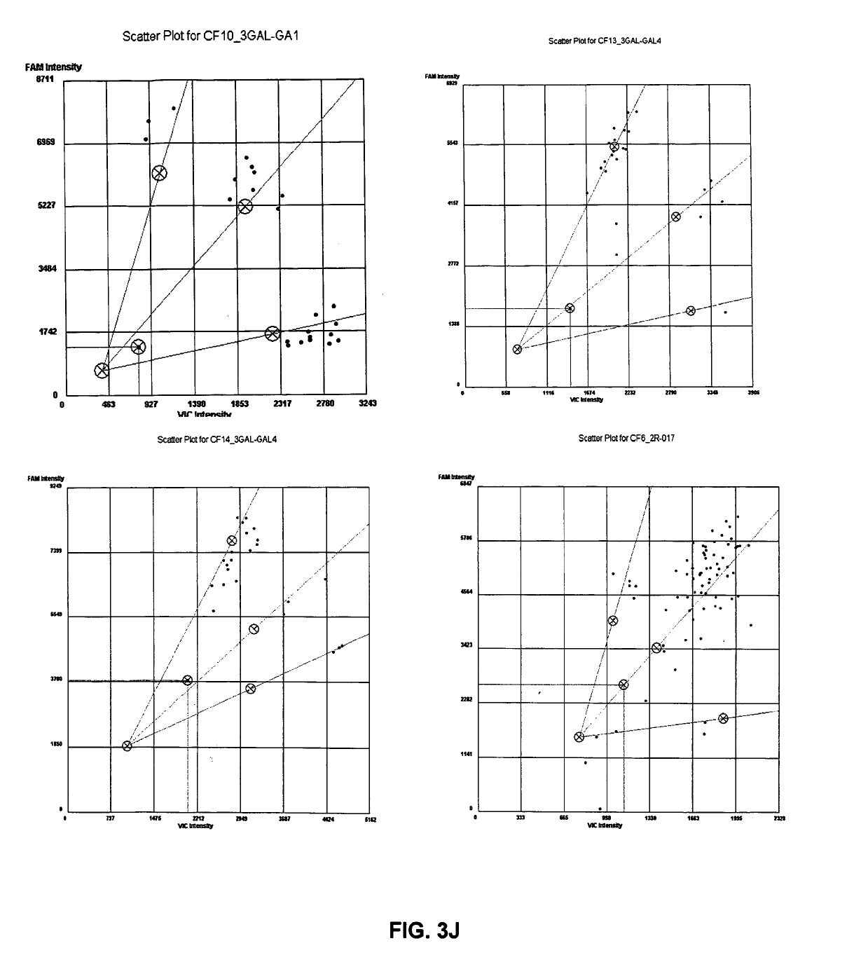

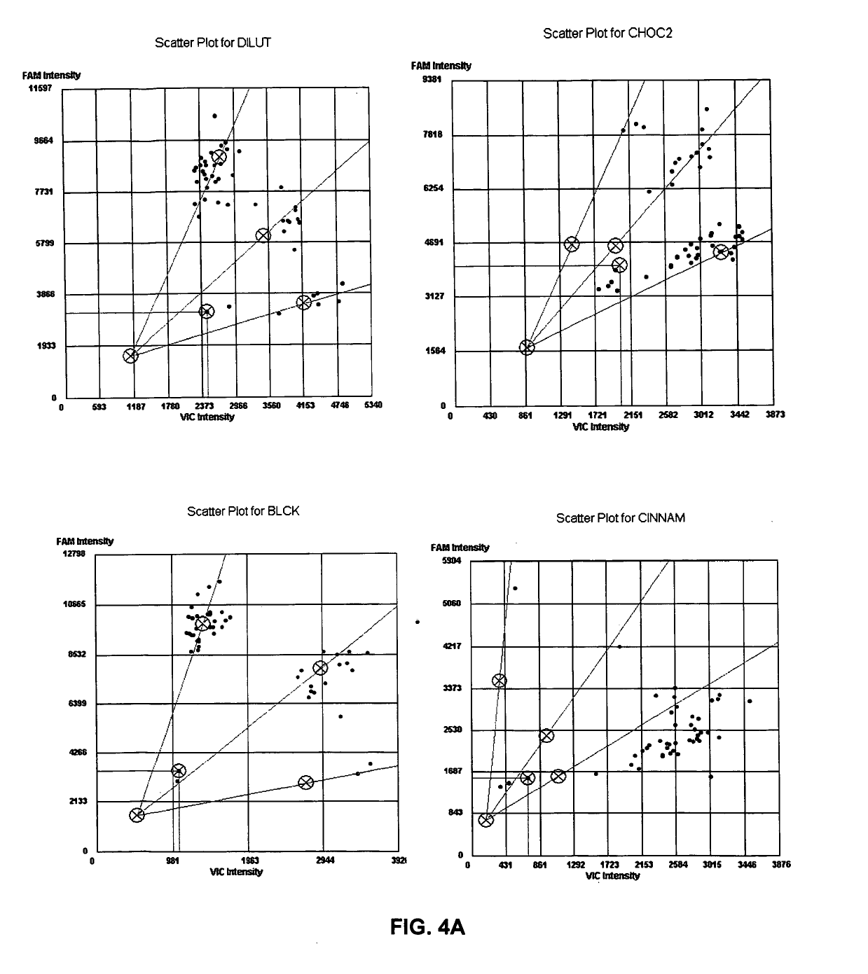

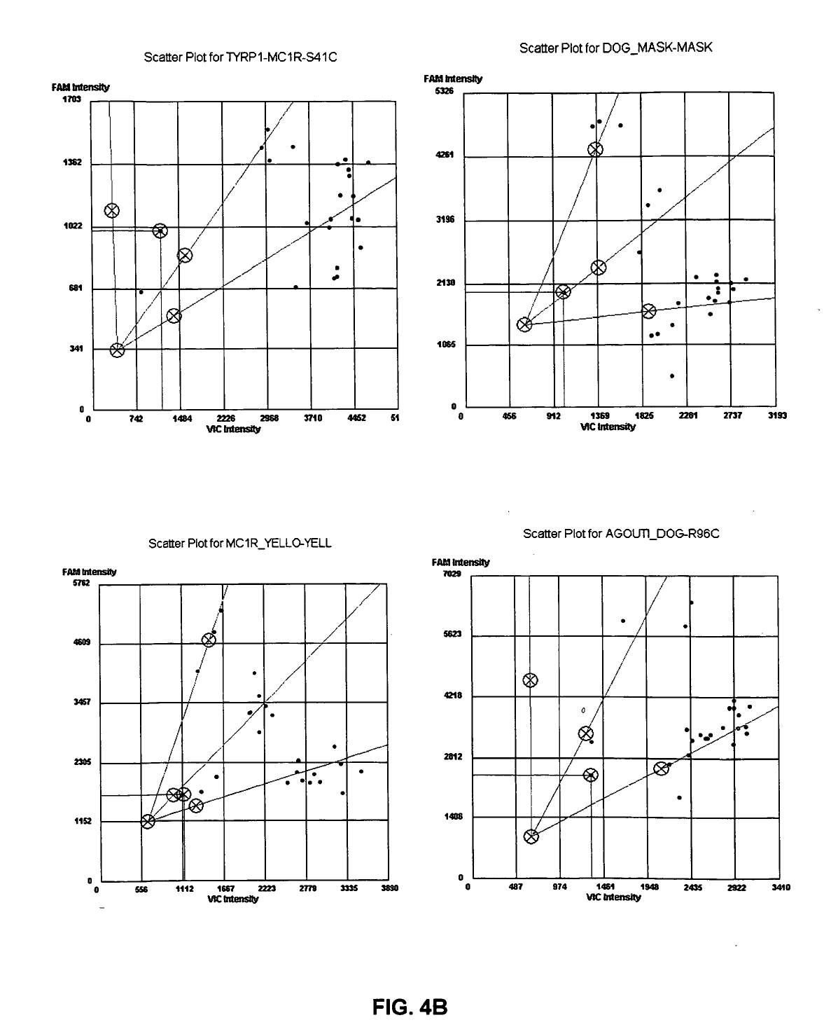

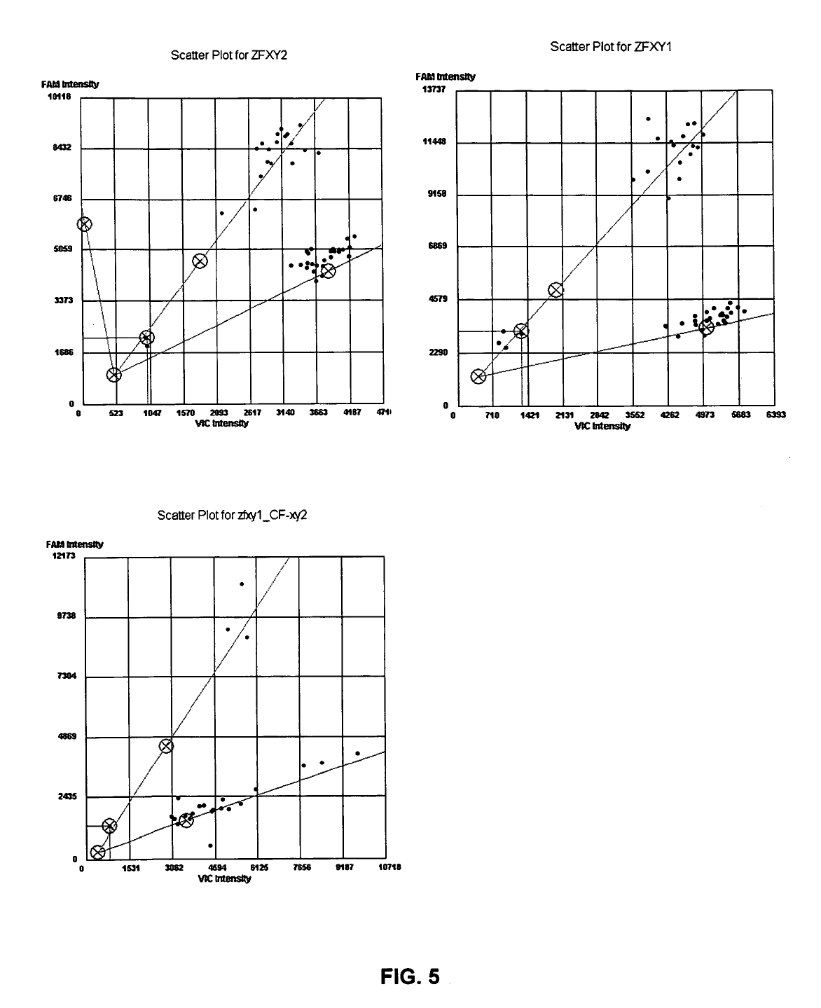

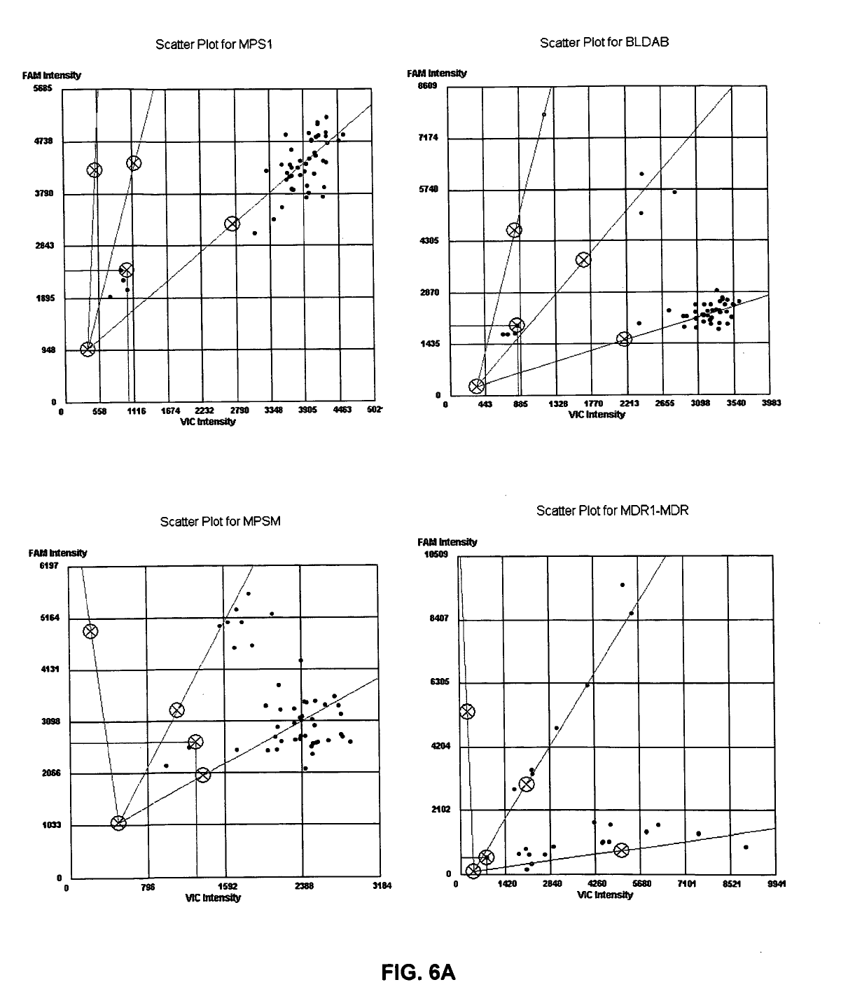

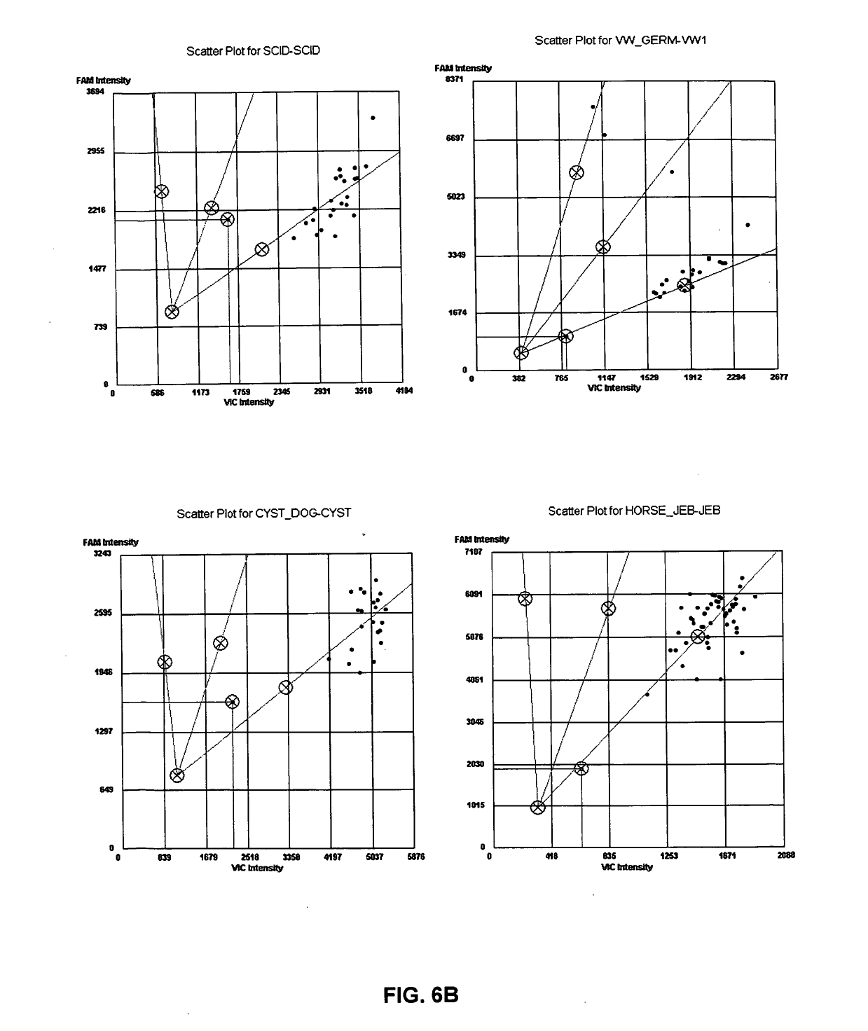

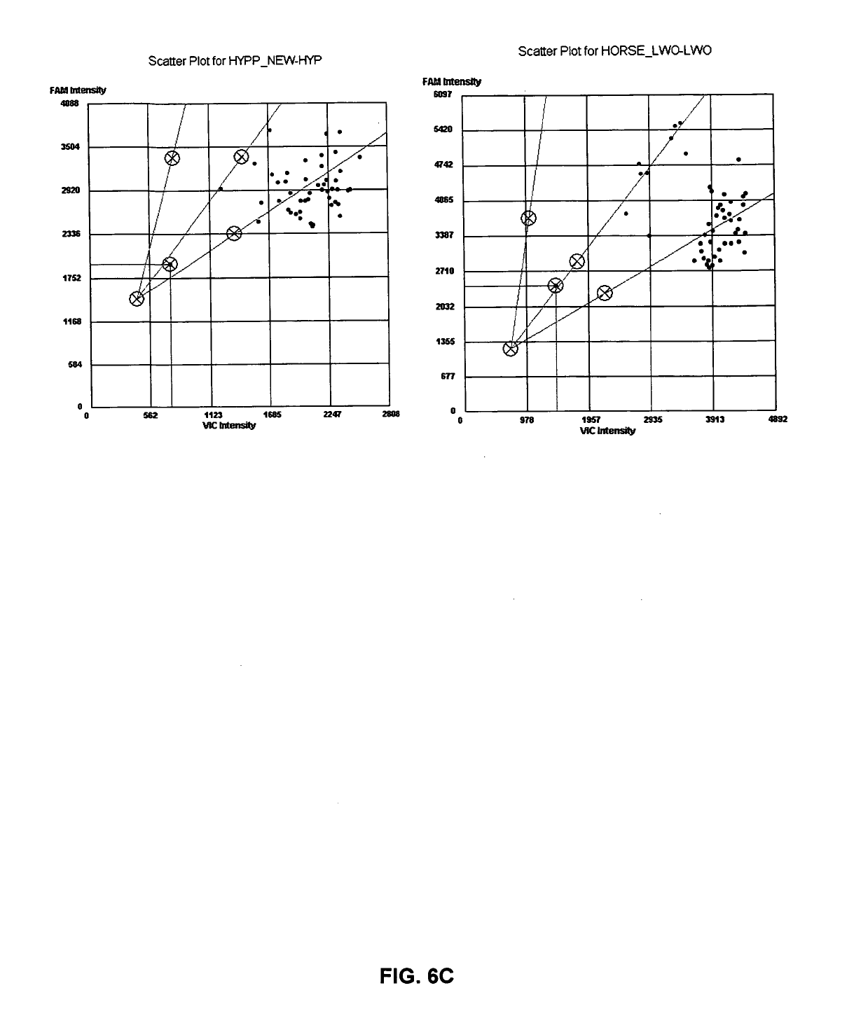

FIGS. 3A-6C provide examples of raw data plots generated by a processor based system from individual markers depicting the presence of nucleotide marker polymorphism using an assay plate according to methods of the invention for groups of 47 and 23 animals respectively comprising cat, dog, horse, and cattle species. The plots give examples of identity and parentage, genotype and/or phenotype including disease diagnostics and traits like color, sex determination where females are homozygous and males are heterozygous, and breed determination. Each individual marker was simultaneously analyzed along with 63 or 127 other markers comprising all 5 of the (i) parentage; (ii) identity; (iii) sex, (iv) genotype and (v) phenotype

What is claimed is:

1. A method for simultaneously identifying a plurality of polymorphisms in a nucleic acid sample isolated from an animal comprising the steps of:
(a) placing said nucleic acid sample in at least two recesses of an assay plate;
(b) hybridizing said nucleic acid sample to a pair of forward and reverse primers;
(c) contacting said nucleic acid sample with a first oligonucleotide probe and with a second oligonucleotide probe;
(d) performing PCR amplification; and
(e) detecting the presence of said plurality of polymorphisms in said nucleic acid sample;
wherein said first oligonucleotide probe is capable of detecting a first allele of a nucleotide marker sequence;
wherein said second oligonucleotide probe is capable of detecting a second allele of a nucleotide marker sequence;
wherein said nucleotide marker sequence is any one of the nucleotide marker sequences as set forth in Tables 1-11;
wherein said nucleotide marker sequence correlates with at least one of the characteristics of an animal selected from the group consisting of: (i) parentage; (ii) identity; (iii) sex, (iv) genotype and (v) phenotype; and
wherein said assay plate is capable of simultaneously identifying at least two characteristics of said animal selected from the group consisting of:
(i) parentage; (ii) identity; (iii) sex, (iv) genotype and (v) phenotype.

2. The method of claim 1, wherein said plurality of polymorphisms correlates with at least three characteristics.

3. The method of any one of claims 1-2, wherein said plurality of polymorphisms is simultaneously identified in nucleic acid samples isolated from at least two animals.

4. The method of any one of claims 1-3, wherein said plurality of polymorphisms is simultaneously identified in nucleic acid samples isolated from at least three animals.

5. The method of any one of claims 1-4, wherein said plurality of polymorphisms is simultaneously identified in nucleic acid samples isolated from at least four animals.

6. The method of any one of claims 1-5, wherein each of said animals is of a family selected from the group consisting of Equidae, Bovidae, Canidae, and Felidae.

7. The method of claim 6, wherein each of said animals of the family Bovidae is of a species selected from the group consisting of Bos, Ovis, and Capra.

8. The method of claim 6, wherein each of said animals of the family Equidae is of a species selected from the group consisting of Equus.

9. The method of claim 6, wherein each of said animals of the family Canidae is of a species selected from the group consisting of Canis.

10. The method of claim 6, wherein each of said animals of the family Felidae is of a species selected from the group consisting of Felis.

11. The method of any one of claims 1-10, wherein said plurality of polymorphisms comprises between about 20 and about 10,000 polymorphisms and extending to whole genome analysis.

12. The method of any one of claims 1-11, wherein said plurality of polymorphisms comprises about 60, 100, 3000, 6000 or 9000 polymorphisms.

13. The method of any one of claims 1-12, wherein said plurality of polymorphisms comprises about 64, 128, 3072, 6344 or 9216 polymorphisms.

14. The method of any one of claims 1-13, wherein said plurality of polymorphisms comprises between about 20 and about 3000 polymorphisms.

15. The method of any one of claims 1-11 and 14, wherein said plurality of polymorphisms comprises between about 20 and 200 polymorphisms.

16. The method of claim 15, wherein said plurality of polymorphisms comprises about 20, 30, 40, 50, 60, 70, 80, 90, 100, 110, 120, 130, 140, 150, 160, 170, 180, 190, or 200 polymorphisms.

17. The method of any one of claims 1-16, wherein each of said plurality of polymorphisms is a polymorphism of a nucleotide marker sequence according to Tables 2, 4, 6, 9, and 11.

18. The method of any one of claims 1-17, wherein each of said plurality of polymorphisms is a polymorphism of a nucleotide marker sequence according to Table 2.

19. The method of any one of claims 1-17, wherein each of said plurality of polymorphisms is a polymorphism of a nucleotide marker sequence according to Tables 2 and 4.

20. The method of any one of claims 1-17, wherein each of said plurality of polymorphisms is a polymorphism of a nucleotide marker sequence according to Table 4.

21. The method of any one of claims 1-17, wherein each of said plurality of polymorphisms is a polymorphism of a nucleotide marker sequence according to Table 6, 7, 8 or 9.

22. The method of any one of claims 1-17, wherein each of said plurality of polymorphisms is a polymorphism of a nucleotide marker sequence selected from the group consisting of SEQ ID NOs 1-58 and 60-382.

23. The method of any one of claims 1-22, wherein each of said primers is about 8 to about 30 nucleotides in length.

24. The method of any one of claims 1-23, wherein said phenotype is a trait.

25. The method of claim 24, wherein said trait is selected from the group consisting of coat color, hair color, hair length, eye color, marbling, tenderness, quality grade, muscle content, fat thickness, feed efficiency, red meat yield, average daily weight gain, disease resistance, disease susceptibility, feed intake, protein content, bone content, maintenance energy requirement, mature size, amino acid profile, fatty acid profile, milk production, a milk quality susceptibility to the buller syndrome, stress susceptibility and response, temperament, digestive capacity, production of calpain, caplastatin and myostatin, pattern of fat deposition, ribeye area, fertility, ovulation rate, conception rate, fertility, and susceptibility to infection with and shedding of pathogens.

26. The method of claim 24, wherein said coat color is selected from the group consisting of cream, red/black, black, silver, tobiano, sabino, agouti, chestnut, brown, dilution, melanistic mask, albinism, recessive black, Siamese, Burmese points, cinnamon, red, and albino.

27. The method of any one of claims 1-23, wherein said phenotype correlates with a disease.

28. The method of claim 27, wherein said disease is selected from the group consisting of LWO, GBE1, JEB, SCID, and HYPP.

29. The method of claim 27, wherein said disease is selected from the group consisting of congenital myotonia, muscular dystrophy, globoid cell leucodystrophy, GM-gangliosidosis, Hemophilia B, hereditary cataracts, phosphofructokinase deficiency, thrombasthenic thrombopathia, retinal dystrophy, type-2 von Willerbrand's disease, and Type III von Willebrand.

30. The method of claim 27, wherein said disease is selected from the group consisting of hypertrophic cardiomyopathy, polycystic kidney disease and mucopolysaccharidosis.

31. The method of any one of claims 1-30, wherein each of said oligonucleotide probes is detectably labeled.

32. The method of claim 31, wherein said first oligonucleotide probe is labeled with VIC®.

33. The method of any of claim 31 or 32, wherein said second oligonucleotide probe is labeled with FAM™.

34. The method of any one of claims 1-33, wherein said assay plate comprises one or more arrays.

35. The method of claim 34, wherein said assay plate comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 arrays.

36. The method of any one of claims 34-35, wherein said characteristics are identified using a single array.

37. The method of any one of claims 1-35, wherein said plurality of polymorphisms is simultaneously identified using one, two or three assay plates.

38. The method of any one of claims 1-37, wherein said simultaneous identification of said plurality of polymorphisms and determination of said characteristics is performed using a processor-based system.

39. A computer readable device having computer readable code embodied therein, said code embodying instructions for causing a processor-based system to identify a plurality of polymorphisms in a nucleic acid sample, comprising:
instructions that cause a processor-based system to identifying a plurality of polymorphisms in a nucleic acid sample according to any one of claims 1-37;
instructions that cause the processor-based system to hybridize said nucleic sample to said primer sequences and to said oligonucleotide probes; and
instructions that cause the processor-based system to detect the presence of said plurality of polymorphisms in said nucleic acid sample.

40. The method of claim 38 or 39, wherein said system correlates said plurality of polymorphism with at least two characteristics selected from the group consisting of parentage, identity, genotype and phenotype.

41. The method of any one of claims 38-40, wherein said system further comprises a graphical user interface for displaying the plurality of polymorphisms within said nucleic acid sample.

42. An assay plate comprising a plurality of recesses, wherein each of said recesses comprises a composition, wherein each of said compositions comprises:
(a) a pair of forward and reverse primers;
(b) a first oligonucleotide probe;
(c) a second oligonucleotide probe; and
(d) a nucleic acid sample isolated from an animal;
wherein said first oligonucleotide probe is capable of detecting a first allele of a sequence of said nucleotide marker sequence;
wherein said second oligonucleotide probe is capable of detecting a second allele of said nucleotide marker sequence;
wherein said nucleotide marker sequence is any one of the nucleotide marker sequences as set forth in Tables 1-11;
wherein said nucleotide marker sequence correlates with at least one of the characteristics of an animal selected from the group consisting of: (i) parentage; (ii) identity; (iii) sex, (iv) genotype and (v) phenotype; and
wherein said forward primer is capable of hybridizing to a sequence that is about 30 to about 60 nucleotides upstream of a nucleotide marker sequence polymorphism;
wherein said reverse primer is capable of hybridizing to a sequence that is about 30 to about 60 nucleotides downstream of a nucleotide marker sequence polymorphism present within said nucleic acid sample;
wherein said assay plate is capable of simultaneously identifying a plurality of polymorphisms; and
wherein said plurality of polymorphisms correlates with least two characteristics of said animal selected from the group consisting of: (i) parentage; (ii) identity; (iii) sex, (iv) genotype and (v) phenotype.

43. The assay plate of claim 42, wherein said plurality of polymorphisms correlates with at least three of said characteristics.

44. The assay plate of any one of claims 42-43, wherein said plate identifies said plurality of polymorphisms in at least one animal.

45. The assay plate of any one of claims 42-44, wherein said plate identifies said plurality of polymorphisms in at least two animals.

46. The assay plate of any one of claims 42-45, wherein said plate identifies said plurality of polymorphisms in at least three animals.

47. The assay plate of any one of claims 42-46, wherein said plate identifies said plurality of polymorphisms in at least four animals.

48. The assay plate of any one of claims 42-47, wherein each of said animals is of a family selected from the group consisting of Equidae, Bovidae, Canidae, and Felidae.

49. The assay plate of any one of claims 42-47, wherein each of said animals of the family Bovidae is of a species selected from the group consisting of Bos, Ovis, and Capra.

50. The assay plate of any one of claims 42-47, wherein each of said animals of the family Equidae is of a species selected from the group consisting of Equus.

51. The assay plate of any one of claims 42-47, wherein each of said animals of the family Canidae is of a species selected from the group consisting of Canis.

52. The assay plate of any one of claims 42-47, wherein each of said animals of the family Felidae is of a species selected from the group consisting of Felis.

53. The assay plate of any one of claims of any one of claims 42-52, wherein said plurality of polymorphisms comprises between about 20 and about 12,000 polymorphisms.

54. The assay plate of any one of claims 42-53, wherein said plurality of polymorphisms comprises about 60, 3000, 6000 or 9000 polymorphisms.

55. The assay plate of any one of claims 42-53, wherein said plurality of polymorphisms comprises about 64, 128, 3072, 6344 or 9216 polymorphisms.

56. The assay plate of any one of claims 42-53, wherein said plurality of polymorphisms comprises between about 20 and about 5000 polymorphisms.

57. The assay plate of any one of claims 42-53 and 56, wherein said plurality of polymorphisms comprises between about 20 and 200 polymorphisms.

58. The assay plate of claim 57, wherein said plurality of polymorphisms comprises about 20, 30, 40, 50, 60, 70, 80, 90, 100, 110, 120, 130, 140, 150, 160, 170, 180, 190, or 200 polymorphisms.

59. The assay plate of any one of claims 42-58, wherein each of said plurality of polymorphisms is a polymorphism of a nucleotide marker sequences according to Table 2, Table 4, Table 6, Table 8, and Table 11

60. The assay plate of any one of claims 42-59, wherein each of said plurality of polymorphisms is a polymorphism of a nucleotide marker sequences according to Table 2.

61. The assay plate of any one of claims 42-59, wherein each of said plurality of polymorphisms is a polymorphism of a nucleotide marker sequences according to Table 2 and/or Table 4.

62. The assay plate of any one of claims 42-59, wherein each of said plurality of polymorphisms is a polymorphism of a nucleotide marker sequences according to Table 6.

63. The assay plate of any one of claims 42-59, wherein each of said plurality of polymorphisms is a polymorphism of a nucleotide marker sequences selected from the group consisting of SEQ ID NOs 1-58, and 60-382

64. The assay plate of any one of claims 42-63, wherein said phenotype is a trait.

65. The assay plate of claim 64, wherein said trait is selected from the group consisting of coat color, hair color, hair length, eye color, marbling, tenderness, quality grade, muscle content, fat thickness, feed efficiency, red meat yield, average daily weight gain, disease resistance, disease susceptibility, feed intake, protein content, bone content, maintenance energy requirement, mature size, amino acid profile, fatty acid profile, milk production, a milk quality susceptibility to the buller syndrome, stress susceptibility and response, temperament, digestive capacity, production of calpain, caplastatin and myostatin, pattern of fat deposition, ribeye area, fertility, ovulation rate, conception rate, fertility, and susceptibility to infection with and shedding of pathogens.

66. The assay plate of claim 64, wherein said coat color is selected from the group consisting of cream, red/black, silver, tobiano, sabino, agouti chestnut, brown, dilution, melanistic mask, albinism, recessive black, Siamese, Burmese points, cinnamon, red, and albino.

67. The assay plate of any one of claims 42-63, wherein said phenotype correlates with a disease.

68. The assay plate of claim 67, wherein said disease is selected from the group consisting of LWO, GBE1, JEB, SCID, and HYPP.

69. The assay plate of claim 67, wherein said disease is selected from the group consisting of congenital myotonia, muscular dystrophy, globoid cell leucodystrophy, GM-gangliosidosis, Hemophilia B, hereditary cataracts, phosphofructokinase deficiency, thrombasthenic thrombopathia, SCID, retinal dystrophy, type-2 von Willerbrand's disease, and Type III von Willebrand.

70. The assay plate of claim 67, wherein said disease is selected from the group consisting of hypertrophic cardiomyopathy, polycystic kidney disease and mucopolysaccharidosis.

71. A composition comprising a plurality of nucleotide marker sequences, wherein each of said nucleotide marker sequences comprises a polymorphism, and wherein said plurality of nucleotide marker sequences correlates with at least two characteristics selected from the group consisting of: (i) parentage; (ii) identity; (iii) sex, (iv) genotype and (v) phenotype;
wherein each of said nucleotide marker sequences is any one of the nucleotide marker sequences as set forth in Tables 1-11.

72. The composition of claim 71, wherein said plurality of nucleotide marker sequences correlates with at least three of said characteristics.

73. The composition of any one of claims 71-72, wherein said plurality of nucleotide marker sequences correlates with said characteristics in at least one animal.

74. The composition of any one of claims 71-73, wherein said plurality of nucleotide marker sequences correlates with said characteristics in at least two animals.

75. The composition of any one of claims 71-74, wherein said plurality of nucleotide marker sequences correlates with said characteristics in at least three animals.

76. The composition of any one of claims 71-75, wherein said plurality of nucleotide marker sequences correlates with said characteristics in at least four animals.

77. The composition of any one of claims 71-76, wherein each of said one or more animals is of a family selected from the group consisting of Equidae, Bovidae, Canidae, and Felidae.

78. The composition any one of claims 71-76, wherein said one or more animals of the family Bovidae is of a species selected from the group consisting of Bos (cattle), Ovis (sheep), and Capra (goat).

79. The composition of any one of claims 71-76, wherein said one or more animals of the family Equidae is of a species selected from the group consisting of Equus (horse, donkey, mule).

80. The composition of any one of claims 71-76, wherein said one or more animals of the family Canidae is of a species selected from the group consisting of Canis (dog).

81. The composition of any one of claims 71-76, wherein said one or more animals of the family Felidae is of a species selected from the group consisting of Felis (cat).

82. The composition of any one of claims any one of claims 71-81, wherein said plurality of nucleotide marker sequences comprises between about 20 and about 10,000 nucleotide marker sequences.

83. The composition of any one of claims 71-82, wherein said plurality of nucleotide marker sequences comprises about 60, 3000, 6000, or 9000 nucleotide marker sequences.

84. The composition of any one of claims 71-82, wherein said plurality of nucleotide marker sequences comprises about 64, 128, 3072, 6344 or 9216 nucleotide marker sequences.

85. The composition of any one of claims 71-82, wherein said plurality of nucleotide marker sequences comprises between about 20 and about 5000 nucleotide marker sequences.

86. The composition of any one of claims 71-85, wherein said plurality of nucleotide marker sequences comprises between about 20 and 200 nucleotide marker sequences.

87. The composition of any one of claims 71-86, wherein said plurality of nucleotide marker sequences comprises about 20, 30, 40, 50, 60, 70, 80, 90, 100, 110, 120, 130, 140, 150, 160, 170, 180, 190, or 200 nucleotide marker sequences.

88. The composition of any one of claims 71-87, wherein said plurality of nucleotide marker sequences comprises the nucleotide marker sequences listed in Table 2 and/or Table 4 and/or Table 6 and/or Table 8 and/or Table 11.

89. The composition of any one of claims 71-88, wherein said plurality of nucleotide marker sequences comprises the nucleotide marker sequences listed in Table 2.

90. The composition of any one of claims 71-88, wherein said plurality of nucleotide marker sequences comprises the nucleotide marker sequences listed in Table 2 and Table 4.

91. The composition of any one of claims 71-88, wherein said plurality of nucleotide marker sequences comprises the nucleotide marker sequences listed in Table 2 and Table 6.

92. The composition of any one of claims 71-88, wherein said plurality of nucleotide marker sequences comprises the nucleotide marker sequences listed in Table 2 and Table 8.

93. The composition of any one of claims 71-92, wherein said polymorphism is located at a position within said nucleotide marker sequences according to Table 2 and/or Table 4 and/or Table 6 and/or Table 8 and/or Table 11.

94. The composition of any one of claims 71-93, wherein said phenotype is a trait.

95. The composition claim 94, wherein said trait is selected from the group consisting of coat color, hair color, hair length, eye color, marbling, tenderness, quality grade, muscle content, fat thickness, feed efficiency, red meat yield, average daily weight gain, disease resistance, disease susceptibility, feed intake, protein content, bone content, maintenance energy requirement, mature size, amino acid profile, fatty acid profile, milk production, a milk quality susceptibility to the buller syndrome, stress susceptibility and response, temperament, digestive capacity, production of calpain, caplastatin and myostatin, pattern of fat deposition, ribeye area, fertility, ovulation rate, conception rate, fertility, and susceptibility to infection with and shedding of pathogens.

96. The composition of claim 94, wherein said coat color is selected from the group consisting of cream, red/black, silver, tobiano, sabino, agouti chestnut, brown, dilution, melanistic mask, albinism, recessive black, Siamese, Burmese points, cinnamon, red, and albino.

97. The composition of any one of claims 71-93, wherein said phenotype correlates with a disease.

98. The composition claim 97, wherein said disease is selected from the group consisting of LWO, GBE1, JEB, SCID, and HYPP.

99. The composition claim 97, wherein said disease is selected from the group consisting of congenital myotonia, muscular dystrophy, globoid cell leucodystrophy, GM-gangliosidosis, Hemophilia B, hereditary cataracts, phosphofructokinase deficiency, thrombasthenic thrombopathia, SCID, retinal dystrophy, type-2 von Willerbrand's disease, and Type III von Willebrand.

100. The composition claim 97, wherein said disease is selected from the group consisting of hypertrophic cardiomyopathy, polycystic kidney disease and mucopolysaccharidosis.

101. A database comprising the nucleotide marker sequences as set forth in Tables 1-11.

102. A method of identifying a plurality of nucleotide marker polymorphisms comprising
(a) contacting a nucleic acid sample with the composition of any one of claims 71-100;
(b) hybridizing said nucleic acid sample to a pair of forward and reverse primer sequences;
(c) performing PCR amplification of said nucleic acid sample;
(d) hybridizing said amplified nucleic acid sample obtained from step (c) to said plurality of nucleotide marker sequences in said composition; and
(e) identifying said plurality of nucleotide marker sequences;
wherein said plurality of nucleotide marker polymorphisms correlates with at least two characteristics selected from the group consisting of parentage, identity, genotype and phenotype.

103. The method of claim 102, wherein said nucleic acid sample is detectably labeled.

104. The method of any of claims 102-103, wherein each of said compositions is affixed to a substrate.

105. The method of claim 104, wherein said substrate is selected from the group consisting of chip, wafer, slide, membrane, particle, bead, panel or assay plate.

106. The method of claim 102, wherein said forward primer is capable of hybridizing to a region within a nucleotide marker sequence that is about 30 to about 60 nucleotides upstream of the polymorphic site present within said nucleotide marker sequence.

107. The method of claim 102, wherein said reverse primer is capable of hybridizing to a region within a nucleotide marker sequence that is about 30 to about 60 nucleotides downstream of the polymorphic site present within said nucleotide marker sequence.

108. The method of claim 1, wherein said forward primer is capable of hybridizing to a region within a nucleotide marker sequence that is about 30 to about 60 nucleotides upstream of the polymorphic site present within said nucleotide marker sequence.

109. The method of claim 1, wherein said reverse primer is capable of hybridizing to a region within a nucleotide marker sequence that is about 30 to about 60 nucleotides downstream of the polymorphic site present within said nucleotide marker sequence.

110. A computer readable device having computer readable code embodied therein, said code embodying instructions for causing a processor-based system to identify at least two characteristics selected from the group consisting of parentage, identity, genotype and phenotype, comprising:
instructions that cause a processor-based system to contact a nucleic acid sample with the composition of any one of claims 71-100;
instructions that cause the processor-based system to hybridize said nucleic sample to said plurality of nucleotide marker sequences in said composition; and
instructions that cause the processor-based system to detect oligonucleotide sequences within said nucleic sample that have hybridized to said plurality of nucleotide marker sequences;
wherein said plurality of nucleotide marker sequences correlates with at least two characteristics selected from the group consisting of parentage, identity, genotype and phenotype.

111. A method of determining at least two characteristics of an animal selected from the group consisting of: parentage, identity, genotype and phenotype, comprising
(a) contacting a nucleic acid sample with the composition of any one of claims 71-100;
(b) hybridizing said nucleic acid sample to a pair of forward and reverse primer sequences;
(c) performing PCR amplification of said nucleic acid sample;
(d) hybridizing said amplified nucleic acid obtained from step (c) to said plurality of nucleotide marker sequences in said composition; and
(e) identifying a plurality of nucleotide marker polymorphisms within said nucleic acid sample that have hybridized to said plurality of nucleotide marker sequences;
wherein said plurality of nucleotide marker polymorphisms correlates with at least two characteristics selected from the group consisting of parentage, identity, sex, genotype and/or phenotype and breed determination.


SEQUENCE LISTING (382 sequences)

SEQ ID NO: 1 - DNA, 401 bp, Equus caballus
  variation (201)..(201): Y represents polymorphism t or c
gccttgtgacatcacagctggatgtgtgtggccatgttcagaacttggtcccaggaactggtgggcactcgctcacatgtgggtctctggctctacctcctgcctgctggcccaaactttgggccagagccacacaaactccttctctttaaacaccactgcttccctcctcctcgctgatctgtagctttcccccgattygggatgttcttactgcacatcctgggcatttctcgtctacatcacctggtttagcgccgtggcatgctggctcacatgtgccaccacagctgcatgagggtttctccaggagcaggagggttgggcgaagaggccaagattcctcgctgagcacttgtcacatggagattgctgagaaaattctgtagatttcaaaggat

SEQ ID NO: 2 - DNA, 401 bp, Equus caballus
  misc_feature (43)..(43): n is a, c, g, or t
gggtagatttagagataaagagagagatgagagtctagggttngattttatggccctcgtaatattatccgcactaggagttgatatgagctctgctgaatatggcctggtcgtaaagagtgtgctgggaggatgctggcacgtgtgctcaatgtctacagccttgaggaagccttgcacatcaggcaccccgagtcaaarggagagtggttcgtggcgggatcaacttacggattggaatctggtgtctttgtagatcgaggctatgaactctagctgggcaccncgaccaccttccctccttgtcacaggcaaaggagccatgccgcatctctgaagaagtgcagggaagatgcgacagaaggcgaagggacccaaacaccaccaagaaggcgcattga

SEQ ID NO: 3 - DNA, 401 bp, Equus caballus
  variation (201)..(201): r represents polymorphism g or a
gctgcacatggttgagttatgaatcaagcttgttctcccgatgcgggaaaatgggccgatgaatccatttccatcacgacactacccataagtcatggattgaagggcacttttcttccttctggaatttccaatgacaaagatttcattatccaaagcaataatttgtaaccagagcaatgcatgatttcactctacctraattaggtccattgtgagagggaggctgggacaacctcagtgtctggaggggcagaagagggtgaaattggactccttctttgctctcgcccctcccccattctttccctttttctctctgtgtgggtgttcactttctttgttctcagtctcctttctcttgagccaacccatctcctggtgctctgcactggccatga

SEQ ID NO: 4 - DNA, 401 bp, Equus caballus
  misc_feature (73)..(73): n is a, c, g, or t
ccaaagtcctaaactctagcctcccgttggttctcaccctcgcgttttagcgttctaatagtgatcttgagantctttggcacggagcaaagctcctctttgacccagtgcagcgagggcctccacaacctgccctacccctttcgattcccttcctccccttagccccacctggctcaggaagagtacagaacgcatcasggcgaggaggggctccgaaagttacaaacctgcgctgcccgccacctgcccctccgagcgcgccgcgcggcttaaagtgccgctggggatgccccccttccccccgtaccccaaccccgaccgtcacctggagccggaagcgctgcgcagccttgtccatgttctccagggccgcttgctcgccgctgtcgcgcccgggc

SEQ ID NO: 5 - DNA, 401 bp, Equus caballus
  variation (201)..(201): r represents polymorphism a or g
gtaaaataagagatgcactatctctctgatattctctgctctggacctgagctgcacctcagaaaggggctctttctagcagagaggggagtgaggttgccattttgctgccactcccaagagggcaggccctgagactatttctgtctctcctctcctgattccccacccctcctgtctagactgagctgagtaactgtrtctccatgctggtggcggggaacgaccgtgtgcagaccatcatcactcagctggaggactcctgtcgagtgacaaaggtgagaggaagaatagctctgcctggggcttcgaagcctcccagtgtggcccggtctctggggtgaaggctcacccatgggatgtaactaaaggttggggcctggagcctggagtgttcaggc

SEQ ID NO: 6 - DNA, 401 bp, Equus caballus
  variation (201)..(201): k represents polymorphism g or t
atattaaaattgttgtctaccctcagaccacaccaggcacagtatcccaagggcatgtgaataggaagtgctcaagccataggcattgccttgatccttccacagagctgctaattagccttgggtcagtcagaaaactggttactcagctttgaaacttctaggcaaattttgctcttaagtatatagcacgacaaaaakaagtgataaagaccttttaacaacgtgtcttcattttacttatactcttcctattgtcatcacctcctgaggaagcctttataaccaaaacacacacttgatgaagagaacgtgagcagtcaaaaaaacacatatgtacaccgaagacacaacttaattttggttgggtcctcatgcagccaggacagagaaaacttttg

SEQ ID NO: 7 - DNA, 401 bp, Equus caballus
  variation (201)..(201): y represents polymorphism c or t
atgtctagattccaattttatgaaacctcagatatgcaatattatttaagaggttagaagtgactttaatttaaattcagataaataagttaatttagagtttttgatatgatgagagtttttgtaacgctggataaagttatgttagaacttgacaaaactcctgaaaggttgataaccaaaagaaaggtagacttgttygttgtcttcttgatatagcttccatttctctttagctccttgacagtaaagcaactntttctattgtaatatcagtacatgttcttcttatttcaatttagaaaaaagctggtctgtatctgcctgtaacatctctgaagggagaaatcagctcacttaacttcccttctcttccccaaatgaatattgtaccttccact

SEQ ID NO: 8 - DNA, 376 bp, Equus caballus
  variation (201)..(201): r represents polymorphism g or a
ttgaagtttcggttgacttatagctcttttttgacctttgctactaatagctggcagagaataaaaccagacatgtcaatcactgtctagattttataaactattgactgtttcttgaaggatggtaatgcttattttattggcctcttgtaccatagaaggggatttatagagtgccaaaaaaatgaaaaccatgcctarctagcatcatccagatctgctggtaataaagtcactgaaattaataactatcaattaaatataaatgagctgaaaccacaccaaggagagaatcaacagcattttaattcctagatctcttttggcattatcatatttaatgctctgtaaagagttgaattaactagttttcctg

SEQ ID NO: 9 - DNA, 401 bp, Equus caballus
  variation (201)..(201): r represents polymorphism g or a
aaaacagcttctttgcaaatgagagcacagccgtgcttccctgactccaaatagccaagtgagaagatgtcaccacaggctgccccttcgtgacctgggctaagccgagtcccccaaatttcctctgaacctctggctacaaagaaatgtgcttagcttgctcaggagagtttgtccaaactcatggaattcatggaggtrttaagtgtcaagctattttcttcagtttccccttttaccctcacatgtcaccccctcttaaaagtttttttaaagtgaaatacaaaatttatccaaagagaaatgagatttcctggataaagcatttgtgtgatactatttctgagtctgtgtctctgaaaaaatggctgggaaatcccctcgtctctacatttaagcat

SEQ ID NO: 10 - DNA, 401 bp, Equus caballus
  variation (201)..(201): r represents polymorphism a or g
gctttccaaagtgttggacaagctaagtcctatgcaattatgcaatacttttaaaaaaaacttgaattttaaaatatgcttgaaaaatagatgctgatccaacaagaacagagactatcgatgaaaagaatgtgttctctgtgcacctaagggaagccaacacacaggcagcctcatgtggcctgaagcttcaggactttrggaagttgcttgcagatgaatttctttgaaatgaatagctcagggcggcatatgccctccctctagatttgacttctgtggtttatgtataagctggggaagacctcagagtctgacctagactcacgttatgtgcctctgaagtctggtgaaaggccagactttaggattccgcagagttgagagttgagtgaggaacc

SEQ ID NO: 11 - DNA, 401 bp, Equus caballus
  variation (201)..(201): r represents polymorphism g or a
atcagtccttcaggttctggaagccctccatgctgaggcaatttatgtttctctgcctagggcacggggaacgccttcacctccactcagacccttctggatttctcccagccaaggaagtatggagctccagaagaatcttacgaaaagtttctgagcataggagagagtacgtttacctttagggcagcgtttcctgcrcgtctgctgcatcaccccagcctagcttaactacctgaacacacggctggacttgagaccctcttaagaattcaagtttgtggggaatggaagctgggagggagttgagcaagaaggaaggttcctatagctctagcagcacgcctagttcagggaggaaggacagacgagagggcatcatactcacaaagaaagtcttc

SEQ ID NO: 12 - DNA, 401 bp, Equus caballus
  misc_feature (40)..(40): n is a, c, g, or t
cccgccattggcggggagacccggcctggtgctcggggcncccggagggtcccagagagagacacggagggcacggaggtctnccagctgccgttgcccgccccntgggactagggattgccggagatctcggagaggaccggggcgggggaactttcaaaggcgggtccggcgacccggtggggaagcgccggagctccrccaggcagcagacaaaactctctgtctgccggtagcagaggggccacgctgagnactcagggctcccggcagaggcccgganagaagcccagagggcggggcgacccccagctgccgttgcccgccccgngggactngggattgccggagatctcggagaggaccggggcgggggaactttcaaaggccggatcggcgac

SEQ ID NO: 13 - DNA, 401 bp, Equus caballus
  variation (201)..(201): y represents polymorphism c or t
ttcataaatcctggagtaatggtgaagcctttttggcttaaattttacttgggttgtaaaattaaccccacttttattgggtaactaataccataaacctatggggagaaagaagtttctcgatgacccagtcttggatctcggaaaccgaagccctgagaccagaatgccccccacgccaccccctgcattacagatgaygctcttccacccccaggcattagcagagccctggatttttgaggtgtcattatgagaacacgtcttcctcccccacattaagctctcaaggcctcaatttatatctcttgggaggacaaaaccctggaattaccccagatataaatcccgacctactgggagttcccatattttatgtgaggcttaggctaaacttctta

SEQ ID NO: 14 - DNA, 401 bp, Equus caballus
  variation (201)..(201): k represents polymorphism g or t
gtcatttgcatctctagcattattacaattctgaaagtcatttcaaataaggtaagttttagaagtgaaaggaaacttctggcatattagacataagtcaaggactcttgtttatgtcaagcaattctaccacatatctttgtatgattaggataattgtttagaatattctcccaggcacattactgtcaattactaaakattatatagtcagagtccccaccttgtattccgtttgaatcacactgttttgcattattttaaatggccacatttttatttttatcgaggggaacgtaactgcaaggaatgtggttatcatgagctaatcttacccttggggtatgtgagatattttctaactctgagattgtgattgctttctgattgccattctgctc

SEQ ID NO: 15 - DNA, 401 bp, Equus caballus
  misc_feature (29)..(29): n is a, c, g, or t
cagagtgttttactccaaagcgtaactcncatcacccagagggctccctgaattccacttcttcctcttggaagtcctcccacacgcgtcagaaaagagctcgtggcttcctcttcttcactccctgccccacctgggtcacccacagctactctcttccatgtttatgactctccatcggccccaatccctggaaatacrtttgttttatagcaagaacaccttgctgctttcctccatcagacgactgcccatccctcagtgctggatatgtcacccataccagttttttgatttatctttgagaacagtctctgccaagaattcttgagtagaatgtcattcaaccattggccataaccattctctcagacagcacatctacaaaggtctctctcgca

SEQ ID NO: 16 - DNA, 401 bp, Equus caballus
  misc_feature (105)..(105): n is a, c, g, or t
aataggaatgcattgctttctgcaatctgtctttgcttggaatcagtaacaatatgttctgcaactgttataaattgaatgcattttctttattgagatacattnctttttttcatataaatatttaattggccctgagagaaaagctgttggcatatttccttcatttgctgtggggttggtgaatgattcagtcttcaytgaatagggcaatttcctggggttgactactgcgagctaaaaagctcatccattttcagtcctcctttagaaaatgaaataactacaaatttgtcctctgtaagccatcagaaaaaatgaaacaattgacaaatcaggttctaagaaggagaaacagtttatattttgtttgtctactactgtcatttaagtgtttacct

SEQ ID NO: 17 - DNA, 401 bp, Equus caballus
  variation (201)..(201): s represents polymorphism g or c
cacttctcctatgaatacttctccaaacgggattcatgtcatctcactccattctcatcctgttttggccacagtacagtcactgcccagtccttgagcgagcaagacacagctgctgtttcatcaagatggtgtcttgatacctgagtttctctgagaccctgggttctgtgagctctgcccagagtccagaagcccttsagtacaatctcgctattaaccctggatctctcttcacatcctttcccccttctaggccacttttcccttctctccatccacttggatccacagcctttaagtccgtcnccagcagtcatcttcacttcaaggtctgacagcacctcaacttagtatgaccaggtggagctcttcattccactgtcaacccccaacttggt

SEQ ID NO: 18 - DNA, 401 bp, Equus caballus
  variation (201)..(201): y represents polymorphism c or t
gcggggagagagctagcacactgagtcggctgcaggctctggctgacgggcgaggcttacctcttgcctaagaaagtggcttccccacatttgagacttaaactcatgcctcagaaacatcaccagcagcccttttgcatgaatctcagaacctccttggcagccgtaaacacactttacaagtggtcaacactggcatgycagaggtctgtgggtttcacacagattccttggcggggcaagctggctgggggacggaagccctctgtggcctgacgcgctgtcgtagccttgaccatggcctttttgtttaaacagacatttccagggaagccctcaaaacatattcgattgggaatgtctcgttcagcaaagcacatctgatagagagagatcttggt

SEQ ID NO: 19 - DNA, 401 bp, Equus caballus
  variation (201)..(201): y represents polymorphism t or c
tcagaaggaacctcccagcctcaccaaccacatatctccttcagtcactgagccttccaattcatttccttgctgttactgtctactttcttctatatatagcacacatccaaggatgagcatttctgagatagccttctttagaattgaaagacagatctcatttaaaagagatgtagtctctactccaaattagaagcyatacaacttcaacatgctagaagtgtcttaaagaactgatgcaatttacatcaatggcaacaacttagtgaacaacttaaatcatatacagtttataattcagatgtcaaaaaaacacttaatataacgtaacatcataaaggngattgtgaaatatatggacctaaatttttgctctctttaagaaacattacaaagtg

SEQ ID NO: 20 - DNA, 401 bp, Equus caballus
  misc_feature (94)..(94): n is a, c, g, or t
gtgactgtggcagaacctcattaccaaaactaagggtccacccttaccttccccaaatgacatgttatgcctgaaggtatccagacaatgtcangttggtagtaaacttttcttttnntatgaccccacttctgaaggtaatcacttctgatttttattttgctcgtgacttcactaacctgactaagactgattttatgycatctgttcctgactctcaatatattttaacaagtcagaaactagngggcttaagtcngcattttctggacacagataaacnttttncttttgttttgtttttaanaattcattgaaaaganncaagaaggaaaacttcctcaagacaagngagtttgatttgttttgttttttactaagttcctccaatatcaaagctg

SEQ ID NO: 21 - DNA, 401 bp, Equus caballus
  variation (201)..(201): w represents polymorphism a or t
ccataatcctgtccttatttctccacttggatgtggcagcactgtagctgcagcatggccttttttcttccagaatcagctcttcctccggcaatccccgcctcagtggatcatccacatgagcaaaaacctggagtcataattgactgactctcctccttcactcccaataaccacatttcagttattttgccttttagwtatttctcaaatctgttttctctttgtgccctggtcctttttcccagcacttgtctttacttatgtccttccttgtggctgaaatgcttctctcacttttgttgttattgttgttaacacagctagtgtgttcttgctcattcttaaagactcncttcaggctgtattgccctttacttctatgcatgtgtctttcttaa

SEQ ID NO: 22 - DNA, 401 bp, Equus caballus
  variation (201)..(201): m represents polymorphism c or a
tcactttttccattttgggctcattgcttgcctaactcaactgcatcacttagtattcttttggctcagtgggaaaatagtaaaatatctaagaacttgaagatccagaaatgccttctttcactctcgttctctttatcaagttacttggaaacattttcttatttcaaataaagtgtagggttcaaagtgctaggagamaaaaatccatcaaggatccatccccctaaagctcttcttgtctctcatgaaaacatggccccacgtggtgggtttaacctgtgagattcaggtcggagtctcctgcttggggacttgcccctgctgacngtttctcctttgtcccttaaaaataatttggctccatatacaattctccacagactcctaattcctggaaa

SEQ ID NO: 23 - DNA, 401 bp, Equus caballus
  variation (201)..(201): y represents polymorphism t or c
cacagaaaggaaagatacccccaaacattttcatgatgcggcagactctacagaaaactcgtgaattaaggcatttcagtaacaataactaattctaccaacaccattactataaaaccattaactaactgaccaaaaaaattaagaaaaaatgaggaaatagcaaaagccataattatgctcctgtaagcagactgaaaytttttgaaaagtacaccatgtacgaactaccaacatatagaagtttgagcaatgggctgagcacaaaggaaaagcttacactcactctttgagggtgtaggggtgtagtgggaagggaagatggtgataaaaaaaacagtagtcccaattctgtattgtgttacctacgcaatgtacctacacaatgtcatcaatgacaa

SEQ ID NO: 24 - DNA, 401 bp, Equus caballus
  misc_feature (44)..(44): n is a, c, g, or t
aacattctaacttgctccaatcagacacaacgccaaggtttcangcaggttaatggagaaccaagagatggcacacagctctgtgagacgatgccaggggacagcccagcacagagcacaggccctgggattctcactggtcacgtgggagtggaggacgcggcatgaagcaagggcatctccgctcagaaggttccctgyggagccccacagacaaggcagcgcgagcaaggcccaaagaacaggctcccccgccatgggctcctctctggccccaacgtgaggacagccatactatgaagacacagcactaaggcaaaaagctcctcatgtgggacagaaacccacaccccaccaagatgggttctgactcctctatcgttttggactccctgagaacc

SEQ ID NO: 25 - DNA, 401 bp, Equus caballus
  misc_feature (33)..(33): n is a, c, g, or t
gggaactgacctactcagatctgcctccaagangtcaggaaggaantgcagacagagaaacnctcacgcacaaactggagagtggggttgggcatggccgatccccggagctggttggcaggtggacaccagcatctagcagtagccaggtggtctgactccaggaccagagctgggctcttccgacaacatgatgctccytgcatggaaagccacgagagctctcctctcttctcttcaggaagccagtggaagaggagaacggaggatcggaagagttgtgcatgcatctccggcagtctgggggtggatgtgagtccaggggggtagggccgactgggaaaataggagcgaggagcctggtggggtggcccccagaatggaggtgtctgtgcctgtgg

SEQ ID NO: 26 - DNA, 401 bp, Equus caballus
  variation (201)..(201): y represents polymorphism c or t
tccaaacagcctggcgggctttccctgatatatcatcctcacccagagccggtctcgtgtcactccaggacaccgaggcaggaaaaagactgacagcctgatgcgatataatgtgagtccccccaccatgggacaccccctgaggttctgtggccagcctggcccatgcccaggagctgtcacccacccagcctgacctcygggctccctctcccaactgtgccgaggatcaaatgataaggagacaaaaagaaaacagggagctggggccacacgtgagatcggcaccacttagtcatcatcgcgcccccaccccatgcttactcgtgaccaggccgatgccggggacgtggtctgccagcagctggagcaggaagaggatcctggccctgcaggcggga

SEQ ID NO: 27 - DNA, 401 bp, Equus caballus
  misc_feature (135)..(135): n is a, c, g, or t
gctcccagcccgatccccagccagcctggaggacactcttcccagtgatctccccctgctgcagagctcactatgggcacagttctgcacatgaggaggggtctccaccaacatctgctgcctggatggtggccngcgacgtccccatccctgacatttgcccagcaccctgttgggccagagccttttccacccatgacytcctttgctccttttgtgaactaccttcctggtctgaccccctcacatgcccccgggcaccctgcacggctcaggacggagacccggggtgggaaagtccagggtgcctcgtgctgggctgggacctggggtggacttgccctcccgaggctnggggctccatgcacanttgccccacaggcctgtgtgcccccaagctc

SEQ ID NO: 28 - DNA, 401 bp, Equus caballus
  misc_feature (71)..(71): n is a, c, g, or t
tagtccgtccctcacacacccaagatggcatccctgtctggtccaaagctgcacaatgtcccacccctctngctgcccattcctgagcatgaggaggtatttctgcttctctgccccattcctgggttctctctctcatcttctgcttgggttggctgcacanactcagagctgcctcaggccactccacagtttggtcaracatcccattggagtgaatcaggatgcctggcgtggtgccttctacctcctgctgcctggaccaccatgtccccttccaccactagcatttcatgaagacacgtgttttccaaggcctgttctgctcctttaaaatgcagtgtacatttgaaagagcgaggggcattctggaggctaagcttgggcatgtctttagggta

SEQ ID NO: 29 - DNA, 401 bp, Equus caballus
  variation (201)..(201): r represents polymorphism a or g
tcagatctttaaccaagaatcgattgatggaggcagcatggccagtgggacaaagaggtagtcagggccactggtgtcacatgtggcactgaaatgggttcttggaggtgccaatagccaaccttccatctgcccagtctttaaagggaaatgaatgggagaatggactgcgtggggcatcaagatcactattattccccrttccctttgcaatattttcaaacagaggatagcatatcaaaataaacaccaagaacaaagacatctctgatgtgcttttgtgctggcaggagagtgttgtctgctctcacagatggacttagctttgtccaatgaagaattctgcaaggggtgttatcacctgagcttaccatagacaccagaatcttgaatgagatggg

SEQ ID NO: 30 - DNA, 401 bp, Equus caballus
  variation (201)..(201): r represents polymorphism a or g
aagcaagaaagggaggaaggaaggaagcaaggaagggagggaggaaggaaggaagcaagcaaggaaggaaggaaggagaaacagtaaaataaaaaaaccaaaggaaaaactcaggcagaagatatgacaaatggaaaagatatgttcttcttgtagatctgaagttctcacttgcaagatgagataatacatctttgcttrttaatctggaattataatgtttgaacccttgaagtcctccaagaaactcaacccttagaaaaacccacagctgtctcctataggttttcaaataattgacaagtatctctcaaacttggaagaatacctttaagacttcagtacacactctctgtcttgactaactgacaaagcagaggaattgaaacagatacttcact

SEQ ID NO: 31 - DNA, 401 bp, Equus caballus
  misc_feature (60)..(60): n is a, c, g, or t
tgcatagattcagaagccagctggtgagacagcgttataaaggaggtattttagaaaganaaaagtcttgagcagagggttttgttcacaaaaagggcaacaaactacctgtgctaataagcttattcacnataaagtgactgctgtgagactntgtgaggtcagctcatcacagaaagctgtcactctactgtattactstaatagacgtttaaatacatgtttcatgcctacatctcactgttgtacgactcgaacacatataaacctcaaatgtcagggctacattcaattcaagacggtatgctgttagctctcacaatcataacttgtattccctgggaggaatgttcagaaatgttccccttccgatgtgaaggcctctctacctccagtccagt

SEQ ID NO: 32 - DNA, 401 bp, Equus caballus
  variation (201)..(201): r represents polymorphism g or a
gcgggcccttggcaggaaggacctagggactgtgccggggcttagagtgcagccttcagtcctgagagctgatcaaggagaaggggcagttccatggctctggagagggtctccccctgcataccctggccacctcgatccaccgctccaggaccttggccctgccctgggccgtcatgtttgggtccccaaggcaggacrtcatgacacaatttgctactctgttgaactgcaccactgtggcccggacggtgggtgccacgttctcatgtccaggctgggttcttttgccccaggtggagctcagatactcagagggcacgacgttcttgaacaatncctggtgaagagggggagagtcactcagccctgtcacagcccanctcagtgtccaggcagga

SEQ ID NO: 33 - DNA, 401 bp, Equus caballus
  misc_feature (20)..(20): n is a, c, g, or t
agaatgccccctctctttangtagaaacgggcatgtgggtgtttcaggcctcgcatttagatcataggagatggaaagatctcccagagcctgttccacactgcagttgtcccccagttcaatgacactattttcttaggagaaaaccaagttataccgccactttgcccttttgaaaactgcgtctgctcatttattttrcttggtgcatatcttagacaggcattaccacagtggtttgtaaccttcgatttgtccgtgttccctctttggcttcaaaatgccacacgacccccttttgaggttggaaagaacctccttttcnctataatttcaaggggaacttgcaaagtatcacgataattgaaaaaaatctgtatcataatatcaggatccaggtt

SEQ ID NO: 34 - DNA, 401 bp, Equus caballus
  misc_feature (97)..(97): n is a, c, g, or t
tcggctcgaaacgttttcaaagtaaacaaatgagttagcaatttaccacttaggattctcaaagtgagagtttatcccaccaaaagtaattttccanctcctccccctcaagcctatgctgtccttttggctacagcatgggccaaaggttgataatacttctgtatacatttagcaaacccaacctctaccaaactaggrgaacggcaaatgataccggtggatagagacccagggtgctttaacgtcaaatgcacaacttgatggccgtctctcaccgtaggacagtggaacaagcaactgcagtgactcaacatgaagggcaggaatctccataaagtaatctcctgttatcaggaaatgtatttataactattttgtagatgggtacccatgtctca

SEQ ID NO: 35 - DNA, 401 bp, Equus caballus
  variation (201)..(201): y represents polymorphism c or t
gaagcaaaccgtgggataagggacctgtcactttataagcagctcagactaactgaaagcgtgaaatacctgtggttagagctaataacaaaataacagttatactcgtcacaaaaacatcttacacaggtgaaaacatgagtagtggaaaatgcaagctgccatatgcaggagcagagctggcaaccctggaagactgtygcttgcctcccgagggttcagcagagggccttgacgccccctctccgtaaggaaaagtccaggacacggagaggggaggcagtttctaccagagaacccatcttactcaacaccctccccccaaagaggatggcagcccctgcggccttgaaaaccccaaagcctcaaagctcggtgcctcccgcctggcccgagaaagg

SEQ ID NO: 36 - DNA, 401 bp, Equus caballus
  variation (201)..(201): r represents polymorphism g or a
tcaggtcctccacatccagttaaatttatcctggaagcaataaaaatgttaaatattacttggttagagtttctcctcctttatctagacgtaactgtgtagtgggggataaatggttgtaatgcagatattcgagaaggttcactgattccttcaggctacctggggccactcatgacatgttaacgagtatttactgtrtgtctactctatgtccctatgcaatttgacccgatatttttagtatttcagcttgagttaccaagtgattcggtaggtatgtgggaaagtttaatatgtctccaataaccagtaacttattaaaaatggatcttctcacatagaacagagagttactctacccaatcaccgaataatttccaaaaattaccccagtttac

SEQ ID NO: 37 - DNA, 401 bp, Equus caballus
  misc_feature (63)..(63): n is a, c, g, or t
gacttttaatatttggatatgtgaagatgtttataaattggtagtatggagagttctgaattntatgcccacgttcttgcccaggcagagtgacatttccccttcctcccgtccaaactaggagcaaaggacttttttaagtaaattatttttagatttcccagagatgctttttaaagagccgtttgttttcgtaaacaygttcgacgtgagttattgtgaatttttgctaatagaggctgacagtacacataacacgagcagacagcaaggtacagccccgggcacccgtctttggtggctgacaggctgagggatgaatgttgagagccccggacagccccgggaccgacgtgtcaggtcgggcatgtgccaggctctcccctctttctcgtctccag

SEQ ID NO: 38 - DNA, 401 bp, Equus caballus
  variation (201)..(201): r represents polymorphism g or a
acagttgatgcttaaattgtccttggaccctgtagctgctagattaggaattcttcctaatctgttcccaactttatgtttagcaccaaaatatctgttttatttcttctacttctcacaaattggagaaataagagtaattcataaatctttcacctcaattattctcttttctataaatgactaaaaaaatacattcargtaggattgtaaaaatctatgaccagaccctttgttttcaaggttatagaaacaacaaaagcttgaaattttacaaggaaattgtaataatttagctcaggtaaagtgtaattttcttcatgaaaaagcaaatattaatctaaatagttttcaagtgagatatcaaaaagagacctcattaagtaaataaaataccaatt

SEQ ID NO: 39 - DNA, 401 bp, Equus caballus
  variation (201)..(201): y represents polymorphism c or t
aacccccacagctgactcgtcagtctgtcttcagccactagtaacaagcccagttctatagatgagcctctcgaggcccagagcagcttgtttatagtcctctagttcgtaagtgatggaaccagtctttacttctcctacctccctcaagcagtgcactgagagatagaaactaaggatcaaagaaaccacaatacttcytagtgcttcttctacagatctgagagtttttgaaaatgagaccaatttgagataaaagccttcagcacttgtttccaaaacttaattcattcaacaaatatacctggcccccactaggaaataacggagtcggggcaaggcagtaaggtctccactctttttgatgtacatgcttggtggtgatggtggtggtggggtga

SEQ ID NO: 40 - DNA, 401 bp, Equus caballus
  variation (201)..(201): k represents polymorphism g or t
acgtattgtgttttctttctaagtctcctaccaattgtaagttctctcccttgaccaaaattgtgcaaattttcttctatatagtcttctgtttttatttttatatttagtgtttacattttgttcatattaaaatttcatttacagcatctggatgtatcttgcgcacaacagcacctaggagtggattaaacgtttttkataatttggaagcattcttttggttttctatcaccttcgggctatgattatacaaacagattttcactgatctgtgattatacttgttaatagagcgccaggccacngtccttcgaagtggctgcacccacctgaaatctattcttacacctgctctaacactgtcctgagtccccattcttgggaattctgacccatta

SEQ ID NO: 41 - DNA, 401 bp, Equus caballus
  variation (201)..(201): w represents polymorphism t or a
ctgccccgggaaagcggctggggaaccagtgttcagggcctatcctcccacagatcccactcagcattcaggctatgctggtcagtggactggggattttccctggagccttctacaacaccaggactcctgctccgtgatcttcggatggaaatccttgtgcccactctcacccctgcccgcccggcctggcttgtcccwagaaggatgatgttctattctttcccatccctggagtccctctagttgattcaaagaagtgggaatcatagtaaaaaagaagagagactcatcttctggtgggtctcggttcaggatttcttaccttcctgatgtgtctccgtttgcaaggtggtcgtaaggatggattcctctggagggagggaaggaacaggaggaag

SEQ ID NO: 42 - DNA, 401 bp, Equus caballus
  variation (201)..(201): y represents polymorphism c or t
caaagctccttttctatgtccaatgaatggacaaatccctcaaacgattaaataattgccccaggatctaggtgtagcttaattcagttagatttagaatgtgaggattataaggaagtcaaatcaaaatatggaaaaaaacaaacaagacttctttctgagctaagcttctccaaaggctttgggaaaatcaacagaacyagtgatttgatttgaaagtcccttcctgtcttagggttgcactttgaatgccccatacttgttcttatggactgctgaccagagtacctccactccttgattttctccttaagaacaaatttcagcatcctagagagaatgctttctaaatggcaattatccttaggttcctgttacctaggaatgtgtttaccagttgt

SEQ ID NO: 43 - DNA, 401 bp, Equus caballus
  misc_feature (82)..(82): n is a, c, g, or t
ttatactttgtattatttaaagtacctgatgccatgctacacaactagtgtgtgtgcatccgttcatctgacagatatttantgactcacctnccagctgactccctgggaagccagagcagtttgccaactaatatggttaattgggatttgataagtgctataaagacaccagagattcagcagtttctcccagctatygaacttattttggctaatggatatcactctcactcccatttcaatcttacaaagggatgctagaagaggattgaccagtcagaaggtggaaacttaataaaaattgnaagtcagagacgggagggaatgaaagcagcagaggaagaaggaggcagagactggacagccagcaaggcctcagtgccctgcacagtttaagc

SEQ ID NO: 44 - DNA, 401 bp, Equus caballus
  variation (201)..(201): y represents polymorphism c or t
cagtgtttgccactcttgccagcattgagaagtctcccaggctggttagggaagcggagtgtggctgcttgtcatgcttttcaccggagggcagtgtctgatcctcctcgtgtccccactccttcccaggtgacccacatcaccactccactttattcctaaatctttgactcgctaaatcctccctaaatctcagaacayggaacatgttgaggagctggcatgcccacaaactcctaaaaggcaacatgggccccagaagggcgccnggcaggcagggaatcctgatctctaagataggattgttaaaccctgcagactcggctccttaggaaatgcactggtctcagagagaacngagaccctgtcgggagctcttgggatttgtctctcactgtccc

SEQ ID NO: 45 - DNA, 401 bp, Equus caballus
  variation (201)..(201): y represents polymorphism c or t
ttagttgtggtgtatgttaaggctaattgcctcccccactaattattgaacagctgtcgatgccatttaataatagtccacattttcccaattgattttaaatgataccttcataatatattcaatttgacttcttgctccgataacgtagcagactgaggaatgtggcctcctcctgctgagcaacgtcaaccaaacttygcaacaacaacaaaagtaattaatcttgaaagaaagaaggaatgggagatgcccaggtgctggaagatgggagggaaaaaccagaaccggaagctattccatgnctgaggaccccaaaaaggcaagatcctcagtgaagagagctcacagctgaaaatagagaccacaggctggaacgatctaccacgagggagagtcag

SEQ ID NO: 46 - DNA, 401 bp, Equus caballus
  misc_feature (148)..(148): n is a, c, g, or t
cagatgaaggaaatatacaagcatttgtaaagccctttttaaaataaaagagaattcaaagacttaaaaatatatcaacttattgtgacaaaacaatattcctcttgtcttaccagacttcaagagaaaacttcgaagatgtcagganaacaggtgtagtgttctctttaagatcggctcagcctctgaatgttgtaaacytgccagcctgacttgcaagagtcaagacgagcacacaggcgtttcttacaggcggcaccagctctctgcccagagggagccagcaaaatcccggaagcctgtacacagtttttctcagaccatgtatatgtttagaagatagtaccnggatggctctagggaaaattattggcttccatgtaaaacccaaaagaaagaaa

SEQ ID NO: 47 - DNA, 401 bp, Equus caballus
  variation (201)..(201): y represents polymorphism t or c
ttgccaccaaaggcagactctgaatttatgttcataatcctgagtctgggtcaccaacgaatgtctttctggtgaggcctgaaatctaaatgttgggaatccaacgggtcttggcagtgcatggaaggctgtttgccaaatatctggatcttatttattccctccagtctccccagaaaatgctcttgttcatttaaatayggacgtgactacatttgttggggaccgtgtacttttttctttaaatagaaacgccatgtgtgtgatgttttctttgaaaaggaaagcccaggaattgtctgcatcagattataaaatgatcccagggtccattcctggctctaagcaagttgagtatacatcaccgcgtttaattcagcaatatcatcaatgtcagtgcg

SEQ ID NO: 48 - DNA, 401 bp, Equus caballus
  variation (201)..(201): y represents polymorphism t or c
ctatccttgggggattagatgttgaatatgtttggtatataaatgtcatactcagataaaattttttgttggcaacacaaaagagcacaacatgcgactaaagctagagaaatgttctgatttctattaacttttatgttcctgtataagctaaagcagttgagacaatagtaattagtcttccaatctcccattcctgaygtaatggcttggcagctttgagggtaggggaagactaatnaagagtaggtaggtcagtgttctgatggacagcagcaatcggcaggaccattaatacaggactggagtccagttggaagtcttattattatggtgctcatcttgcttgttcagttgctgacccacaacccaattttaccctttaccttttcttccgtgga

SEQ ID NO: 49 - DNA, 401 bp, Equus caballus
  misc_feature (93)..(93): n is a, c, g, or t
tcttcacttcaagagcagttatcagaacttaggggtctgaactacagacttttagagaactagatggcagatgattcccgtcactgggaatgnggcaatatcccaatnacacagctgttgccaccagctgtgagaacacagacactacactggatcaactggaaacacaaatgaccccgggtacagaccacacgctagggsgccacgaccataacaaaaatacctgctgagaggaggagaactggccaacaacgctacctcatgcaaaagacacacttctgaggaacacgcctgtgtcatcaagatgatgtccacttggggccatgctaccttctgagccacttctgtttctctggcagaatgtatccagccttctgattcagggcgtcaggatcacatct

SEQ ID NO: 50 - DNA, 401 bp, Equus caballus
  misc_feature (80)..(80): n is a, c, g, or t
tttggactaatttaatggcttaatttcaacatgatcacgattaaattggatctgctgttgaaaaaataaagaaagaaaanacactcacccagaacacttccaaaagagtggatttgaaaaggaaaaaccaaccaactcaccccagggccccagcccccgctaaggttcagcctcagcgttaaacacattaacatggaggaraggtgtgtgtttcttgctctgtaacaggcacacgtttcaaacataggattcaacatatgagatttgaaacatctttggcattttgaaaaaatttacaaactagaatcccagctcgttggttttctgagttgtttaggaagtcacttaaaggaaaataaacattctccntgtgttcgatgaatgctacagaacttccgtgc

SEQ ID NO: 51 - DNA, 401 bp, Equus caballus
  variation (201)..(201): y represents polymorphism t or c
ctttacattgcttcttgctgatgatgagtactttcatgataccctgtgcctgatctacacataatcagaacccttcatttcgttcggatgtggttccatttataaaatgtcaggggtgcagaccagcccagaacatcctaaaacttccagaagacaataaagtttaggcaacggagaagttggtcgggtgttcctccctcygtccctcgaggtacaagcttcggttctgcacgactcagctttccaaggtgagctgcgttgtcaggttggaacacagttaggtctcagactgacttagcgctcacacccacccctgtaggcctcttacttctgtcctcatgccaccctcccatcctctttcttggcaacaacgccatgaaaaacgtgcagatgggctccat

SEQ ID NO: 52 - DNA, 401 bp, Equus caballus
  variation (201)..(201): r represents polymorphism g or a
tagagtggtagcatattaaacaacacacaatattctatggcttgaataatggtgtttattttctttaaatacgcttaaatatgaaacaacagacagtagttctgtaaactcaaaactaaaagctggacggtaatcagaaaatcagtgctgatcatacaaatcaaaatctgcataagagaatcagaataattcacaagacarttgcaattagcaaaggactcggagactatgcgaattgcctcgtctgctctgagtaatcagtcacagggcacctgtcattccatgatgatcaaacctttttccttcaccaaaaaaaaaaagggttatagaggtttcctcctctccattctttgttgtgcggaggcatcttcagttagaagacatctgtcagtgaaccaggg

SEQ ID NO: 53 - DNA, 630 bp, Equus caballus
  variation (72)..(72): r represents polymorphism g or a
ctttgattgctgaccgaaggaagaagctgacctgggccataaccatcaccatgataggtgtggttctctttrattttgctgctgacttcattgatgggcccatcaaagcctacttatttgatgtctgctcccatcaggacaaggagaggggcctccaccaccacgctctcttcacaggtagggaatattccggaaagtctctcctttagctccccagacagggaggttcttacactgaagccatccagtgtctctgcatgtcaaagtttttgaatgaatggatcagctgatggaatgctctcatcacgcgggctcagtctcccagtgcatttctctaaataaagtcaacttgtgaccaggctgaagggttttgcaaaggaagtttacgtaagagctttctgaagaactatttgtggagacatttgcaagtgaaatgaaaagaggcatggtgtacttttggggtgattttttctttttaaatcagcaatgtgttttttttaagacagcaatgtagcatcggtattatttaggagcttgttaaaaatgcagttccattgggccggcccagtggcgcagtggttaagtgcataccgtgttgcttcggtggccccagggttaccagcctggatc

SEQ ID NO: 54 - DNA, 763 bp, Equus caballus
  variation (623)..(623): y represents polymorphism c or t
caggtgagggccccaccatccagcgtacacccccttatcccttattaccaccactcactcttcctcaaggggagaaggaaccaccactccctgtgaggaagcatggtgtacaggaaggagcccagacttggaagttaaacaggcctggcttgcagtcttgctggtgagaccttggaggaagtagcctaacctttctgagcctctgaaaagtaggaaaattaatacctgccctgtgggggatgttgtcaggattagagacaatgtgagtaaagctggttctgaggcaagagtgtaataaaggatcatattgatgattgttattaataagataaaaagtggaggaggttggctgaactgagttcttcacctgtaagaggggcagatccccaggcctggaatgccaacgtcctcaaagcagggaagcttgtagagtgagaggggaatggacagaggttaccatataaacaagagaaatgaaccctgtttgtgaggagaagaggaggcagctaggatcaaggccaagtaaacctgggatgtgggtgtgtcctcttctttggagaagcacagacaggctgcccttgtccattgcttaccagtttccttcttcttctcccaaatcaggygcagacttatgaagcagggctcagctctcccccttccccagctgccacacggtagaacccactggctgcgtctgccctgggtcttccgctcttccccctttggtgagagcagccccctcctcagtgggcagcaggtctga

SEQ ID NO: 55 - DNA, 1460 bp, Equus caballus
  variation (786)..(786): s represents polymorphism c or g
caagcgctcatttaacggaacgagaagccctaatgtctgaactcaaagtcttgagttaccttggtaatcacatgaatattgtgaatcttctcggagcatgcaccgttggaggtaaagccgtgacccgcttgcattttatcacctgtcgaattatcagaagggggagattttgatatgattttgacaatgcttgattataagctccttgcaagatttttacccaagttgttgttacctcttgctagagtcccccctgcaagagttattgttgttaagagtgtatattttagttttctcatcgtggggtgggatagtccatgacataccctagttactatcacgtatgttgcatccaaagcattgacctcttgaatatctcgagaatgtctcatcttgtgcccaaagcttttctttttatcttgcaagctttgttttgctggatcatatttagtcactaaagggttaatattcatttgcatatcataattaaaaataggcctttaagcctatcagctcacaaatatacaccaaatgaagcaaacacctcactcctgtaaaaaataaatttccaattctaaagcattagtcaagcctcccatcaaggatttctgtggtgtttccagaaagcattttggtcttaaagaaacaaagattagtaaaacgagctgttccttgagaactggagagatccatggtcacgttgacagctttatataatttcttaaagcagaccccatacctttttgcctcaccacgtcatgactcattcgtgagaaatttccgccsgagttaattagttgcttgttaccttcagagctgcagttttaggcattcacaacaccaaaaaacattttcgcctagtagtgctcagaacgcaggtgacgccacctcatttcaggttgtcacccgctttccagtctcgtctcaacgagctgggctgttccgtgtggttggttttcctcttgtgccgcagtttgcgctctcatcgtttccgtaataacgttaaccgagaggcgtgatgccccactgtaaactgaaggaaatcaaaggatttatgtaggacggttgatagtagcgtaagtaactcctgtctgtaaaaccagcagtcacacctggcccctaatctctcagaaattcaggtaaaaaggggcttgctcatttagttttactccaaataatgccgggttttgataagcaatgttaatagagaggattgtattggaactaagtaggctcatccattgaacctgaatattaatgactctgatcacccttgggtattttaatgggaggcaagaattgtctatatgtctcacctctttctacccttttcctatatgctcgtagggcccaccctggtcattacggaatattgttgctatggtgatcttctgaattttttgagaagaaaacgtgattcatttatttgctcaaagcaggaagatcacgcagaagcagca

SEQ ID NO: 56 - DNA, 1440 bp, Equus caballus
  variation (1120)..(1120): w represents polymorphism t or a
acgagctggccctagacctggaagacctgctcagcttttcttaccaggtggcaaagggcatggcgttccttgcctcaaagaacgtaagtgggaagagtccttttttttttccttaatcgtggagcattttagagccctagttagaatgcagagtgtcattttgaagtgtggtaaccaaaagcacaggaaatttagtttcttcatgttccaactgctgtctctttggaattcctgttctcatttataagctttaatgtgtaagcctgtctaaatgagctttctatgaatatatttttgtatgcaatgaattcatgtaaaacttttggcttttaggatataggagctgctctgagaaaatagagaaataattattttatcagcaaaaggagcaggtacctcatgtagttgcagtgcttggtgaagcatatacttgagtcttattaaagttagaccccaaatattgcgtgtgggtttgtgtagtgtaggggaagaaccaatcaggataataaacatttgggaaaaagacgggggggagagagaatgatggaaccataacatggaacatggtccctggataggagagaggagttccctaggacatgggactagcagaatagaataagattacagattctgcccttaagtgtcgttggtgacatttccaaacaattaccaaactaaaagaggatataggatggctgaaatagcctcttccctgtgtccttgggagatgtcaaattgaagttgcaaagacattttagaaactctgtaaaaagacagtgaaagagaagcatgcaaaatgagtctcagtttaaaaaatatgatacaactatttataatgtattttcctgtgaatgaaagccgtcctaagaagaagaaaagcatttattggagttggttttgaaagtgattatagtaattaaggtcctaacgatgagaaacacaagttttgaacatcattcagagcataatttagatatttatttttggtgcactgaatagtttaaatgtaaagcaaaaagtattggattggtagaacataagcagcattctagcattaaacatagtttcactctttaaaagtttaaaataaatttaaatggctttcttttctcccccwctctcctaatagtgtattcatagggacttggcagccagaaatatcctccttactcatggtcgaatcacaaagatttgtgattttggtctagccagagacatcaagaatgattctaattatgtggtcaaaggaaatgtgagtactcactctctgcttgacagtccagtgaaggattttagtttcacatttttataataagtgttttttatgattttcgtaatgcaaatgctccctttgagatagcatgcattttagcagtcaaattaagtgtacttcagcaaaatttgtgtggtattgctgaaccttactacaactaacat

SEQ ID NO: 57 - DNA, 360 bp, Equus caballus
  variation (134)..(144): Alternative includes deletion of residues gaaaagaagca
cctcccaattctctgcagttcatggggtaaggggcggtgggggaagagcaagggggaaaagaccagaaacatctggctttgctccttttgtctctctttgaagcattgaacaagaaatccaaaaagatcagcagaaaagaagcagaaaagaagaagagatcttccaaggtaggccttggacttctcattgtaggggtgggaccagacttaaaaggggaggaccctgaccctcaagctctggctaggaactaaatgaaggatttttcaggcctacatgaacaaaagaagctgaaagctaccaaaaggcttcctggcctggagccctgaaccagaccccacagaagctcagggagctgatgt

SEQ ID NO: 58 - DNA, 877 bp, Equus caballus
  variation (201)..(201): y represents polymorphism c or t
caccctcccagccaccccctacctcgggctgaccaccaaccagacggagcccccgtgcctggaagtgtccattcctgatgggctcttcctcagcctggggctggtgagcctagtggaaaatgtactggtggtgactgccatcgccaagaaccgcaacctgcactcacccatgtactacttcatctgctgcctggccgtgtycgacctgctggtgagcatgagcaacgtgctggagatggcaatcttgctgctgctggaggccggagtcctggccacccaggcctcggtgttgcagcagctggacaacatcattgatgtgctcatctgcggctccatggtgtccagcctctgcttcctgggcagcattgccgtagaccgctacatctccatcttctatgcgctgcggtaccacagcatcatgatgctgccccgtgtgtggcgtgccatcgtggccatctgggtggttagtgtcctctctagcaccctcttcatcgcttactacaaccacacggctgtcctgctctgtctcgtcaccttctttgtggccatgctggtgctcatggcagtgctgtacgtgcacatgctcgccagggcgtgccagcacgcccggggcatcgcccggctccacaagaggcagcaccccatccaccagggctttggcctcaagggtgccgccaccctcaccatcctgctgggcgttttcttcctctgctggggcccctttttcctgcacctctcactccttatcctctgccctcaacaccccacctgcggctgtgtcttcaagaacttcaagctcttcctcaccctcatcctgtgcagcgccatcgtcgaccccctcatctatgccttccgcagccaggaacttcgaaagacg

SEQ ID NO: 59 - DNA, 10 bp, artificial
  synthetic construct
gggggggggg

SEQ ID NO: 60 - DNA, 240 bp, Equus caballus
  variation (108)..(109): tc may be replaced with ag
gcgacgcaccctccctcctcccccgtgcgaaagaaccatcgagatcaaggagactttcaagtacatcaacacagtagtgtcctgcctagtgttcgtgctgggcatcatcggaaactccacactgctgagaatcatttacaagaacaagtgcatgcggaacggccctaatatcttgatcgccagcctggctctgggagacttgctgcacatcatcattgacatccccatcaatgtctacaa

SEQ ID NO: 61 - DNA, 72 bp, Equus caballus
  variation (41)..(41): m represents polymorphism c or a
acgtgcccgacctgggccgccttctggaggtcgacccgtamctgaagccctacgccccggacttccagcgca

SEQ ID NO: 62 - DNA, 171 bp, Equus caballus
  variation (78)..(78): Deletion of c
tgttactcaggggatgagaaccctgacatccctgagtgtgctgactgccccattggtttctacaacgatccacaagacccccgcagctgcaagccgtgcccctgtcgcaatgggttcagctgctccgtgatgcctgagacagaggaggtggtgtgcaataactgcccccag

SEQ ID NO: 63 - DNA, 288 bp, Equus caballus
  variation (83)..(87): Deletion of tctca
taggagctcactttataagttggtcttgtcattgagctgtggatatagtcattctctaatattatttttaggtaatttatcatctcaaattccccttaagagacttctaaaaacctggacaaacagatatccggatgctaaaatggacccaatgaacatctgggatgacatcatcacaaatcgatgtttctttctcagcaaaatagaagaaaaactgactattcctccagatgatcatagtatgaacacagatggagatgaagattccagtgacagaatgaaagtgca

SEQ ID NO: 64 - DNA, 96 bp, Equus caballus
  variation (66)..(66): s represents polymorphism c or g
ggggagtgtgtgctcaagatgttcgccctgcgccaaaactacttcaccgttggctggaacatcttsgacttcgtggttgtcatcctgtccattgtg

SEQ ID NO: 65 - DNA, 401 bp, Equus caballus
  variation (201)..(201): y represents polymorphism c or t
agcttgctaaagtacatttttcttttttttgcaatttggaaactgcatgtgactgattggcttatccaatctgtaggtagtaaaagatccgtattgtatttgatgccaagcccacaaatcctcgaaagagggaagaattcagaaggaattactggacagctcacaaggaatttcagcctcacaaaacttctaaaagctagyttctaagtaacacatttttgtctcgcattatatctaaaccttggataggtttttcttttatgcaatgtaataaanattttcctaaaatacatagcctggctattctatactcgctgggaggttgcatttttacnacttaacnagtaaagatnaggaaacgatcttcataaaatgtaagctagagacatggcacatgcata

SEQ ID NO: 66 - DNA, 401 bp, Equus caballus
  variation (201)..(201): k represents polymorphism g or t
cagcccggcatctctgcttcgcttgctttcttcccttttttccacccgctcttcttctttcttttttttacacttggctacggaacattagatttgacgaagggaaagcgatttaacatcttttaactgaaggaggttgattttcatttgagaccaaatggctcatttttacctttttagctcctggaagagtaaaaagtkagaggtcagccctgcgtcaccatggcaacccacacagctcgggccaggagcagatgaacggaggtgataatcggggggaagtttctccctcttccttacatggcattttcttcccattcagggatgtgcctggctgtatgaaaacaattangtctttttggaaatataagttttaaaatattactgctaaattgtctgac

SEQ ID NO: 67 - DNA, 401 bp, Equus caballus
  misc_feature (78)..(78): n is a, c, g, or t
ccattcacaggaggagattagcatctggtcccgcttggactgtccccagcacttgatgcattcatggctggtcttgancctgccctgctcctcccctcccatcgccacaccttcgtgtgtgccgtggcccgtgcctgttcttccctcttttgtcctgtctgtccctctgaatccttcttcccaaggtgctgctcgtccctsacctgttcccaaagttcctcccactcctgcagctcccctgggcgcttccttcaccactcttaagggctctgaggcattgggaagtaccgtgaaggaccctaggaaattcaactcccttgtttttacagctctagaaacgcaggagcagcgagaagtgacctgaagaagagcagcaacggtacctcctcgacttagctgtt

SEQ ID NO: 68 - DNA, 401 bp, Equus caballus
  variation (201)..(201): y represents polymorphism t or c
gagccaaactatatctttctcgccccaaacccaggctctctttccccaaagaaagtatttggtcttctccaaaccttgaatgtcagcctttcccttggaagtgttgctcagacacaaaatttttttcacctcttgcccatgaaagtttatatattactcctgatatcaattttacagtttttgcccaaatcaccagtagcycgatgatagcaattttcagaccctctatgaattacaggtgcgtgaaaatggtcccttagttattggagaagaaatgaattcagggttaatccatctgacctctttcttcatagagagcagagtttataatagacttttatgttgaactgttggtacatgttttaagaggaggaatgtatccatacctgggattttaaata

SEQ ID NO: 69 - DNA, 401 bp, Equus caballus
  misc_feature (34)..(34): n is a, c, g, or t
tgagggtggaggcaaagtcatttcaaagtattcnggtatctactgggtgccctggatccaatatagaacagtactggtggagagaggaagcataagacaggcaattttagaaaagttctcatgagggaagtgactgatgcatatgtggtacataaggttgcnagcccgaaggtctcacttagggacccagtccccatcagrgacttgggtaaagtctgatacaggatgcaccagggggtccccatgacccggagtggactgagctcaaggaagacaaatccagcagtccacatcttgcattgcattagcaatggtttgtgcaatgcataccagggagttgggagttgtccagtcaggcaataactggaaantgtaaggaatacagatgaattagcaatgtg

SEQ ID NO: 70 - DNA, 401 bp, Equus caballus
  variation (201)..(201): r represents polymorphism g or a
ccactccatcctctgtggtcagacttttgtgtctacacgtagttttatggagacaacatttagttcttcattaattccgtctgcaggagacttctacctaccctgcccacacacatctaggacatgcccgattatatgtttcattttaactcttcaaacccagaaaactgtccttcattcattgatttatttgttggtagrttccacaagcatccatggagtctgttccatgcctggaattgtactaggtactggatactaataagatgtctgggtgctatccctgcccccaagggaccttgaggacagtgggaaatccaggtacatgggtgatggcagctcaacagtcacatcctattgtgataataaagatttattgagactttatgccatgttctaaa

SEQ ID NO: 71 - DNA, 401 bp, Equus caballus
  variation (201)..(201): y represents polymorphism c or t
ctgtgctctggtgcagatccaaggtcttcgcagcaaagaagggaagaccggaagatggaccaggctgttgtggatttaaaaagggatttttggaatatgtttcccacatacattgtttattcctgaattatcctttcccaggaagcagtatatccatacctagatcttcatgcagaagaaagaaactgaggaagtctgtaygtttccttcttttagaagggagagccagggacgctaattgttacttagtggaaactaaactatattttcccttcccctgacaaatcctcccatagttttcttgctttgtcattcaaaaaatactggtgtttctaagagcctgagttcttgagacagtctttccagatgagttaaggaagaatctagagaggaagtccctg

SEQ ID NO: 72 - DNA, 401 bp, Equus caballus
  variation (201)..(201): y represents polymorphism c or t
agcacaccaacagcaaacgtttctgcagaccaactggagtttaatggtctttctcagtgaaaaaatatttaaatatctattgtttatgcctattttcatcaaataaattagtgaaattggcgagttttcaatcattactcatgttaataacctttcacttgaactcccactaaggcttctggtcattaaaaattgaacacyggggctggtttgtgttccctcccctggcttcctcattccgcttctctttttcctttacatggtgtccagttctcaagcacacgttcccccacccccacgcctgacctgtcacaaccaaacctcagggaatgagtcattcattctcattagacggttggtgaccacttaagcattgctgggtcctttcctgaggtatgtgg

SEQ ID NO: 73 - DNA, 401 bp, Equus caballus
  variation (201)..(201): k represents polymorphism t or g
atcaagatttccccaacttgagagtgaaaagaaggagaggtgagaagaagatgagtttgatttcgaatgtgctgaatttgaaaaatctcaagatagagtgtctagaagggagatatgggaatgagcaacacagagataataattagaatggatgatacaagggacatattataaaatgaaaaaagatttcaccataaagckaagatgttggatcatcttgctttaggcatactaagctattcctcccagagactggtcacatattggcaggagaatctgaagacctctaattcaacagcaataaagagaactaaacaatgggatcatgctccagagtttggcgtctagccttagcaaactcgtttgtaatgaaggcgccagctttgtagttaaacatacct

SEQ ID NO: 74 - DNA, 401 bp, Equus caballus
  variation (201)..(201): s represents polymorphism c or g
acccacagtccttttggcgaattcttatctcgtggggcagtgcagacaatgggggtttgtgctgacggctgctggtactctgctaatagtcctggaagcttccacgttgcttatttgggcatcttttcacaccatcacctgcaaccagcagtccctggtgatcccgactttgtcagcctggcacccccatccagatacgtscggaggaaagagcttggtctgaatgggggcggaggggagctgaggagcaccgggaggaaggagtcaaatactgttatctgggtgttttcgcattttgtttctcctgcacacccttccctccccttcccacccccgctcccagtatcatttctctttgaaatgtcatgaacttggcgctttcagaccaaatcgccgggctc

SEQ ID NO: 75 - DNA, 401 bp, Equus caballus
  misc_feature (10)..(10): n is a, c, g, or t
ggagaggagngcagggaagacaaatcgcctgtttgcttgaaagggaaaggctacaaatcaacgctgggggagcgagaggaagaggggggcaaaggtcaggggtaggaacgagggggagacagggcagctacagcgcaggagtaggacaggaagcatttaaacgaaatcccagttttacacctaaaaatataaagctgtcarttttcattaaggatgaagcaaatgaaatctagagggtgccaaataattaatgacttttaataaccctaataaatttggatttcatcaaaaccatgttgcctatcatggacgatcaccagcaagaatggggagctgtcaaggggggctaacacctgtgtgaaaatggacgccnctgacatccttcccaggtacatcaatta

SEQ ID NO: 76 - DNA, 401 bp, Equus caballus
  variation (201)..(201): s represents polymorphism g or c
tccacgcaacggttcttctctgaacagcaacagagcaaacagataggaggcaaagctcagaaagtggacagtgattccagtaaacccgaagcgctgactgaccctcctggtgtctgtcaggaaaaaggagaagaaaaaccacctcctgcacctgccctagccgccaaacctgtcagaactggacccatcaagcctcaggcsatcaaaactgaagaaacaaaatcttaaaggctgtggtttattgccagggattgggggaggggagaggggaacgagggagaatgaagtcagataatgccagcagccaaaggggtaaaacggtctgtgacattatcctgtccagagcttggaggtgcacaagggacataggagcaatttacactgacacacagctgctacac

SEQ ID NO: 77 - DNA, 401 bp, Equus caballus
  misc_feature (92)..(92): n is a, c, g, or t
taataaaccactttcctcatttcagtactgataggcttatgggaatatgccatctttggaatctaatctttgcattatcttatctatccttngtattatctttacaaccaatgttataaggccatagaaacagagtataccctctcatgggcaatgtgtggtaaattttccccctttgtttatttctcttcaggccttcawtgtagcaccctcccaaaccatcccttcccgctcagcctacacacccttcactcttacagttcaaatatagtactcctaatttttactgaaaaataaggccaggattttttcactctcacttgcttctcttgccactctggccacaacaatttacatgcctctcacaaaagcctcatctcattatatcactggctcaaagt

SEQ ID NO: 78 - DNA, 401 bp, Equus caballus
  misc_feature (3)..(3): n is a, c, g, or t
tcntaggacaagactagtggggaggagacctgaactccagtgaattgtcccatcaagttcattatctctggaattgtttttactcatgggagttcttacagtaactcctataaggaggttaatgggagggagaggtggcctatgaagncagggagaggggaatcctgacagcggaaataaaccctctccacaaaagtcagrgctgtctattgacagaaaggctgtgtgtgtgtgtgttgcacacgcatgagtatcgatgtgtatatgtgtgcgtttctcatctacttctcctaaacttgctctcagaaagaaccactttttcttctttttctttttaacgtatttggtttccactaaatcaggattagtggccatattcagcctcgaaagacagttggaag

SEQ ID NO: 79 - DNA, 401 bp, Equus caballus
  variation (201)..(201): m represents polymorphism c or a
ttgtaaaaagtttcaactttaattttaattgaagtgcaaactacaaccatgagatatgcttttgtttatcagattggcaaaccttaaacacatcctttatcagtggttcatctactgctggtaggaaggaaaaaatggtacagtatttctgttacttctggtgtgaatattattgccaaaataagatttagaagataaaamgggtttgtggactgtgttttgtgatgcaccatgtgattgcagactgctcttgttttttccagtgataaaaagttgattgcagaaggccctggggagacagtgctggttgcggaagaagaagctgctcgcgtggcgcttcggaaactctatgggttcgctgagaatagacggccctgggactattccaagcccaaagaggg

SEQ ID NO: 80 - DNA, 401 bp, Equus caballus
  variation (201)..(201): s represents polymorphism c or g
cagtaatcacattctggcttagcccttccgtagaagccacaggcaaaggcaatggtctctgttttcagtttctgccatgaagaatcacaagtctctggagaggacacatgtctcttaggtttacagaacattcaagaaacaaggctgtgagtttgggtcctaagtcacggtggtcacttgaagacgtggttctggaagtcsccatttcgcattagccacagtccagctttgccacaatccagattcaaaagagacgtattccggcaattctctgagaaacatactgcatatgttgctgcattagaaactaagccaggccccacaggaggccctaaagaaatggggctcaggcgcgtgcagacacaagggggtggtgagagctgttattcgacacgtgcact

SEQ ID NO: 81 - DNA, 401 bp, Equus caballus
  misc_feature (41)..(41): n is a, c, g, or t
agcctgccatgcaggagcagatgcccactcccggggacccncagacacccccacacccccagccaatcctgcagctcctcctgtgcagccccccggctccccatgcctgcccccacctgctcataggccatgaacttgatagccgactcaggggcaatcttgagcacgttgatcccgttgccacgccacagggaacgcacrcccccttctcggatcatgctccgtaggccccccaagatattcagccggttggtcttggaggcgtggacctgcgcggggagacgaggtggccttggggtcccctccggggggcccaggcccaggaggaggtgggggttcctccaggccccctcacctgcatgaagaccttgaggcggtccagaggggcggtgcctgtccga

SEQ ID NO: 82 - DNA, 401 bp, Equus caballus
  variation (201)..(201): m represents polymorphism c or a
gttctggacatcactgtacttcagcaataagtggtgtgtgtgtgcggagtggggtggcggatggatggaggcttggagaggggatgatagggcttctagaccgccaggaaaaatccccccatgacatgtggggagaggcctccattggccagctctttgcctccacttccaggaaggagatggatggtgtttacctcccgmtttccacgccctggccgggactgtaccagaacaactctacaaggaacaggattcacccatgctggccactatttccatggctttaacttgtcaccagtgtaccagggaaagctgacacttatttaatcctcacagtggtcttgactgtgtactcacgaaaacattgtatgcttttggaagacgtttgtttccaagcagct

SEQ ID NO: 83 - DNA, 401 bp, Equus caballus
  misc_feature (84)..(84): n is a, c, g, or t
cttggtactctcatcttagagcctatcttacatgactgtggatcttaacttactgttaagtagttactgaatgctgactatgcnatggaccctgtaatggacacaaagaggaataaggtgctgtcctgcctgagggaccgagaggctattcagagacccgtgtgttcaaatgtatctatttacatcccacagtcagctgtytccaaagtcaggggggctagggcagctcacagaaggaggatgctatatgtgctagattgagttaggccagtccagagaggacatggcacttggcagttgaattgctgtctagggagaagcctaagaattcattcactccacaagtatttatttttaatttattgtgtctcagaaacaaaaagggaaaattaatttaatta

SEQ ID NO: 84 - DNA, 401 bp, Equus caballus
  misc_feature (91)..(91): n is a, c, g, or t
tgaggcaggacttccctgagctccgagatgcttgtgtgcactgcactgtccagggcgcttggctgtttcccaggctcgcgagagcatggcntctgatttttacatggcctcctgtttagcccccacaccccctcacactccctcacactgttctggcgctgctggggcagacgtatggaggaataatcatcgtgaggggcrttgaactcagggaaggtaccaaagtgcattgtggggtttggccccagtgagcttaaaacagtctttttcagtgagctctgaaccccttcccgtcccagtgctgttcttgttcagaagcctgggatgaaccccagcttctttccagagacgatttcaggcacacagggatcttttatccttgtttctcctggtaccttgga

SEQ ID NO: 85 - DNA, 401 bp, Equus caballus
  misc_feature (18)..(18): n is a, c, g, or t
ttctgtttgtttagaacngcctgatgagagaattggatcctgagctctcatagggacatcgccataaaatcatctgcccgtatcgttggagagtgggaaaaccttcctcnagagagtaaatagtctaaacaacattgtttagatttagntagtctcgtttatccacagctcagagacctaaaatagttctgcaaaaggacrcatgagtaggaaaacccctaggctcctaggatgccgtctgtggcccaggtggcaggtgtcctcccggaacaccctgtgaccgggaggantcatgggaaaangaggctctgcngaagccaccaccncctcccagaacctgctgtccaggagcccactgtttatttctattttttcacttcatttgtttttaatactaggat

SEQ ID NO: 86 - DNA, 401 bp, Equus caballus
  misc_feature (272)..(272): n is a, c, g, or t
gatatgtttgaggtggtggtttcagccattttggtgagttactcagcttgcccgagcaactcccatgtatacattgacaaatagaaatggcaagtaataggatatatgggagtacatgaggaagccatttggtgtaaacctaattcggcctgactttgctattttttccaaaagggcctgaccgaggctgttgagcatgcrttgtatatctgctttagatattccctatggcaagaacaaaggcccttgagataaaggtgcaacttccctcnccctcccaangtagacatttccttaaggattaagcatctttccttaggctaggaactgattgctttgcttacctgtgaccacccagctggagacaatagacttgcctcctgctacgcccacagagatag

SEQ ID NO: 87 - DNA, 401 bp, Equus caballus
  misc_feature (57)..(57): n is a, c, g, or t
ccaaactgaactaagaaaacatgtggtggcgagggggcatctggtgctataagacangggtggaggaaaccacagccccaatgctgtagagtgactcgataagcttgtccctgtgggagcgaaaggagagacacagggagaagggcatctgaaaggggtcttctcctgaaagagcttcattcccctcccctctggcgggcyggattctttgcacagataattctaaacctgtctttcccagcactttcggctggagaggtagtctgcaaggttttctgccttatcctattttattaccaacctacctccacacattccagttttgaagagaaaactaataaggaaatggctgaaatagtgtcttgatggcagaaggaagaaatagctccttaaaatttgtt

SEQ ID NO: 88 - DNA, 401 bp, Equus caballus
  variation (201)..(201): r represents polymorphism a or g
agcatagcttaataatacacttcattttcacatttatttacttcaaagtgtattttcatgttttatgtacttatctatatttttgcttttagcacaaagaacagaaaagtaagattaccacagcattccaatatgttaacaaaatttggaatgaactatggatcaaaccttgggcactgaaactcttcagggccctttaargaacagtttgaaaaggttctgtgaagtttnaatagttaggctagaagtccctcaaagaagaggaatagaacgcacaacctcccaaatagcagaggcaaaagaagagtgagaaacccacagcaatcctaaggagaacaaactacatattttactgattttttttttcccatcttcctcaattagaatgtaatcgctaccaa

SEQ ID NO: 89 - DNA, 401 bp, Equus caballus
  misc_feature (144)..(144): n is a, c, g, or t
ttgatctgaagctgtaaattccccagtagccaaccctgttgactcacctgatacttcatgatcaggcagactaacttgcttccagtcaaataaacagaaaggaaattatttgaaaatcaggaaaaaaataatctgggatagtcngtgatctggagttgtcagtaagaaaaactagcattttagaaatgtttaatttatccrttgacattttgttctgatttttctgtgtgtgtttgtttgtttttcctatttaggagaaaagggaagatgtagccattttaagaaaaaatagacccattgaacaaatctgaaaatatcagtatgtatgctgatggcagaagagtataagagagactctgtatttgtaaacataattgatggttaagtaaagtactcaataa

SEQ ID NO: 90 - DNA, 401 bp, Equus caballus
  variation (201)..(201): y represents polymorphism c or t
tctagagagagaagtgtgcttatcctgaaagtaaaaaaacccaggagagaataggaaaggacaaatatttttggtagaacctggttgatctgaactcagaatctcctggagattcacggggttccccggcttggaaggacaaagggagaagccagcacctggagcagagtgaccgtcctttgtgatctgtgtgcaccacgygtcagaaatgtacccagccagccaccggatgtcactgttctctcgtgctcctcgtctgggcatcccacaacgtctctgcgtgaatnttcggtgtcctgctttgcagcctgacagtggcgcaggctcacccgcacgggcctcgcaatgtgggatccctgactatccccacataaccatgattttcgttccagagcagcaca

SEQ ID NO: 91 - DNA, 401 bp, Equus caballus
  misc_feature (19)..(19): n is a, c, g, or t
ctataacggactccatganctgagagctcatgtttcaaacggcagggngggggagtgggaagtcactagattgctggttcttagacagtcttagggccagttctcattctctctcctgcgctgtaggcttctgaagacctcctcaaggaacactacaacgacctgaaggaccgtccgttctttgccgggctggtgaaatayatgcactcggggccagtggttgccatggtgagtgtgcacgtgtgggaagtcactgtgaatggctcagttgggggtgaagggtgggtgttgtgattcctgcttctcatgccgggtcctcatggatgcggagcaagctggggctgggagggttagacanttaggatgtgtcagctcgctgacgcaccaaagacaggatgaac

SEQ ID NO: 92 - DNA, 401 bp, Equus caballus
  variation (201)..(201): y represents polymorphism c or t
taataggttcaacaaagttgtggctgactcctttagttgcttatccaaaatccattccctacttcttcctcaatttaacattaatttgatttgggacagcaataggtttaggtccacagtgatggaatgggaccagcctaaactaatcacagcctcccttgcagctacaggtggccaaatcaatgcatatagccatagaaycctcttctttcagagatctcaggcatacttttaggagagtatgctttgagactattggacctaggctaactgacttacaaattagatgaacgcacttcctgaccaaccaagccagaatgtattcatcttttaggcatttctcctataaaacatttactctggtttctctcattaaatgaaaacctatgcactcattcatg

SEQ ID NO: 93 - DNA, 401 bp, Equus caballus
  variation (201)..(201): r represents polymorphism g or a
tcttacttttatccacagagcttaagaagttagtcgggccaaagactgccctctacttctccctagctcactattacccctcaagaagaccttgggtgaatggcccatccaagttctgctctgagcagagagacaaaatccttccttcattgatatgatcttcctttaaggaactttccccaggattggctcccctcaccrctgacactgaagaagtaagggacttagggcccaagaaaagcctncctctagctgagacaaagaaaatctcttcataatccaacatcaggagaccacatggaggatgaagcaaaagcttcaaagactgtcataaagattgtgcattcactcagctttgtcctttgttggagacctctccagggcatgactaaagcaggaca

SEQ ID NO: 94 - DNA, 401 bp, Equus caballus
  variation (201)..(201): r represents polymorphism g or a
tgtaatcaaaactacagggcctgacgaaggaattcaaattcacaaccaacttttgtgacttctggaaggataaccatagtgacttcctgtaataaaatcagcccctgaccaccaagagaagcctgtggaaccccgagacttcaagcctgtaaggacagcttcagccagtcccttatcatttagtagaaaaccccttaggarcttcagacatgtactaggacttttggccaatctatacactgactagaaaacttatgcataagttcagcacaactattgttcttgatatttagagggtcctcaggaaggccatgtgctggaaacagagtatggctaggaaagttctttggagggaaggtggactttccaccccctgctcaggggcagtgttctctgttcct

SEQ ID NO: 95 - DNA, 401 bp, Equus caballus
  variation (201)..(201): k represents polymorphism t or g
gacactggaataacagcaagaccatgcgaaacatggacgacaccgtccctgccctcctggaggtaacgacatgaggaaggggttatacgtacaggccatgtggttacgagtgtccagaggttggcaaggggctgcccaggatccagaacccccagaagctgggaagcaaagcagattctcacacaccctcactctctccckggctcagtgcaataagctgtgagtgcagtcagctatttccttagaagactgataaattgataattacacagagatgtaacacatataattaagcaatattctacaggaacaggagttttgctgcttttttccttaatcaaattattcctttctaaaggtctctgttctgaggaagaagagacaggacaccgattctaagc

SEQ ID NO: 96 - DNA, 401 bp, Equus caballus
  variation (201)..(201): s represents polymorphism c or g
caggtaagccccttctactttgttcggcctcttctttccctcccttatgctgtcagttctctcccagctctgccctctcttcctgtagacctgggtgaatgaaggctacttcccggatggtgtttattgccggaagctggacccgcccggtggacagttctacaactccaaacgtattgactttgatctctacacctgagsctgctgagggcccggtttggtggccccttctttcctggacnctgtggaggaggccccacgtgcctcaggcagtgaggatattgggggccacttttcagtcaattttcctttcccaataaaagcctttagttgtgtattatggccttggctgtgctgagggccaaaagccttcttcacagctccngtggactcacctccat

SEQ ID NO: 97 - DNA, 401 bp, Equus caballus
  variation (201)..(201): k represents polymorphism g or t
ttttagggaggtggtgggagctgggtggacaggagaagggagacttttctcagtataacttactgctattttaaaatttttgaaccatccgaatgtattacctattcaaaaaaattagatgacaaaagtccccacaaggctagaataagagcaaactaaacagataaattccatgctggtgcagtaaaatggagacaagtkccaatctcagaaggtgaccctggcaacaaagttgtggatgtaggctagctagccctggtcactgtaaaccaatgaaagaaatgtgtggatggaggatgggcatacacacatgcacacacaatgccttaccagacacaagctggaaagggttcccacaattggagatgtctgcatgatcaggcaaaggctaagggagagag

SEQ ID NO: 98 - DNA, 401 bp, Equus caballus
  misc_feature (124)..(124): n is a, c, g, or t
gaggaagtagaattcaaggaggaaaaaaaatcctaagacgttagtttgtgaaacgtgaacagaaggcaaacttgctgcaacccacgccaggccagctcgcccaccgaaaccgcggctccaggangcgnggactcaaacacccctgcctgggcatccttgcccncggtgggagaggtgcctgaaactcagaggggcggtggrgtgctatgatctgcgcttctccctctgggtctatccaggtgttttgtacacgaaaagatttcaacacagtgaaataatcaataacttataaggcatgtctatacttgcaaagtgaaatgtggaattgagatggtttggagagaaaaagagattttaaaaacagacccaaatagtccatnanaataaaaaacttatacttt

SEQ ID NO: 99 - DNA, 401 bp, Equus caballus
  misc_feature (29)..(29): n is a, c, g, or t
agaaccctagaggcccaatgttctacagnggctgtttccaagagggaggaggctacagcnactgccacactggatacactcagaggagaaggaaaggggaggaccccgaatgcctctcatgtataaaagcgcgggatggcaggaggcctgtgtggcccaaggagccagagggagcaaattatgggcaaaagaattgcaacycaaggctggaagagaagagctctgccctcagctgtgtccctggctctctggcatctgcttctaatgggcaggcgccattttcngaagcatcaggcactggaaagaagacctctgctgctgggattgaaaggaagtaacanccaggagggggagatgcaagggcgaggctggtcccagaaacaaggcctataatgcagaca

SEQ ID NO: 100 - DNA, 401 bp, Equus caballus
  variation (201)..(201): r represents polymorphism a or g
taggccaaggcccttcatcatcaattcgggaatcagctgttctgctcattcataatccaagtggataaaagcgatgttaattccctttctagttctctcaactagaaagttacggaaggtgcgttattatctaggcagaaacatgtctccacttaatattgcgggtttgctccctggaaatgaaatggtgaagtggggcartatctacgtatgtgccactcttctctcctctaagaagactgngagatcagactcagggtgagaaccctcacnccagcaaaggccaggacacccacaaaactgtctccgtgaatacatgtctcagaatcaccatttttgttccatctttctcacctctacttttctttctacagtcttctctttaagaactaagtgtcctg

SEQ ID NO: 101 - DNA, 401 bp, Equus caballus
  misc_feature (52)..(52): n is a, c, g, or t
aactatttctgaggaatagcaatagcaatagtctcatgagttcggtacttanagggcagccaatctaagtcagaaggggataggatctaccctgggtctccctggaccacgattggatcccatgtctcctgagtttgtgtggctgtgcttcttaagcgctctgatcctcgtggatgccagcatggtagtcactgttccttmccagacaaggcctctcttctgtagcaaccctctccaaaggcacagttaccaacctgcctgctcctccattcagggtgggcatatacaggggtgcaggtggtttcgctgcagctgccctgtccccaactgcccctcaggtgggtgggatgatctatctacccttggcttgtgtggaaatcacatagtagctgtggaagcat

SEQ ID NO: 102 - DNA, 401 bp, Equus caballus
  variation (201)..(201): y represents polymorphism c or t
aacatggaatgatttttctttggaattttgcatctgtcttcttctatccctcctaccacaatcataactcaaaccataattcatttatgcctgggcaaatactattgtctaccagctgaattctcatccacctccttctaaactttcacactgtttctttgctaagtttttgttttcttattctgtgcttgaaagcttcayacgatgtcccattatcaacagcatagaatccagtctctttacttagcatcagataccttatagtccagtcctgacattactggttaatttcatctcttcaatgtgtacattgatatacacgcttctatttatcatacttccagcatgccctatgatattggttttgccttacatttgctcttcagtcatggaaatttttc

SEQ ID NO: 103 - DNA, 401 bp, Equus caballus
  variation (201)..(201): y represents polymorphism c or t
ggccactgctaaaaactgcccgctgttcctggccacacagcctgtcccaacatgttcacttgcttccttacaccagcaaacattctctagtgcggtctgctaggaagatggagtcttacatagacagaacctatcaagaaagtagcatccatctcttcactatatcctattggagagaagaaagccacccagccatgacgyaccacttgattccacggctggacgggaggctcactgagagcggcggcggagaggctgagctggacgccaagctccgctcgctcgcagcctccttccctgccccagctcctctgcttccgggccgcatctgcaggagcacaagccccggggccggtcctgctgctcttcctgagcctcccctgctggtgctaccttgggcc

SEQ ID NO: 104 - DNA, 401 bp, Equus caballus
  misc_feature (56)..(56): n is a, c, g, or t
gacccccaccataccagggaacatgaagtgtaggcagccctgaggcccctgactgnggagatgcgctcatgggtccattaagctgggaggtcacactgcaccgctggctggccaggacctgcgcacagggtttcttctgactacatatttgtttttcaatttgaatagttgtccatgtttaaccatctccagatttctagsttatcttgaaaaatgagcccacacaggatccaggatttcacatgagctgggtcccaacccctcccagtggggtggggtgggcgtgcttcaggtgccgccactccctgcttctcctgagtggcccacattactaatttaggtgattgcctcgtccccgtgggaccacgtgagcagcgcctacttcaatgttcctgacacca

SEQ ID NO: 105 - DNA, 401 bp, Equus caballus
  misc_feature (161)..(161): n is a, c, g, or t
attagattcagagagaggcggagtaacttgctcacactagtaagcagagtttaaactgaggttaaatgaatagaaagcctgagttctttccactaaaaataattctgtgtaactcaaaattttgttcttatttatttatttagcataaaaaagtttcttangcctggtattggaacttggtactctgataaaagtatcacrgcttctgaaagcagcctaaccttccaagaaatacagtgaatcagaagcctgttgtctctatcgactccccaagaaagctctaaatctcacctacatgctttccagagttgctaagtgcagccctcctttcttagcaagggattcacccaatcactagtccatgcactattgaattgcagtttcaaatgctgagtgtagag

SEQ ID NO: 106 - DNA, 401 bp, Equus caballus
  misc_feature (142)..(142): n is a, c, g, or t
ataactagaaaggatgggaaatgccttagtgcttactatttttaaaaagctacttcctcgatagaaagactttatttacaaataaattgaaaagggtttctgaggaaaacaacatattacaaatacatttttgttaactttntttnaaaaagcatcaccacaacatatgtctactcaaagagccttcaaaactccattttsagaaaaagagcaaagaatctttatttccaagccaacaanctaaagggncagtttggtcanacgcgtaacgacaaggaaccttagcttcctgatccagagcaaacccagcagttcctttaaaggtgacacaagaaggtaatgaggagatctagagagtggaaacgcaaggcctgctgaaagtctgcgcttgcttaagcagc

SEQ ID NO: 107 - DNA, 401 bp, Equus caballus
  variation (201)..(201): k represents polymorphism t or g
attcttttcatttctctgtaggtatttaccatatcccttcctggatttgatcattataaatcttatatcatggttattttaattgattctgagctttctcctgtccttactttatttatttatttatttatttattgaagtacttcatccagaaacagcatcctacacacgggctgccattgtctgctactagagttgctktaatttaactctcccccttccctgtcaaaattacatcctaagtatgctctgggctcaaaccagaatgatcttaatgtggatgcaattgtatttgaaccaagggtcagcttagaggcagctggcaaaatgttcaacagacaggaccaaaaggaagaattgtaacagaaaatgtgattctgtctggagcaaagtgagaaagt

SEQ ID NO: 108 - DNA, 401 bp, Equus caballus
  variation (201)..(201): r represents polymorphism a or g
cataggctgagtggcagaggacccttacgaatcccccaattctttgatacctaaggcaactgtgtacaagctgataatataaatcttgggaaataataaccggagaaattctagggcgctcagttctggaaataacttttgtagagctctggatctaagggcctacctttcttggacagcctacacggtcccatgcaagcrtaccactgctgggatggtctagttcaaaggaagaaaacaccttccctgcctttctgcttcaatttgccctaaccattttctgatttgagaaaagcaacatagcaaggggtactaacacttctgtactaatcggccagggtgaggagtgaacaattagagttgctcttttaactctaatggttgtcaaggcagggagatga

SEQ ID NO: 109 - DNA, 401 bp, Equus caballus
  variation (201)..(201): r represents polymorphism g or a
ctgtgtggatttataatatttcactgtactttccactgacattaattgaataaaataatcttatagagaggtttggcttcagtttagtcacaatctgatgattaagtttgatttaatgctatttattattctgtttaaaaggagtcttcagaattgtgtcatctggagtccaatgggctagttatctggtatgcttatccrttctccaattttgattattttcttcaaatcattttaaaatgttatttttcatgaacatttcctaacactgcacttgcattttccatttcacacctctctaatctatgtacagtaaggctggtatgacancctatttagtnatccacagtgtggtnttnatgaagctcatgttacagttccattccctacacagaacctaa

SEQ ID NO: 110 - DNA, 401 bp, Equus caballus
  variation (201)..(201): m represents polymorphism c or a
aacaagaccttgatcatgtgccactgcgagaggaactcgacgcgccagctcatgcggccgtggcctgagttgggaaatggccaagaggagtgagctggagctgtgctcatggcagtagtgtccggtgggattctggtcaagtcagctggaggttccgccggcagggtggaggccagaggtataggggtgtgggtcagaggmcccagggaatgtgctggagtcatggtgagagtagcacctgccacaggctttccgcgtggctgctgggctctggcgtatgctgtgttgctcgcctccccagggtagccttgacatgatgagcgatgggagcatcccttggaagacagcctccctggggagctgcgggagccaggaggcacaagctgcagccgggagcagag

SEQ ID NO: 111 - DNA, 401 bp, Equus caballus
  misc_feature (28)..(28): n is a, c, g, or t
tatcctcatctaaagccaatgataaggnttttcaacatgttgctaagaagaagtctattgcaatagattgttcctttctaattctttgtcacctggtttcttttctccttgaacatgggtgggaaagatgggccacaggtttttctgcccacctggaaggaatgactctacaccaccccctctagcaggactagcactgarctctgacaagatagtctttgtccagggttggtgatcttgggacctgaggcaatgagatacaggacaagaagggatcattggaaaggtctaacaaaggaaagtggggagccactttcttggtatatttgagtcacaaggctttggatgctcaccttgctatttaatttttacaattccattttgtgataatcaanatctgt

SEQ ID NO: 112 - DNA, 401 bp, Equus caballus
  variation (201)..(201): r represents polymorphism g or a
caggcaagaagcgtctcaagggttgaaaaaaatacaatctgccaatgtagactttcaaacagatcccttttttctttgattaaaaaaaaaaatggagttattccaagatagaagttactgcaagatagaaggttgacagtactaaatgacagtcaaaaaacatataacctgaataagtaaaaagaaattaaaatgaagtcrttggtatttttatgaatttatgaagtcagcatggtctgtgggtatagatgcagctaaaacctatgtactcaagtttaaattgcaggttgatttttctacccacacatatttaagtcagttgctttattctcatttggagtttagctcccaaccttgcacaagatcttgaacttaatctcatgtattctagaattcaagat

SEQ ID NO: 113 - DNA, 401 bp, Equus caballus
  misc_feature (35)..(35): n is a, c, g, or t
tgcacatcctgatagcagcaaagacgaaagtgtgngaggggaagggattnatcccgaggcagccagctcatcatcngcaaaactgggataggaaaaaagctcgggtccttctcccacaacttaagctcgcatctcctaattttcataattgagtgattttcccactctttccatcattttggctggatcctgctgagaaaratggctttttttcagagctggaataaagactcttcaagttgatattgggtttaagccacagatgctaagatgtcatcaagttcaaagtcggaatcttctagaatctttgcctgcagacagagatgctgagccagctggcagacgtggtggtgaggacatgcagagctcccatacactccacttgtccatggaattgtacg

SEQ ID NO: 114 - DNA, 401 bp, Equus caballus
  variation (201)..(201): m represents polymorphism a or c
ttttgtgataaaggatttctttgcattttttcctctagtcaagtaaattgcttgtgggttcttcctaagaaaaataatccctctggtgctgcttttaatttgatcaggtttaaaatgttttcagaagagttaagcttcctttacattggtgtctggtgtggtcagatggaggaatagctttggaatgaactagattttttmgtgatgcaccgtttgaccttcccacagaaggttcagtacaaggaatcagtcaaaacaacagcaccattttcacttgacctcgagacatgtggtgtataccctttaccccgacagatagaacttcctaagcatatttttctttgactcatgttgtaagagtttatgtttcttatgatatatatccattgtgtccaactgtc

SEQ ID NO: 115 - DNA, 401 bp, Equus caballus
  variation (201)..(201): y represents polymorphism c or t
aaattctttttgaatgtttacattacttttctggttaatagttttaaaattctgtgaaggagcatctctgaatttatctgaaatttatagatactttccttattcaaacaaaaacaaaaccacaacacaaacgcaaggaaaaacaagggtccaataaagtggaaacttctgttatggtctaacttttggtcagcagtatgyaagcataattttggttcaggactaacgctaacgagaggcaaagctgaggctacggctacgggatgatggctgaggctcatattgtattactggaggggcccagggggaagttaaaatgagacactagctcctgtgcatcaggaccgtcagctctagaggtgtcaggggcccctgagttggagcagtgaggaatcccctcc

SEQ ID NO: 116 - DNA, 401 bp, Equus caballus
  variation (201)..(201): m represents polymorphism c or a
cccccagccctctgctgggttcctaccaggctccagcatattgaccccctgacttcatgcctctgttcagaccagggtagatgaactgacagccgcccaaggagctgccccttccccccaccaccacctaaccatgtcccgcagaggacacgcaaataaaagggccctctgaatggacttcaaatgcaaagacaaattctmaaaaggctgtgcatacaaaatgcacacattggttgccagagatactaacgttcattagtatttattagaaatcgtgacactgacacttagttcgagggtcagtctccgtgaaggcggctggccgtggctgggtgtggccagccagcccccctactcctctcctgganggagatggcctgtggggagctgtgcccccaagc

SEQ ID NO: 117 - DNA, 401 bp, Equus caballus
  variation (201)..(201): r represents polymorphism g or a
cccacaggactcggcttctaggcggcagggagtgactccaggaccagagagcaggcagcaggaaccctgagggactgcaggaagccaggctgcccactcactcagtggtttgcaggcagagggagcagctagaagcccaggagacccttgtccaccagccgcctcctgggcccaacagccgcccgcgggcaggcccggtgrgaatgctcatccgacctgcgaaggtctccatactgccagtctgggcagactatgcggggctgacagttgcccccagatgttttacagcagccgtgaaagggcctcgaactccacagatggcgagcgactcgcagccactggttgtgggtgttccttgctaacatctgcacacacacacatgcacacgtgcacatgcatgc

SEQ ID NO: 118 - DNA, 401 bp, Equus caballus
  variation (201)..(201): r represents polymorphism g or a
aaaaggtcactttccaaactgcttttgctcccaggctctgctctgaataattcaagtcatcctcagtaagagcagcaggctttggggtgatctccagcctgtttgacaggaacggtgctgacttaagctaacaagaggtcattgtctgagcagaagaatggcatcctagcttcttgatgagcagaaccaggcatgggaacrtaaaacaagaccaactgactcattgtaactgaaccagaggcagccaagtgccttcccaaattcccttcttagcaggaccaagcccttgaggaggaggaggcatttattggggattgctacaaattcggctttactgccaccgcctttataaccacaatataaagtaactcccacaacacagtgaagataagctctttaaa

SEQ ID NO: 119 - DNA, 401 bp, Equus caballus
  misc_feature (161)..(161): n is a, c, g, or t
actgtgtgagcagctgaccatcctctctgggtggaggtagagtgtagacagacagataaaaggttttgtctggatgaaaccttcatcatctgtgacctgcttgaaatgtaggaactgctactcggcagtgaggacacccaccaggcccctgtgacctccangggtgtgttaccagcacaggcagagcactcacagtgatgktcctcttcacagaggtgagtcgggggagattcttgggccacatctctctacagtctagnctttgtagtgtgtcctgcatgtggtggtgtggggactgattctggtggggaccccttgctgcattaggtctcattcacacccttatttcagtttgtactgtggctcccatgcctggttacctgcagtgcagtggtgactct

SEQ ID NO: 120 - DNA, 401 bp, Equus caballus
  variation (201)..(201): y represents polymorphism t or c
aattacaattttaaggaaaaaatctatctccttgtcatcacatttcccgaccttcctttgagagagattctagagtccattatttctgtggagattgctgaaaatatttttacaactctctccaatatatttctactattaaatatctccatcttattttactaattcatgtttgtctaaattctgaggttttacaggctytttggtattgcaattctcttatccagctctccatctatggaacattaggaagtctctgaggccggtctgaatctcataatttggtagtgaatcagaccaacgatttaagaggtggttttcagagaaagtccaattgtgtttnaaaacatctgcaaagtacgtttttcccctcagcatctagaaggcacacatgaagttga

SEQ ID NO: 121 - DNA, 401 bp, Equus caballus
  variation (201)..(201): k represents polymorphism t or g
tctgtggattttcagtctatatcctgcttcctttatggggctctggcctaagggtgtggtcacatggtcactcatggcaccaggccagcttggagggtgtgctctgcccttctctctttccgtgacattggcatctcattgagtcctccgtctcctttctctgctctctttccatttcattccctcctcaaatctttgttkttcccattgaggagagaaagcctcgctttccagtggggcttgagtatttcctactcaggttgatgcgcctcttttgggaatataaattttgctttcctttcagttttctcctctgttttcagttcaagacaatcttgtgtacatggccaaataaaacagcgtgttgtcaaatcccgggacaggatggtgatggttattga

SEQ ID NO: 122 - DNA, 401 bp, Equus caballus
  misc_feature (167)..(167): n is a, c, g, or t
ccacatgctctttcccacgctgtcctgaatttatataccattatgtaattttattgagaaagatataggtatcaataccttgagcagtgaaattagcttcttcactctctcatgcttaagctaacgttacaatgagtcagcatgcccatcatccacagtaactcacngaggacgaaccagtctcgactggcacatgagatkatgaatggactagtgtaggattaaaagtttgcctgatatattaaatgatgaaatcgtgaaaaatgcatacctggtaggaaaaacttaatgggtaggatgcatgatgaactcaatatgagggaagtaggggccactgcagaatatggaactgaggggtgattcttagttatggggaaaacccaaaggtgtgcctttcctca

SEQ ID NO: 123 - DNA, 401 bp, Equus caballus
  variation (201)..(201): r represents polymorphism a or g
accacctttctgagtccatgccactggtggaggccctgccagctgccagaccgccgggcagggtgcagtccaaggggctccagctgtggtctctgccctcagctcagtggagctgaggaaacatgccatacagccagaccttacgtggggagcagccctggccaagaatgagataaacggggggtcggcaaggtcccagcragggcatcttgcacacctggatggccagggaccacaagggagccaagtcattgggctgaagggcaggtcagaaggcagccgagccactcaccagcatcctggaagccacacaggtgctccatncgctggtcaacacagcatggggtgggcacctcctgcagggncatgggggcagacccaggccccagtgacttcaccag

SEQ ID NO: 124 - DNA, 401 bp, Equus caballus
  misc_feature (110)..(110): n is a, c, g, or t
ttccatgcgaccaggctaaggacacgagcaactctcagtagcaaatatgaaaatccaagcaaaagaaagaaagactacattcagctcttgtaaatctcagtctcgctcancgccaggtggacatgacagttcattgncgcgaccgtggatggcgggtgtgtccgggtgcctatgctctataaaatacaggaggcaccacaygtctgagattgtcgaatgtctagtgctaataaacgtcacaccctgcagcttctgaggagaaagaaactgtcccagtccacggctggctctgctaatcaaggccagctgcttgggcctgtgtccagagagtcaggnaagggtggcggggagggaagaggagaaacaggggccccatcaccgccacggggggactcccctgt

SEQ ID NO: 125 - DNA, 401 bp, Equus caballus
  variation (201)..(201): r represents polymorphism a or g
aaatggaataacaaaacaaagaaaacaacaactgtgtttgtcgttgtagctgagcaggcatggccttttcatagcgcatctgaaagtgggagagagtgtaggatttgtcctgagcattgttcccgggatttgccgtcacagaacaattctgatttcagtgggagggtagggccaagcaagactgcttttgctctcagcctrtttaaagaaaatggtgagcttgcctaggaaatacccagtgttctgcaggcccactgtggcttgttgcattaaccacccaaacaaaaaaatgttactgtgcatcctttcttaaagaaatggaacaatgagactgatgttggcttcttgaatggaaatctaggggcataagccacccattctctacaaaacaaaacaacatg

SEQ ID NO: 126 - DNA, 401 bp, Equus caballus
  variation (201)..(201): k represents polymorphism t or g
aatcctccacactaaaatctaagtctaaagactggaagccctgatttttctgccagttagtcactcatttatttattcaataaatatctatcaagtatttactatgtgctaggtcctattttaggtgttaggaaaacaaaaatgattaagacacaaaatttcttgagctcttcccatatgtcagacactctcaaaaggaaktcataacccagtggaggcatagaaggcaaattgcccaaatgactgttgtacagtgtcctataatacaggctggaagagggttaaaggagagatcaatgagatgagagagatgtcgtacaagccctataagacaggagtcaaaaaggacctctagaaagagcagcagcctcatttacaacccactatggtccagggtgcta

SEQ ID NO: 127 - DNA, 401 bp, Equus caballus
  misc_feature (10)..(10): n is a, c, g, or t
tgatggtgcnaagatttcatgattgcttttcatccttgctggaaggatcaatgagactggggatatctctggaatctgattaaaatttctggcaggatcgagttgtgggcacaggagagaaggtcaccatctctttatcaacccacctcagagtcctacctcatgatctaggtcctaaaaggaaaggcagcaccacaggcraaaaatattctaacaatggaaaactgcttccacagtcagctgtaggagtcagcagagccatgctcattttgtgtaatctgaaggtcttagaaagaaagacgagtgacagaaatgtgatcccacacctctactctcatctcctgccaggctttctcccatgacagctccatttctctaggccaagcaattctttccttcta

SEQ ID NO: 128 - DNA, 401 bp, Equus caballus
  variation (201)..(201): y represents polymorphism c or t
taagggcaagaactctctagatttcccaaaatgaggcataagcaggaacccttggtggtaaggaaaccccaaagtttgctttcaacctgagtatgctaaacaaatcctggataatttgaacttttgcaccgggtatcatggcaagaatttaaaacctatgacctgttcatgatgggtaatcaaatgggaaactccctgcaytaaaatagaattgcaaactgtgaatgtgtaataaactttgctgtagggaagggagacagagggaaaattacccaactcattttaggcacttggtagaagttcaaaaacaaacaaacaaatcaaaaaaacaaaagaaaaaagtaaaacctcatctgataattctggaaggaaatatcagactcaaacaggctctggttcca

SEQ ID NO: 129 - DNA, 401 bp, Canis lupus
  variation (201)..(201): w represents polymorphism a or t
gattagacctttaatgttacagcaaatatggtttatgattcttttttaaaatttcaaataaaactttatgttgagagctatgactgcagttctttctcttgtcctcccttacctaatgccccaaattactttggttgtcttctactgaagtttttatttcttaaaaatccgcaacatataggtctaggtgttgtctcagawgccatgtaggatttaaacatcccaacagagtgaaatgctatttcaggaaatacggtgcacgcttgccacctagtggtgagtgtggaaacaagtgaggatttcaaagcaatcccaaagaacgtgaattcnnagaaanactaagttcactagttatnttcaaatatagtaaggtataagtgttatgtgaaaactattatttt

SEQ ID NO: 130 - DNA, 401 bp, Canis lupus
  variation (201)..(201): r represents polymorphism a or g
actcagccccagccctcaatgtgcacgttatctcatgggggaaggaaacacatggacaagtggggggtgtcagaggtactcaatggtggtacatagggacaagattggcatgcagtgagcaggggcaaatccccccaagtgtgggtaggtgaatgtgctggaagcaggttggaggggaggagtcaggaagatatgcaagargaagtatcccaaccagttccagctcccagcttgacccatagaagggggaagacataattaaactgcctgggggcatccaggaaaatactggtgaagactcagcaaggtttctccatccttcagtcagtgcactgaagaagtgcagctgaggaaagagcagtaagttagtggacaatgaccacacacaccaaggtgtgcgg

SEQ ID NO: 131 - DNA, 401 bp, Canis lupus
  variation (201)..(201): r represents polymorphism a or g
ccttccctcagcacagcccctggctcttcacggtcacttggaggctgcctcatggctcccttggagctgtgcttgcctgggcaatgggctagttccttcagggttcagagggctggaatgagaccctacttgctgttggcttagtagactctaccctggagctgacaaggggaggtggctccacgggcagccctgctctcrctgcccgactacctgtggacacgtgtggacaccggcgtgcgagtggccctggggcccctggacctagcattcttcccagcctccacttcagaactgggatctcttaacacctctccccacgtctgcctctggcatctgctcttcgggccctcccccgggggagggggcgggggggagtggggggaatgttgctcttgcta

SEQ ID NO: 132 - DNA, 401 bp, Canis lupus
  variation (201)..(201): r represents polymorphism g or a
tgtgaataatctcttataaaagcagtaaagatcatgccattatacctgttgaatttgctgcagttttagttctattttaaacaaggtgtcatgaaaagcacagacttacctgtacggtagacaaagttgccttcggtttctgatgatgagggagacaccaattcttcctcaaattcattggaactaaaagatcccgaatgrtttctttgccttgtctttcccatcatggcagcatttgtggccatgacatgtctcaaggagtcgttaaggtaaccgaattcaaataaagctgctcttgtattngggggggtgaatacgtagtcctcactggcctgtgactctggcctcactccagcttttatgactgagctttcacttttantcacaggatgatgaactgg

SEQ ID NO: 133 - DNA, 401 bp, Canis lupus
  variation (201)..(201): s represents polymorphism c or g
ggataattgcaagtcataaaagaattaagacattttcttcctgaaaagactaattgaaactctaagaaatgtgagttacatagaacatgctggccaccatttcagccatttttgtctttattgaaagggctgatattttatttccaaggaattgcaagtgtagtttttaaaatacatggttgaaaatatgatagacgttasaatgctgaattagagaatgactgatttgaaaagaggtgccataaagctgttacattaacccttcgttgaacatcatatgtttgatggtcaaagtctccacgaagatagaccgccaatctcataaggcacactagggcgctaggtgaagctcacagatgatctcatgagctggagcctgcaggaggaagcgttggtgggca

SEQ ID NO: 134 - DNA, 401 bp, Canis lupus
  misc_feature (100)..(100): n is a, c, g, or t
cttcctgcctataatttccatagaccaaaagtcttctttccccttaaactagaataatttcttcttttctcaattcagttttcctattagaacagactanaagggaggttttttttaagattctgggctctcaacttttttttttaaggcaacagagacatcttttggccaattantgcaactgcatggtgatagtaatgmaaagttaatacactatgagctgcattggtgagccattttctatgatctgttcagtgattcctcagtccngtgacgtttcaaagctgatacagcattggcccactgaccacaataggaagtttttctgataaagaaaggcaagagtcaggatctggatccactacctgaaatgcagttcgatctgaatggatccttgggtg

SEQ ID NO: 135 - DNA, 401 bp, Canis lupus
  misc_feature (37)..(37): n is a, c, g, or t
cacatcacggacacatttcctatgatctctactcccnctcttttgtcctgtaaagtaggaagcagtaaaaggctataatctgggacacattcttctatggatggatttggggaaaataaaactttttcactttttctcaggtatagtgctattacactatgttataattaaacataaattgcaaatatcacgaacataacygtgataccttattgaattcaggaattagccttctcgtgaatcttcatggtttgtgtaatgaaagctggggcagtagggaacattgttgcttcagtgtggtctccttctggctgtatggctgctgtcccatttcacttcaagcattatttatcaggtagtttcagcctcaagatctttatgagacccttttaaaatatgtt

SEQ ID NO: 136 - DNA, 401 bp, Canis lupus
  misc_feature (116)..(116): n is a, c, g, or t
acatgcacaaaacaggaaaactggttgaaactcactggtggcacctgggcagtcactttatgggcctactgaatgtttccatggaagtagccaagggagacacacactgcagagcntcgtaagttggctcctgaccacagtttggcaaggtggaagccatattatgggacatctaggaggaanccccttgggagcaaggtrgtgagggtctcaaaagacacaaagtgttctagggcttacttatcttttttaatggtttgtgtggatttgagaaaatagtcaaaatgaaggataatagagggatgaaactgtcctacagagcaagagaccccaccagtggaacaaaactacacacaagatattagatattaggtataagatagaacaagagtatacttccc

SEQ ID NO: 137 - DNA, 401 bp, Canis lupus
  variation (201)..(201): r represents polymorphism a or g
ggtaataccaataaaacatctattggtaacctacttcttccctattcaaatgggcgcatgaaccagatgcaacagggagatggaaacaatttgcctgactagttgcttttcaggagagtagggtgaaagttctagttatcctgtggggttctggggctttaactctactgcctgtacatttaatgtgaatgaacctattcrgttgttagaatttaaaatatgtagaagttgtttatagtttgctatatttctttcctaacgttgcgggttttttaagagaatgattagtaggtagaactttaaaacgttcatctggatctgaaccgaatcctattttataaatcccttgctttgctgaaataggtgcaggaaaggtacgctacacttgattttaaattaag

SEQ ID NO: 138 - DNA, 401 bp, Canis lupus
  variation (201)..(201): k represents polymorphism t or g
ttgtgcccacacaggacctccagggctccccttagcctgctctatacatcagtcagatggcctcaggtttttagctatttaaaaagtaatcatctaaaaaataagatttgtacggtgtagttgttaccatcattttggcgacatttataaagctgcatcactggtaatgtagggctttccacctacatttatctttaacckcatgcaaattggaatcaagaacagctgatgcagcctagtaaacccatggtagaagtttccaaaagaaaggaaataagtacacggtcataaatgcactcttatttttatcaataacatttaaacattaaatgctaattatgtaaaaactcccatcaataaaaccccatttataatttgcaaggatcactagaagttggatt

SEQ ID NO: 139 - DNA, 401 bp, Canis lupus
  variation (201)..(201): r represents polymorphism g or a
agaactcctctgttctttcttgtcctcaaatggtggtgactgttttcatcaacctatgcctccccgctacaggcttctcaagtttgcaaatactgctgagtttataaacggttacacacaagctgtacctaccatggtgacaatgagcacgaggagcttcagtaaatactgacaggttttggtgggaggcccacccttctrtcctatttcacactcaaggaacctgccccatcaatcctggggcttcctcccttcctcaggcacctggaccctgcatcttccctctagccaaacgagacattcctgcccaaggacagcgaggcaacttgtttctgcacttgcagcactttgcagatcacaaggccttccgagagtgggaatcagtgaacacccagagatcc

SEQ ID NO: 140 - DNA, 401 bp, Canis lupus
  variation (201)..(201): r represents polymorphism a or g
gatctcaaaacaggtccaccctggctcatgcaatctagccgagttggggtgacaccagctctgatcacttgaggggcgcagccagcagtattgagcctctctatgtactgggcactgtgtgtgcgtttacaccagttctcacaattcagtgacacacatgcaggagggcggaggggaatagtaataaaagaagtttccagraatagtaagaccaacttttaacagcgggtagatagggaataaaaaaacgttttaaaattgtcaaaccatttcctttctatttctcaacgtagggcattgccgggaggggcacggatcnaagaacnaagtccaggcctgcctcgttggtgtcggagnacagccctagaaaacaacgtgactctggggatgtacgtcaggga

SEQ ID NO: 141 - DNA, 401 bp, Canis lupus
  variation (201)..(201): y represents polymorphism c or t
acaccgggctccctgcgtggagcctgcttctccctctgcttgtgtctctgcctttctctctctctgagtctctcatgaatcaataaataaaatactttaaaaagtaaaataaaaaagaaacagtgctcttcctacatagggagactaaatgatagctgctgtttggggtgagtctccagaccagaaccagaccagggttgyccaatctaatatagattgaggagtgggccagatttgcagaggcagaggggaggagggacagacagggactcatgggacctgtgtgaagcctgttacgcacattatgtcattcaagcgtaaacagacctgtgggtaccggtgctagaattacctccatttcacagatgtggaaagtgagactcagaccccaagagctcgtt

SEQ ID NO: 142 - DNA, 401 bp, Canis lupus
  misc_feature (67)..(67): n is a, c, g, or t
tcttcccagtaggccaatgtcagtggcaccatctcaaccataagggaagttaaaggatccctgtccnctgctccatttcactcccaggagagaagaactttgatgaaaatcaagaggagatacagtggtgctcctgtcttagcaggcagagctaagctgtgagaaacctctgctggagatgaccacnctcatctggattgyttatttggccaatcttttatgatctttgctctcaggtagtatctgggctgccttctgctgaggagggcctccttccctgagattccaagctggattgtcagagggaccagtggagagacngagttgaggggaccgcacagactgggtctgtggcctcagcggagactagcttgtttccctagcttagtacgcctgtgcac

SEQ ID NO: 143 - DNA, 401 bp, Canis lupus
  misc_feature (182)..(182): n is a, c, g, or t
actgcactgctttctccttacaaaaatttcacgatttttgtgctctccttgcttacatcttctaagtcttctgggtccagtctttatttcaatcatgggccaaagcagttcatagtgagtgactaggttctatgtctcctacgaaaggctgccactcccagggtgagttaagatgactctgntaaggccggttttgaatgytccgtggtgaactcaagactgctgctccaaagcaagaacttgggctcataaaacaatagtgttattacgatcgacgcaaaaaggtgtcttattagttaagttgctgccctgctgccaagagcagtgatctagcttctaattttcctttttcaagatgatcaggatgagactcatcagatggttttgctcagattaaagga

SEQ ID NO: 144 - DNA, 401 bp, Canis lupus
  variation (201)..(201): r represents polymorphism a or g
gccacctctctgaatcttttctggtgttctcactatttcctttaactttttgtcacattcacacattgcttaaaaagtgtggaaaaggtacctcttttggagaaagagcttaagagtgaacactcagctactctgtctcttgctttactctgggttgggaagcatacctggcccaacttggtgccagtgcccaccaaaacraggacatccctggcccagttcatgtcaggcntcaagagcaggaagacgtgagggaaagaaagggacatggaggttagagctattagagcaaatcaccctgtgcttcctaggggtctgtgctgatccttttccacctcctgagggtcagggtactttcacttgggatgtgctctacgacaggcagcatacaaacacaccca

SEQ ID NO: 145 - DNA, 401 bp, Canis lupus
  misc_feature (62)..(62): n is a, c, g, or t
ctcttggtgtgcatgaggggggaagagatatttgtgtacatgaaggccacacctgggccagnttccatgaactcctgtgctgactcctgagctaagatctcctgcctccctgtacctccctgggcagctctcttgaaagcagcaattccctgatggccccagtttcttggtcgagggcctagtggccgctcttccctgctkggncctgatgggtcagacatcggctcccccttgccagaataatggttcggccgcagaaacccacacaatctccctgcgagacaggtctttgttgttctgatgtccgtggtcccagacattcagcagcttgcacactcaggctcagtgtacacacttanactgtcttgtctggaatcgctaactgagcctgacnccttagg

SEQ ID NO: 146 - DNA, 401 bp, Canis lupus
  misc_feature (137)..(137): n is a, c, g, or t
tagtaagttaatgagcccctgacatatttccaggaaatgttcatcttcaccaaaaatgagccatttctcttcactcaagagttactcttcattctactgttttcatggttttcagatactttatacattacttgagnctcgccacgatcctcagaaatgcacaggaacccatcagttggcgtcctccgcagacgaagccasggttcagaggcgtcaaacagcacgtctggagcctcgtggttaatcagcagtggatttgggttaaacccaggcccttgttctccagacctgttcacgctcaggctgacacgctcaggctccgggaccacggtcactgtcacaatgcttccctcccttgctcatcattttcaactatttaaaaggaaactcaaggggntgga

SEQ ID NO: 147 - DNA, 401 bp, Canis lupus
  misc_feature (174)..(174): n is a, c, g, or t
ggcttcctctgtcctacagctcatcccagagcaatgaatctgcctgaatctgttggtgacaagctaagggcatgggcttctctgggcaatttgtggtaggataagactggtgctggagacaggagaaattgcgggaaccctttctgcctttgagccacttcattcccagttccngaggagaaggctaggggtgggggtagyttagcccagggcctcctcctgggggtggagttgaccttgagaggagcagtggcatctaccatcccttcctcttggggctcttcagtgccaatctgaacagtctggaaatggaatctctgggacccctccccatcactattattataataaactccaggattgtatctgcggagcctcaggcttcaggaatgcagcctgta

SEQ ID NO: 148 - DNA, 401 bp, Canis lupus
  variation (201)..(201): w represents polymorphism t or a
gtgtagaaatggaatggaaaattcagttgaaacacacacacaatgtcaaaattcagttggttttaccataggagattaaattagccaaacaagggttctcagtctctattttaggtcaattttgaggttgattatggtttgtgaatatttagtgtactgtcagtttcataaaaataaaaggtaaaacttttctccctgatwgtctgaagtgatgagaatttatatattagcaggcaaccccaggaagcagtgtctctagtggtatatcaaaggccaacattaaagtattaacttccctaaacttaggtttattaggttttatatctggtatcaacagactcttatgcttctagtcagaagatttttaagaggatagacattctaaacaatgccaggatcaa

SEQ ID NO: 149 - DNA, 401 bp, Canis lupus
gtgtgagcgcggatgcataaggatggcgcagagctctggactcaagcagatgaaacagggtgggagtgaacactggacgctggaaggacaggctcaggcaggagcagtggggaagcacgccctccccggtgctgctatctttccgtgtcaggacacagccgcacagtggcttttgctcatgcacgtgcagctatgtgtgtygacaattccagctagaagggtgcagaaactaagcagaacttgactgagtaggacagcgggcagcaggagggccgcccccgtcaccgggagggtcacacgtgcagctccagccagaggaagctggggcacgtcggtccggacctccgcagtatgtccgcggtttgtccacggcatcaggggacaccgaggcagggtagcca

SEQ ID NO: 150 - DNA, 401 bp, Canis lupus
  misc_feature (81)..(81): n is a, c, g, or t
ctagatcctcttttacaggtgtcatccactctcttcttgatcacatccaatgagattatgatacttactcagctctcgcangccaagagagctgagtcagtatcatttctttgttctattgttttagtngctagaaagttcttaacagtaagggatatctataattttcacacattttccaagttttcacccatcnaaagrggaccaaaggtttgaagaaagtttccaagtgtgtcttaattgttttactccnggtgaaatatccaagatctttcagcnatagataataaagtgtacaattagaanattattatctacttcaatcagggcactttatttctgcaaatgggaacaacattnagcactattaccttctttagcagctctgaccacttgattgt

SEQ ID NO: 151 - DNA, 401 bp, Canis lupus
  variation (201)..(201): y represents polymorphism t or c
aaatcaagttcaaggaaatattatttcctgcacgccaggtagatgtcaggcactctgctggtgctctgtgtgggcgatcccctttcctcctccccatattctgaatgagagggactgtcccatcctttcctaggaggaggagccttaggttctgtggggtgagggaccaagtctactttcacacccatgctccttcctctygttgacaccttcttgaaaatacaacaatccacatttcgagtgctatcgtaccaggtcagacagcccacacacccttaaaaatatttccttctccctccagtcttttcctcagagtaaaagctttcgggaaaggcccagatgtgctataagggaaactcagcaattatgcaagtgagaagcacagcccaatggttagagcg

SEQ ID NO: 152 - DNA, 401 bp, Canis lupus
  misc_feature (82)..(82): n is a, c, g, or t
gccgtgggatcacgtgatgcagttcctgggaagtggttggtgagtcagtttcgtgctccttgcccagcattggtagtttggntgcatgcagactgggagtatccctgaggtgaagggagcttgatggttatatgttcatagtgatgaattgcaatcatgctggagcccgagtctgtttccccaaagtgcctcattccagasgcccgcaggcatctgtctcttggagcaccctaccncgtttgcatagggcagtgcattcaaaganacttgggtggaggacatctggcatcatgtcttccatatgtcaaggccgaatcnagcaggtggatagaaacaggactaaancagcatccntgancatacttcctnagtgaccagcagtcctgctctgatttacccag

SEQ ID NO: 153 - DNA, 401 bp, Canis lupus
  variation (201)..(201): y represents polymorphism t or c
tgggcataactgcccacagtgggcagcacatggccccagtcaagtaaaagactccttaccaccttccctgaatctttctccccagtccctgtatattggagtagaagagacacaggaagaggaggatgtatccccagaatgaaggagcagaccacagcccaactccccactaaggaacctagagccattggaaagagctayactggagggaanggaggagaaaggcagggccaaatttactngaagtttcactgcatcagactgaaccaggctttgtgaacctaagtggacatangggtctgagatttgcccacgatatgatcaaggggtggggaaaggggctttaatggccagtaagtgagggaaagtattatatgcttatatttttcctgctaagtcag

SEQ ID NO: 154 - DNA, 401 bp, Canis lupus
  variation (201)..(201): y represents polymorphism t or c
aagagcagctctgcctagtggtaaggcaagggaggttgacatttggatgggtatctgcctggtccctgctggcagtagcccaaagagcactactgttcatggggatggcttccatgacctgagccaggggttggatggcagccttagctcccagggtctgctggtgacctctgcgtcaagggggtgataactgggtcccayagtgccttgtccacctcttttacacattaggttggccttcttgtagaagcagagtgacttctcgggccatggaatgtggctcttttcccaggaggccagactaggtctgggacaaaagctttgggccaggggtagagctagctttggagtgagcacaaatatgcacgtgtgtgtgcacgatgtgtgtgtgtacctgtgtg

SEQ ID NO: 155 - DNA, 401 bp, Canis lupus
  misc_feature (92)..(92): n is a, c, g, or t
tttaagaggctggtcttctgaggaagaaatcacaaaaattataaatcataagatggcattactgacatgagagtgaatcacactggtacatncacaagtgacgggctggtgcaagagactttaaaataaatgtttgaaataatcaaagacanaaatgaaggactaagtgcatcctgcaagaagagggcagtttgagaatgkctgctttgcaaacaatgacaacaacaaaacagattttagaatctaaataaatagtattttaaaaaatgtacaatagaaaaaaatggatcctgatgataattttgaggtttctttctggcaataactgatataagaagctccagaattctgagtacatagtgtgtgtgtaggtgcacacacatgcacgtgcacatgtctgt

SEQ ID NO: 156 - DNA, 401 bp, Canis lupus
  misc_feature (74)..(74): n is a, c, g, or t
ggactcgatcccaaacttcaggatcatgacccaagccgaaggcagatgcttaactgactgagccacccaggtgnccctaggcatgactgtaagacttttgattatcctcatgctgtagaggtgggtagtatatatatcagtgccatgtgattcatttgagcaacttcctactgagaatttatttacaaggatgtgtatatsctcctggttgagagctctgaaaatgatgtaaggcaacatgaagatcctagagaatattttatttacaccccctagaaacttttaatgtcagcagatgctagctaaggtgtacttagcgtctgcttgtgtacccgatgtgctaggtaactctgtgcctctgtttcccattgacaaggtaggaatagtaagagtgtctacat

SEQ ID NO: 157 - DNA, 401 bp, Canis lupus
  misc_feature (130)..(130): n is a, c, g, or t
gtttgggatctggctcttggatgttaccaagtatctggggacaaaactcagaataactaactttcttgcttctcctcgaaatgggaacatagcttctttggcttattcaaaggcaaaatatgggtcgtgngtaaacagggcctagtggaaaaacagtagagatggagcctttctaaaatggattcccttcagtttctctaygtcatgaaggctcactgccaccgcctctcaagcagagccacagtttcactggagtggccgcctggcgtggccatngggccagacaccaaatgcaagaagcacaaatgcgtgacagaaagagcttcccctttctgctcccttctgtggatcgcagatgctgcatttgagttttgggacagctgttttcccccggggggctc

SEQ ID NO: 158 - DNA, 401 bp, Canis lupus
  misc_feature (21)..(21): n is a, c, g, or t
accggcttgtaactggcagancccantaggcattttctnaaggatactggcactgggacatccaccantctaanagggaaggtattatctagtgctgcttgatnntatttcnggatatgcctgcttccttttctgcttacagagttcctatgtcccttcttcttgttgttgtaccgtgtgctatgactgccatcacttcamtctgctggttgtttggaggtttgttttntctcgacagaactcagagatacagggtaggagatgggatcggtcgtgtgcaccctcagcctgacaggcacatangcacnggcatcgaggaggactatgggttgacttctcatgagagtaacagaatcctgaggagaagagtcatacgatccactctttcatcagattctctt

SEQ ID NO: 159 - DNA, 401 bp, Canis lupus
  misc_feature (76)..(76): n is a, c, g, or t
tttattaaataagatatcccttcaacattggtctgtaatgcttccattagcatgtagttaattcagagacacatcnatgcatattccattacattttaatgcacatcaattattttgtcagtaccacactgtcaattgtcaaagctctaatgaagtgacatatgatgttattccattacagtatctcaagatgtgaactaycagttctgggtcatcttacctttccttgcaaccatcccccncgcccnccccccncacacacacactttatttgtgnaggataattccgaattaaaaatagcaaaactctacactgtcctgcnagatgtacaattaaaangatgaagaaaaccaaagacccagttttgtcttccccagctaccagagtgagcccaaaaata

SEQ ID NO: 160 - DNA, 401 bp, Canis lupus
  variation (201)..(201): k represents polymorphism t or g
aaatggcatttccatgtcacattcctatgtccccaaatcaggatttgaagccggttgttctgacttacggcccaaaactcgtttcaccacaacagacgtacaagaagaaagactaacaaatccattttatgaagtaaaggatctaactacaatttttgttccaaagcttattgacaccaggacccagtgggctgggacackgatatattttatcttcctgtacntcacagctgtccaaatcttgatctcttcaatagtgacccattacacagtctcatccagtcttctggatttaaataccatgtatattactaataattatacattttatctctaacgtgacnctttncattagataactaacatttcaatattattcatccaaaatcgaggtactgata

SEQ ID NO: 161 - DNA, 401 bp, Canis lupus
  misc_feature (126)..(126): n is a, c, g, or t
tggtcacaattgttgacacttccagccccagttcctcctaaaagggataaaagaaaggaaaggatatctagtggcaggaaagtatgaaaagggcaaatcctctgacttagcagagggactgacacngagaaacttaggtcagggtagaggtagaaaaggagcacagaggaagtagcagtgtctctgaaggaagcattgccrtcaccaagccaaatttatccaaggactcacacatttctgtgacaggatccctcaaataagaaggcaagtttcctgtagagagacacaaatgagaaaggcagggaccttttttagcagagctgagattttctggaaaacctggggaagcacacacttctcaacaattcagttaaattctttacactatctttagttcagaa

SEQ ID NO: 162 - DNA, 401 bp, Canis lupus
  misc_feature (159)..(159): n is a, c, g, or t
ctcccacgccgcctctctttctatcccaacacctcccatactaccttgagaagcaggttcctgctctggggattgtcctgggagcacagtttttcaaatgcttgaatcttcttcctgaggagagagaaagaaggcaggacggatgttggaacactaggntcgggttgagggcagaccctgtgatctgagacctcgaaggakttcaactgctggatcagggtttccttgggttcctctgagcactcaggtcgttgaaggacacgggagggagctctttggagggtcatccggtgcatccgntgccctattaattgaacngctctgctttggtcagttttggtttcaattgcgaagagactctagttgctgtcatgttcgcatccaaaaccttgtacctcagc

SEQ ID NO: 163 - DNA, 401 bp, Canis lupus
  variation (201)..(201): r represents polymorphism a or g
caacacaaattatttataccatagtgagttgaatataagaatatggagagaaaaacaactcaaaatgtatgtttgacaatagtgtattttaatgaatttagtgatatacaaagtaagataattgcttgttactacaaagagttattttgatataaagaaccaactacacaaaattatagttttatgtgtattttagtcaaraaatcatcttgacaagtttaatcttatcaagggttaaaatagatatattacaaattgatataaagacctatatttcatatagangtaatatacagggatgcctgggtagctcagcgattgagcatctgactttggctcagagcatgatcccaggtctggggattgagtcccacatcagggttcctgcaagaagtctgctt

SEQ ID NO: 164 - DNA, 401 bp, Canis lupus
  variation (201)..(201): m represents polymorphism c or a
attgtatttgcatagcccctctgacgacttgcattagctcgattccatacaaacccgtgtgccccagtttcataaagccttctctccttggccaaatgaaatcagcctctccaagtgaccctcaactttaacacttcaaagtaaagcacagagttactttgattattaccacagtacttgaccacaatccagagaaagtcmatgaaaaccaggaccagatagagttaatgctttcatagaaacaaaatgccgcctgtggatgctgagtgccagcacatcattaagggaaggataggaataaggccttcttaagagctgacattaaaaattgaaatccattctgtaaaagacaggccttgtgtattttttaaagcccagagctatagcagctgaagggtaat

SEQ ID NO: 165 - DNA, 401 bp, Canis lupus
  misc_feature (95)..(95): n is a, c, g, or t
attagcaagaaaattgcacggggaggtggcatgggaaggaaagaccagagccacagcctccctgcaagggctcttgcctccgctctcatctcccnggggcagagatctcggtccccttcccgggcagantctccctggggcaaccttgccagagagaaaatgcttcctccgggtggtgctcagacatgccntctagaacgkcccaccggtgtgtctcagcaggtacgcggatgggatggcagtgatggggagcctnggagattccctgcagacnatccagacaggagctgaggctgtgcaagggacactgagcctgccctctgaccgccactgactcctggttcccattgcatcctctgggtgctgcctcctgtgcaccctgtcctatttccctgaagtaa

SEQ ID NO: 166 - DNA, 401 bp, Canis lupus
  variation (201)..(201): k represents polymorphism t or g
gaggccttggagatactcgtctcagaggtatttggagaataaccttgcaagaactcactggctggtgacagtaattcagttagttctacatctttctagtggaatctagagtaaaatatacagagccaggtgataaattttgaaaaaggccctgatatggcagtgccatgcattttaagtgtacatcaagctttctggagkcagcttgagatggatgttcagtgtattggtgtcacatttttagacatgttcgcaaggtctcattttttttctgtctcttcattcttgcatttcagcagctagaaatggctttgctgcttcaaaactctgtgatatctctttatgataaatttagattttaaatgccattgtcacttgctgaatgcatttgtaagaaatgt

SEQ ID NO: 167 - DNA, 401 bp, Canis lupus
  misc_feature (30)..(30): n is a, c, g, or t
tggctaccttggcggaaagctctggtcctngcacaggtggcttcacccaggccctgccactgcctgcggcnccctagtgaggcagggtcccatccctgcaggtgcggcccncgatgccagtggatgctccttctagaacagctcacccaacacgtgcctttgccttttccatgttttttcaatgtatctctgcctcttccstgcaagatttctcttgttctagagatgngattgtacaccgagcggaaaggggtggatctggcgggaccngagggcaccccacccccgtgtgcctcaccctgccttctgnacacccctctgtgagcaggaccagagctgcgggcgctggggttcccaagtctgtgcgtccatcccgagacctattctgcaaaagggggatt

SEQ ID NO: 168 - DNA, 401 bp, Canis lupus
  variation (201)..(201): k represents polymorphism t or g
ccacgacccgagccacagcccagagtcagaggctaagtgactgagccacaggcatcaaggcccgggttcctgccgtgggcactcctgccccccacggcccccgagagctcgggctgcgggctgcgctcccaccccaggcctcccggcggctccatgcacgtcccctctgtcccaactcagggtgcaagggcctcggccggkaaggcgcctcccatctgcccgacgaagcccagccggacggagctgctggagcaggaattccaacaactgctgcccgccttgctgcgcagggatgtcatctccgttttcatctttttagacaactgtcatggatttgccaccaccgacgaggtgctggatctgctgtttacgagagtgagcacctgngcctccgcagccca

SEQ ID NO: 169 - DNA, 401 bp, Canis lupus
  variation (201)..(201): r represents polymorphism g or a
atgtaagaataatcagaaggaagtgaaatatataaaaaacaacttactggtgaaaaatctccagtacaggtttttttattcattaaatcctctcttggtaagggtaaacttacctgtggtaaggcaaggagataagcaagagccaatgtcatgtcatttggtaaagcatcacttgccagctgcaagagaactgaaggcagrgggaaaaaaatgcattattattcttgaggatctatatgacatttgtagttactgagtgccatgtttttaatcttgcatgattaccacagataattagcttcagaggactctttaagaccttttacaaatgcctcttagtaccatccaaatacacatcataggaaaaattgttattaaatagtaaccctgtcttaattcag

SEQ ID NO: 170 - DNA, 401 bp, Canis lupus
  misc_feature (79)..(79): n is a, c, g, or t
gagagaagtcttctaagcgcatatctggcctttcatagggatgtataaaagggaaaaaaacattgatagcatacaaaangacatttaatgatttgcttctctgagatcttaactgtatcnggcttcttttttacattctatatccctgacctcttcacccaaatgcctagagttcttccagcttctaaataaacagtatgyacttgttgccatattaaaccttgattcagatcctgcttaatttccataatctttttagacccctacatctcacttagataatgaccctttagcttgaattaaactatgtaccattgatcatttacttgttttctcttggagtttaatttaatcattctagtttagttcaaaaggacaaatagctttccacacaatgtttg

SEQ ID NO: 171 - DNA, 401 bp, Canis lupus
  misc_feature (135)..(135): n is a, c, g, or t
cataactccaccccaatcctcacacaaatgattcctcctaaagtcaccaatgacacccttgccacagaatcagaggatccttttcagccttcctctgtcataaagtctcaagagcaaatcataccttcactgttntccttacataaaagattatcttaccccagaacctcctgattttctccttcttccatcctgatctcyacttctcaatttccttatgggctcactctcctcaccacagcttttaattcctgggagcttcagtcctttatgtcctccctttgcacactcccgataagatatttaatatactacagcacaatcctaagggtctaagaataatttttaacacattcctttccctcatatcactctgtatccacttccattacgtagctcta

SEQ ID NO: 172 - DNA, 401 bp, Canis lupus
  misc_feature (58)..(58): n is a, c, g, or t
attgtaagcttgctaaccgaagtgagccatccttctcaggagagggaagacagcaagnaggccttgcctgagggaaataaacttagtccagtaggtacgttctgtgagaatttggcaaccctctgatgtggacacacagtgcctatggacgaagtacttagtgacagttgcataagtgattggaaaaggcactccacaccwgtccacccacagaggccccttgggcttccagtttctactctgctttggagaagagattccactgtggtgagacatgtccattcaggggacctattntcatgcatctctccacttgtggggagttgaaatggccatggtctttagacctggagatcatccagggacaatttctcatcatcaactggactccttctaccatt

SEQ ID NO: 173 - DNA, 401 bp, Canis lupus
  variation (201)..(201): y represents polymorphism c or t
cttactttcaattatgaagaaaacacattcccttttcggtttaatccagtttcacccaatcacttttaatccaaagaaatttggctaatacatatcattgccaccaccacacattataaatgtgtaagtatttacagccacttttcaggaagagatttgttttcaaggaaatttctcttgctagctttccaaatgtttaayacctactttgaaaagtaagagacagaaagtgcaacctgctcttgcaaatgtgccctctgacatgctaagcctagtcagtcccagtagaatcagctaatcagaactgtgcaagacctggtcctcagttgccaggagggaaggggtagtttgccttcacaactcacggaaatagggcagtagaaattgacgaggccttaggt

SEQ ID NO: 174 - DNA, 401 bp, Canis lupus
  misc_feature (103)..(103): n is a, c, g, or t
tcaggccacccctgcaagcacctgtctccccctctgatttaagcgacacgctttcaatcccaccccacggagggcctcccgtactttcaagcgagagctggcnggatgcctctcttctttccttggatttcccaccttcctcgcctccgcagatccccgagcatctcaggctgctggccaccactgagtcaccaggagtayagcactgcagtacctagctctacccgccatcaactaccgccagagtcagagcactgcaccccgcacttcccaccccacaggaaagctcggctgtcctacggggctggaccaaaagggggnaaaaaatgtttttgtacttctaatggctcccctctgaaccagtgcagctgaaatccccacagttctagagaacgaggtnc

SEQ ID NO: 175 - DNA, 401 bp, Canis lupus
  misc_feature (102)..(102): n is a, c, g, or t
tgctggagcgtcaggcaggagaggcccagtggttagagacacaaagcaggctgtgccccaagcctcctgctcccctgtctgcctcctgagccaggcttttcntgccccggcccccaggccttgtcgtgactccccgtgtgcctcccngcgtcaccaccaggcaggagggagcagatactgtgtgggnggccctgccgagcrgctccctgagctgtggccatgaagcacaatgtgcttgctgccctcaggaggcttctggcctctaagagagcagccagtgggactgcaggagcaaggaggagctttgaactgatcaggggtaggggttatgatggggcaagtggggtggggagcagtcttcagccaggtgtgcagggagggcctctctctggggaggagac

SEQ ID NO: 176 - DNA, 401 bp, Canis lupus
  misc_feature (187)..(187): n is a, c, g, or t
tcctcccaacagttgtccagcagcccctcggagccccgtggctgcctgtggagcttccctggccctttcctctacgtgcctcctcgtccccgattacttggagtttggtgggagagacaactctacccagggcttctgcggctgctcaacaggcactggagggacagctggggactcggagggaccnagagtcacgtgcasggtggcnagtgagatggaatctaagagctcttcgggctcacagcctttccgctctgcaggagggaaggcctgtctctcggggcagcaaccagccatcccgcagtcccnggctctccctcccttacaacgcccaggaggctccagtgggtgcttgggctctgaaacgctgttcttcccccctctttacaccccccctcccc

SEQ ID NO: 177 - DNA, 401 bp, Canis lupus
  variation (201)..(201): r represents polymorphism a or g
tgttccattacctttctttttgatccctgtggctcagccaagttaaaagagtgagaaacatggaccttttttgtctctgttccggctgtccaaggacaaggcctgttcctgggatgcgaaagctctgaggcagtcaaggctgacttctccttcctcctttatcttgccagaggctggcccaactgtgtctctcccaccttrtgagctcatgtaccccttgggctctcgctactcccaacccagtattcaaatttgtttttttggggtttgcttattttgctttgttttaatatggaaggnggtgctctccctactatgccagagttgtccttgnngatgggggcgagactacacttgacctcttgacctactgtgancactttgcagagtctctggtcctt

SEQ ID NO: 178 - DNA, 401 bp, Canis lupus
  variation (201)..(201): k represents polymorphism t or g
cagactaagtagatacataaagaaatagagttctattcttttacttactatgacacatggcctgctagggaaatacgaatttaacttaaacccgagtgacaagaggaatatagcaaaatatgggcagttgaataatgatatactttaggaaatctgtaaaacattaagaactgtcttatttatggaaatcctatacagttktccagagtcttgaatgtagatctgttttaaaaggaactcgtggaccatttcaacgtcgtttctagtggcctgacttgtcacaagtcatttcattcctggggaacccctggtggcaattagaagtgagggaattggataaggaaaaattaagcctaattaaaatccttgtacaagaagtcaggaaagaataaacaaacttg

SEQ ID NO: 179 - DNA, 401 bp, Canis lupus
  misc_feature (82)..(82): n is a, c, g, or t
tgttgcttgaagatatctggaagtgtaacaagtttaaataaaatgagttctgttggaattaggaaaaagaataaaatgtctntgttgagtaaaacatttttaagaggggctactttcctctttctgtgtgcattgttattaattgctcaggaaaatggttccatgtaaaaatctcatgtgtaactattncttatctatatytcacaatctaatacacttttctagaaagtcttaagttaattttttgtttcgatgcctaaattaaaataaaagattaaaaccttggatgcaattagcaaacatttttgtagttgtgcagagtaaattaaaggacatttgtgtgcttattttcaantctaatggagagcaaattacattnttttttgtttttttnaaattta

SEQ ID NO: 180 - DNA, 401 bp, Canis lupus
  variation (201)..(201): y represents polymorphism t or c
agtgccgggaaggaaatcaaaaaacaaatgtggctggcttgggacacttagatttctgaaagctataaatgtgaagtctcatgttttcagccttaatagccagaggactttgccttgtgttcaaacaatgttctagctaatagaatcacagtcgtggaaaaagacttcaagaagttctgatagttctttttctgcctcgtycaagaacagatcgcaattcagtctaatgggaaggtcatgctcatttagacagactaatttttaaaggccactatgaaattattttctttatgatcattaaacaaaaatattcgaaaatcaaagaaaaagctgaatgattctggtctcctgcaggactgtgggctgtggaactgtggtcaaagatcactacagtgaccttt

SEQ ID NO: 181 - DNA, 401 bp, Canis lupus
  misc_feature (142)..(142): n is a, c, g, or t
ccatagggagcctgcttctccctctgcctgtgtctctgcccctctgtgtgtgtctctcatgaataaataaaaagtaaatcttaaaaaaaaaaaaaaaaaaaagcccacagtggtaaatacatacacaagaatgatctggccnctcaggccaccaggatagaaggtgccctgtgctctggacagtttcacgaagccattgtktagactgctctatagaacacatagacctgtgcagcccccttctctcctcacaaaaagccaaaacaaaacaaaccctctcaaataaggtcaggaaacttttgcctaagcaaaatttaaaaagattatttcaaagcacaaaactcaagaggctttaatatgccaatctgcactgtgaatttctaagaagcagtagactgttg

SEQ ID NO: 182 - DNA, 401 bp, Canis lupus
  variation (201)..(201): r represents polymorphism a or g
aatcccccagagcagcagttccaatcgatgaggagctggcaacatccgggctgggcatggaaaggtgaacaaacattgtccattaccctgccaatcgccagtcccctaattctgttattttttttccttgggtaaatttggtttcctgaaattaattattcaacaggaggtgacagccggtgtgtagcagcactgttggarcagagaataaagaggcacattggacacagcagctgcacctcccagaccctgaaatttaagatctttataaatgatctgttaaaactatagtgacgataagcttatgaatcatgatctatattaatcagggctgctgatatggaaagattaattgaaacgtgcagttctacacaaatgataaagtggtaacaatttaatat

SEQ ID NO: 183 - DNA, 401 bp, Canis lupus
  variation (201)..(201): m represents polymorphism a or c
aggaagccgtgagattagaacataagcttctgcatccaggggaaatttccacagagggaaattgtggccctggtgctcacttatacctgattcttgcctctctttcacacgggaatcatgggttgggtttgaaaaaactgcagaactgtataaaacctctccttcctttctttttcaagctaggagaacacagtgttcacmatatagcctcccactcacttcacagaattgacaagggaaaacagtgtgcttgtgggcctgagcacgacttaagcagggtgaagtctgggacaagactgcaccaggatccttcctcccctccctttaggttctttgcctataggattctaaaggctcaaggccatgggggcagtgacacttgcttagggagacccagccac

SEQ ID NO: 184 - DNA, 401 bp, Canis lupus
  variation (201)..(201): y represents polymorphism t or c
ggttgataaaatcagggcttcattgtcttttgccagcctcagtttggccacttgagaaatgacaacattggaccaaataatgaatttctgatgcttctagagtctgtgatttccacatgctgtgactgtaagagcagagtcatcaaggcttggttttctgacaaacaattccagggaaatagagctggtgggggaggggaycccagcgctcaccccaccgcagcccccacagagggcttcccgagctgccacccagctggttgacccccaaaggagcaatttgcactttctgctttcctggcctaagataaaaatacccctgtcacattggattagcatctcccctttcnctgagaatcttctcacggatgcagcccccttgctttgtcaatattttcaga

SEQ ID NO: 185 - DNA, 401 bp, Canis lupus
  misc_feature (18)..(18): n is a, c, g, or t
aaactaaatgctcacaanggcaaaaagcaatgtgagnngaccttgcaggggcagggcgagtcatggagcgattgaaaaagaaagaaaaaaaagcaacaatttttaatagaattcngaaagtctgctgcctcgtctggtttacaaataggcattgttngaggagacagaataaataagagctaactacagcatgnattaccmaacactaancccatcaacgagtcccggtggcagcacagatcactcaggcacgccttggtcactctccncatatttatttattaagaagacagtggagtctggctaatgcgatacaaaattaatatcanctgtaaagaaacataacccatacattcaaagcgataactctaccgacaccctcccccccaactcaatcaagt

SEQ ID NO: 186 - DNA, 401 bp, Canis lupus
  variation (201)..(201): r represents polymorphism a or g
ctagagccaggaatgttccgatgtcaccggcaactcacggtaccaccacgtccaaggctgcttcctattccacgtgcggcagaggctgcccgcctgcctcccccctcccaggggctgcctccccacctcaggtggcaccgtcctcagaactggggcacagaggatgcaagccaagctcatcaaatcctctccccgagaccrccgtctgctgtagaacacngccgcccagagacatgtacnagaacccctaaccggctcgtgtcggcccgtgtgtctatggaggcgtcatggatgagctcttacacactcgcccgtgactccaccatcacaaagtagaaacaaaccaagaacgctgtaacgatggaaaatctactgaccctgaccccctacccctccccgct

SEQ ID NO: 187 - DNA, 401 bp, Canis lupus
  misc_feature (3)..(3): n is a, c, g, or t
gcncagggactgccaccgaagagccctgagcatcttctgaccagtccaggctgatgtggcttcactcctgttgctgcacatctcaccattctctcttgctgttggccagtcttctgccctcactttgtgctttcccatgtcaacatggaattggagctgctcagacttgagctcagggtatcgatggtcctagaaatggartgtcaggaacangtgagtgggtttatgtgttttccccaggaaatcaagaactgatggtaaacagaagcangaaacaccatttgagttactggcgtgttagtggaaaaccagtacatcacccctgccagtgcaggtaggtagctgacccacatagttccgtatcaccttcttgttgaaattagtatcttgcatctattctt

SEQ ID NO: 188 - DNA, 401 bp, Canis lupus
  misc_feature (26)..(26): n is a, c, g, or t
acagaacatggctcctcacatgggancagcctcactnacccacaacatttcagataaggaggaagtgagacaaaaacccctgggtccttttatctggtcctcttcattgagaagcttctgtggagcttacagtcaggatcagatctggtgttgaccagcaggctcttaccatggagaaggtccacagggagttaccagtgrtggcagggtcacttgttgcctatcctgggacctgcagctgtcaagttccggaatnattctttttcnttcctggttcctgccctgagaccctcatgagaggctctgagttgtgtgtttcacacagaataagaggtggctttgacgtccagtcccctagatctgtcagccatgaggtttgcacatgcacatttacacgtgtc

SEQ ID NO: 189 - DNA, 401 bp, Canis lupus
  misc_feature (57)..(57): n is a, c, g, or t
gtacgcacacacgtatatacgtacacacgcatacgtgtatataacacgcaacagacntatgaagacccgcacagagattagaacgcgattagagatcagagagcgaacctcaaggggcctggcccggaaactaatanatgtggaaagtcactgaggggctgacggaggctggacnttcagacgaaagccagactaaagggrcgagaactggtctgtaagcagcctctatgtaacggtgcccggaccagccctgccgatgactggacaccccaacttccgccgaaggccagacagtcacgccgacaggagtggagagggttttaaatcccccagagaaaaaagagctgaaagccccaggtagggcaagngggagagaacgaggccacgggggcagccacaca

SEQ ID NO: 190 - DNA, 401 bp, Canis lupus
  misc_feature (23)..(23): n is a, c, g, or t
tccagagggcagcggcaacacanggtgagtctccaggggtgtggcacgggagaccccacagaccaagaccacggcgggctccgccttanngcatcaatccncngagagcggtcccgngccacgttgcctctctggagcatgatgcaaaggcanacgnctgctgccngagactctcagggctgacaagtctccagccaaggsctcacatgtccttggcntgtcaaccgtgatcgcgagcagcagcntggcccgatgcccgtcncttcnttgtggnagccagactggcttcgcaactcnactcacctgctacgcgcccaggactgntgaagccggggccccctgcggtcctgcccacnctgcnagcttccagggtcgtggcaancggactcccgccaacacct

SEQ ID NO: 191 - DNA, 401 bp, Canis lupus
  variation (201)..(201): y represents polymorphism t or c
agccttcagagggggctccggccatcaggtgggccgcaaatctccgcacaatgggatggtaatgtctctgaaggacaaacaggacaaccacattttgcacgtcagtaatttataaaagaccaaccacagcaaagatattctttaataaacactatttcttaaaatcacagatacgtatgtaattcacagtaatcacatacygaaagaaaaaatactctttgctcacatgattacattaatctgatgaccaagttaccagtgacatagcttagtctagcttttacagtatgaaaagagtaatctaaaagcaatttctcttttagagtggaaaaagttagcttacaacaggtttcctgagacatatctgctataagtctccctctatgtccacactcaaggat

SEQ ID NO: 192 - DNA, 401 bp, Canis lupus
  misc_feature (15)..(15): n is a, c, g, or t
tttggtaaatctcanattttgacattttatantgcctaaattccaagctgtctgtgtttttnttttttntttttttttctggtcctaaaatacgtaattctccaactcagtttttcaacctccagaaatattttagctctcctcctcctcttcatccttctcttccacatctctaatcctcatgtctgtgttgctcttgaycttgactttggagccaaccaattcactctttagccaacactgggctggnccagagaagggagcgtggataaaaaactaatgaatattgtcagttatccacattctgaaattaatggaattattgccaacaacttggaagacctgccagagagggaaagtgacagactcctggcaaagacaatgataggatgagcagtctt

SEQ ID NO: 193 - DNA, 401 bp, Canis lupus
  variation (201)..(201): s represents polymorphism g or c
gtgaccaagcactgtggtgtgcccctcactcctttatcgtccgttgactctaagcatcaggacatcatggaaaagacgtgtgagattcctctgccctttcccctctgctattctctgataatttttcctcctcccaaactgcctgggagccctgcttcgctctgctacacatcctggccacaaaggaaaagcaagacttgsagagtgtggttatctggggtcctttccccctggctttttctccctggcctgtcaacgatgcaggcctggggattttcagcctgggctatgccatggactcngagctaaaatgctttccatggctgaggctcagaaagcaggtaagaagtcctggtttagaggcaaaatcttcttttctcatccacagaaagcccccttgt

SEQ ID NO: 194 - DNA, 401 bp, Canis lupus
  misc_feature (30)..(30): n is a, c, g, or t
tttaactaaagctccagtcccccatccccntcccccatccctgccccggagcctccagcatctaaccggcatttacgaagaagaggtggacggtcgctcctcccctcggatagtgtgggtttagggcttcggggtccagtacancacggcctgcacgcagtctggctctctcagggccttgcgacctgatctgggcctgtygtcatcactgcacatcccccgggcgcccacctggcaggtggcaggctcccccngcaggtggcaggctcacctggcaggtgacaggtgacgtgctgcccacctgggcgcggcaggtggaggagcacccagcaccatccccgtaagtgggcgcagtcggccctggggtttcgcggggccagtgactcagccagtggccactc

SEQ ID NO: 195 - DNA, 401 bp, Canis lupus
  variation (201)..(201): r represents polymorphism g or a
gaaatggtagtaaaagggtgcacgccttataatttaggccaggcctgcatgacctcaaagcacccaagcaactcattgaacagaagaatcaatcaagtttgataccggttgacaaacgaataataggacaagatcatttgcttgctttgccagtgccccccaggtgtgctcatggggcatgagctttagtgcaagtgccarcgaaagaacctgtatttcctgttagcctggtggtctcttcagagggcaaacttcaataatactgatggtgtggattactcgaatttgccatttgctacttgacacagtacccttaaaatagcccgtcagccaagcagccgtgattgtgtttctcatgccaccgtcctttgcaaagtaggtttgtggatggtatttcgggg

SEQ ID NO: 196 - DNA, 401 bp, Canis lupus
  variation (201)..(201): y represents polymorphism c or t
ttttctttcctcaaattcctacaaaggccagacttattttaccaggatgccttccaattgaactggctataggcccagcctttcaagaaaccaacagcaaatgctggcctctgaagaggtcagtattgaaaggtaaatcttatattcacctaggacttctagggtgttgtcctctcagaggccagaattacctctcagggycatgagaatggccttttggggaccaggattctgatggcaagagcctgggctccaatgagcttcaaactgatcttcttttctatccattaaccactggtatttctgaaagtcagccctgaatttctaatctctacttgggagttaattaccacctataaagacagtggctgcaaaaaaaaaaaaatctctattttccaccc

SEQ ID NO: 197 - DNA, 401 bp, Canis lupus
  variation (201)..(201): r represents polymorphism a or g
ggaggggataaggcaacccccttgggccctactttggagaagactttgtagagatgaacaactgtccgcaggcctaggatgcaagtgtcaaggccggaccttccctgggttcctcagccaggtccactgtggagtctcccacgcatggccttaaatggccacgcccgggcctgacagggcagtcacgatcccggaactacrgtcactcggctttaagaagcccattgtaagcctgaccaccagggaagagttggccaaactccatcccaagactggacggtagcccgggagattaaatcctaaataaatactccaactaaatgccttgactaagaagccatgctggtctctagttggaaataaggcaagaaagagcngtgataacatcaaccacacaaggg

SEQ ID NO: 198 - DNA, 401 bp, Canis lupus
  misc_feature (10)..(10): n is a, c, g, or t
tggtgacccngtggnggggaggcgggcggggaggcaggtgggggtccatgtgggcacttcccggcttgggcctgtccttcagcgggagcagagaccagagccgngccgggggccacgcggagcctcaccggggtgggcgtccgggatcccgcctaggagggggttggcgggcagggcccatcccngccctgccccgtgccrctcgcccgggttctgcagggcctggcgctcatttctgcgcctctgcggcagcggagctccccgagccccaaccgtggtgtctccggagcccccncgggagacacgaccacgttctcccgggagcacctacagnggcccccnnaggagcagcccttccagcttggtgtctgggctccgtgcccttgcaccagaagtttcca

SEQ ID NO: 199 - DNA, 401 bp, Canis lupus
  variation (201)..(201): r represents polymorphism g or a
tcttgaaacatggacaaggcaaaatcaagaacaaaatcatccttttgacagacaacacagtaattgagaagcacctagagaagtatggtgttatgtgctggaagacctcattcatgaaattgcctttctggggaagaatttccaggcaatttctgagttcttaatgcctttccatctctcatgctaccaagaagagagtgrgccttctcaaagaggtgggcttacttggctatggagatgaatgcaccaatcaactcatttggctgctgaactaaactgagaactctgaaagcacagtgcagtggaggcatgtgttttgttttttggaattgttatccagtatcttcagaaaagattattttctgctatatcttcaacaactagatagaagggtcaggaaa

SEQ ID NO: 200 - DNA, 401 bp, Canis lupus
  variation (201)..(201): r represents polymorphism g or a
aggaaactgacaccactgataccccctggggttggcatgcacctcaaaacttatagataaatacataaataagtaaataaataaataacaattaaaataataaaataaaaaagaaatttctagcaaagatagtttagaagtaatatgtgtctttcatattactaattctccccacaaaggagagctatttcttttaactgrtcaatacaacctcagttatttcacatcttnacctcacataaattttttttaatgtcatattgtattattttgaatcctgtttncatgtaagttttgtgtgtnttcccaagtaaactgtaaattttcttctctatattgtgggtgccaaccctgaaacctgacnggagtggtccttctttgtgngcctaggccttctagct

SEQ ID NO: 201 - DNA, 401 bp, Canis lupus
  variation (201)..(201): r represents polymorphism a or g
gatcgtaggatagttttatttttagttttttgaggaacctccctgctcttctccagagtggctgctctagtttgcattcccatcaacggttcaagagggtatgcctttatccgaggatgttaatttcttgctctcaattttgttatcatttggaaaccatttctttgagtaacttaatggttaggggtgcagagggaagarcaattactttgctgactggatgaagtgttttgaggcagcgtgtacatacctatgatgggcagcgtgacaacggacacctcagggatgctgatttttccagttggtgagtgagtntgtcttcatgtttgaatgaactgggctggcatgcagagtatatagcantatgactagtctcagtttattggtaatgtgaatgtaag

SEQ ID NO: 202 - DNA, 401 bp, Canis lupus
  variation (201)..(201): y represents polymorphism c or t
tgctggcctcccttgctggtgtcccttgtctcctagctcaccctcacctcctatgtggtttgtcatactggccccctctccagcccccgccctgcccttgtggcctaattccttggcagagctcctggctgaccagacacttgcatggctgttgattcctttggctggattcccttaatccctgcaagctctcttcctcgyttcagaccctcagcagaacccggactcaacttctggaaacaaccaaatggtagctctcagctccatggcggtacattcttgacaaaggccacatttttctatgattgggagctccagccttagggctgtgcagtcccctaaagtcatgatttctgggccctgccagtcagccatctgcagggaggtaatttatcagttac

SEQ ID NO: 203 - DNA, 401 bp, Canis lupus
  misc_feature (120)..(120): n is a, c, g, or t
gtgtctgcgtgtgtgtccgtgtctctgtttctcggtcagtgtgcgtggacacaccacatgatttcagttcagtcggggtcaggctggagagtcagggcctcctgtctacgttcagttctncgactganggcaggtttcctagggcccaggtcagggcgacccactgggggcaccgccgtncacagctcacccaggggaaasgagggaagggcccggaacgcacgacaggcaggggttcnggatgaggacggcctcaccccttctaggccaagaccccgacaccttctgtagggtttgccttggattcaggagcctaggatcgtggggagctattgcccatccctgcccnggggggactgtcatcttctgggcctccagaatgagggtaccggtgcccntgt

SEQ ID NO: 204 - DNA, 401 bp, Canis lupus
  misc_feature (92)..(92): n is a, c, g, or t
atttaaataagttgctcccaatattataatagacacaaagtgaagcttattggtaattactcattatgagacaaaatgattaagaaccccanggaaaagaagagaacagatttgaatcattttttatgtataggtaggacatactcccaggttttatgagcaatgtggcttgaggttactaataccttaagaaaattaagygctaagtgtgtcttaactgcatcaataatttaaagtgtcctaaacactatggtaaaaatgctcccaagattcctactcaaacattttgagaccttaagctaataggtattactgtcctcccccactccctgcatagaacacgggctaccaggcagatgtcgaatctgaagaaaagaggatattagggcccaataatcaga

SEQ ID NO: 205 - DNA, 401 bp, Canis lupus
  misc_feature (156)..(156): n is a, c, g, or t
gctaattaatccatcccactgggaggcacacttatcattaaaaggaggcagctgatttcaaagcttcataccctcccttcccagctcagctttctatttggtttccagagtaatacgggttgcctgagagctgggaaaaaagaaacctcatgttcnttttctgtagtgggttttgatcttgtagctgcttatttcttaagmattaggaaaagggtactttacagctggaatgggagattgtagactggaatgtgtaaaaaggtgatatgaatcttcaggctgcattagctctaggaagacctctcagtttaaagaatgatgttcatcttcaagagagagattagaaagccngtagctgtatttgcttgaggatgcaagtgagattcagtgatctggaaatg

SEQ ID NO: 206 - DNA, 401 bp, Canis lupus
  variation (201)..(201): w represents polymorphism a or t
gcagctgtctcaagcacagcatgtgcatccttgggctgcaggtgattgtcactgaaagaaaaatctttagcttccctaagttaataaacttttaactaggttaaaaggtaaacgaagtgtgatgagcacagccaggagtctgatcccgtcagagacttgtcagggcctcctgcctggacaagtgacaagtctgtgattggwtgggacagtctgcctgcccagaaacctagctgtaggaagttagtgcaaaatggaaaccagtanaggtattgtataatgactccattgtgatttagaaattccngatcatttcatatgacatttctttttaatcacttaatgtgaatatataaagagtttgacatttgtttagatatttttctccttttgatgctatctct

SEQ ID NO: 207 - DNA, 401 bp, Canis lupus
  variation (201)..(201): m represents polymorphism a or c
atttactcgagctttagttcatttcttttgtgtgtcatacaattattctagtttgtcagttggtaattgacattgaacaagctagagagattttggttttggggagaagtaagggaaactgagtatttgtaaagaaatgcttcagtagctcggggctccccagcctgtcctgtcaagaactctgttatctttgcatcatcmtatcagataatacagtcatcattttaatgccaaatgtcacttttgtctctttaaagaaactaacatgttgttatcactactgatgtcagatcagctggtatttatcttcaacttgataaaaatgtgcagtggttccctgttctcacattaagacccagaaagatttaatgaaaagtattgtgtggcagccttacttggtc

SEQ ID NO: 208 - DNA, 401 bp, Canis lupus
  variation (201)..(201): y represents polymorphism t or c
gtaagctctctctccctcatgaaaataaataaattaattaattaaattaaataaataaaataaaaatcttaagaaaaagatgtaaggaataagtaggagaaagataagcaaagaagatagttctaaggtccatagtctggattaggaccccatctctatttctgtctaggacaggatgctagctactgggaggcaatggcytattctttaggttacataagtgtatttgtaagcccttgagggagcaccaaagactaagaacattttcttgagtactgaagtatagcctaagagtcttgggtaagcttgtgctactcatggagaaattaaaaggataccactttccactctcaacctccatgaaaccccaagaaactaacatgaatatccaaatctgcttc

SEQ ID NO: 209 - DNA, 401 bp, Canis lupus
  misc_feature (194)..(194): n is a, c, g, or t
aatagcccagcaactcctgagtggattaggatgacttgtgtggatcagtttactgacatggtaagagtagtggagggatgtcccagctataaccaccgcttaatggattttcatgttcttagtatggatttggtaatcatgacaaaatcacagggcaggttcatcttttccaatgaaaattctcactctgcctntatactkcaagcaactgcaaacctcctcaaatgatggcttgtttcagactgcaaagaaaagcagactcagttgagccantgctaggagcgaatcaacatggtagctaacttcttgaaacattctgtcaaaatgtagttgatgtggtattttaatcaccttaataaccaaattagattaaatagatatctgatctggtcaataattca

SEQ ID NO: 210 - DNA, 401 bp, Canis lupus
  variation (201)..(201): y represents polymorphism t or c
gaaggtgactgccccatgggagatgcttgagcttctctcccacgttactaggtcccccggtgacaacagctatgggtgcggggcatctcccttcacggtgctgccacatgggtgcctcactggtcccgttgccttgggacagaatcactttctcttgttgtggcagcggggtttgaggcaaaaccgaagtaacaagatgayaaggagatgacccgggctcttgtaccctgacggggaggggcatggcgggtgggtctgctcttgtcaccatgcaggaggagcgaagcaccacgcagtttcaagaggcagaactcgcctgtgcaagaagatgagctctgtacagtagccccgtctcgggattagaccagatgaccccgaaagccccatcgttagggttgaag

SEQ ID NO: 211 - DNA, 401 bp, Canis lupus
  variation (201)..(201): r represents polymorphism a or g
acaatagtaactacctgccctggttgagatagaacatctctcagggtgatgattttttttttttcaacacaatacaataattgaggtaggagcctgagtttcagatgggttcctgtgttgtgggacctgtgataaagcatataccttccaatgatactagcattttctaaaacgtgactggcctcttgaaccctgtctgargacagggacagaccaaggacacgacactgtcaatacggactcctaatcctgcctgttgctcctacatacagtggctttatcttctcttacacacaagagccacccctttantcttctgttatcttgaaaagatacctgaaaagagccctgcctcaggatcttggacttaaatctcagttgttcttccaaccatttatggg

SEQ ID NO: 212 - DNA, 401 bp, Canis lupus
  misc_feature (63)..(63): n is a, c, g, or t
agttccctcttagtcccctgaatggaccattccttgttgttgaattaactactatgtgccctngacttttctagcagtcacaaaggcagactgggacatttattggatcttaggtctcttattagccaaggtccttttcagcctaagctgttgaacaaatcaggttactacctcagccagacagaaatggtgagagcttartgcaggcagaaatgttagtagaagtgcatacatttctgcccttagcaggatagcaccagccttctctctgggatgtgaagtntaggagtagacagaggaggtgagagctgcttcctccacccctgcagggagggttggagagcaatgactctctggtacttactctccttgcaacggccttgggcttcctggcttccttc

SEQ ID NO: 213 - DNA, 401 bp, Canis lupus
  variation (201)..(201): y represents polymorphism t or c
tcccatgctaacactcactgtggtcatgtcaaatgcacgtaccccttccagattaagtgttgtaagtcccaacgcgtggttccgcctcaaactgttgttgagggcttatgtgagaaaatgaagatattaaaatccatgttgaccttagcatgagcaacgaaatgtctagaagcccaagaccaaaagtagattccctttcaygtgggcccttcaccattgaattgatgaacctgttgagcaacgcgctcagtaacaatgaccctccactcaatcttccagattacaggatgcaacttcatcttccagatgccaggattacaacttcaacaccgctagatctgaggtgtcactctccacaaatgagcttatcctgactataaagagtaaccatatcaccacag

SEQ ID NO: 214 - DNA, 401 bp, Canis lupus
  variation (201)..(201): r represents polymorphism a or g
tgttgcattaatggcttattttgcatatctgtttgattgtaccatcttaaagattcatttaaacctggggaggacagcatgactcctacctgcttctcattaataacatgcttttcagtgagtggataatgaatgacctggatagaaattagcagaaaatgcagattgccattaggtgtagaagtggggagggagtccacrttttccaccataccgacaaacaatttcaagtcagaagatattgaaaacaagcctcaaagagccataactgtctaggaggatttttaattaaatatcctgttgtctttacatgtagaactgtgaaggaaaatgcatcctaataaaaatcaaaatttgcaagtgacttaaaaaatcttgggttaatagaaacaagctatcta

SEQ ID NO: 215 - DNA, 401 bp, Canis lupus
  variation (201)..(201): w represents polymorphism a or t
actcaaaggaatgtactgaggtttctgaggcatgagagcaaaagggtctaggtgacagacaacactcaaagtctgatatgggttgtcatcctggttctcagttattagtcttataataagaactctgaccatatctggaaattccataacccagaactcaacttcctgagaaggaacttgttagatctaggcagacagacwagatagtcttcatttgcaccaagaaactgaggcagaagttcaatcatctagactgaatcaccatgggttaagggacagaaaggccacagggacataagtccaggggtcactccaggctcactggacaccttgcatggggaagaataactaagaccaaacctattaattggaagagataaagctcctaatacactcccagc

SEQ ID NO: 216 - DNA, 277 bp, Canis lupus
  variation (92)..(92): k represents polymorphism g or t
cgagacagacgtgaggacaggtggggtggacgtggccggcttgggcagccctggcgtttccctgcagaaaaaggcttcgatgaagaacgtgkctcntccccggcccccgccacccaccccctgcgtggccactcgcaacagctgcaagtccccggcgcccgcctgctgtgacccctgcgcctcctgccagtgccgcttcttccgcagcgcctgcacctgccgcgttctcagtcccagatgctgagcgcgcccagcggcctccagggggttggctgat

SEQ ID NO: 217 - DNA, 287 bp, Canis lupus
  misc_feature (92)..(92): n is a, c, g, or t
cgagacagacgtgaggacaggtggggtggacgtggccggcttgggcagccctggcgtttccctgcagaaaaaggcttcgatgaagaacgtgnctcrtccccggcccccgccacccaccccctgcgtggccactcgcaacagctgcaagtccccggcgcccgcctgctgtgacccctgcgcctcctgccagtgccgcttcttccgcagcgcctgcacctgccgcgttctcagtcccagatgctgagcgcgcccagcggcctccagggggttggctgattatctaagaa

SEQ ID NO: 218 - DNA, 287 bp, Canis lupus
  misc_feature (92)..(92): n is a, c, g, or t
cgagacagacgtgaggacaggtggggtggacgtggccggcttgggcagccctggcgtttccctgcagaaaaaggcttcgatgaagaacgtgnctcntccccggcccccgccacccaccccctgcgtggccactygcaacagctgcaagtccccggcgcccgcctgctgtgacccctgcgcctcctgccagtgccgcttcttccgcagcgcctgcacctgccgcgttctcagtcccagatgctgagcgcgcccagcggcctccagggggttggctgattatctaagaa

SEQ ID NO: 219 - DNA, 360 bp, Canis lupus
  variation (128)..(128): r represents polymorphism g or a
ggaggtagatgagcctctggggacgcccccctcctgctgcccagggccgaggggcccccggtcctctctgtgaggctgactctgactctcctcctcttgcccctgcctgcacctgtgaagaaaaagcrcctctccttccacgacttggactttgaggcagactctgacgactccacttggtctggaagtcaccccccccactcgtccccagtctcagtggccacagacagcctgcaggtcagtgggctcatttctggccccccagccttcccgggataacctgagcgacaggtacgtgggccccaggtgggggacggggcgctctgggaaggagtccgatggccatatcaagcttcgggg

SEQ ID NO: 220 - DNA, 954 bp, Canis lupus
  variation (790)..(790): r represents polymorphism a or g
atggtctggcagggcccccagagaaggctgctgggctctctcaatggcacctccccagccacccctcacttcgagctggctgccaaccagaccgggccccggtgcctggaggtgtccattcccaacgggctgttcctcagcctggggctggtgagcgttgtggaaaatgtgctggtggtggccgccattgccaagaaccgcaacctgcactcgcccatgtattacttcatcggttgcctggctgtgtccgacctgctggtgagcgtgacgaatgtgctggagacggccgtcatgctgctggtggaggcaggcgccttggctgcgcaggctgctgtggtgcagcagctggacgacatcattgacgtgctcatctgtggttccatggtatccagcctctgcttcctgggcgccatcgccgtggaccgctacctctccatcttctacgcgctgcgataccacagcatcgtcacactcccgcgggcgtggcgggccatctccgctatctgggtggctagcgtcctctccagcacgctcttcattgcctactacaatcacacggccgtcctgctttgtcttgtcagcttctttgtagccatgctggtgctcatggcagtgctgtacgtccacatgcttgcccgcgcccgccagcacgcccgaggtattgcccggctccgtaagcggcagcactccgtccaccagggctttggcctcaagggcgctgccacactcactatcctgctgggcattttctttctctgctggggccccttcttcttgcacctctcactcrtggtcctctgccctcaacaccccatctgtggctgcgtctttcagaacttcaacctcttcctcaccctcatcatctgcaactccatcattgaccccttcatctacgccttccgcagccaggagctccgaaagactctccaagaggtagtgctatgttcctggtga

SEQ ID NO: 221 - DNA, 420 bp, Canis lupus
  variation (173)..(173): y represents polymorphism c or t
cattttctttctctgctggggccccttcttcttgcacctctcactcatggtcctctgccctcaacaccccatctgtggctgcgtctttcagaacttcaacctcttcctcaccctcatcatctgcaactccatcattgaccccttcatctacgccttccgcagccaggagctcygaaagactctccaagagntagtgctatgttcctggtgaggctgcaggcttgaggccagggtgctggccagaggggggtggtgattgatacccatgtgactggggcagtcacttgcagaaaaggacagatgagctgatctgtggtgtggtggatgcatggaccctctggggccagagaaaggaataaacaaaaatctccaggagttgctgtggagaatggagcaggctgaggagatggtggggccaca

SEQ ID NO: 222 - DNA, 420 bp, Canis lupus
  misc_feature (101)..(101): n is a, c, g, or t
tacatatcccatccttttcccaggtactgagggtgggccaattaggagaaatccagctggaaatgtggctagaccaatggtgcaacgtcttcctgaaccanaggatgtcgctcagtgnttggaagttggtttatttgacacacctcctttttactccaattctactaacagtttccgaaacacagtggaaggtaagtaaaagaaatcagtgctttgaattcacagttaactgaactattcacattcagatctctttgaaaaatctttgaaaaaccatatagatcctgtgaatttacatgaatgctgcctccagttatgatgtagtcacaattctctgctcgagaaagaacttcttaaagaaaagtgtcagaccgtgaaactctttttaattatcatagaggagaagtgcttagaaattat

SEQ ID NO: 223 - DNA, 480 bp, Canis lupus
  variation (121)..(121): y represents polymorphism c or t
atgaaagctcataaactcctctctctgggaagcatcttcttgttcctgctttttttccatcagacctgggctcaattcccaagagagtgtgccactgttgaggccttgagaaatggtgtgygttgcccagacctgtccccagtgtctgggcctgggactgacccctgtggctnctcatcagggcgggggaggtgtgaggcagtgatagcagactccagaccccacagccaccattacccncatgatggcagagatgatcgggaggtttggcccacacggttcttcaacaggacctgccactgcaatggcaatttctcaggacacaactgtgggacttgccgtccaggatggagaggagctgcctgtgatcagaaggttctcacagtcaggagaaacctcctggccttgaatacagaagagaagaaccactttgtccaggccttggatatggcaaagcgcacaattcaccctcagtttgtc

SEQ ID NO: 224 - DNA, 360 bp, Canis lupus
  variation (161)..(161): y represents polymorphism c or t
tcttgctatgtgtaaaaattaaagggcaaagatcagatctctaagtatcctataaatatttacatatcccatccttttcccaggtactgagggtgggccaattaggagaaatccagctggaaatgtggctagaccaatggtgcaacgtcttcctgaaccayaggatgtcgctcagtgnttggaagttggtttatttgacacannncctttttactccaattctactaacagtttccgaaacacagtggaaggtaagtaaaagaaatcagtgctttgaattcacagttaactgaactattcacattcagatctctttgaaaaatctttgaaaaaccatatagatcctgtgaatttacatga

SEQ ID NO: 225 - DNA, 240 bp, Canis lupus
  variation (91)..(91): y represents polymorphism c or t
ccattcctctctgcctcccttccctgcccctcccatctctctgtctctctctcccctagtagcagccatactattacactgacatgctgayggtgggctgtgctgtaggagttggctgttgttttgggacgccacttggaggcaagtgatttacccctcctacatcagtccgctgcttgggcttgctccccagccaggttttgtcagcatccccaagtgtgacattaccagttacaacaa

SEQ ID NO: 226 - DNA, 240 bp, Canis lupus
gccgcgtggggtcggcccgcgtcaggccacctctcacggagctgcctcctcctgccgccagcgtcctgccaggagtgcaccaagtacaaagtgagcacgtgccgggactstgtggagtcggggcccggctgcgcctggtgccagaagctggtaagagcccccccccagggacctcgcgcccgtcctgcccgtcccgcgttcccgtccccgttcctgtccccacgccctccctctgcctct

SEQ ID NO: 227 - DNA, 153 bp, Canis lupus
  variation (97)..(97): Polymorphism includes deletion of c
ggaaaatacctgaccctagaggatgtggctgaactggtccggccatcaccactgaccttccgcacagtccaaaaatggctctcagcagctggagcccggaactgccactcggtgaccacacaagactttctgacttgctggctgagtgtccga

SEQ ID NO: 228 - DNA, 240 bp, Canis lupus
  variation (72)..(72): y represents polymorphism c or t
ctaggaaacacatttaaccaaatggcaaagtgggtaaagcgggacaatgaaacaggaatttattacgagacgtggactgttyaagccagcccaacaaagggggctgagacatggtttgaatcctatgattgttctaaattcgtgttaaggacatacaagaagttggctgaacttggagcagagttcaagaagatagaaaccaactatacaagaatatttctttacagtggagaacctacc

SEQ ID NO: 229 - DNA, 197 bp, Canis lupus
  variation (143)..(143): y represents polymorphism t or c
gagaacgtagcagttcacctgtccaatgtgctcttccggacatttgacttgtttttggccatccaccatctcttcgcctttctgggatttcttggctccgtggtcaacctcggagccggccactatctggctatgagcacgcycgcttctggaggcgagcactcccttcacctgcatttcctggatgctcctaaagg

SEQ ID NO: 230 - DNA, 480 bp, Canis lupus
  variation (274)..(274): y represents polymorphism c or t
acgcccaccctccaacccgcacggtgctcggtgctgctccccggctcctgcccccacagtccgctcgagagcttagcggttgcttctgagtgacccactttatgtccccccttgtgccatagagacctcagcaaaagggggctcccttccccgtcgcagcttctgtctttttccaaactccccgagccgaccagccgagccgtctcccgagcggcggagatcatggaagcctccgtgcaggccgtgaggacacgggtctatgggaagctggggygatcttggcctctcaccggtaagcgccccgaggggcaggccccagggcctcgacccaggacagcatggccaagggaaggtatctgggttcccctgggaggtcctgcagccccctccggacgtggggaaacggctcaagcccccaggcagccccgtcctggcatcagagagtgtgggggtgttggcgcggcccggacggaaggtggg

SEQ ID NO: 231 - DNA, 184 bp, Canis lupus
  variation (113)..(113): y represents polymorphism c or t
aggtattcaagagaaactggactacatcacaactttaaatataaaaaccatttggattacttcattttacaaatcatcccttaaagatttccgatatggtatcgaagacttcygagacattgatcctatttttggaacaatgaaagattttgagaatctgcttgcagccatacacgataaaggt

SEQ ID NO: 232 - DNA, 300 bp, Canis lupus
  variation (158)..(158): r represents polymorphism g or a
gtccggcaccaaactggaggactcccccccttgtcgcaactggtcatctgctccggagctgaatgaaactcaagagccctttttaaaccccaccgactatgacgacgaggaattcctgcggtacctgtggagggaatacctacacccgaaagaatatragtgggtcctgatcgctggctacatcatcgtgttcgtggtggctctcgtgggcaacgtcctgggtgagtctggccccgggcagccctcccgagggctgtcacggcccctctgcgcgggcggggctgccggggctctgaagac

SEQ ID NO: 233 - DNA, 300 bp, Canis lupus
  variation (123)..(127): Polymorphism is represented by presence or absence of gccag
ataaagagtaacactcttaaggaatgatgggcatgggttgtcaattaaaaatcagaaatgaagtgaatcttgtgaaatattgtaaattgatttatatttatttttatgtgtgtgtgtttcaggccagacctgtttgattggaatagtgtggtttgccagcagtcagccacacaacgcctggaacatgcattcaacattgccaaatatcaattaggcatagagaaactgcttgatcctgaaggtcggtacatttctggactaccatagtttttagtatagtttaatatttataatctcaga

SEQ ID NO: 234 - DNA, 141 bp, Canis lupus
  variation (80)..(80): m represents polymorphism a or c
gggttgccatggtcatttcctggatggataggaaaaggtttcaactggccttacgtgaatcttcagctgactgcctactmtatcatgacctggattgtgggtgccaagcattatcatgatttggacattgattatatcggg

SEQ ID NO: 235 - DNA, 208 bp, Canis lupus
  variation (156)..(156): r represents polymorphism g or a
atcgactctatctcctgtggctcttgcttgtcaccattgcctataactggaactgctggcttataccactacgcctcgtctttccatatcaaacaccagacaacacacactactggtttattacagacatcacatgtgatatcatctacctttgtratatgctattaatccagcccagactccagtttataaaaggaggagacataat

SEQ ID NO: 236 - DNA, 239 bp, Canis lupus
  variation (101)..(101): r represents polymorphism g or a
atgcttcccagaggacattcacaattgactacagccacaaccgcttcctgaaggacggccagcccttccgctacatttcgggaagcattcactattccccrtgccccgcttctactggaaggaccggctgctgaagatgaagatggctgggctgaatgccatccagacgtaagtaagagggcgctgggctctcacctgggcctagacacccatacctggagagagagagcagctggatc

SEQ ID NO: 237 - DNA, 300 bp, Canis lupus
  variation (162)..(162): r represents polymorphism g or a
ggtgcctaaggtggctggcactgacttgccgtaccctccccatgtctccttgtgtctgcagtgggtgaatggggtccatgtggcagagcacgaggggggtcacctccccttcgaagctgacatcagcaagttggtccagagcgggcccctgtcctcctgccrtattacccttgccatcaacaacacgctcaccccccacactctgccgccagggaccatcgtctacaagacagacgcttccaagtgagcagcactctgctcccctgccccccctgccccccacccactgggcttccgact

SEQ ID NO: 238 - DNA, 360 bp, Canis lupus
  variation (147)..(147): r represents polymorphism g or a
ccgagccacgtgccttcggtccacgaagttcaccatttataacaacatgttctgtgctggcttccatgagggaggtaaagattcatgccagggcgatagtgggggaccccatgtcaccgaagtagaaggcataagtttcttaactgrgattattagctggggtgaagagtgtgcgatgaaagggaagtatggaatatataccaaggtgtcccggtatgtcaactggattaaagaaaagacgaagctcacctaaagaataatgtatttccaaggttgacacgtttagggtagaaaatggacaaggtcctttactaactaatcactttttttatctctttagatttgactatatacattctc

SEQ ID NO: 239 - DNA, 241 bp, Canis lupus
  variation (121)..(121): Polymorphism is represented by the presence or absence of c
gtgcagggagaagggcctggcactgctcaaagaagagccggccagcccagggggggaaggcgaggccgggctggccctggccccaaacgagtgtgatttttgcgtgacagcccccccccccactgtccgtggctgtggtgcaggccatcctggaagggaaggggaacttcagccccgaggggcccaggaatgcccaacagcctgaaccaaggggtcccagggaggtacctgacaggtgagc

SEQ ID NO: 240 - DNA, 159 bp, Canis lupus
  variation (70)..(70): r represents polymorphism g or a
aggagttttcccgtttccacgaagagatcctgcccatgttcgaccgactgcagaacaacaggaaggaatrgaaggccttggctgatgagtacgaggccaagctgaaggccctggaggaggagaagcagcaacaagaggacaggacgacagccaagaaag

SEQ ID NO: 241 - DNA, 894 bp, Canis lupus
  variation (324)..(324): r represents polymorphism g or a
gtgttctggggatgcgtaagagggctctggtctttcaaccagtgactgagctgaaggaccagacagattttgagtgagtacatctgcttccctggtagtttcagggtctgctcttcccagcctgtgtgctgccttcaatcctctcatcctaggactaacaccgtcatcacacctatttcagatcttaaccccgtgccctaaaatccggcctcttctactcaacttctttccataagctttggatagaagtcagttgggttgctaaaagctgaaatcatcatctctctcatttctctgtagtcaccgcatccccaaggaacagtrgtggctgaagctgaggcccatcctcaaaatcctagccaagtacgagattgacttggacaccacagagcacgcccacctggagcacatcagtcggaagcgatctggagaaacttctatctaaccctctttggagtgagggtcatggattgtctgatcatggtcagctcaccccctgatagatccaagtccatgtatccccaagtattttagctcatttttctttaggtttccttttattctgcaactgtagccatgaccagctctggccagggagctggggcagcgggcagtgagtagaggctccttttaggtggaatttatcaacttctaccccagcttcatctgtcacacaagactgggctcctctagtgctactgctagatttcagctactcggttagaattttcctgaaaataagctttatttatttctttgtgataacaaagtcttggttcctctattacttttactgcagtgacaaacaatagctacactaataaatgccaactggtcactgtgcttttggttctcctgttgtcactttcacaagtgaatgtcatcctgtcaacc

SEQ ID NO: 242 - DNA, 284 bp, Canis lupus
  misc_feature (64)..(64): n is a, c, g, or t
cagagcctgaagtcgtcctgccggagccctgggtggccaagctcaggcctcagcagcactcttnggactgagccgcccacggggcagccgccaggaccgcagccatgaacgggasggagggcccgaacttctacgtgcccttctccaacaagacgggtgtggtgcgcagccccttcgagtacccacagtactacctggctgagccatggcagttctccatgctggctgcctacatgtttctgctgatcgtgctcggcttccccatcaacttcctcacgctctac

SEQ ID NO: 243 - DNA, 359 bp, Canis lupus
  variation (194)..(194): s represents polymorphism g or c
gcggcacgacttgctggtgggcgcgccactgttcatggagagccgcgcggaccgcaagctggccgaggtggggcgcgtgtacttgttcctgcagcctcgaggtcaccaggcgctgggcgcccccagcctcctgctgactggcacacagctctatgggcgattcggctcggccatcgcatctctgggcgacctcsaccgggacggctacaacggtaaggggcagagaggagcaccgcttgcttcagactggttaacagccagaaccaagaccgccgatttgaccagagggcagccagagcggggaagggcttttctctggaagagttgaatgggaccagtttgtttgcattggtccaggc

SEQ ID NO: 244 - DNA, 288 bp, Canis lupus; identical to SEQ ID NO: 63
  variation (83)..(87): polymorphism is represented by presence or absence of tctca

SEQ ID NO: 245 - DNA, 269 bp, Canis lupus
  variation (191)..(191): polymorphism is represented by the presence or absence of c
ccttcaggatcctaacttgttcaggccaggggaatgaccacacacacacacatatctccagtgatcccctgggctccggagaacctaacccttcacaacctgagcgaatcccagctagaactgagctggagcaacagacacttggaccactgtttggagcatgttgtgcagtaccggagtgactgggacccgcagctggactgtgagtgacttgggtcatgaaggtggcagcaaaggccaagcaaatagggataaaggattcaatcagc

SEQ ID NO: 246 - DNA, 360 bp, Canis lupus
  variation (150)..(153): polymorphism is represented by the presence or absence of cctc
gtttctaaggttctttccaccggaaactatgacagaaggaaatgtgtgggtggggaggggtaatgggtgaggggcccaggttcctgacagtctacacccagggaacgaagagcaagcgccatgttgaagccaccattgccactcagatccctcttattcctgcagctgtctctgctgggggtggggctgaactccacggtccccatgcccaatgggaatgaagacatcacacctggtgggaaacatgggactggaaggggttggtgagaggggagcctgtgggaaggggtcgcatagaaatcttgaacctgccatggggcattagaaggatgtgggcagagtttaagagtgctgtggaga

SEQ ID NO: 247 - DNA, 219 bp, Canis lupus
  variation (121)..(121): Polymorphism inlcudes presence or absence of a
ggaaggctaagtggagcaaataaatgtttgttctgaaacattaagaattacttcattgactttttaacagaatatgcaataaattaaatatttcttatctataggagaaagaaaaaaaaaacaaaggaagatagaaatcttaccaaagatgtttcacttctagacctggatgattgtaagtgttgaaatttaaattttttcttctctttttagtagtag

SEQ ID NO: 248 - DNA, 360 bp, Canis lupus
  misc_feature (141)..(141): n is a, c, g, or t
ttagcccttttctttcacagcttgaaggttactggactgaaaaactccgtttgcttctgtaggtttttttcttacttccgaggagtggaggtcactgacaatgcccttgttaacgtctacccagtaggggaagattactangcctgcacggagaccaacttcattacaaagattaatcctgagaccctggagacaattaagcaggtaggacgaaatgctcaggcgacgttgctcaagaatttagaatttgcagtttagatttaactgcaattttggggaaagctcatgagggccaaatagattgtctcgctgccttgctttgtcatcaactactagccatgtgacacgaggcactcttta

SEQ ID NO: 249 - DNA, 357 bp, Canis lupus
  variation (241)..(241): r represents polymorphism a or g
gggatatccgataccggggtggcaacaggaccaacactggactggccctgcaatacctgtccgaacacagcttctcggtcagccagggggaccgggagcaggtacctaacctggtctacatggtcacaggaaaccccgcttctgatgagatcaagcggatgcctggagacatccaggtggtgcccatcggggtgggtccacatgccaatgtgcaggagctggagaagattggctggcccartgcccccatcctcatccatgactttgagatgctccctcgagaggctcctgatctggtgctacagaggtgctgctctggagaggggctgcagatccccaccctctcccccaccccag

SEQ ID NO: 250 - DNA, 360 bp, Canis lupus
  variation (183)..(183): r represents polymorphism g or a
tgtcgctccctctcttacccggaggaggactgcaatgaggtctgcttggaaggctgcttctgccccccagggctgtacctggatgagaggggagattgtgtgcccaaggctcagtgtccctgttactatgatggtgagatctttcagcccgaagacatcttctcagaccatcacaccatgtgrtaagtgcgagcagcatgaccagggacctcaggaatggcggagcttgtaaggaaaatggtcttctgggtccttcatttcacggttgggaaactgaggcccaggaagggaagtgacttgccctgagttgcacagctcgaatgatttccttacatcgctggaaactagagcagactgcca

SEQ ID NO: 251 - DNA, 360 bp, Canis lupus
  variation (212)..(212): r represents polymorphism g or a
ggcacttacttattgtttcaatgactggaataagtttgctcatggaaatcttgtgtttgtaggtgtgtgtccaccgaggcaccatctaccctgtgggccagttctgggaggaggcctgtgacgtgtgcacctgcacggacttggaggactctgtgatgggcctgcgtgtggcccagtgctcccagaagccctgtgaggacaactgcctgtcrgtaaggggagcagaggggctgggcgctgcctggagcaggcaagggagagagtgggggagtgggggttctgggaaggggcaagagaccccttgagtaatttctggttcagggccagagatgaggggaaggagaggactgaggttgggtg

SEQ ID NO: 252 - DNA, 291 bp, Canis lupus
  variation (216)..(216): polymorphism is represented by the presence or absence of c
gaagggaaaaatgagtgagtaaattatattttggggaagatttttttgttgttgttcatttgttacgtccttggggagagttctccatgagatgggattaatgatgtacatcagatgattagaggtaaatatcccggcttttttggtaataatcatagttactgactcttttctctttcagggggtttccaaaatggcaaaagagtgagcctctccgtgtatctcggagaatttttcgacattcatttgtttgtcaatggtaccatgctgcaggggacccaaaggtaagtc

SEQ ID NO: 253 - DNA, 201 bp, Felis catus
tagtcagtcttggatacattcggccacagagtccttcaaaaattgcctttcagtcctatgttgacaaaggtaagtccagggcatttcaaggtgcccaacargagtgctaatgtgtagtcagggtcagagatattgggagggagctatcctcacttatgggacaagaggaacatggagttacacacataggataaatgaaaa

SEQ ID NO: 254 - DNA, 201 bp, Felis catus
ggatgtggagagatcggaagccgtccgccccggtggtgggattgccaaacggtatagctgttctagaacacagcccgcggtccccggaaaagttactatargactgttacgtgtccccgcaagcccacctccgacgcccgacaggactgacagcagggtcccggagagaggcacctacgtccgcaaaggtaagtgcgggag

SEQ ID NO: 255 - DNA, 201 bp, Felis catus; identical to SEQ ID NO: 254

SEQ ID NO: 256 - DNA, 201 bp, Felis catus
taacacctctgagctgcatttcccttcatttggggctgaatgacgagaggtgcagaatgttctttccaaggttttggagagaattcagtgagacagtggcraacggtgcccgatacagtaagtgctcaataaaatactaaagcggaatctagtggaaactgctcaacaccaccagcggtttggggagctaagaaggcaaca

SEQ ID NO: 257 - DNA, 201 bp, Felis catus
aagttcccaggatagctgcacaccaggtacagcgagaagactgggtcagatcaagaggctctggggagacagtcttcaggggcagacaaggatatactgtcygatgcatctgaaccaatcagacatggtgacaggcttcttcacctgataagaagattcaactggcaagaagcacacagacaaccaagttagcaaagcaga

SEQ ID NO: 258 - DNA, 201 bp, Felis catus
agggaggaaataaagatgtttgatttattactgataaccccgaggtttgagtgtgcacccaaagggatgtgctgtgaatctccgcttctgaatgagacacrctcaacagccaggacactggtacagctggcaaaccacaagctacccctgtaggaacaggcgccttgctgcatggcggaaagctaaccggaaacccccact

SEQ ID NO: 259 - DNA, 201 bp, Felis catus
gtgtagagtgagcttgagtactttggcttttgtgttttgtactcaaacccacggtcctctggtttcaaatctgtggtagagaactactcttccttaggtcrttcttggattcacgccaacccttctctctagtccttacccattgtcttgttctcccttgatgatatcaggaatcttctcctatctcagggtctgagtgtt

SEQ ID NO: 260 - DNA, 201 bp, Felis catus
aagcctatgatagaaacgaagagcccattccaacctaacaggtaccctcaaatcaccctggtgcaagggcaaaaactcgggaagcaggtcagtcgtggttkgaaacccatttctgttgctttctagcaattgttttttgaaactttctgcacctcggttgccttacagcgctgaagtgagggtcacacgattgagggtcta

SEQ ID NO: 261 - DNA, 201 bp, Felis catus
gtgagcaaaagtgggtcagggtacagacaggcaggggggttccaagcaaagcaaaacccacatgaaggctcagggctgtgcctaccccatctgcacaatgytaaaaatctcacctaatgtatttaaacatcccttttgtctagaccattctcatataggtgtcaggacgacccctaaaacaatcagaatgtatcactatat

SEQ ID NO: 262 - DNA, 201 bp, Felis catus
cttggaaaaatagaattttacagttggaaggactgtacagtcttccaaatcactcactttcattacttgttccactgacagctttcactaaaagattactmtggaaaaacagcttcccttctcatccctgaagtgagtcaaattgccttttcatgtatctgttccagagggccatctgctctgcccccaggagaagcttct

SEQ ID NO: 263 - DNA, 201 bp, Felis catus
gaatttctgcccttgtgttctgtggcatccctctccttgaatttctgacttttcacttcttttatatcatttaattctcataacagtcctgggaggtaggygagcagagattattaacgtttttgaagatgagcacactgaggccacattatttagcttctctaaacgtttctcatctgttaaacgaggagcaggacaaga

SEQ ID NO: 264 - DNA, 201 bp, Felis catus
ggtcccagtcttacttactgtaaaccaggaatagcactacctgcacctttgcattgcattgcaaacaacaatgactgacattaagaaaatcctcagtaaaygttggcattttttgttaaattcttgaccctatcatttactagctaagggaagtccgtgtaagagacttcattctcttcacaccacagttctcctctatgc

SEQ ID NO: 265 - DNA, 201 bp, Felis catus
aaaagtggaaatgtgtattacagaggcagtcccaggcatggcaggctctgacagggtgttggagatgatgggtgggcttgaataccctgcctgtgggggasgggggtgctgggagaagcacaaggacccaggggagatggcaccctgcatgtctgggcctgggtgggggatttaagtggtccacgtccctatctaggaagc

SEQ ID NO: 266 - DNA, 201 bp, Felis catus
ttatcactggcctcttactgtggccagcccccacgagaactccagtgagacagcaggcaatagctcatgaagtgaaatcattcaatgccaaaaggacttcygggcccccgccatgtgcagggagccacttacaccccagccacacggagggaagcaggagtgctacaccggtggcagagaagagccacccccccaggccgt

SEQ ID NO: 267 - DNA, 201 bp, Felis catus
tcccttgagggcacccgccttagatcacaccttctctcaaggtgcacgtgacagggcaaatcttttgcttccagcccaagcttggtagcttaagtgaatgmatttagttttatgtagattctggtctcctgaccagaaatcactaggaaggaacaggtttgtctaacatagcttgtaagtgccgggtccctgctggccatt

SEQ ID NO: 268 - DNA, 201 bp, Felis catus
ggtcacagaccccaggccaccacccacctggatgccggaaggctgggcactcctggaatggccgggtccagcctggttattccctcgcccgcagccaagtycatccctcccggtggggtccacaccatctttcttccaaaccccacaggtgcaaagggcctcttagcagcaactacttccggggagggagcaggtgacagc

SEQ ID NO: 269 - DNA, 201 bp, Felis catus
tgattcttcaacaaactcacaatccactttagtaatggaagcagctctacctttggcaaacaaaagcagaaaaagtacaaccatggctgtgtagaagtccyatatatcactgcatgcttaaactttactcagcaaaacttttaattctttggggaaggcaagagagaaatagtacctgaaaaccaagtattagtattctca

SEQ ID NO: 270 - DNA, 201 bp, Felis catus
tggactctagttctaccaatggttactctgagataatgcttatcctatcttatgctgggaggaagctggtcaagtaggtatgagcctgtacaactgctgcrggatcagagctgcctcaggcctctggttttagaggcctcgtgatttccaagggaggaaaaggccagcattgcttgtcctgtcagcctccctcctggtttc

SEQ ID NO: 271 - DNA, 201 bp, Felis catus
catccagaagtatttaaaaacaatctttttgcatgtcatgtttatttatttttgagagagacagagcacaagcaggggaggtgcagagagagacggggagrcacaggattcgaagcaggctccaggctccgagctgtcagcacagagcctgatgtggggctggaactcagactgtgagatcatgacctgagccaaagtcga

SEQ ID NO: 272 - DNA, 201 bp, Felis catus
cccaggcatgaggagggcaagagggtagggctgtagttgtcagtggggcgagccctgtccccctgagcccttggtggggctctgactccctaaaactttaragggaagataacctaccctcaaatagtgagtgtttttcgcccttcctcctcaactctgaaacatttgcagtcaagggtagtaggggaccctaaccacagg

SEQ ID NO: 273 - DNA, 201 bp, Felis catus
atcgggctccacgctgacaacgtggagcctgcttgggattctctgtctccctctctctgcctctctttctctctcaaaaataaataaataaacattaaagraagaaaagaaaaagaaatggatttgaggaagtatatcaagcaaacaaaaacacgggttggcgcaggagtaacaaagtggcaaagtgtcgcctaaatagca

SEQ ID NO: 274 - DNA, 201 bp, Felis catus
gctaaacattctacaacgtacacaacaaggagtagtcattcctggtccaaaatattgattgtgttgagcctgagaaactcctatttaaataactgagttcycttttcatttagtacaagtatttctcacactattgtacaattcctacaattaaaactatacaatattcctgatcaccctgctaacttcacccatctttct

SEQ ID NO: 275 - DNA, 201 bp, Felis catus
acgcagacagagttactgggccccaaagccacaacccggcttcccttccttctccctttgccttctgtcaagttttaattcaattaaattagggagaaacyggggcacctggcctagctcagtcggtcaagggtcccgcttcggctccggtcacgatgttacggcgacatacacagggctggcaaaggtgagccttcccgc

SEQ ID NO: 276 - DNA, 201 bp, Felis catus
aatcattaaaaatgatgaaattggagaatacttcatgacacaggaaaatgcaaaatcatcttaattgaaaaagcatttcttcaatatatatgtgtagttcrtaatcaggggaaaaaaccagtcccataaaattctcctattccaaaagaaacatcacatcatagaacatagggcccttccctcttttcctgagagcttcaa

SEQ ID NO: 277 - DNA, 201 bp, Felis catus
ccattcatttcctcttcagtgcagggaaccacccaatcagacagttgcacaatgaaaagaaggtctgagaatgccaaggtgtgtcactggttgtggtcccrcagccagaaagggcaaaggtggattgccaaaccagggctacgtaacttcagctgagaaaccctgctgccttcaggcctgcaatttctctagacctcagtt

SEQ ID NO: 278 - DNA, 201 bp, Felis catus
tttcagcaagcatttactaagtgtctgtgatgtaaggctggttaggttctgagtgtaggaaataaactggacgtggcatccttaaggaactcatgatccarggtatcattaatccccaaagcagtaagaaagaggccattgcagaatagggggtgggggtgggcaacacctggaagttgaagaccaggtcaggaaaggtca

SEQ ID NO: 279 - DNA, 201 bp, Felis catus
ttccgactcacatcatcctccactgatgtcctaatagagggcactgtgctttggtcatacatgagttttgatcaagagttatattttctagttaaaatgasaaactgtaaaactggatatgaggccttcagctgtatttactaattaatcatactgagttttgatccagatgtgggaggaactgaaaattcccctgtgtaa

SEQ ID NO: 280 - DNA, 201 bp, Felis catus
atagtggtaataatattattaatcttgtagaatgcttaagtagattggtttttttgttttcttgtttttgtttttgtttttgttttacatacataagcctsttagcatagtacctggtatacatttctgtattcaaggaatcatgtcaattgtctatttatgtatagtaacatagactctgggcccatcccttctctttca

SEQ ID NO: 281 - DNA, 201 bp, Felis catus
ctctgccacagcacggatgagtcttgaggacgcggtgccgagtgcaggaagcctcggaaaggtgcttgccaggggctggaggcgggggaggtggggagtgrctaatcagcatccctcaagtttcggccaagcaagatgaatgagctctagagacgcgctatatacagcactgtgcctggagtcgacggtaatgctttgtgc

SEQ ID NO: 282 - DNA, 201 bp, Felis catus
attccagtggctggtgtcattataggaggccacgcgaagacacagagacagagaagaccaccatgtgaccacggaggcagcagttggagctccctgcaacmagtcaaggaacgtcagaaaccaccagaagctggaagagacaggaaggattcttagagcctccggagggagtgtggccctgccaacatcttgatgtcagaa

SEQ ID NO: 283 - DNA, 201 bp, Felis catus
atccgtgtcacgttcagtcctcatgaccgctttggtctgccctcagcctcgctcccacctttgggctctgaacacccatcaggaggctctgctcggatggygtgagtgttccgagatttgaggcatcatcaaaacggtcaattacacaagtctggtaagaacgcagctgtttgctttactttcaaaagtctttattagggg

SEQ ID NO: 284 - DNA, 201 bp, Felis catus
tagccttgactcctggttattttcacataggctgcctgcatttgatattatcctcagaaagtctcgcttttacattttcgcacatagatatcatcccttcycattaaagtgctctgatgacacttctgtgtttgtttattagagctcagcaggaattatgagagaagtcgttttaagaaaagaaaaaaaaaacaacctttt

SEQ ID NO: 285 - DNA, 201 bp, Felis catus
ccggaatgaggcaagcctggtactgagagcagatccctgaagcctggatgggcagagcttggtgtaaacaaattaagtagtgaaagtctgtggagcactgrttcttataggtggatggacaaatgtttatgctgggaggctggggaggaaaatgccacctgacagctcctttgttcctggaggggtctcccagttatctct

SEQ ID NO: 286 - DNA, 201 bp, Felis catus
agcggctgaataataggtgtttttttatggatcattgaaggtaggggctgctgatgccagggtagaccgaggtttgtttgaagccaatgttctggaacatrtttgggatctgactcttctgagatgtgatcaggtgtttgggagcctaggacaaaaactcagagaatgatgaactttcttgcttccctcttaacagtggga

SEQ ID NO: 287 - DNA, 201 bp, Felis catus
tgttctctcttctgtccacatatcgatccaggactatggtaggagtcgactcattgccttctcaaacaggggtgtcacggtcaggatttggaatgacagtycaaggggcctccagctttgccattgctgcaggttaggtcgggaagctgcggactctgtgactgagattaccattcaggacatttaatagggggtgatttg

SEQ ID NO: 288 - DNA, 201 bp, Felis catus
tatccggggtgtctgggagaggcgtctttgaagaagtttacattggagatgagatctggaggatgcgagggtggggagaggaacagagatcctgaggatayggatttgagtgtgtgtgtgtgtgtgttggagtattcaatggctccctattgtcctcacaataacacccaaactcctccctctttccagggacagagggac

SEQ ID NO: 289 - DNA, 201 bp, Felis catus
ttaataaattgagtcaaataattctccctctcttgtctgagccagtgcttttctgcttgaggaatgagtagcttagatgattgataacagaatccataacmtttcccctccaagtcaccagcttgaacccaacctagttgagcaatgagagacatttgtttccccaggcagcttatgagaggtttgcatgaaatgaatggg

SEQ ID NO: 290 - DNA, 201 bp, Felis catus
ccaaccacgatataattgcactcatccaaaaacagaagtgaccggggccacagtaaatgtgcctcttttgcaactctcattcctcttaaatctcaaataargattaaaaatgtgcatttgaggagggccaccgtggtggctcagtcggttaagtatctgactcttgatatcgactcaggtcataatctcatggtttgtgag

SEQ ID NO: 291 - DNA, 201 bp, Felis catus
agcacagctggggatcttccatcccggtgctgtttccagcagctcggtgcggaagcaccacctttctggcttgtaaactgagaatactgatccagcccccrtgaagagacattacctaagaaacctcccttatgaagccttcaaggtgagagtattttacagggaaatccacaggctaaaaataaaaacacaactatacct

SEQ ID NO: 292 - DNA, 201 bp, Felis catus
aggaaaaggagaacattaacatgattcttggaggttcaacggtgttaagtccaacccctcacaggactccacgcatcccttagcaggagttaagggagaayggtaaccctcacagtaggacaatccccctgcagacctcctccatcatcaaggtcaagtgggaactccagaactctgagtgttttggcaagatgctccctc

SEQ ID NO: 293 - DNA, 201 bp, Felis catus
gagatgaatgactgccctgagtgccaacaacagaaagtgcataggataaagagagagttgacacaggaactcatacccctgaggtaacataccatacaccraaagttagcagtggacaataagtagcttagagccctgaacttgtcctgtcatatatccaatgtcagcaccactctggaaacacaatcccacataatcccc

SEQ ID NO: 294 - DNA, 201 bp, Felis catus
acaccaaaggacaaaggccaagggcaggcttactggcttggatgagtgatggagggctgctcttagggaacacggggctggggcgagggccggtgacacartgtgtgatcaggcggggctttccagcagcctcaatgctgaggggggcaggaggccaaaggcagcgccttaagaagcaggccaaacaggggcatctgggtg

SEQ ID NO: 295 - DNA, 201 bp, Felis catus
actaaatcatgtgcttcttctatgaggactccaaatgagttcatagcttactcatcactaacatgagcaccacactaggtagttcgtatactgtttcattygactctaatggccttataaagtagatgtaatcattatcccactttgcagagggaaaaaacagaaacttagcttaagcaatttgcctgtttacattactag

SEQ ID NO: 296 - DNA, 201 bp, Felis catus
ccctcccccactctcgctatacgtgtgtgtctctctctcaaaaataaacactaaaaaaatgttttaaaaacacaacgtggttaattcatgtggaattcctyatgttacaagttatttgcaaaatttttttcttcttcttctcattcagttttacctgggactggacggagccagacatttgtgatccaagcttctactaca

SEQ ID NO: 297 - DNA, 201 bp, Felis catus
tttcccatgagactcatgctctaaaaggagatgcagacccaagtgtgaggaatggaatgacgaagactgaagcaccctttttttgagagagagaaagggcrgggggggggtgtactttaagcagtttccatgcccagcacagaccccaacgcggggttcaatcccctgaccttgagatcatgacctgagacaaaaccaaga

SEQ ID NO: 298 - DNA, 201 bp, Felis catus
gttctttgctgagcataaaggattggctatggggaattttcttttatctttgaaaatctgttgcgtatcttttagaaatagtttttacctggttttctctmtttttgtttaatttttttttcttttggggggaattatatggggcggaagttttataccaagaggcacacagcatattctatgttagcattgactcctctt

SEQ ID NO: 299 - DNA, 201 bp, Felis catus
ttgtttctatctaattttttatcttcttccctgatacttaatttttatttgtcttttatctccaactgtctgtaatagttacttccaaaaaaaaaatcaawcatgtttaaaacagaattcacagcccactaaccccaaataaccccaaaacatagccctcaactgtttcattaagaagatgttctctggagtcaaaaagaa

SEQ ID NO: 300 - DNA, 201 bp, Felis catus
acagcgggctctggggctcgagctggctcccttggtcacacgcagctttccatgatgcttccagttctccagaactctccgattgatcctgccctccccayggggcagagcgtccactcctggtgactcctggtcccctgtatctgtgccaacgggggccggtggcggggggggaagcggcgtggcttggctggaggggta

SEQ ID NO: 301 - DNA, 201 bp, Felis catus
gggttttgggcttgtcatgggtaaacaagggaggcatctaaggtggttctgtgcagtaaaccatttccaggaacacaaaagggcggggggagttttttagraaaaataattaatgtttatttttcagagagagagacagagagacagagggtgagtgggggaggggcagagagagaaggagatacagatctgaagcaggct

SEQ ID NO: 302 - DNA, 201 bp, Felis catus
gaccctgtgtgaggttttaagcctggtttctctactcactagttgtgtgaccttgagaaaccaacttaatctcaaccgcatatggttgtagagagaattaygtaagataataaaaaattgagagcactgccgggcatgtaaaagctcaataatattaaatgttgtcattgctattgtcattaatactggccaggatccagc

SEQ ID NO: 303 - DNA, 201 bp, Felis catus
gggaatcaaggaaaacccaaagtactaaatggatattataaatataggagatgaattttttttagtagatatatatagagagctgataaatggcaagggtstaaaagaaccaatgagtataataaaaaatatttcttagcaaataaagtatttagatgtttgaacagtgcttttcaattttttattgattaatttgacaat

SEQ ID NO: 304 - DNA, 201 bp, Felis catus
aatgacccctttctatttgacagaattcacatcaaaagccaatgagatgaggccaggagtttgctttccctgttgtgaaggtgggaggggaaccagcagcrgtgataagtggctgtgatccctccaacctttgccaaggtgaacctccacccacccccctcaccgtgtctcagatagaaatgcttgtttctgatgtttttc

SEQ ID NO: 305 - DNA, 201 bp, Felis catus
acaaatttcattgcatttgagaaaagcgctgtgctggggaagacctttttgtttttggaacacactactagcatggtgagcctcacggagtctttgcttaygaacgtataaatatgcttgtaggtcaatggcatcataccagaatacactgcaatagaagcacatctttcctcgtattaaaaggataggtatctgtgcata

SEQ ID NO: 306 - DNA, 201 bp, Felis catus
aaaataatcaaaatctggggcgcctgggtggcgcagtcggttaagcgtccgacttcagccaggtcagaatcttgaggtccgtgagttcgagccccgcgtcrggctctgggctgatggctcagagcctggagcctgtttccgattctgtgtctccctctctctctgcccctcccccgttcatgctctgtctctctctgtccc

SEQ ID NO: 307 - DNA, 182 bp, Felis catus
  variation (78)..(78): r represents polymorphism g or a
ttagccgattggaggagtacaatagccgtcaggctttatgtgatggaactccagagggaccattactgcgcaatcccrgaaaccatgacaaagccaggaccccaaggctcccctcctctgctgatgtggaattttgcctaagtctgacacaatatgaatcgggttccatggataaagctgca

SEQ ID NO: 308 - DNA, 178 bp, Felis catus
  variation (121)..(121): k represents polymorphism g or t
acactgcttggagggtctgaaatctggaaagacattgattttgctcatgaagcccctggtttcctgccttggcacagactcttcttgttgctgtgggaacaagaaatccagaagctgacckgggatgagaacttcactattccatattgggattggcgagatgctaaaagctgtgaca

SEQ ID NO: 309 - DNA, 182 bp, Felis catus
  variation (113)..(113): polymorphism is respresented by the presence or absences of c
ttagccgattggaggagtacaatagccgtcaggctttatgtgatggaactccagagggaccattactgcgcaatcccggaaaccatgacaaagccaggaccccaaggctcccctcctctgctgatgtggaattttgcctaagtctgacacaatatgaatcgggttccatggataaagctgca

SEQ ID NO: 310 - DNA, 420 bp, Felis catus
  variation (160)..(160): r represents polymorphism g or a
tgaccctgctattcgaagccttcacaatttggctcatctattcctgaatggaacagggggacaaacccatttatctccaaacgatcctatttttgtcctcctgcacactttcactgacgcagtctttgatgaatggctgaggagatataatgctggtgaracatttcctatgttaacaagatgtctttggcatattttagatgtatccacatttccattggaaaatgcccctattggacataataggcaatacaatatggtgccattctggcctccagttaccaacatagaaatgtttgttactgctccagacaaactgggatatacttatgaagttcaatggccaagtgagtattgaaaatgtatcttttctgtggaaattaccaaaactacatttgctaccttttaaggtaatgacag

SEQ ID NO: 311 - DNA, 300 bp, Felis catus
  variation (120)..(120): y represents polymorphism c or t
caggtgtgaggcagtgactgcagactcacgaccccacagcctccattacccgcatgatggcagagatgatcgggaggcttggcccacgaggttcttcaacaggacatgcygatgcaatggcaatttctcaggacacaactgtgggacttgccgtcctggatggaaaggagctgcttgtgaccagagagttctcataggtaagtggggatctgcatgtacatacagttcttcatgagactctatgcatttaataggaacctaaatcatttgaactggaagcacatctgaaaatcatacaac

SEQ ID NO: 312 - DNA, 236 bp, Felis catus
  variation (85)..(85): y represents the polymorphism t or c
gctgtggctgttggtttcctccgccgtctccatacaacgattctgcgataccctcatcagacccaccgaccaagaccctctggcyctttgatattgatcaggacccagaagaaagacatgacctgtcaagagactatccccatattgtcgagcagctcctttcccgcctccagttctaccacaaacattcagtgcctgtgcatttcccggcacaggacccccgctgtgaccccaag

SEQ ID NO: 313 - DNA, 150 bp, Felis catus
  variation (100)..(100): r corresponds to polymorphism g or a
catgacctgtcaagagactatccccatattgtcgagcagctcctttcccgcctccagttctaccacaaacattcagtgcctgtgcatttcccggcacagracccccgctgtgaccccaagggcactggggcctggggcccttgggtatag

SEQ ID NO: 314 - DNA, 569 bp, Felis catus
  variation (307)..(307): m respresents polymorphism c or a
ttcttcctggtcaacgactggctgtcggtggagactgaggccaatggcggcctcgtggagaaggaggtgctggcagcaagtaagggcctgggcccgtccctgcccgggctggccgaggggtggcctgtgccactggcctcctgaagccagctgtgccctttctgcaggcgacgcggctgtgcggcggttccggcgcctcctggtggccgagctgcagcgtggcttttttgacaagcatctctggctctccctctgggaccggcctcctcggagccgcttcacccgcgtccagcgggccacctgttgmgtcctcctcgtctgcctcttcctgggcgccaatgctgtgtggtacggggtcgtgggagacgccgcctacaggtgggtgcccgaggggggcccgatgatctcctcctgcccgacccctcctaccccccacagcctctcccagcccgggtctctctcctctcctgccacacagcgcggggcccgtgtccggtctgatcccgctgagtgccgacacagttgccgtcggcctggtgtccagtgtggtcgtctatcccgtctacctg

SEQ ID NO: 315 - DNA, 263 bp, Felis catus
  variation (65)..(65): s represents polymorphism c or g
ctcagccttcagcaagaagccaaggtcagtggaagtggcagccagcagctctgctgtgttcgagsccgagacagagcggtcaggagtaaaggtgcgctggcagcgggggggcagtgacatcagcgccagtgacaagtatggcctagcagccgagggcacgaggcacactctgacagtgcgggacgtgggccccgccgaccagggaccctacgcagtcatcgctggctcctccaaggtcaagtttgacctcaaggtcatagaag

SEQ ID NO: 316 - DNA, 180 bp, Felis catus
  variation (46)..(46): y represents polymorphism c or t
ggctacatcctggagcgcaagaagaagaagagcttccggtggatgyggctgaactttgacctgctgcaggagctgagccacgaggcacggcgcatgattgagggcgtggtgtatgagatgcgagtctacgcggtcaatgccatcggcatgtccaggcccagccctgcctcccagcccttc

SEQ ID NO: 317 - DNA, 140 bp, Bos taurus
ctggagtgcgtttcaaaatggaacagataaaaaactagtaagtacataagtacatatctactggcctttgatctgactagttccccagtctcaggtctrtttgctgttaatcaccagtgagagaaggtcctaccctatct

SEQ ID NO: 318 - DNA, 175 bp, Bos taurus
  misc_feature (98)..(98): n is a, c, g, or t
tctgggagaggtacacggggtgggggaggggcgagtggctgcctcgggaggcacgggagaggtgaaaagcagctgagggatcacggatgctttgaacngggctgcaartagttgatacgaagtcaccgtgattgctttcgaccggtatgatttgtacaaaccagctcaacccttg

SEQ ID NO: 319 - DNA, 210 bp, Bos taurus
  variation (101)..(101): s represents polymorphism g or c
aaccgtgacggcatcatctgcaagtcggaccttagagagacctactcccagctcggtgagggcacccgtctcctgcctggcccagcccctctcacaccagsgacccggctcagagctgctgcctgncccngcctgtactcctgtcctggctcacaccacccacccacccctagaacacattccttccncttcacttcccccacccagtga

SEQ ID NO: 320 - DNA, 150 bp, Bos taurus
  misc_feature (31)..(31): n is a, c, g, or t
ggaagaggggcaagggagagctaaaggcctngacgggatcagtgagagaccagccagctgagtgacttagmaggggaggatggagccacctccaggagagttggctngaaaggatttcttctcngcccatcgatttcctgcctcactcct

SEQ ID NO: 321 - DNA, 140 bp, Bos taurus
  variation (73)..(73): r represents polymorphism g or a
tcttgaaaggttgctctgccacctgctgcttaaccttctcagcccctgtggtgtttccaaagggctggtcacrgtcctcaggcttgggtgtggcctgggtcttggagagggatggtgctgcgggcaagtctgtgtccatg

SEQ ID NO: 322 - DNA, 130 bp, Bos taurus
  misc_feature (60)..(60): n is a, c, g, or t
gagaggggaggggaggggagggttgccctcctacacctggccccacctgtacctccttcngtkagcctttgttctagctagaagggcccctgaattctccaggtaacccctgagagggaaggaaatgcct

SEQ ID NO: 323 - DNA, 145 bp, Bos taurus
  variation (71)..(71): y represents polymorphism t or c
tgtcagataaaatacaggatgtccagttatatttgaatattaagtaaaacaatgaatattttttcagtgtygcatgtacattgttccatgtaggacatgcttatattttagaaattattcattgtatactgtaaattccaatcga

SEQ ID NO: 324 - DNA, 150 bp, Bos taurus
  variation (79)..(79): y represents polymorphism c or t
cggtccatgccatgttactgtctgtaaccctgtgggcactgctagaacctcacttctgaccataactgaagcccagggygatgagaggtgatggagctctgactattaggccgcccagctctggtctgggttcttaaccacttctcaaga

SEQ ID NO: 325 - DNA, 142 bp, Bos taurus
  variation (62)..(62): y represents polymorphism t or c
gcagactcagcagccactaacaaggggctgcgagccatcaaaggggtccgtggaaaagactgtagagagcaygggaaaggagccactatgcaggaagtcacaggaagctttgcagaaaaactaacatttgagctggctccag

SEQ ID NO: 326 - DNA, 150 bp, Bos taurus
  variation (76)..(76): y represents polymorphism c or t
gtgccactccaaggtggtgaagaacaatctgaaccccagctgggagccgttccgtctgtccctgcactccctgtgyagctgtgangtccaccggcctctcaaggtgaggccccacgagtaagggcagcctggtagagcagccagcctctg

SEQ ID NO: 327 - DNA, 150 bp, Bos taurus
  misc_feature (56)..(56): n is a, c, g, or t
gacttcagaatggaaaccctctctccctaaagaaagccatacccagggagtccacntgggctgaataaccccraggactggcagaagggaagggaagaatgtagctgcagcctgaacttcactgttgtctnatccatgcccnactgcctt

SEQ ID NO: 328 - DNA, 150 bp, Bos taurus
  variation (79)..(79): y represents polymorphism t or c
gtcagagcccaggctggtccgaggccgcacccgctggcctccctgccccgtgagagggggaggcaggaacatcccatcyggaagtagccgctcttccaagtctggaatcaggaggagctcagtaaatgctggttgaatgaatgaatgaat

SEQ ID NO: 329 - DNA, 150 bp, Bos taurus
  variation (72)..(72): r represents polymorphism g or a
tgacctggtcactttctatgtggcttcctgtattccctttgttgtctaatgtcagaaactataactatctarttcacactaggttctctataaattatttgctgaacaaaatatttcttcttttgaaaataagagaaacatagagtttac

SEQ ID NO: 330 - DNA, 137 bp, Bos taurus
  variation (63)..(63): y represents polymorphism t or c
ctgggagtggagagtggattgggaagcctggggaactgtggacctgtgggcaatcccttagcytttctgagcctcagtttctccatctgtacaaaaggggcaatcataccnatttcacagtcaggtgaactgtgcag

SEQ ID NO: 331 - DNA, 170 bp, Bos taurus
  variation (88)..(88): k represents polymorphism g or t
gccgtctacacatgcatatctgaaaggaatgaccctcctaggcagaggaggagaaagaatcatttaaatgtctttacagtaatgctckaaaatttttggctggccagcatggctgctttattacatctctccaaagagcacantacctgccatcaagtgcagatacgcat

SEQ ID NO: 332 - DNA, 147 bp, Bos taurus
  variation (94)..(94): k represents polymorphism g or t
atgcttgttctcgcttgtgcagaaaacattgttccagattcaatcgactgggttcatgtcccctcacatagtttttaaggttatttatttaaakgtctaatgtattttattgtaacagacattgttttgccaacattgcctatttca

SEQ ID NO: 333 - DNA, 180 bp, Bos taurus
  misc_feature (28)..(28): n is a, c, g, or t
gcaggccttcgagtccatgctgcgctanatctactatggcgaggtcaacatgccgcccgaggactccctgcatcctagcctcagggcttccrgccacgcccggcagccttggaccccgccagccaggggctgggcngtttcccaaggattcntggtggccccgtctcccctgagctctct

SEQ ID NO: 334 - DNA, 160 bp, Bos taurus
  variation (78)..(78): y represents polymorphism c or t
gagcgcctgatggaagagggtctgggggcctgggttcttgggtccagaggatgaagtggcaggggacgccgattcttyggtcccaagagaggaagggcctgagtcttgctgaaggaggagactgggacctcaatttctgggtcccaaaagagaaatgttg

SEQ ID NO: 335 - DNA, 170 bp, Bos taurus
  misc_feature (25)..(25): n is a, c, g, or t
tgtcttccactttctactgcgattngtcacttcttcatccatgggagggagaggagagctcttctcagattgcctgatttcckattcctttcatcctcagccggctcttcccagacaaggagagcatgcttgatgcggctttcaccttggcagctgagatttccagcaag

SEQ ID NO: 336 - DNA, 128 bp, Bos taurus
  misc_feature (22)..(22): n is a, c, g, or t
ctttctgacttgaaaccatttngaggagacaggggggatctttaagaggtaacttcagtcttcgaggttagggtccccactttgtagaggggatgagaarttggttttgcagctgatgggtccagtgt

SEQ ID NO: 337 - DNA, 142 bp, Bos taurus
  variation (102)..(102): r represents polymorphism t or c
cctactcccagtccaagcagtttttactggactcaggtgagacccagagctgagccctcagctcccagctagtctggaccagcttcgagctgattgccaccycctgctccacctcccccaggctacttctctgacaacatcc

SEQ ID NO: 338 - DNA, 170 bp, Bos taurus
  variation (61)..(61): m represents polymorphism c or a
tccgtgcctgccctcagcctgcccagcggggaagctctggtgggtgtggaatggtggtggcagaaagggtmccgcgggtctcccattngtcttcccctgagtccctgcccagccgggactgcctggatctgagaggtgggacaaggaggtggcttngccgcaggtcaccg

SEQ ID NO: 339 - DNA, 180 bp, Bos taurus
  variation (87)..(87): s represents polymorphism c or g
ttctcatacaaaaagactttgttctgacagctgctcactgcagaggaaggtgaggggcagtagccagcctaccctacctcaggcctsgacagggacccgtcctctccccaggagctgagcccaggggtacccctccttcaccccaggatcctggaaatcaggaccacaagagctcatcag

SEQ ID NO: 340 - DNA, 170 bp, Bos taurus
  variation (88)..(88): y represents polymorphism c or t
aagcagaggaggtccagagaagcctctgccccacccctcaggcctctgttggctccctggccccactctcaaaatctgagacttgtaytcaacccttttctttcccaggaggttctagatctcctcagattccttcaacagcctcttcctgtaggaatgacccctcctgc

SEQ ID NO: 341 - DNA, 160 bp, Bos taurus
  variation (82)..(82): w represents polymorphism a or t
ccccagcacagccccttgccaaggtatacatctatggatatctttggggtaatgaggaggaaaaggtcttaaatggggtggwtccagagtaaaaggcacctgtcttttctccacagaaaaagggggtaaagcggaaagcagatactaccacccctacacc

SEQ ID NO: 342 - DNA, 170 bp, Bos taurus
  misc_feature (52)..(52): n is a, c, g, or t
ttgaacacttactatatacttggcattgttctaggttctgtgaatacagttntgaacaaaacagagcaaaaatccttgccttcatgggrtagggaatggggagacagacaatatacataataaataactaaatgtattttttgtgttaagaggtaataaatacagtggaa

SEQ ID NO: 343 - DNA, 170 bp, Bos taurus
  variation (87)..(87): r represents polymorphism g or a
ttctacccacctaaatgagacattccttttatgtctagccctcactcccagtgtaaacctgttgtgtttccctgtattttcctgcartgttgtcacagaggaagtggggtagtgtagttctgccttgatcacgctntctctrtcttagctgttrtttaaaattttattgt

SEQ ID NO: 344 - DNA, 148 bp, Bos taurus
  variation (81)..(81): y represents polymorphism t or c
aatcagttgacccttaaagacacttgtatgtatttcagaagttttctcatgccaagctaagagcagtttcacgtagtatayttcagggtcaggaaggcngcccataaagggcattgtttgtgatcctttgaggacgttgaggtgctgt

SEQ ID NO: 345 - DNA, 157 bp, Bos taurus
  variation (86)..(86): y represents polymorphism t or c
tggagcagcaccccagctgccggtacgatgctctggagatctttgccgggtctggaacctcgggccagcaactcggacgcttctgygggaccttccgacccgcgcctgtagtcgcctccggcaaccaggtgaccctgaggatgagatcggacgaggg

SEQ ID NO: 346 - DNA, 127 bp, Bos taurus
  misc_feature (22)..(22): n is a, c, g, or t
gccttgtcagtgggtggggtgnggttgttgatggcagcggtggtgaagtgataacaaacagccatctactgcctttttctcctggtaacygttagcatttcctcttgtcctcactagacttccctgg

SEQ ID NO: 347 - DNA, 225 bp, Bos taurus
  misc_feature (36)..(36): n is a, c, g, or t
cttcttttaatttccaggtctcagtctgggagcccngggaactgtgcccctgtttctgactsttgttgccttgagggttttggaggttgacatggtcagcaacccgaagaagcacctcagatggtctgtgagtttcagacacagtgataatcacagctggcccacagccagaggaggaggcatcctactaacaagagcagtggatgctttggttatgcaaatgta

SEQ ID NO: 348 - DNA, 329 bp, Bos taurus
  variation (160)..(160): r represents polymorphism g or a
tgaagtgggagtagggtgccggcaaggcaaggcatagagcatgggaaaggaggcagggtttggaaagcggaggcaggctggacagagacaagggttagtctctggaatgttgacatatgtggagcccaggggaggagggcctcatgtgccatgctaggcratacaagattctcttcaagtctggaggaactgctcaatgcagggggcttagagggccctacctgggtccttttctcatctctgccctgcaagctncccatgtgttctttaccccaaggtgtcggccatcacccatcccatggctatcaatctttcttcctggctttgag

SEQ ID NO: 349 - DNA, 472 bp, Bos taurus
  variation (349)..(349): r represents polymorphism a or g
gagagcaccctctcatccctccttcagcttgagcagaccatcatcttccagtctctccagggcaagacaatctgggaattaccctttacaagagaaaagcttaaaatattatgaaacttaaaataattagtgacactttctaaaaatgtaataaatctgcaaagactattttaactctagagagattctttaatttacaatttgttgagcttcataatcctattagaagtctgcccacaaaatactcaaaaatcccattcaaagcagaacagagaactgttcaacatctttccaacaggacagaagtcaacctgggaacagctgttctctctgcggctcaggtagtgaratgcacaggacctgtcctgggtactgtccctgtgagtattcaagctggactcattagctgcagttactttgctgccagtcatgattccatacatacacacacacacatgcacgcacatgcaca

SEQ ID NO: 350 - DNA, 488 bp, Bos taurus
  misc_feature (26)..(26): n is a, c, g, or t
tatctgtgcactcatcatgttcatanaaggattatttataatagacaaaattgtagaagaaacccaaaatgtccattgacgtttggaataaaaaatgcatgattggattcacaggtgaatgaataaatgaaaagtagtttatacatatgatggagtattattagccttaaaaaaaatctgacacgtgtgacaacatggatgaatcttgaggacattatattaagtgaartaagccaatcgtaaaagaaaaatgctatatggtttcacttatatgaggtatccagaacagtcaaactcatagaaacagaaagtataatggcagttgccaggggctggtggggaggtaaagtgaggagttaatgggactaaaatttcagtttttcaagataaaaaagttctgaagattagatgcacaaaatccgagtgcacttaacactgctgaatattcacttaaacggtaataaagtttatgccatgtatcctttgcg

SEQ ID NO: 351 - DNA, 418 bp, Bos taurus
  variation (152)..(152): r represents polymorphism g or a
aaaattgtgagtaaacataaaaggcaaaaagttatgacactgaaagatgagccacccaagtcagaaggtgtccagtatgctgctggggaagagtggaagacaactctaatgtggctgggccaaagcagaaataacattcagttgtggatgtrtctggtggtgaaagtaaaatctgatgctataagcaaaatgaactgagaactacctctactttagaactacctctagaaacatctacctgtttcattgacaactctaaagactttgacaatgtggatcatgccaagctgtgaaaaactcttaaagagatgggaataccagnccatcttacctgtctcctgagaaacctgtatgcaagacaagttgcaacagttgtatagaacaactgtatagaacaactgactggttcagaattgag

SEQ ID NO: 352 - DNA, 199 bp, Bos taurus
  variation (75)..(75): y represents polymorphism t or c
ttctatagacatagacttagaagagaggacatttgtttccctcctaggttcttaaaggaaaatagctttcaaaayttaatttttattatgtttttgcaaacttgctaaacctagagaaaagcaatgttgttagggggaggggaagtaagtttacataatacttataaatatttccttgatatctataaatatttgctca

SEQ ID NO: 353 - DNA, 316 bp, Bos taurus
  variation (109)..(109): r represents polymorphism g or a
caagctttaagagccactctctgccccctcataatctggtctcccccaccacccagacctgtctccgccgggccgctcagttcccctcctcctacagactcactgaacrtgctgcctgactccctggtgtttccccccaccccccacagtactgtgcagccctggactccctgatcagcatctccaactgcagtgtcatccaaaggaccaagaggatgctgaatgcactctgtcctcacaagccctcagctaaggtaaggcccccctgtcctttgacctggcacccacttctgccagccctgggctcaccctggga

SEQ ID NO: 354 - DNA, 324 bp, Bos taurus
  misc_feature (11)..(11): n is a, c, g, or t
actttggggtnaaacttccagaataaatatttgcaggggaagggtcactcagcccagcccctccactgcctgggcatctgcattcttggaaggaaggaggtgatgtagggaggagggagagtgaggatgccacgtggagccnagctgcatttcacrtcacagttgatgtcaccacttggcagatgtcccagggcatggggggaatggtagactgtcaaagcaggtgtgaaacactgaactaaaacagacatttagcaaatcaaacaaggagaaagtggttaaganccagctgaagacactggcaaggcaagttctgatgctggc

SEQ ID NO: 355 - DNA, 264 bp, Bos taurus
  misc_feature (88)..(88): n is a, c, g, or t
ttactattatgcatttctgatcaccgacctaaactggggtttcaactcttattagcatttttagtatgatcttacttgtttctgttgngacctcattctcctacttactgtaaaactanntctatcttctctattgytcagagtcttcattgaactaaactggtaaacaaaccacacagatgtgaaaataaatttctaatctattaagttaaattttgctatataaaaacacttgttaaaatttaaaaaatagaactaccattt

SEQ ID NO: 356 - DNA, 267 bp, Bos taurus
  variation (118)..(118): y represents polymorphism c or t
aactgtagaatctatatgcttagcttgtttagtggttcacagggtaatcaatagtaaaagtgggttaaattcaatgtaatccagacaactgcaaggtatgtatatatattatacatayataatttaaatgttgatcataaaactcacaaagaaatttatattaaaaaatagaatgctatcatgcattattttatttagaccttttagtcaccaagaggaggtaatttttccttagtctgtaaaattcaggaaattgaccatttaaag

SEQ ID NO: 357 - DNA, 172 bp, Bos taurus
  misc_feature (5)..(5): n is a, c, g, or t
caganagttataaatgctaatttaagagtataatgccatccaastgcctaattgagaacaatgtaagaataaatttgaggaaaataaatacttgcaaactgtgctaattatatatttgatcagaaaattcacatagaaaatattttaaattggtgtttttccacagtggatg

SEQ ID NO: 358 - DNA, 215 bp, Bos taurus
  variation (87)..(87): y represents polymorphism t or c
gattggagtgttgcatgtataagccagggaacaccagcaattgtcagcaacaccagaagctaagagaaagacatggaacagattctytcctggacccttcagggaaagcatgatcttgttaacattttgaactaatagcntccagaactgtgagaaactgggagtttctattgtttaaagtgaaagtgaagttgctcagtcgtatccaactcttt

SEQ ID NO: 359 - DNA, 220 bp, Bos taurus
  variation (111)..(111): y represents polymorphism c or t
accacgctcaaagctcaggtcctgagaatatgaccctccccaccaggccccagattctgcagccaatgtgaccttgattcttctgggaactactcaaaggcccaaggctcytctacccaagggttgctgaaaatccatcaagagcccagcaagagggagaggcagggtgtgggtctgagctccaacccacaggcaacaagtcttttngggggagagaggg

SEQ ID NO: 360 - DNA, 360 bp, Bos taurus
  variation (240)..(240): r represents polymorphism a or g
ctatgtcagaacgtgtgcttgcctgaaatggaatctgaataggcatcctgcatcatatccaccagcataagagaatggggagagtcctgaggttctgaggcctgacaagatgccataagtgcccatgaaccccccccacccccagaccagatagtacaccctgactatctcccaaatcctggcaggtcaggcagttgcgttcaacgtgaccttccggagggccaagggctaccccatcgrcctgtactacctgatggacctctcctactccatggtggatgacctcgtcaacgtcaagaagctggggggtgacctgctccgggccctcaatggcatcaccgagtcgggccgcattggtga

SEQ ID NO: 361 - DNA, 117 bp, Bos taurus
  variation (55)..(55): y represents polymorphism c or t
gtgcaaatggctgaagaacattctgaatttgtgattggttttatttctggctccygagtaagcatgaaaccagaatttcttcacttgactccaggagttcagttagaagcaggaggt

SEQ ID NO: 362 - DNA, 597 bp, Bos taurus
  misc_feature (169)..(169): n is a, c, g, or t
cttattaattaaggacaaacgtcatttcactggactgggtaggggttggaagtccaaggactagcaagatttgtgcacaagcctggcccacctcatgggcccctgggtcccctgaagaagggccagcctggggtgggtcactggtacaggagcccccagccctcaccanacatgtcctctttaggtgtttcagcggcgccctctccagaagaagaggagggtagtgcacccacagcaggccctgacgtggaggagtggatggtgacacaagtggggcctggcgtggctgctgtccccatcggggaggagacgactgcaatcccaggcttcaccgttgagccagaaaacaagacggaatgggaacttgcctacaccccagcgggcactttgccactacggcacaggtccgtccgggctctcctgcatgtcctgctgcctccctgggccagggtgtggcctggaaggggggaggaggaagtgttctctccctgggacccgtgatctgttccccagccctgacccccagccctgattttatttaagtgtgctgccatgggtaacttcagccacctcagcaggcatccaggaccaatccta

SEQ ID NO: 363 - DNA, 279 bp, Bos taurus
  variation (180)..(180): k represents polymorphism g or t
cggtgtctgcgcttctgaccgtctgtccttcccgtctgtcctgcaggccctggccgacctggtccactcacacctccagtccaaggagcgctgctccacacggctgactctgagctgtccgctctgcgtgaaccccacctgcagggagaccaaatccttcttcaccagccagcagctgtkaccctggcggcacgccgctgggaggtgcgcgtgcccgcctcccgacacctccgaggaggaggagggcgcatccggccgttagggaggaggttacatccg

SEQ ID NO: 364 - DNA, 330 bp, Bos taurus
  variation (199)..(199): y represents polymorphism c or t
tccctgcagctctgagtcctggaaaataaggctcagttgatgcttggcaaaaggctcagactgagcctggcttggctcatacagggagcaaaagctcagtgccattggctgcctagatgaagaggaaagcaagtagacagagcatgccctctgactggcctgtcctattttgcaggtgctgctgataaagctggaactygagaaaaccaggttggctgggaattgctgtcttgctgggaaggagggaaggccacaggcctgagccacccttgagtttgctccctgctaagttttctgaggctttcttttgtgaggagaccctggaggttc

SEQ ID NO: 365 - DNA, 360 bp, Bos taurus
  variation (236)..(237): polymorphism represented by presence of cg or at
ctgttaataaccaagacgatcagagccaccggagagccacgttcacatctgaagctcttagtccttttcggccccctgttgttatatccctccttcctgccccactgtccctgggagtcagggagccctgaatctcctccatttggagaagcgtcaaactgggagacttgattctgccccaggccagatgttcatttgctttgctcatagggaccaacgcttgtggcgtgaacaacggcggatgcactcacctctgctttgccagaacctcggactttgtgtgtgcctgtcctgatgagcccgacggccggccctgctccctcggtgagttggactgacgggcccccctgcaacagcgga

SEQ ID NO: 366 - DNA, 360 bp, Bos taurus
  variation (271)..(271): y represents polymorphism c or t
tggtcacccgtcacgatgtccggcaaaggctccgtggttctggcctacagtgggggcctggacacctcctgcatcctcgtgtggctgaaggagcaaggctatgacgtcattgcctacctggccaacatcggccagaaagaagactttgaggaagccaggaagaaggcgctgaagcttggggccaaaaaggtgttcattgaggacatcagcaaggagtttgtggaggagttcatctggccggccatccagtccagcgcactgtacgaggacygatacctcctgggcacctctctcgccaggccctgcatcgcccgcaagcaggtggagatcgcccagcgagaaggagccaagtatgtgtct

SEQ ID NO: 367 - DNA, 480 bp, Bos taurus
  variation (271)..(271): k represents polymorphism g or t
tttttaaaattatagattgtaaaggcaatatcactatgggaaaaaaaaatgattctaaggttttttcaaaagctctcctctgtaatccccaggaatggaaatggttgcatttttaccttaaggtctaagagtgggctctaaacatgtattttgtaaaatattataggaattaaacttgtgttgtttctttttgttcagtggccctcagattctcaagagcttaattctaaggaactttcagctggctcacaatttgtaggtctcatggcakttctcacagcatgtttttccagtggctttgctggggtttactttgagaaaatcttaaaagaaaccaaacaatcagtgtggataagaaacattcaacttggtaagttttaaatgttttctaacattacttttaaagtgattatattgttatatttaaagatttctatgtatctttaattaaataaaccttataaaaactgcttgttgttg

SEQ ID NO: 368 - DNA, 654 bp, Bos taurus
  misc_feature (188)..(188): n is a, c, g, or t
ggggagccatgagttgagcaggaccctgagagcaagcaccccttcctgctccctgcgggacgatgcctgcacttggctcccagaggcggctgctgggttcccttaactgcacgcccccagccaccctccccttcaccctggcccccaaccggacggggccccagtgcctggaggtgtccatccctgangggctctttctcagcctggggctggtgagtctcgtggagaacgtgctggtagtggctgccattgccaagaaccgcaacctgcactcccccatgtactactttatctgctgcctggctgtgtctgacttgctggtgagcgtcagcaacgtgctggaganggcagtcatgcygctgctggaggccngtgtcctggccacccaggcggccgtggngcagcagctggacaatgtcatcgacgtgctcatctgcggatccatggtgtccagcctctgcttcctgggtgccattgntgtggaccgctacatctccatcttctacgccctgcggtaccacngtgttgtgacactgccccgagcgtggaggatcattgcggccatctgggtggccagcatcctcaccagcctgctcttcatcacctactacaaccacaaggtcatcctgctgtgcctcgttggcctcttcatag

SEQ ID NO: 369 - DNA, 654 bp, Bos taurus
  misc_feature (188)..(188): n is a, c, g, or t
ggggagccatgagttgagcaggaccctgagagcaagcaccccttcctgctccctgcgggacgatgcctgcacttggctcccagaggcggctgctgggttcccttaactgcacgcccccagccaccctccccttcaccctggcccccaaccggacggggccccagtgcctggaggtgtccatccctgangggctctttctcagcctggggctggtgagtctcgtggagaacgtgctggtagtggctgccattgccaagaaccgcaacctgcactcccccatgtactactttatctgctgcctggctgtgtctgacttgctggtgagcgtcagcaacgtgctggaganggcagtcatgcngctgctggaggccggtgtcctggccacccaggcggccgtggngcagcagctggacaatgtcatcgacgtgctcatctgcggatccatggtgtccagcctctgcttcctgggtgccattgntgtggaccgctacatctccatcttctacgccctgcggtaccacngtgttgtgacactgccccgagcgtggaggatcattgcggccatctgggtggccagcatcctcaccagcctgctcttcatcacctactacaaccacaaggtcatcctgctgtgcctcgttggcctcttcatag

SEQ ID NO: 370 - DNA, 360 bp, Bos taurus
  variation (160)..(160): y represents polymorphism c or t
tataaaatatgaaagaacttttattgttacccttaaacattttaagtcaccttcagaacataataatatattaatacaaactgattatgtctattaacaaggtgtctttgacatattttagatatatccacatatccactggaaaatgcccctattggayataacagacaatacaatatggtaccattttggcctccagttaccaacatagaaatgtttgttactgctccagacaacctgggctatacttangaagttcaatggccaagtgagtactgaaaatgtatttttactgtggaaatttccaaaatcaaacttgttacctttaaagtaatctcagttttctgagataaagtaacc

SEQ ID NO: 371 - DNA, 209 bp, Bos taurus
  variation (115)..(115): polymorphism represented by presence or absence of c
gcagatcgtctgcagcagactggaggagtacaacagtcgccaggctttatgcaacgggacgtctgagggaccattactgcgcaatcctggaaaccacgacaaagccaggacccccgaggctcccctcctcggctgatgtggagttttgcctgagtttgacccagtatgaatcaggttccatggataaagctgccaatttcagctttaga

SEQ ID NO: 372 - DNA, 109 bp, Bos taurus
  variation (45)..(45): m represents polymorphism c or a
gaaggcctcaaattccattgaagattccagcctacaatgggcagmcgtagcattgccagcattcttttctcttgtgatcgggtttgcttttggggccttttactggaag

SEQ ID NO: 373 - DNA, 360 bp, Bos taurus
  variation (189)..(189): s represents polymorphism g or c
agcatcctcggggcgtctgagctggccctcataagataacccctgggactggggtctctggacttgcccttgtggaggcctcctgacctgggccagggaaggacaggccccagggatagaggctgggcaggtcagtggccgccagcccctggcagtgccgttttcctacagctcctcggagtggaacgscgtggacccttacatgcgggagcagctccgggtcaagatggaggatggggagttctggtgagcagccccctcctcagtctgagtgggcaccccagctcccaaccccacccccctgaaaaccagctgtgccatgtctcttgatgcctcgactgggcatcctggttcactctc

SEQ ID NO: 374 - DNA, 300 bp, Bos taurus
  variation (162)..(162): s represents polymorphism g or c
attttgaactctcatctttcaacacttaagtcctacctagaatggcagttatttgtttttctgttaaaacggcacctctgtgtggcatcagcaggtattgcaatttgcttgtgtgattcttgctgaatttggaaggaaggaattgcattgtttcaaattttstacccaaagtgaaatttgtcacatgtaaatcatactaatttaaattctcacaattgactacataaaacacaagtgttatgaattgctttctactcctcagagaaaagtagcaatatgtgtcatattattaaccccatg

SEQ ID NO: 375 - DNA, 360 bp, Bos taurus
  variation (193)..(193): m represents polymorphism c or a
ggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttmgatgtccagagagatgccagcagtgacggctccttggaagacgatgactaccacgccaggacggaaacggtcattaccatgcccacggagtgtgagtagtcctgctggtgcagagcaacgactctgctgactgctgttctagtgttcatgagaaaccgatctatttt

SEQ ID NO: 376 - DNA, 360 bp, Bos taurus
  variation (169)..(169): m represents polymorphism a or c
agtattcacgagactgtcagggacttaaagaggctatttgctagacggcaccagatctgattcttggtatttgttttttgtagatattttcagggctgttggtaaatctcaaaaccgtcgtgccttggttgtcatggcttcaatacttgagcattcctcgatacggctmtgcggtatgttctccttatctgtcaccgtgctggttcattgtccccatgctggaaacagccagaataaagcctctcatatccttggccatgagctgtgcaagttttaggacaatgaaggagagtttcctattaagccttgggtcaagttgataatcacctgggatttctctagtcaccttgttgtctgagg

SEQ ID NO: 377 - DNA, 359 bp, Bos taurus
  variation (197)..(197): y represents polymorphism c or t
gcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccaycgaagcagtagagagcactgtagctactctagaagmttctccagaagttattgagagcccacctgagatcaacacagtccaagttacttcaactgcggtctaaatactctaaggagacatcaaagaagacaacgcaggtaaataagcaaaatgaataacagc

SEQ ID NO: 378 - DNA, 101 bp, Bos taurus
  variation (40)..(40): y represents polymorphism t or c
agtagaggcagaaatcgtcactgatcctctgacagccgaygtagtgtcagaagaagtattggtagcagattgtgcctcagaagcagtcatagatgccaacg

SEQ ID NO: 379 - DNA, 100 bp, Bos taurus
  variation (68)..(68): w represents polymorphism t or a
atgtggctgcccacaagggtaaaaaaatgcaccagtgtagacattgtgactttaagattgcagatccwtttgttctaagtcgccatattctctcagttca

SEQ ID NO: 380 - DNA, 272 bp, Bos taurus
  variation (137)..(137): s represents polymorphism c or g
caccaagtgccgttcacctgaactggagactttctcatgtcactggacagatggggctaatcacagtttacagagcccaggatctgtacagatgttctatatcagaaggtatgggcttcatgcttttctgatttctstccatgaattttctgatgaaaatccattgagtgtcatgcagtrgtgggaatggaaataatcttctttggtgatctaaatgcattcacccattcattcatttaaatatattagttaagcccttactatatgttggg

SEQ ID NO: 381 - DNA, 181 bp, Bos taurus
  variation (74)..(74): m represents polymorphism c or a
gatgaactccaggataaaatccacccctttgcccagacacagtctctagtctatcccttccctgggcccatccmtaacagcctcccacaaaacatccctcctcttactcaaacccctgtggtggtgccgcctttccttcagcctgaagtaatgggagtctccaaagtgaaggaggctatgg

SEQ ID NO: 382 - DNA, 1709 bp, Felis catus
atggggaaaaaactggatctttccaagctcacggacgacgaggccaagcaatctgggaagtggttcagcgggactttgatctgagaaggaaagaagaggaaaggctggggggattgaaggacaggattaagaaagagagctcccagagggagctgctctcggatgcggcccacctgaatgagacccactgcgcccgctgcctgcagccctaccggctcctcgtggcccccaagaggcaatgcctggactgtcacctcttcacctgccaagactgtagccacgcccacccggaggaggagggctggctctgtgacccctgccacctggccagggttgtgaagatgggctcactggagtggtactacgggcacctgagagcccgcttcaagcggtttgggagcgccaaagtgatccggtccctgtgcgggcggctgcagggtggaggtgggcctgagccaagccctggagagggaagtggagacagtgagcagacagaagaggatggagaactggacacagtggcccaggcccaaccccttgggagcaaaaaaaagcgcctctccattcacggcttggactttgatgcagactctgatggctcgactcagtccggcggtcaccccccatatctgtccccggtccccatggccacagacagcctgcaggccctcacaggtgaatcccgtgccaaggacacctcccaggaggccgtggtcctggaagaggctgatgtcggtgcccctggactccaccctcatccagaagagcagacagacagcctctcagctgccagacaggacaccctcactgagccccgcttccccagacagtcctgcacaacagccctggggttggctgtcacacccggtccaggcgtcatcagcagtagtgagcggctctcctcccggtacccggctgacgaaggcacctccgatgacgaggacaccggggctgacggtgtggcctcccagagcctcacgtggagggactgcgccccggctgagagccagcatctcaccggccaccagcccacagacgccgacagagaagaagagaccctaaagaggaagctggaggagatgaccagccacatcagtgaccagggggcctcgtccgaggaggaggggagcaaggaggaagaggcaggactgaacaggaaaacctccatcgaggacctccccggggcagccccagaggtgctcgtggcttcgggccaaacgtccagacaggaaacaagtccccggggtcctcaggaactcatgcagcccggcagaaccacggaccaggagctgctggagctggaagacagagtggccgtgacggcctctgaggttcagcaggtggagagtgaggtttctaacatcaagtccaagattgccgccttgcaggctgccgggctctcggtgagaccctcgggaaagccccagcggaggtccaacctcccgatatttcttccccgactcgttgggagattgggccagacccctaaggatccaaacgcagagccttcggatgaggtcaaggtgatgactgcaccctaccttctgagaaggaagttcagtaatcccccaaaaagtcaagataaggctggcgactcctttgaccggcagtcagcgtaccgcggatccctgacgcagagaaaccccaacagcaggaagggagtggccaaccacagctttgcaaaacccgtgatgacccagcggccctga